(19) United States
(12) Patent Application Publication
(10) Pub. No.: US 2026/0265199 A1
(43) Pub. Date: Sep. 10, 2026

(54) CRYSTALLINE FORMS OF (R)-2-AMINO-5-(4-(2-(3,5-DIFLUOROPHENYL)-2-HYDROXYACETAMIDO)-2-METHYLPHENYL)-N-ISOPROPYLNICOTINAMIDE AND METHODS FOR USING THE SAME

(71) Applicant: HIBERCELL, INC., New York, NY (US)

(72) Inventors: Ruiping WANG, Beijing (CN); Meiting SHI, Beijing (CN); Travis HOUSTON, West Lafayette, IN (US)

(73) Assignee: HIBERCELL, INC., New York, NY (US)

(21) Appl. No.: 19/471,272

(22) PCT Filed: Mar. 29, 2024
(86) PCT No.: PCT/US2024/022325

§ 371 Date: Sep. 30, 2025

(30) Foreign Application Priority Data
Mar. 30, 2023 (WO) PCT/CN2023/085125

(51) Int. Cl. C07D 213/82; A61K 31/4418; A61P 35/00

(52) U.S. Cl. CPC C07D 213/82; A61K 31/4418; A61P 35/00

(57) ABSTRACT

Provided herein are crystalline forms of a compound of formula (I) and pharmaceutical compositions thereof. Also provided herein are methods of using the crystalline forms and pharmaceutical compositions to treat a cancer and viruses (e.g., coronaviruses), and other diseases and disorders involving protein kinase R (PKR)-like endoplasmic reticulum kinase (PERK).

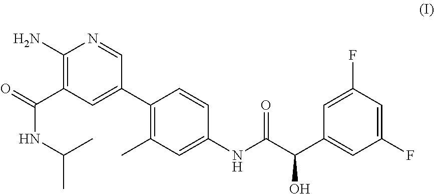 [SMILES: Cc1cc(NC(=O)[C@H](O)c2cc(F)cc(F)c2)ccc1-c1cnc(N)c(C(=O)NC(C)C)c1]

### CROSS REFERENCE TO RELATED APPLICATION

This application claims the benefit of, and priority to, International Application No. PCT/CN2023/085125, filed Mar. 30, 2023, the contents of which are incorporated by reference herein in their entirety.

### BACKGROUND

Protein kinase R (PKR)-like endoplasmic reticulum kinase (PERK), an eIF2 kinase involved in the unfolded protein response that regulates protein synthesis, aids cells to alleviate the impact of endoplasmic reticulum stress and has been implicated in tumorigenesis, cancer cell survival, and viral infections (e.g., coronaviruses). As such, PERK modulating compounds (e.g., PERK inhibiting compounds) are believed to be useful in the treatment of a wide range of conditions, diseases, and disorders, including but not limited to various cancers. Developing solid forms, including crystalline forms, of PERK inhibiting compounds that provide the physicochemical properties necessary to manufacture a drug product with the required stability and efficacy represents a significant challenge due to the unpredictability in the outcome of solid form screening given for a compound and the subsequent unpredictability of the physicochemical properties of any solid forms identified. Thus, there is an unmet need for new solid forms of PERK inhibiting compounds that provide the required physicochemical properties.

### SUMMARY

The present disclosure provides crystalline forms (e.g., crystalline salt forms and crystalline free base forms) of a compound of formula (I)

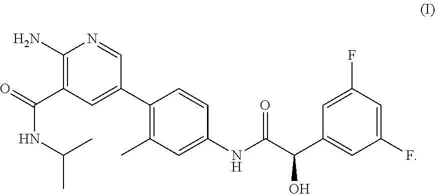 [SMILES: Cc1cc(NC(=O)[C@H](O)c2cc(F)cc(F)c2)ccc1-c1cnc(N)c(C(=O)NC(C)C)c1]

The disclosure additionally provides pharmaceutical compositions comprising a crystalline form (e.g. crystalline salt form or crystalline free base form) of the compound of formula (I) described herein. The salt forms of the compound of formula (I) or pharmaceutical compositions of the disclosure are useful in the treatment of a condition, disease, or disorder described herein (e.g., a cancer).

In one aspect, the disclosure provides a fumarate salt of a compound of formula (I)

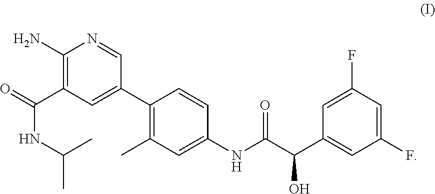 [SMILES: Cc1cc(NC(=O)[C@H](O)c2cc(F)cc(F)c2)ccc1-c1cnc(N)c(C(=O)NC(C)C)c1]

In another aspect, the disclosure provides a crystalline fumarate salt of a compound of formula (I)

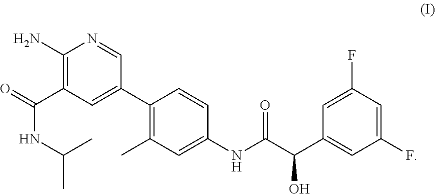 [SMILES: Cc1cc(NC(=O)[C@H](O)c2cc(F)cc(F)c2)ccc1-c1cnc(N)c(C(=O)NC(C)C)c1]

In another aspect, the disclosure provides a crystalline mono-fumarate salt of a compound of formula (I)

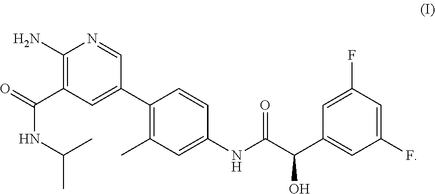 [SMILES: Cc1cc(NC(=O)[C@H](O)c2cc(F)cc(F)c2)ccc1-c1cnc(N)c(C(=O)NC(C)C)c1]

In another aspect, the disclosure provides Form A of a mono-fumarate salt of a compound of formula (I)

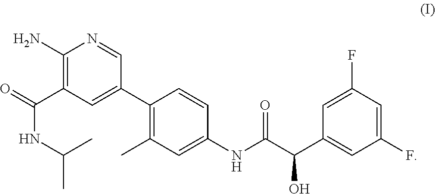 [SMILES: Cc1cc(NC(=O)[C@H](O)c2cc(F)cc(F)c2)ccc1-c1cnc(N)c(C(=O)NC(C)C)c1]

In another aspect, the disclosure provides Form B of a mono-fumarate salt of a compound of formula (I)

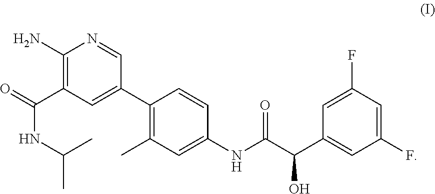 [SMILES: Cc1cc(NC(=O)[C@H](O)c2cc(F)cc(F)c2)ccc1-c1cnc(N)c(C(=O)NC(C)C)c1]

In another aspect, the disclosure provides Form C of a mono-fumarate salt of a compound of formula (I)

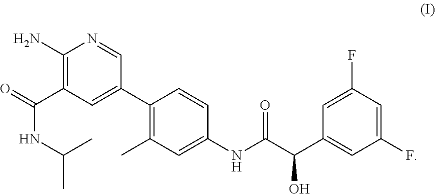 [SMILES: Cc1cc(NC(=O)[C@H](O)c2cc(F)cc(F)c2)ccc1-c1cnc(N)c(C(=O)NC(C)C)c1]

In another aspect, the disclosure provides a crystalline hemi-fumarate salt of a compound of formula (I)

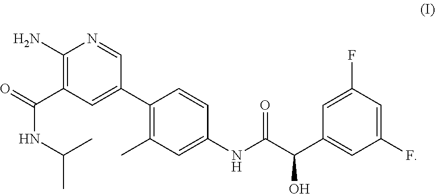 [SMILES: Cc1cc(NC(=O)[C@H](O)c2cc(F)cc(F)c2)ccc1-c1cnc(N)c(C(=O)NC(C)C)c1]

In another aspect, the disclosure provides Form A of a hemi-fumarate salt of a compound of formula (I)

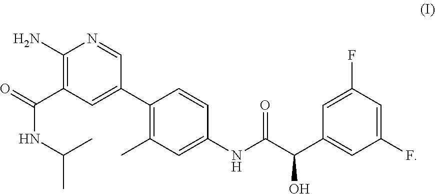 [SMILES: Cc1cc(NC(=O)[C@H](O)c2cc(F)cc(F)c2)ccc1-c1cnc(N)c(C(=O)NC(C)C)c1]

In another aspect, the disclosure provides Form B of a hemi-fumarate salt of a compound of formula (I)

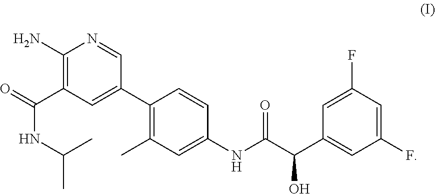 [SMILES: Cc1cc(NC(=O)[C@H](O)c2cc(F)cc(F)c2)ccc1-c1cnc(N)c(C(=O)NC(C)C)c1]

In another aspect, the disclosure provides pharmaceutical compositions generally comprising a salt form (e.g., a crystalline salt form) of the compound of formula (I) described herein and a pharmaceutically acceptable excipient.

In another aspect, the disclosure provides dosage forms (e.g., capsules) generally comprising a pharmaceutical composition described herein.

In another aspect, the disclosure provides methods of treating a cancer in a subject in need thereof, the methods generally comprise administering an effective amount of a salt form (e.g., a crystalline salt form) of the compound of formula (I) or a pharmaceutical composition described herein.

### BRIEF DESCRIPTION OF THE FIGURES

FIG. 1 is an exemplary X-ray powder diffraction (XRPD) pattern of Form A of the mono-fumarate salt of the compound of formula (I), prepared in accordance with Example 3.

FIG. 2 is an overlay of differential scanning calorimetry (DSC) and thermogravimetric analysis (TGA) thermograms of Form A of the mono-fumarate salt of the compound of formula (I), prepared in accordance with Example 3.

FIG. 3 is an exemplary XRPD pattern of Form B of the mono-fumarate salt of the compound of formula (I), prepared in accordance with Example 8.

FIG. 4 is an overlay of DSC and TGA thermograms of Form B of the mono-fumarate salt of the compound of formula (I), prepared in accordance with Example 8.

FIG. 5 is an exemplary XRPD pattern of Form C of the mono-fumarate salt of the compound of formula (I), prepared in accordance with Example 9.

FIG. 6 is an overlay of DSC and TGA thermograms of Form C of the mono-fumarate salt of the compound of formula (I), prepared in accordance with Example 9.

FIG. 7 is an exemplary XRPD pattern of a crystalline fumarate salt of the compound of formula (I) crystallized from 0.5 or 1.1 equivalents of 0.1 M fumaric acid in methanol solution at room temperature, obtained in accordance with Example 10.

FIG. 8 is an exemplary XRPD pattern of Form A of the hemi-fumarate salt of the compound of formula (I), prepared in accordance with Example 11.

FIG. 9 is an overlay of DSC and TGA thermograms of Form A of the hemi-fumarate salt of the compound of formula (I), prepared in accordance with Example 11.

FIG. 10 is an exemplary dynamic vapor sorption (DVS) isotherm plot of Form A of the hemi-fumarate salt of the compound of formula (I), prepared in accordance with Example 11.

FIG. 11 is a stack plot of XRPD patterns of Form A of the hemi-fumarate salt of the compound of formula (I) obtained from slurrying Form A of the hemi-fumarate salt of the compound of formula (I) and Form B of the hemi-fumarate salt of the compound of formula (I) in isopropanol, carried out in accordance with Example 14.

FIG. 12 is a stack plot of XRPD pattern of solids obtained after adding Form A of the hemi-fumarate salt of the compound of formula (I), prepared in accordance with Example 15, to water, FeSSIF, and FaSSIF solution. Sampling of the solids occurred at 24 hours.

FIG. 13 is an exemplary XRPD pattern of Form B of the hemi-fumarate salt of the compound of formula (I), prepared in accordance with Example 16.

FIG. 14 is an overlay of DSC and TGA thermograms of Form B of the hemi-fumarate salt of the compound of formula (I), prepared in accordance with Example 16.

FIG. 15 is a stack plot of XRPD pattern from a competitive slurrying of Form A of the hemi-fumarate salt of the compound of formula (I) and Form B of the hemi-fumarate salt of the compound of formula (I) in isopropanol at room temperature and at 50° C., carried out in accordance with Example 17. Sampling of the solids took place after 3 days.

FIG. 16 is an exemplary XRPD pattern of the L-tartrate salt of the compound of formula (I), prepared in accordance with Example 19.

FIG. 17 is an overlay of DSC and TGA thermograms of the L-tartrate salt of the compound of formula (I), prepared in accordance with Example 19.

FIG. 18 is an exemplary XRPD pattern of Form A of the tosylate salt of the compound of formula (I), prepared in accordance with Example 20.

FIG. 19 is an overlay of DSC and TGA thermograms of the Form A of the tosylate salt of the compound of formula (I), prepared in accordance with Example 20.

FIG. 20 is an exemplary XRPD pattern of a solid material comprising the tosylate salt of the compound of formula (I) crystallized from 1.1 equivalence of 0.1 M p-toluenesulfonic acid in MeOH, prepared in accordance with Example 21.

FIG. 21 is an exemplary XRPD pattern of a solid material comprising the tosylate salt of the compound of formula (I) crystallized from 14 V of EtOAc as an anti-solvent and a seed of the tosylate salt of the compound of formula isolated from salt screening experiment (Example 18), prepared in accordance with Example 22.

FIG. 22 is an exemplary XRPD pattern of Form A of the succinate salt of the compound of formula (I), prepared in accordance with Example 23.

FIG. 23 is an overlay of DSC and TGA thermograms of the Form A of the succinate salt of the compound of formula (I), prepared in accordance with Example 23.

FIG. 24 is an exemplary XRPD pattern of a solid material comprising the succinate salt of the compound of formula (I) crystallized from 1.1 equivalence of 0.1 M succinic acid solution in MeOH, prepared in accordance with Example 24.

FIG. 25 is an exemplary XRPD pattern of a solid material comprising the succinate salt of the compound of formula (I) crystallized from 1.05 equivalence of 0.1 M succinic acid solution in MeOH and the seed of a succinate salt from the salt screening experiment (Example 18), prepared in accordance with Example 25.

FIG. 26A is an exemplary XRPD pattern of Form A of the compound of formula (I), prepared in accordance with Example 27.

FIG. 26B is an XRPD pattern of Form A of the compound of formula (I) calculated from single-crystal X-ray diffraction data, prepared in accordance with Example 27.

FIG. 27 is an exemplary DSC thermogram of Form A of the compound of formula (I), prepared in accordance with Example 27.

FIG. 28 is an atomic displacement ellipsoid diagram of Form A of the compound of formula (I).

FIG. 29A is an exemplary XRPD pattern of Form A of the compound of formula (I) prepared by storing Form A in a mixture of water/acetone (80:20 v/v) under ambient conditions for four days, as carried out in accordance with Example 29.

FIG. 29B is an exemplary XRPD pattern of Form C of the compound of formula (I) prepared by storing Form A of the compound of formula (I) in a mixture of acetonitrile/water (92:8 v/v) under ambient conditions for four days, as carried out in accordance with Example 29.

FIG. 29C is an exemplary XRPD pattern of Form H of the compound of formula (I) prepared by storing Form A of the compound of formula (I) in a mixture of isopropanol/water (90:10 v/v) under ambient conditions for 27 days, as carried out in accordance with Example 29.

FIG. 29D is an exemplary XRPD pattern of a mixture of Form A and Form H of the compound of formula (I) prepared by storing Form A in a mixture of isopropanol/water (94:6 v/v) under ambient conditions for four days, as carried out in accordance with Example 29.

FIG. 30 is an exemplary XRPD pattern of Form B of the compound of formula (I), prepared in accordance with Example 30.

FIG. 31 is an exemplary DSC thermogram of Form B of the compound of formula (I), prepared in accordance with Example 30.

FIG. 32 is an exemplary thermogravimetric analysis (TGA) thermogram of Form B of the compound of formula (I), prepared in accordance with Example 30.

FIG. 33 is a cyclic DSC thermogram (multiple heating+cooling cycles) of the compound of formula (I) (starting material Form B of the compound of formula (I)), as carried out in accordance with Example 31.

FIG. 34 is a stack plot of XRPD patterns from a variable-temperature XRPD study of the compound of formula (I) (starting material Form B of the compound of formula (I)), as carried out in accordance with Example 31.

FIG. 35 is an exemplary XRPD pattern of Form C of the compound of formula (I), prepared in accordance with Example 32.

FIG. 36 is an exemplary DSC thermogram of Form C of the compound of formula (I), prepared in accordance with Example 32.

FIG. 37 is an exemplary TGA thermogram of Form C of the compound of formula (I), prepared in accordance with Example 32.

FIG. 38A is an XRPD pattern of a mixture of Forms B and C of the compound of formula (I) obtained from acetone, as further described in Example 33.

FIG. 38B is an XRPD pattern of Form B of the compound of formula (I) obtained after storing a mixture of Forms B and C of the compound of formula (I) under ambient conditions for 13 days, as further described in Example 33.

FIG. 38C is an XRPD pattern of Form C of the compound of formula (I) obtained from acetonitrile, as further described in Example 33.

FIG. 38D is an XRPD pattern of Form B of the compound of formula (I) obtained after storing Form C of the compound of formula (I) under ambient conditions for 25 days, as further described in Example 33.

FIG. 38E is an XRPD pattern of Form C of the compound of formula (I) obtained from a mixture of acetonitrile and water, as further described in Example 33.

FIG. 38F is an XRPD pattern of a mixture of Form B and Form C of the compound of formula (I) obtained after storing Form C of the compound of formula (I) under ambient conditions for 9 days, as further described in Example 33.

FIG. 39 is an exemplary XRPD pattern of Form D of the compound of formula (I), prepared in accordance with Example 34.

FIG. 40 is an exemplary DSC thermogram of Form D of the compound of formula (I), prepared in accordance with Example 34.

FIG. 41 is an exemplary TGA thermogram of Form D of the compound of formula (I), prepared in accordance with Example 34.

FIG. 42 is an exemplary DVS isotherm plot of Form D of the compound of formula (I), prepared in accordance with Example 34.

FIG. 43 is an exemplary XRPD pattern of Form E of the compound of formula (I), prepared in accordance with Example 35.

FIG. 44 is an exemplary DSC thermogram of Form E of the compound of formula (I) prepared in accordance with Example 35.

FIG. 45 is an exemplary TGA thermogram of Form E of the compound of formula (I), prepared in accordance with Example 35.

FIG. 46 is an exemplary XRPD pattern of Form H of the compound of formula (I), prepared in accordance with Example 36.

FIG. 47 is an exemplary DSC thermogram of Form H of the compound of formula (I), prepared in accordance with Example 36.

FIG. 48 is an exemplary XRPD pattern of Material F of the compound of formula (I), prepared in accordance with Example 37.

FIG. 49 is an exemplary XRPD pattern of Material G of the compound of formula (I), prepared in accordance with Example 38.

FIG. 50 is an exemplary DSC thermogram of Material G of the compound of formula (I), prepared in accordance with Example 38.

FIG. 51 is an exemplary TGA thermogram of Material G of the compound of formula (I), prepared in accordance with Example 38.

FIG. 52 is an XRPD pattern of a mixture of Form H and Form A of the compound of formula (I) obtained after storing a mixture of Form A and Form H in a mixture of heptane/ethanol (67:33, v/v) at ambient temperature for 21 days, as carried out in accordance with Example 39.

FIG. 53 is an XRPD pattern of Form A and Form H of the compound of formula (I) obtained after storing a mixture of Form A and Form H in a mixture of isopropanol/water (98:2, v/v) at ambient temperature for 11 days, as carried out in accordance with Example 39.

FIG. 54 is an XRPD pattern of Form E of the compound of formula (I) obtained after storing a mixture of Form A and Form H of the compound of formula (I) in ethyl acetate at ambient temperature for 11 days, as carried out in accordance with Example 39.

FIG. 55 is an XRPD pattern of a mixture of Form E, Material G, and Form A of the compound of formula (I) obtained after storing a mixture of Form E and Material G in ethyl acetate at ambient temperature for 3 days, as carried out in accordance with Example 39.

FIG. 56 is an XRPD pattern of a mixture of Form A and Form H of the compound of formula (I) obtained after storing Form A and Form B of the compound of formula (I) in ethanol at 21° C. for 4 days, as carried out in accordance with Example 39.

FIG. 57 is an XRPD pattern of a mixture of Form H and Form A of the compound of formula (I) obtained after storing Form A and Form B of the compound of formula (I) in ethyl acetate at 21° C. for 4 days, as carried out in accordance with Example 39.

FIG. 58 is an exemplary XRPD pattern of crystalline camsylate salt Form A of the compound of formula (I), prepared in accordance with Example 40.

FIG. 59 is an exemplary DSC thermogram of crystalline camsylate salt Form A of the compound of formula (I), prepared in accordance with Example 40.

FIG. 60 is an exemplary TGA thermogram of crystalline camsylate salt Form A of the compound of formula (I), prepared in accordance with Example 40.

FIG. 61 is an exemplary XRPD pattern of phosphate salt Material A of the compound of formula (I), prepared in accordance with Example 41.

FIG. 62 is an exemplary XRPD pattern of phosphate salt Material B of the compound of formula (I), prepared in accordance with Example 42.

FIG. 63 is an exemplary DSC thermogram of phosphate salt Material B of the compound of formula (I), prepared in accordance with Example 42.

FIG. 64 is an exemplary TGA thermogram of phosphate salt Material B of the compound of formula (I), prepared in accordance with Example 42.

### DETAILED DESCRIPTION

As generally described herein, the disclosure provides salt forms (e.g., fumarate, L-tartrate, tosylate, and succinate salts), including crystalline salt forms, and crystalline base forms of a compound of formula (I). The disclosure also provides pharmaceutical compositions comprising a crystalline form (e.g., crystalline salt form or crystalline free base form) of the compound of formula (I) described herein and a pharmaceutically acceptable excipient, and methods of using the crystalline forms (e.g., crystalline salt forms and crystalline free base forms) and pharmaceutical compositions described herein to treat a condition, disease, or disorder disclosed herein (e.g., a cancer).

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10%, ±5%, ±3%, ±2%, or ±1% variation from the nominal value unless otherwise indicated or inferred from the context.

Due to sources of experimental variability that are well known to those of ordinary skill in the art, the values of each XRPD peak are typically preceded with the term “about” or proceeded with an appropriate range defining the experimental variability. For purposes of data reported herein, that value is ±0.3° 2θ unless otherwise stated. XRPD peaks cited herein are generally reported with this variability of ±0.3° 2θ unless stated otherwise and are intended to be reported with such a variability whenever disclosed herein whether the word “about” is present or not, unless context dictates otherwise.

Due to sources of experimental variability that are well known to those of ordinary skill in the art, the values of each differential scanning calorimetry (DSC) endotherm or exotherm are typically preceded with the term “about” or proceeded with an appropriate range defining the experimental variability. For purposes of data reported herein, that value is ±10° C. unless otherwise stated. DSC endotherms/exotherms cited herein are generally reported with this variability of ±10° C. unless stated otherwise and are intended to be reported with such a variability whenever disclosed herein whether the word “about” is present or not, unless context dictates otherwise.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “pharmaceutical composition” or “pharmaceutical formulation” refers to the combination of an active agent with a carrier, inert or active, making the composition especially suitable for diagnostic or therapeutic use in vivo or ex vivo.

“Pharmaceutically acceptable” means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

For therapeutic use, salts of the compounds of the present invention (e.g., a compound of formula (I)) are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates, fatty acid esters, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, PA (1975).

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is an adult human. In certain embodiments, the subject is a non-human animal.

As used herein, “solid dosage form” means a pharmaceutical dose(s) in solid form, e.g., tablets, capsules, granules, powders, sachets, reconstitutable powders, dry powder inhalers and chewables.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or immunotherapy). The compound of formula (I), or a pharmaceutically acceptable salt thereof, can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

As used herein, “fasting state” means at least 1 hour before food or at least 2 hours after food is consumed by a subject.

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”).

In general, an “effective amount” of a compound or composition (e.g., a compound of formula (I), a crystalline form of a compound of formula (I) described herein, or a pharmaceutical composition described herein) refers to an amount sufficient to elicit the desired biological response, e.g., to treat a cancer described herein. As will be appreciated by those of ordinary skill in this art, the effective amount of a compound, crystalline form or pharmaceutical composition of the disclosure may vary depending on such factors as the desired biological endpoint, the pharmacokinetics of the compound, the disease being treated, the mode of administration, and the age, weight, health, and condition of the subject.

### Salt Forms of a Compound of Formula (I)

The compound of formula (I), as depicted below, is a selective PERK inhibitor, and also known as (R)-2-amino-5-(4-(2-(3,5-difluorophenyl)-2-hydroxyacetamido)-2-methylphenyl)-N-isopropylnicotinamide:

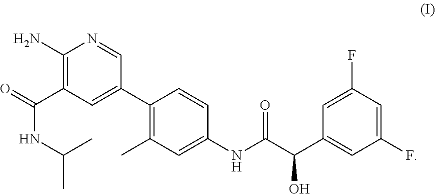 [SMILES: Cc1cc(NC(=O)[C@H](O)c2cc(F)cc(F)c2)ccc1-c1cnc(N)c(C(=O)NC(C)C)c1]

The compound of formula (I) may also be referred to as HC-5404 throughout the present disclosure. A method of chemically synthesizing the compound of formula (I) is described in Example 2.

In one aspect, provided herein is a salt form of the compound of formula (I)

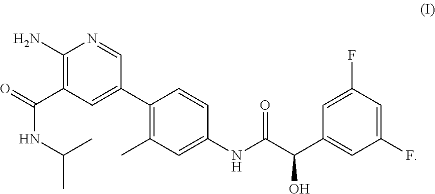 [SMILES: Cc1cc(NC(=O)[C@H](O)c2cc(F)cc(F)c2)ccc1-c1cnc(N)c(C(=O)NC(C)C)c1]

In various embodiments, the salt form of the compound of formula (I) is a fumarate salt of the compound of formula (I). In certain embodiments, the fumarate salt of the compound of formula (I) is a mono-fumarate salt. In certain embodiments, the fumarate salt of the compound of formula (I) is a hemi-fumarate salt.

In various embodiments, the salt form of the compound of formula (I) is an L-tartrate salt of the compound of formula (I).

In various embodiments, the salt form of the compound of formula (I) is a tosylate salt of the compound of formula (I).

In various embodiments, the salt form of the compound of formula (I) is a succinate salt of the compound of formula (I).

In another aspect, provided herein are crystalline salt forms of the compound of formula (I).

In various embodiments, the crystalline salt form of the compound of formula (I) is a crystalline fumarate salt of the compound of formula (I). In certain embodiments, the crystalline fumarate salt of the compound of formula (I) is a crystalline mono-fumarate salt. In certain embodiments, the crystalline fumarate salt of the compound of formula (I) is a crystalline mono-fumarate salt. In certain embodiments, the crystalline fumarate salt of the compound of formula (I) is a crystalline hemi-fumarate salt. In certain embodiments, the crystalline salt form is a crystalline camsylate salt form. In certain embodiments, the crystalline salt form is a crystalline phosphate salt form. In certain embodiments, the crystalline camsylate salt form is crystalline camsylate salt Form A of the compound of formula (I).

In certain embodiments, the crystalline salt form of the compound of formula (I) is Form A of the mono-fumarate salt of the compound of formula (I). In certain embodiments, the crystalline salt form of the compound of formula (I) is Form B of the mono-fumarate salt of the compound of formula (I). In certain embodiments, the crystalline salt form of the compound of formula (I) is Form C of the mono-fumarate salt of the compound of formula (I). In certain embodiments, the crystalline salt form of the compound of formula (I) is Form A of the hemi-fumarate salt of the compound of formula (I). In certain embodiments, the crystalline salt form of the compound of formula (I) is Form B of the hemi-fumarate salt of the compound of formula (I).

In certain embodiments, the crystalline salt form of the compound of formula (I) is a crystalline L-tartrate salt of the compound of formula (I).

In certain embodiments, the crystalline salt form of the compound of formula (I) is a crystalline tosylate salt of the compound of formula (I). In certain embodiments, the crystalline salt form of the compound of formula (I) is Form A of the tosylate salt of the compound of formula (I).

In certain embodiments, the crystalline salt form of the compound of formula (I) is a crystalline succinate salt of the compound of formula (I). In certain embodiments, the crystalline salt form of the compound of formula (I) is Form A of the succinate salt of the compound of formula (I).

### (1) Fumarate Salt Forms

In one aspect, provided herein is a fumarate salt of the compound of formula (I)

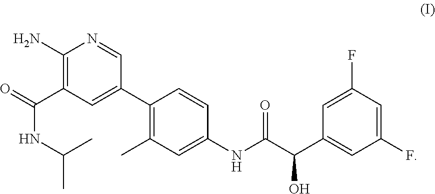 [SMILES: Cc1cc(NC(=O)[C@H](O)c2cc(F)cc(F)c2)ccc1-c1cnc(N)c(C(=O)NC(C)C)c1]

In certain embodiments, the fumarate salt of the compound of formula (I) is a mono-fumarate salt. In certain embodiments, the fumarate salt of the compound of formula (I) is a hemi-fumarate salt.

In another aspect, provided herein are crystalline fumarate salts of the compound of formula (I)

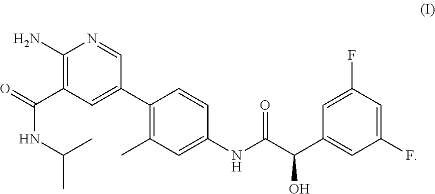 [SMILES: Cc1cc(NC(=O)[C@H](O)c2cc(F)cc(F)c2)ccc1-c1cnc(N)c(C(=O)NC(C)C)c1]

In certain embodiments, the crystalline fumarate salt has an X-ray powder diffraction (XRPD) pattern comprising one or more peaks at about 16.9°, about 17.9°, and about 24.1° 2θ.

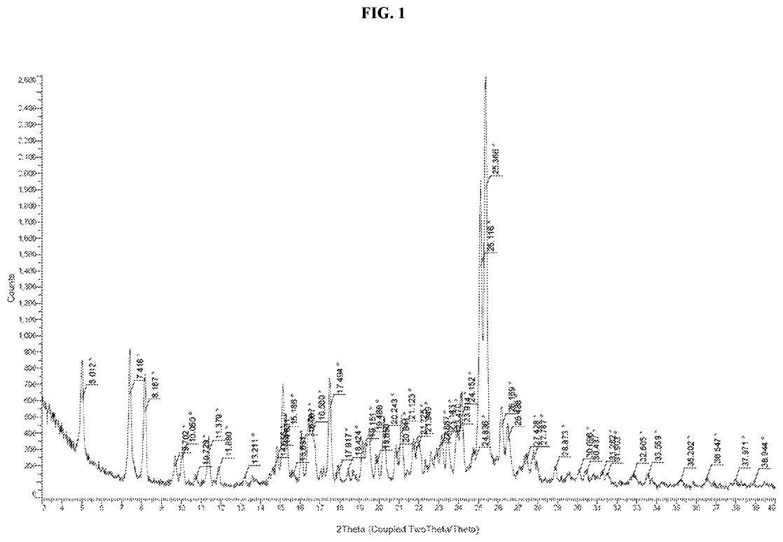

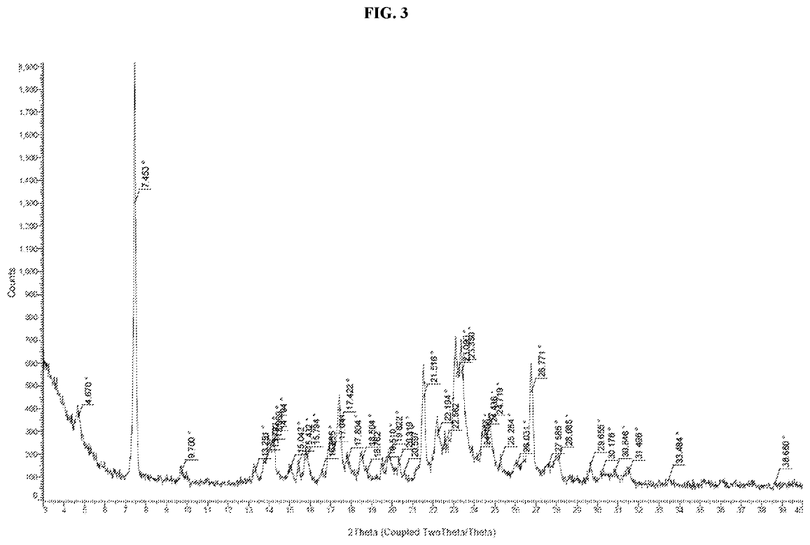

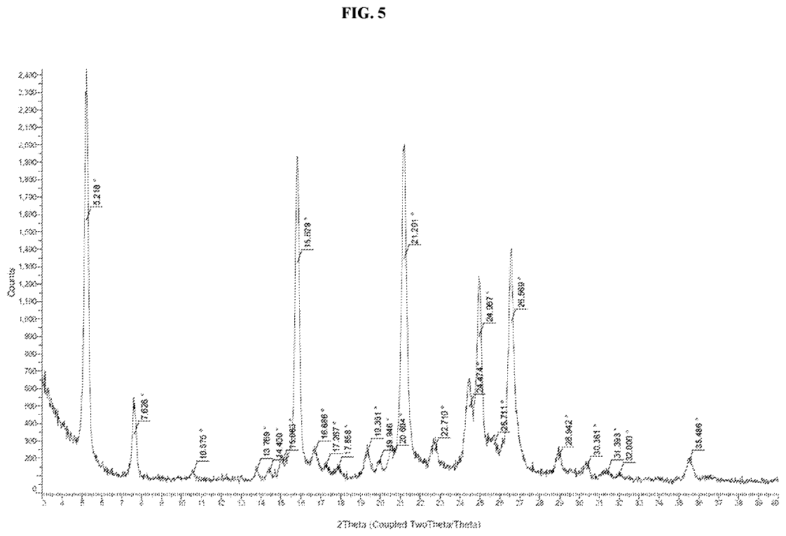

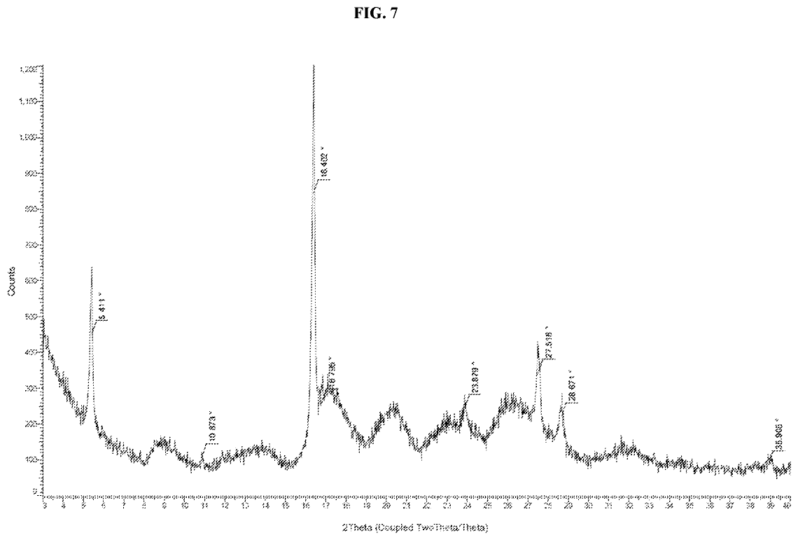

In certain embodiments, the crystalline fumarate salt is a crystalline mono-fumarate salt. In certain embodiments, the crystalline fumarate salt has an XRPD pattern substantially the same as shown in FIG. 1. In certain embodiments, the crystalline fumarate salt has an XRPD pattern substantially the same as shown in FIG. 3. In certain embodiments, the crystalline fumarate salt has an XRPD pattern substantially the same as shown in FIG. 5. In certain embodiments, the crystalline fumarate salt has an XRPD pattern substantially the same as shown in FIG. 7.

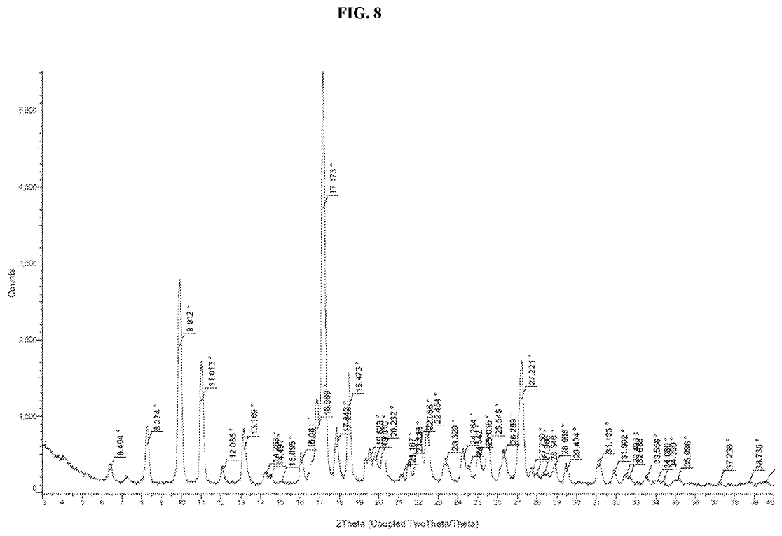

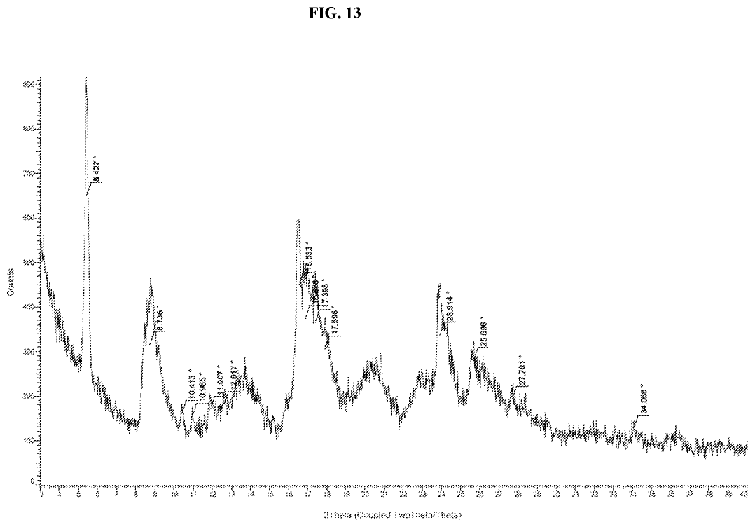

In certain embodiments, the crystalline fumarate salt is a crystalline hemi-fumarate salt. In certain embodiments, the crystalline fumarate salt has an XRPD pattern substantially the same as shown in FIG. 8. In certain embodiments, the crystalline fumarate salt has an XRPD pattern substantially the same as shown in FIG. 13.

In certain embodiments, the crystalline fumarate salt of the compound of formula (I) has an XRPD pattern comprising one or more diffraction peaks (2θ) disclosed in Table 1. In certain embodiments, the crystalline fumarate salt of the compound of formula (I) has an XRPD pattern comprising one or more diffraction peaks (2θ) disclosed in Table 8. In certain embodiments, the crystalline fumarate salt of the compound of formula (I) has an XRPD pattern comprising one or more diffraction peaks (2θ) disclosed in Table 9. In certain embodiments, the crystalline fumarate salt of the compound of formula (I) has an XRPD pattern comprising one or more diffraction peaks (2θ) disclosed in Table 10. In certain embodiments, the crystalline fumarate salt of the compound of formula (I) has an XRPD pattern comprising one or more diffraction peaks (2θ) disclosed in Table 11. In certain embodiments, the crystalline fumarate salt of the compound of formula (I) has an XRPD pattern comprising one or more diffraction peaks (2θ) disclosed in Table 19.

In certain embodiments, the crystalline fumarate salt of the compound of formula (I) is an anhydrous crystalline fumarate salt.

In certain embodiments, the crystalline fumarate salt of the compound of formula (I) is a crystalline hydrate. In certain embodiments, the crystalline hydrate is a crystalline monohydrate.

### (A) Crystalline Forms of the Mono-Fumarate Salt

In various embodiments, provided herein is a crystalline mono-fumarate salt of the compound of formula (I)

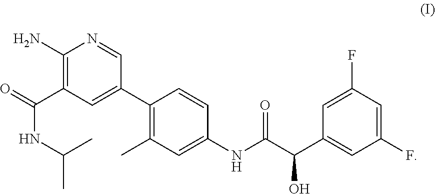 [SMILES: Cc1cc(NC(=O)[C@H](O)c2cc(F)cc(F)c2)ccc1-c1cnc(N)c(C(=O)NC(C)C)c1]

In certain embodiments, the crystalline mono-fumarate salt has an XRPD pattern comprising one or more peaks at about 5.2° 2θ.

In certain embodiments, the XRPD pattern further comprises one or more peaks at about 7.6° and about 14.4° 2θ.

In certain embodiments, the XRPD pattern further comprises one or more peaks at about 15.8°, about 21.2°, about 24.5°, and about 25° 2θ.

In certain embodiments, the XRPD pattern further comprises one or more peaks at about 15.1°, about 16.7°, about 17.3°, about 17.9°, about 19.4°, about 19.9°, about 20.6°, and about 22.7° 2θ.

In certain embodiments, the XRPD pattern further comprises one or more peaks at about 30.4° and about 31.4° 2θ.

In certain embodiments, the crystalline mono-fumarate salt has an XRPD pattern comprising one or more peaks at about 5.2°, about 7.6°, about 14.4°, about 15.1°, about 15.8°, about 16.7°, about 17.3°, about 17.9°, about 19.4°, about 19.9°, about 20.6°, about 21.2°, about 22.7°, about 24.5°, about 25.0° about 30.4°, and about 31.4° 2θ.

In certain embodiments, the crystalline mono-fumarate salt has an XRPD pattern comprising peaks at about 5.2°, about 7.6°, about 14.4°, about 15.1°, about 15.8°, about 16.7°, about 17.3°, about 17.9°, about 19.4°, about 19.9°, about 20.6°, about 21.2°, about 22.7°, about 24.5°, about 25.0°, about 30.4°, and about 31.4° 2θ.

In certain embodiments, the crystalline mono-fumarate salt has an XRPD pattern comprising one or more peaks at 5.2°±0.3° 2θ.

In certain embodiments, the XRPD pattern further comprises one or more peaks at 7.6°±0.3° and 14.4°±0.3° 2θ.

In certain embodiments, the XRPD pattern further comprises one or more peaks at 15.8°±0.3°, 21.2°±0.3°, 24.5°±0.3°, and 25°±0.3° 2θ.

In certain embodiments, the XRPD pattern further comprises one or more peaks at 15.1°±0.3°, 16.7°±0.3°, 17.3°±0.3°, 17.9°±0.3°, 19.4°±0.3°, 19.9°±0.3°, 20.6°±0.3°, and 22.7°±0.3° 2θ.

In certain embodiments, the XRPD pattern further comprises one or more peaks at 30.4°±0.3° and 31.4°±0.3° 2θ.

In certain embodiments, the crystalline mono-fumarate salt has an XRPD pattern comprising one or more peaks at 5.2°±0.3°, 7.6°±0.3°, 14.4°±0.3°, 15.1°±0.3°, 15.8°±0.3°, 16.7°±0.3°, 17.3°±0.3°, 17.9°±0.3°, 19.4°±0.3°, 19.9°±0.3°, 20.6°±0.3°, 21.2°±0.3°, 22.7°±0.3°, 24.5°±0.3°, 25.0°±0.3°, 30.4°±0.3°, and 31.4°±0.3° 2θ.

In certain embodiments, the crystalline mono-fumarate salt has an XRPD pattern comprising peaks at 5.2°±0.3°, 7.6°±0.3°, 14.4°±0.3°, 15.1°±0.3°, 15.8°±0.3°, 16.7°±0.3°, 17.3°±0.3°, 17.9°±0.3°, 19.4°±0.3°, 19.9°±0.3°, 20.6°±0.3°, 21.2°±0.3°, 22.7°±0.3°, 24.5°±0.3°, 25.0°±0.3°, 30.4°±0.3°, and 31.4°±0.3° 2θ.

In certain embodiments, the crystalline mono-fumarate salt has an XRPD pattern comprising one or more peaks at 5.2°±0.2° 2θ.

In certain embodiments, the XRPD pattern further comprises one or more peaks at 7.6°±0.2° and 14.4°±0.2° 2θ.

In certain embodiments, the XRPD pattern further comprises one or more peaks at 15.8°±0.2°, 21.2°±0.2°, 24.5°±0.2°, and 25°±0.2° 2θ.

In certain embodiments, the XRPD pattern further comprises one or more peaks at 15.1°±0.2°, 16.7°±0.2°, 17.3°±0.2°, 17.9°±0.2°, 19.4°±0.2°, 19.9°±0.2°, 20.6°±0.2°, and 22.7°±0.2° 2θ.

In certain embodiments, the XRPD pattern further comprises one or more peaks at 30.4°±0.2° and 31.4°±0.2° 2θ.

In certain embodiments, the crystalline mono-fumarate salt has an XRPD pattern comprising one or more peaks at 5.2°±0.2°, 7.6°±0.2°, 14.4°±0.2°, 15.1°±0.2°, 15.8°±0.2°, 16.7°±0.2°, 17.3°±0.2°, 17.9°±0.2°, 19.4°±0.2°, 19.9°±0.2°, 20.6°±0.2°, 21.2°±0.2°, 22.7°±0.2°, 24.5°±0.2°, 25.0°±0.2°, 30.4°±0.2°, and 31.4°±0.2° 2θ.

In certain embodiments, the crystalline mono-fumarate salt has an XRPD pattern comprising peaks at 5.2°±0.2°, 7.6°±0.2°, 14.4°±0.2°, 15.1°±0.2°, 15.8°±0.2°, 16.7°±0.2°, 17.3°±0.2°, 17.9°±0.2°, 19.4°±0.2°, 19.9°±0.2°, 20.6°±0.2°, 21.2°±0.2°, 22.7°±0.2°, 24.5°±0.2°, 25.0°±0.2°, 30.4°±0.2°, and 31.4°±0.2° 2θ.

In certain embodiments, the crystalline mono-fumarate salt of the compound of formula (I) is Form A of the mono-fumarate salt of the compound of formula (I). In certain embodiments, the crystalline mono-fumarate salt of the compound of formula (I) is Form B of the mono-fumarate salt of the compound of formula (I). In certain embodiments, the crystalline mono-fumarate salt of the compound of formula (I) is Form C of the mono-fumarate salt of the compound of formula (I).

In certain embodiments, the crystalline mono-fumarate salt of the compound of formula (I) is an anhydrous crystalline form.

In certain embodiments, the crystalline mono-fumarate of the compound of formula (I) is a crystalline hydrate. In certain embodiments, the crystalline hydrate is a monohydrate.

### (i) Form A of the Mono-Fumarate Salt

In various embodiments, provided herein is Form A of the mono-fumarate salt of the compound of formula (I)

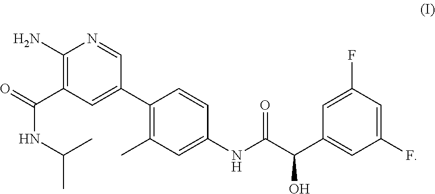 [SMILES: Cc1cc(NC(=O)[C@H](O)c2cc(F)cc(F)c2)ccc1-c1cnc(N)c(C(=O)NC(C)C)c1]

In certain embodiments, Form A of the mono-fumarate salt of the compound of formula (I) has an X-ray powder diffraction (XRPD) pattern comprises one or more peaks at about 5.0°, about 7.4°, and about 8.2° 2θ.

In certain embodiments, the XRPD pattern further comprises one or more peaks at about 9.7°, about 10.1°, about 10.7°, about 11.4°, about 11.9°, about 13.2°, about 14.7°, and about 14.8° 2θ.

In certain embodiments, the XRPD pattern further comprises one or more peaks at about 15.2°, about 16.1°, about 16.6°, about 17.5°, about 19.5°, about 20.2°, about 21.1°, about 23.9°, and about 24.2° 2θ.

In certain embodiments, the XRPD pattern further comprises one or more peaks at about 17.9°, about 18.4°, about 19.2°, about 19.9°, about 20.8°, about 21.9°, about 22.9°, about 23.1°, about 23.5°, and about 24.8° 2θ.

In certain embodiments, the XRPD pattern further comprises one or more peaks at about 25.1°, about 25.4°, about 27.4°, and about 27.8° 2θ.

In certain embodiments, the XRPD pattern further comprises one or more peaks at about 28.9°, about 30.1°, about 30.4°, about 31.3°, about 31.5°, about 32.8°, and about 33.6° 2θ.

In certain embodiments, Form A of the mono-fumarate salt of the compound of formula (I) has an XRPD pattern comprising one or more peaks at about 5.0°, about 7.4°, about 8.2°, about 9.7°, about 10.1°, about 10.7°, about 11.4°, about 11.9°, about 13.2°, about 14.7°, about 14.8°, about 15.2°, about 16.1°, about 16.6°, about 17.5°, about 17.9°, about 18.4°, about 19.2°, about 19.5°, about 19.9°, about 20.2°, about 20.8°, about 21.1°, about 21.9°, about 22.9°, about 23.1°, about 23.5°, about 23.9°, about 24.2°, about 24.8°, about 28.9°, about 30.1°, about 30.4°, about 31.3°, about 31.5°, about 32.8°, and about 33.6° 2θ.

In certain embodiments, Form A of the mono-fumarate salt of the compound of formula (I) has an XRPD pattern comprising peaks at about 5.0°, about 7.4°, about 8.2°, about 9.7°, about 10.1°, about 10.7°, about 11.4°, about 11.9°, about 13.2°, about 14.7°, about 14.8°, about 15.2°, about 16.1°, about 16.6°, about 17.5°, about 17.9°, about 18.4°, about 19.2°, about 19.5°, about 19.9°, about 20.2°, about 20.8°, about 21.1°, about 21.9°, about 22.9°, about 23.1°, about 23.5°, about 23.9°, about 24.2°, about 24.8°, about 28.9°, about 30.1°, about 30.4°, about 31.3°, about 31.5°, about 32.8°, and about 33.6° 2θ.

In certain embodiments, Form A of the mono-fumarate salt of the compound of formula (I) has an X-ray powder diffraction (XRPD) pattern comprises one or more peaks at 5.0°±0.3°, 7.4°±0.3°, and 8.2°±0.3° 2θ.

In certain embodiments, the XRPD pattern further comprises one or more peaks at 9.7°±0.3°, 10.1°±0.3°, 10.7°±0.3°, 11.4°±0.3°, 11.9°±0.3°, 13.2°±0.3°, 14.7°±0.3°, and 14.8°±0.3° 2θ.

In certain embodiments, the XRPD pattern further comprises one or more peaks at 15.2°±0.3°, 16.1°±0.3°, 16.6°±0.3°, 17.5°±0.3°, 19.5°±0.3°, 20.2°±0.3°, 21.1°±0.3°, 23.9°±0.3°, and 24.2°±0.3° 2θ.

In certain embodiments, the XRPD pattern further comprises one or more peaks at 17.9°±0.3°, 18.4°±0.3°, 19.2°±0.3°, 19.9°±0.3°, 20.8°±0.3°, 21.9°±0.3°, 22.9°±0.3°, 23.1°±0.3°, 23.5°±0.3°, and 24.8°±0.3° 2θ.

In certain embodiments, the XRPD pattern further comprises one or more peaks at 25.1°±0.3°, 25.4°±0.3°, 27.4°±0.3°, and 27.8°±0.3° 2θ.

In certain embodiments, the XRPD pattern further comprises one or more peaks at 28.9°±0.3°, 30.1°±0.3°, 30.4°±0.3°, 31.3°±0.3°, 31.5°±0.3°, 32.8°±0.3°, and 33.6°±0.3° 2θ.

In certain embodiments, Form A of the mono-fumarate salt of the compound of formula (I) has an XRPD pattern comprising one or more peaks at 5.0°±0.3°, 7.4°±0.3°. 8.2°±0.3°, 9.7°±0.3°, 10.1°±0.3°, 10.7°±0.3°, 11.4°±0.3°, 11.9°±0.3°, 13.2°±0.3°, 14.7°±0.3°, 14.8°±0.3°, 15.2°±0.3°, 16.1°±0.3°, 16.6°±0.3°, 17.5°±0.3°, 17.9°±0.3°, 18.4°±0.3°, 19.2°±0.3°, 19.5°±0.3°, 19.9°±0.3°, 20.2°±0.3°, 20.8°±0.3°, 21.1°±0.3°, 21.9°±0.3°, 22.9°±0.3°, 23.1°±0.3°, 23.5°±0.3°, 23.9°±0.3°, 24.2°±0.3°, 24.8°±0.3°, 28.9°±0.3°, 30.1°±0.3°, 30.4°±0.3°, 31.3°±0.3°, 31.5°±0.3°, 32.8°±0.3°, and 33.6°±0.3° 2θ.

In certain embodiments, Form A of the mono-fumarate salt of the compound of formula (I) has an XRPD pattern comprising peaks at 5.0°±0.3°, 7.4°±0.3°, 8.2°±0.3°, 9.7°±0.3°, 10.1°±0.3°, 10.7°±0.3°, 11.4°±0.3°, 11.9°±0.3°, 13.2°±0.3°, 14.7°±0.3°, 14.8°±0.3°, 15.2°±0.3°, 16.1°±0.3°, 16.6°±0.3°, 17.5°±0.3°, 17.9°±0.3°, 18.4°±0.3°, 19.2°±0.3°, 19.5°±0.3°, 19.9°±0.3°, 20.2°±0.3°, 20.8°±0.3°, 21.1°±0.3°, 21.9°±0.3°, 22.9°±0.3°, 23.1°±0.3°, 23.5°±0.3°, 23.9°±0.3°, 24.2°±0.3°, 24.8°±0.3°, 28.9°±0.3°, 30.1°±0.3°, 30.4°±0.3°, 31.3°±0.3°, 31.5°±0.3°, 32.8°±0.3°, and 33.6°±0.3° 2θ.

In certain embodiments, Form A of the mono-fumarate salt of the compound of formula (I) has an X-ray powder diffraction (XRPD) pattern comprises one or more peaks at 5.0°±0.2°, 7.4°±0.2°, and 8.2°±0.2° 2θ.

In certain embodiments, the XRPD pattern further comprises one or more peaks at 9.7°±0.2°, 10.1°±0.2°, 10.7°±0.2°, 11.4°±0.2°, 11.9°±0.2°, 13.2°±0.2°, 14.7°±0.2°, and 14.8°±0.2° 2θ.

In certain embodiments, the XRPD pattern further comprises one or more peaks at 15.2°±0.2°, 16.1°±0.2°, 16.6°±0.2°, 17.5°±0.2°, 19.5°±0.2°, 20.2°±0.2°, 21.1°±0.2°, 23.9°±0.2°, and 24.2°±0.2° 2θ.

In certain embodiments, the XRPD pattern further comprises one or more peaks at 17.9°±0.2°, 18.4°±0.2°, 19.2°±0.2°, 19.9°±0.2°, 20.8°±0.2°, 21.9°±0.2°, 22.9°±0.2°, 23.1°±0.2°, 23.5°±0.2°, and 24.8°±0.2° 2θ.

In certain embodiments, the XRPD pattern further comprises one or more peaks at 25.1°±0.2°, 25.4°±0.2°, 27.4°±0.2°, and 27.8°±0.2° 2θ.

In certain embodiments, the XRPD pattern further comprises one or more peaks at 28.9°±0.2°, 30.1°±0.2°, 30.4°±0.2°, 31.3°±0.2°, 31.5°±0.2°, 32.8°±0.2°, and 33.6°±0.2° 2θ.

In certain embodiments, Form A of the mono-fumarate salt of the compound of formula (I) has an XRPD pattern comprising one or more peaks at 5.0°±0.2°, 7.4°±0.2°, 8.2°±0.2°, 9.7°±0.2°, 10.1°±0.2°, 10.7°±0.2°, 11.4°±0.2°, 11.9°±0.2°, 13.2°±0.2°, 14.7°±0.2°, 14.8°±0.2°, 15.2°±0.2°, 16.1°±0.2°, 16.6°±0.2°, 17.5°±0.2°, 17.9°±0.2°, 18.4°±0.2°, 19.2°±0.2°, 19.5°±0.2°, 19.9°±0.2°, 20.2°±0.2°, 20.8°±0.2°, 21.1°±0.2°, 21.9°±0.2°, 22.9°±0.2°, 23.1°±0.2°, 23.5°±0.2°, 23.9°±0.2°, 24.2°±0.2°, 24.8°±0.2°, 28.9°±0.2°, 30.1°±0.2°, 30.4°±0.2°, 31.3°±0.2°, 31.5°±0.2°, 32.8°±0.2°, and 33.6°±0.2° 2θ.

In certain embodiments, Form A of the mono-fumarate salt of the compound of formula (I) has an XRPD pattern comprising peaks at 5.0°±0.2°, 7.4°±0.2°, 8.2°±0.2°, 9.7°±0.2°, 10.1°±0.2°, 10.7°±0.2°, 11.4°±0.2°, 11.9°±0.2°, 13.2°±0.2°, 14.7°±0.2°, 14.8°±0.2°, 15.2°±0.2°, 16.1°±0.2°, 16.6°±0.2°, 17.5°±0.2°, 17.9°±0.2°, 18.4°±0.2°, 19.2°±0.2°, 19.5°±0.2°, 19.9°±0.2°, 20.2°±0.2°, 20.8°±0.2°, 21.1°±0.2°, 21.9°±0.2°, 22.9°±0.2°, 23.1°±0.2°, 23.5°±0.2°, 23.9°±0.2°, 24.2°±0.2°, 24.8°±0.2°, 28.9°±0.2°, 30.1°±0.2°, 30.4°±0.2°, 31.3°±0.2°, 31.5°±0.2°, 32.8°±0.2°, and 33.6°±0.2° 2θ.

In certain embodiments, Form A of the mono-fumarate salt of the compound of formula (I) has an XRPD pattern substantially the same as shown in FIG. 1. In certain embodiments, Form A of the mono-fumarate salt of the compound of formula (I) has an XRPD pattern comprising one or more diffraction peaks (2θ) disclosed in Table 1.

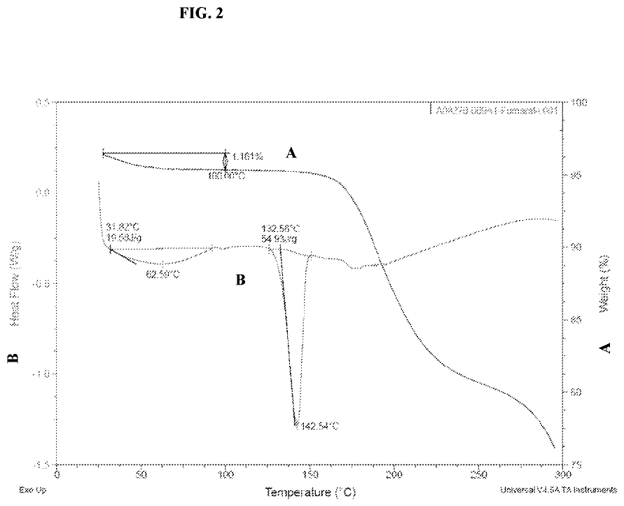

In certain embodiments, Form A of the mono-fumarate salt of the compound of formula (I) has a differential scanning calorimetry (DSC) thermogram comprising an endotherm with a peak onset at about 32° C. In certain embodiments, Form A of the mono-fumarate salt of the compound of formula (I) has a differential scanning calorimetry (DSC) thermogram comprising an endotherm with a peak onset at about 133° C. In certain embodiments, Form A of the mono-fumarate salt of the compound of formula (I) has a differential scanning calorimetry (DSC) thermogram comprising one or more endotherms with peak onsets at about 32° C., and about 133° C. In certain embodiments, Form A of the mono-fumarate salt of the compound of formula (I) has a DSC thermogram substantially the same as shown in FIG. 2.

In certain embodiments, Form A of the mono-fumarate salt of the compound of formula (I) exhibits a weight loss of less than or equal to about 1.2% wt. upon heating Form A from about 25° C. to about 100° C. The weight loss exhibited by a crystalline form (e.g., Form A of the mono-fumarate salt of the compound of formula (I)) can be determined, for example, using thermogravimetric analysis (TGA). In certain embodiments, Form A of the mono-fumarate salt of the compound of formula (I) has a TGA thermogram substantially the same as shown in FIG. 2.

In certain embodiments, Form A of the mono-fumarate salt of the compound of formula (I) is a crystalline hydrate.

### (ii) Form B of the Mono-Fumarate Salt

In various embodiments, provided herein is Form B of the mono-fumarate salt of the compound of formula (I)

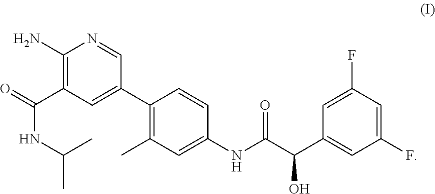 [SMILES: Cc1cc(NC(=O)[C@H](O)c2cc(F)cc(F)c2)ccc1-c1cnc(N)c(C(=O)NC(C)C)c1]

In certain embodiments, Form B of the mono-fumarate salt of the compound of formula (I) has an X-ray powder diffraction (XRPD) pattern comprising a peak at about 7.5° 2θ.

In certain embodiments, the XRPD pattern further comprises one or more peaks at about 4.7°, about 9.7°, about 13.3°, about 13.8°, about 14.0°, about 14.2°, and about 15.0° 2θ.

In certain embodiments, the XRPD pattern further comprises one or more peaks at about 17.4°, about 21.5°, about 22.2°, about 22.6°, about 23.1°, about 23.4°, about 24.4°, and about 24.7° 2θ.

In certain embodiments, the XRPD pattern further comprises one or more peaks at about 15.4°, about 15.8°, about 16.6°, about 17.0°, about 17.8°, about 18.5°, about 18.8°, about 19.5°, about 19.8°, about 20.3°, about 20.6°, and about 24.1° 2θ.

In certain embodiments, the XRPD pattern further comprises one or more peaks at about 15.4°, about 15.8°, about 16.6°, about 17.0°, about 17.4°, about 17.8°, about 18.5°, about 18.8°, about 19.5°, about 19.8°, about 20.3°, about 20.6°, about 21.5°, about 22.2°, about 22.6°, about 23.1°, about 23.4°, about 24.4°, about 24.1°, and about 24.7° 2θ.

In certain embodiments, the XRPD pattern further comprises one or more peaks at about 26.8°, about 28.1°, and about 29.7° 2θ.

In certain embodiments, the XRPD pattern further comprises one or more peaks at about 25.3°, about 26.0°, about 27.6°, about 30.2°, about 30.8°, about 31.5°, and about 33.5° 2θ.

In certain embodiments, the XRPD pattern further comprises one or more peaks at about 25.3°, about 26.0°, about 26.8°, about 27.6°, about 28.1°, and about 29.7°, about 30.2°, about 30.8°, about 31.5°, and about 33.5° 2θ.

In certain embodiments, Form B of the mono-fumarate salt of the compound of formula (I) has an XRPD pattern comprising one or more peaks at about 4.7°, about 7.5°, about 9.7°, about 13.3°, about 13.8°, about 14.0°, about 14.2°, and about 15.0°, about 15.4°, about 15.8°, about 16.6°, about 17.0°, about 17.4°, about 17.8°, about 18.5°, about 18.8°, about 19.5°, about 19.8°, about 20.3°, about 20.6°, about 21.5°, about 22.2°, about 22.6°, about 23.1°, about 23.4°, about 24.1°, about 24.4°, about 24.7°, about 25.3°, about 26.0°, about 26.8°, about 27.6°, about 28.1°, about 29.7°, about 30.2°, about 30.8°, about 31.5°, and about 33.5° 2θ.

In certain embodiments, Form B of the mono-fumarate salt of the compound of formula (I) has an XRPD pattern comprising peaks at about 4.7°, about 7.5°, about 9.7°, about 13.3°, about 13.8°, about 14.0°, about 14.2°, and about 15.0°, about 15.4°, about 15.8°, about 16.6°, about 17.0°, about 17.4°, about 17.8°, about 18.5°, about 18.8°, about 19.5°, about 19.8°, about 20.3°, about 20.6°, about 21.5°, about 22.2°, about 22.6°, about 23.1°, about 23.4°, about 24.1°, about 24.4°, about 24.7°, about 25.3°, about 26.0°, about 26.8°, about 27.6°, about 28.1°, about 29.7°, about 30.2°, about 30.8°, about 31.5°, and about 33.5° 2θ.

In certain embodiments, Form B of the mono-fumarate salt of the compound of formula (I) has an XRPD pattern comprising a peak at 7.5°±0.3° 2θ.

In certain embodiments, the XRPD pattern further comprises one or more peaks at 4.7°±0.3°, 9.7°±0.3°, 13.3°±0.3°, 13.8°±0.3°, 14.0°±0.3°, 14.2°±0.3°, and 15.0°±0.3° 2θ.

In certain embodiments, the XRPD pattern further comprises one or more peaks at 17.4°±0.3°, 21.5°±0.3°, 22.2°±0.3°, 22.6°±0.3°, 23.1°±0.3°, 23.4°±0.3°, 24.4°±0.3°, and 24.7°±0.3° 2θ.

In certain embodiments, the XRPD pattern further comprises one or more peaks at 15.4°±0.3°, 15.8°±0.3°, 16.6°±0.3°, 17.0°±0.3°, 17.8°±0.3°, 18.5°±0.3°, 18.8°±0.3°, 19.5°±0.3°, 19.8°±0.3°, 20.3°±0.3°, 20.6°±0.3°, and 24.1°±0.3° 2θ.

In certain embodiments, the XRPD pattern further comprises one or more peaks at 15.4°±0.3°, 15.8°±0.3°, 16.6°±0.3°, 17.0°±0.3°, 17.4°±0.3°, 17.8°±0.3°, 18.5°±0.3°, 18.8°±0.3°, 19.5°±0.3°, 19.8°±0.3°, 20.3°±0.3°, 20.6°±0.3°, 21.5°±0.3°, 22.2°±0.3°, 22.6°±0.3°, 23.1°±0.3°, 23.4°±0.3°, 24.4°±0.3°, 24.1°±0.3°, and 24.7°±0.3° 2θ.

In certain embodiments, the XRPD pattern further comprises one or more peaks at 26.8°±0.3°, 28.1°±0.3°, and 29.7°±0.3° 2θ.

In certain embodiments, the XRPD pattern further comprises one or more peaks at 25.3°±0.3°, 26.0°±0.3°, 27.6°±0.3°, 30.2°±0.3°, 30.8°±0.3°, 31.5°±0.3°, and 33.5°±0.3° 2θ.

In certain embodiments, the XRPD pattern further comprises one or more peaks at 25.3°±0.3°, 26.0°±0.3°, 26.8°±0.3°, 27.6°±0.3°, 28.1°±0.3°, 29.7°±0.3°, 30.2°±0.3°, 30.8°±0.3°, 31.5°±0.3°, and 33.5°±0.3° 2θ.

In certain embodiments, Form B of the mono-fumarate salt of the compound of formula (I) has an XRPD pattern comprising one or more peaks at 4.7°±0.3°, 7.5°±0.3°, 9.7°±0.3°, 13.3°±0.3°, 13.8°±0.3°, 14.0°±0.3°, 14.2°±0.3°, 15.0°±0.3°, 15.4°±0.3°, 15.8°±0.3°, 16.6°±0.3°, 17.0°±0.3°, 17.4°±0.3°, 17.8°±0.3°, 18.5°±0.3°, 18.8°±0.3°, 19.5°±0.3°, 19.8°±0.3°, 20.3°±0.3°, 20.6°±0.3°, 21.5°±0.3°, 22.2°±0.3°, 22.6°±0.3°, 23.1°±0.3°, 23.4°±0.3°, 24.1°±0.3°, 24.4°±0.3°, 24.7°±0.3°, 25.3°±0.3°, 26.0°±0.3°, 26.8°±0.3°, 27.6°±0.3°, 28.1°±0.3°, 29.7°±0.3°, 30.2°±0.3°, 30.8°±0.3°, 31.5°±0.3°, and 33.5°±0.3° 2θ.

In certain embodiments, Form B of the mono-fumarate salt of the compound of formula (I) has an XRPD pattern comprising peaks at 4.7°±0.3°, 7.5°±0.3°, 9.7°±0.3°, 13.3°±0.3°, 13.8°±0.3°, 14.0°±0.3°, 14.2°±0.3°, 15.0°±0.3°, 15.4°±0.3°, 15.8°±0.3°, 16.6°±0.3°, 17.0°±0.3°, 17.4°±0.3°, 17.8°±0.3°, 18.5°±0.3°, 18.8°±0.3°, 19.5°±0.3°, 19.8°±0.3°, 20.3°±0.3°, 20.6°±0.3°, 21.5°±0.3°, 22.2°±0.3°, 22.6°±0.3°, 23.1°±0.3°, 23.4°±0.3°, 24.1°±0.3°, 24.4°±0.3°, 24.7°±0.3°, 25.3°±0.3°, 26.0°±0.3°, 26.8°±0.3°, 27.6°±0.3°, 28.1°±0.3°, 29.7°±0.3°, 30.2°±0.3°, 30.8°±0.3°, 31.5°±0.3°, and 33.5°±0.3° 2θ.

In certain embodiments, Form B of the mono-fumarate salt of the compound of formula (I) has an XRPD pattern comprising a peak at 7.5°±0.2° 2θ.

In certain embodiments, the XRPD pattern further comprises one or more peaks at 4.7°±0.2°, 9.7°±0.2°, 13.3°±0.2°, 13.8°±0.2°, 14.0°±0.2°, 14.2°±0.2°, and 15.0°±0.2° 2θ.

In certain embodiments, the XRPD pattern further comprises one or more peaks at 17.4°±0.2°, 21.5°±0.2°, 22.2°±0.2°, 22.6°±0.2°, 23.1°±0.2°, 23.4°±0.2°, 24.4°±0.2°, and 24.7°±0.2° 2θ.

In certain embodiments, the XRPD pattern further comprises one or more peaks at 15.4°±0.2°, 15.8°±0.2°, 16.6°±0.2°, 17.0°±0.2°, 17.8°±0.2°, 18.5°±0.2°, 18.8°±0.2°, 19.5°±0.2°, 19.8°±0.2°, 20.3°±0.2°, 20.6°±0.2°, and 24.1°±0.2° 2θ.

In certain embodiments, the XRPD pattern further comprises one or more peaks at 15.4°±0.2°, 15.8°±0.2°, 16.6°±0.2°, 17.0°±0.2°, 17.4°±0.2°, 17.8°±0.2°, 18.5°±0.2°, 18.8°±0.2°, 19.5°±0.2°, 19.8°±0.2°, 20.3°±0.2°, 20.6°±0.2°, 21.5°±0.2°, 22.2°±0.2°, 22.6°±0.2°, 23.1°±0.2°, 23.4°±0.2°, 24.1°±0.2°, 24.4°±0.2°, and 24.7°±0.2° 2θ.

In certain embodiments, the XRPD pattern further comprises one or more peaks at 26.8°±0.2°, 28.1°±0.2°, and 29.7°±0.2° 2θ.

In certain embodiments, the XRPD pattern further comprises one or more peaks at 25.3°±0.2°, 26.0°±0.2°, 27.6°±0.2°, 30.2°±0.2°, 30.8°±0.2°, 31.5°±0.2°, and 33.5°±0.2° 2θ.

In certain embodiments, the XRPD pattern further comprises one or more peaks at 25.3°±0.2°, 26.0°±0.2°, 26.8°±0.2°, 27.6°±0.2°, 28.1°±0.2°, 29.7°±0.2°, 30.2°±0.2°, 30.8°±0.2°, 31.5°±0.2°, and 33.5°±0.2° 2θ.

In certain embodiments, Form B of the mono-fumarate salt of the compound of formula (I) has an XRPD pattern comprising one or more peaks at 4.7°±0.2°, 7.5°±0.2°, 9.7°±0.2°, 13.3°±0.2°, 13.8°±0.2°, 14.0°±0.2°, 14.2°±0.2°, 15.0°±0.2°, 15.4°±0.2°, 15.8°±0.2°, 16.6°±0.2°, 17.0°±0.2°, 17.4°±0.2°, 17.8°±0.2°, 18.5°±0.2°, 18.8°±0.2°, 19.5°±0.2°, 19.8°±0.2°, 20.3°±0.2°, 20.6°±0.2°, 21.5°±0.2°, 22.2°±0.2°, 22.6°±0.2°, 23.1°±0.2°, 23.4°±0.2°, 24.1°±0.2°, 24.4°±0.2°, 24.7°±0.2°, 25.3°±0.2°, 26.0°±0.2°, 26.8°±0.2°, 27.6°±0.2°, 28.1°±0.2°, 29.7°±0.2°, 30.2°±0.2°, 30.8°±0.2°, 31.5°±0.2°, and 33.5°±0.2° 2θ.

In certain embodiments, Form B of the mono-fumarate salt of the compound of formula (I) has an XRPD pattern comprising peaks at 4.7°±0.2°, 7.5°±0.2°, 9.7°±0.2°, 13.3°±0.2°, 13.8°±0.2°, 14.0°±0.2°, 14.2°±0.2°, 15.0°±0.2°, 15.4°±0.2°, 15.8°±0.2°, 16.6°±0.2°, 17.0°±0.2°, 17.4°±0.2°, 17.8°±0.2°, 18.5°±0.2°, 18.8°±0.2°, 19.5°±0.2°, 19.8°±0.2°, 20.3°±0.2°, 20.6°±0.2°, 21.5°±0.2°, 22.2°±0.2°, 22.6°±0.2°, 23.1°±0.2°, 23.4°±0.2°, 24.1°±0.2°, 24.4°±0.2°, 24.7°±0.2°, 25.3°±0.2°, 26.0°±0.2°, 26.8°±0.2°, 27.6°±0.2°, 28.1°±0.2°, 29.7°±0.2°, 30.2°±0.2°, 30.8°±0.2°, 31.5°±0.2°, and 33.5°±0.2° 2θ.

In certain embodiments, Form B of the mono-fumarate salt of the compound of formula (I) has an XRPD pattern substantially the same as shown in FIG. 3. In certain embodiments, Form B of the mono-fumarate salt of the compound of formula (I) has an XRPD pattern comprising one or more diffraction peaks (2θ) disclosed in Table 8.

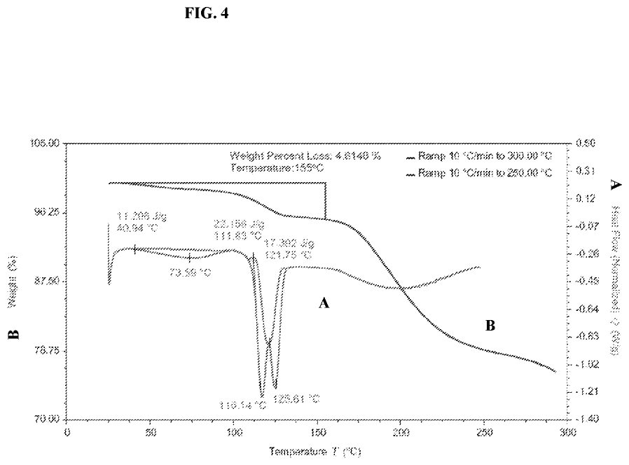

In certain embodiments, Form B of the mono-fumarate salt of the compound of formula (I) has a differential scanning calorimetry (DSC) thermogram comprising an endotherm with a peak onset at about 41° C. In certain embodiments, Form B of the mono-fumarate salt of the compound of formula (I) has a DSC thermogram comprising an endotherm with a peak onset at about 112° C. In certain embodiments, Form B of the mono-fumarate salt of the compound of formula (I) has a DSC thermogram comprising an endotherm with a peak onset at about 122° C. In certain embodiments, Form B of the mono-fumarate salt of the compound of formula (I) has a DSC thermogram comprising one or more endotherms with peak onsets at about 41° C., about 112° C., and about 122° C. In certain embodiments, Form B of the mono-fumarate salt of the compound of formula (I) has a DSC thermogram substantially the same as shown in FIG. 4.

In certain embodiments, Form B of the mono-fumarate salt of the compound of formula (I) exhibits a weight loss of less than or equal to about 4.6% wt upon heating Form B from about 25° C. to about 155° C. The weight loss exhibited by a crystalline form (e.g., Form B of the mono-fumarate salt of the compound of formula (I)) can be determined, for example, using thermogravimetric analysis (TGA). In certain embodiments, Form B of the mono-fumarate salt of the compound of formula (I) has a TGA thermogram substantially the same as shown in FIG. 4.

In certain embodiments, Form B of the mono-fumarate salt of the compound of formula (I) is a crystalline hydrate.

(iii) Form C of the Mono-Fumarate Salt

In various embodiments, provided herein is Form C of the mono-fumarate salt of the compound of formula (I)

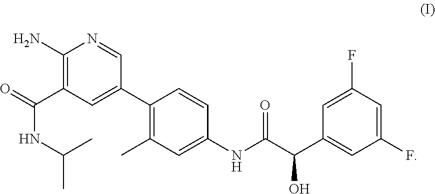 [SMILES: Cc1cc(NC(=O)[C@H](O)c2cc(F)cc(F)c2)ccc1-c1cnc(N)c(C(=O)NC(C)C)c1]

In certain embodiments, Form C of the mono-fumarate salt of the compound of formula (I) has an X-ray powder diffraction (XRPD) pattern comprising a peak at about 5.2° 2θ.

In certain embodiments, the XRPD pattern further comprises one or more peaks at about 7.6°, about 10.6°, about 13.8°, and about 14.4° 2θ.

In certain embodiments, the XRPD pattern further comprises one or more peaks at about 15.8°, about 21.2°, about 24.5°, and about 25.0° 2θ.

In certain embodiments, the XRPD pattern further comprises one or more peaks at about 15.1°, about 16.7°, about 17.3°, about 17.9°, about 19.4°, about 19.9°, about 20.6°, and about 22.7° 2θ.

In certain embodiments, the XRPD pattern further comprises one or more peaks at about 15.1°, about 15.8°, about 16.7°, about 17.3°, about 17.9°, about 19.4°, about 19.9°, about 20.6°, about 21.2°, about 22.7°, about 24.5°, and about 25.0° 2θ.

In certain embodiments, the XRPD pattern further comprises one or more peaks at about 25.7°, about 26.6°, about 28.9°, about 30.4°, about 31.4°, and about 32.0° 2θ.

In certain embodiments, Form C of the mono-fumarate salt of the compound of formula (I) has an XRPD pattern comprising one or more peaks at about 5.2°, about 7.6°, about 10.6°, about 13.8°, about 14.4°, about 15.1°, about 15.8°, about 16.7°, about 17.3°, about 17.9°, about 19.4°, about 19.9°, about 20.6°, about 21.2°, about 22.7°, about 24.5°, about 25.0°, about 25.7°, about 26.6°, about 28.9°, about 30.4°, about 31.4°, and about 32.0° 2θ.

In certain embodiments, Form C of the mono-fumarate salt of the compound of formula (I) has an XRPD pattern comprising peaks at about 5.2°, about 7.6°, about 10.6°, about 13.8°, about 14.4°, about 15.1°, about 15.8°, about 16.7°, about 17.3°, about 17.9°, about 19.4°, about 19.9°, about 20.6°, about 21.2°, about 22.7°, about 24.5°, about 25.0°, about 25.7°, about 26.6°, about 28.9°, about 30.4°, about 31.4°, and about 32.0° 2θ.

In certain embodiments, Form C of the mono-fumarate salt of the compound of formula (I) has an XRPD pattern comprising a peak at 5.2°±0.3° 2θ.

In certain embodiments, the XRPD pattern further comprises one or more peaks at 7.6°±0.3°, 10.6°±0.3°, 13.8°±0.3°, and 14.4°±0.3° 2θ.

In certain embodiments, the XRPD pattern further comprises one or more peaks at 15.8°±0.3°, 21.2°±0.3°, 24.5°±0.3°, and 25.0°±0.3° 2θ.

In certain embodiments, the XRPD pattern further comprises one or more peaks at 15.1°±0.3°, 16.7°±0.3°, 17.3°±0.3°, 17.9°±0.3°, 19.4°±0.3°, 19.9°±0.3°, 20.6°±0.3°, and 22.7°±0.3° 2θ.

In certain embodiments, the XRPD pattern further comprises one or more peaks at 15.1°±0.3°, 15.8°±0.3°, 16.7°±0.3°, 17.3°±0.3°, 17.9°±0.3°, 19.4°±0.3°, 19.9°±0.3°, 20.6°±0.3°, 21.2°±0.3°, 22.7°±0.3°, 24.5°±0.3°, and 25.0°±0.3° 2θ.

In certain embodiments, the XRPD pattern further comprises one or more peaks at 25.7°±0.3°, 26.6°±0.3°, 28.9°±0.3°, 30.4°±0.3°, 31.4°±0.3°, and 32.0°±0.3° 2θ.

In certain embodiments, Form C of the mono-fumarate salt of the compound of formula (I) has an XRPD pattern comprising one or more peaks at 5.2°±0.3°, 7.6°±0.3°, 10.6°±0.3°, 13.8°±0.3°, 14.4°±0.3°, 15.1°±0.3°, 15.8°±0.3°, 16.7°±0.3°, 17.3°±0.3°, 17.9°±0.3°, 19.4°±0.3°, 19.9°±0.3°, 20.6°±0.3°, 21.2°±0.3°, 22.7°±0.3°, 24.5°±0.3°, 25.0°±0.3°, 25.7°±0.3°, 26.6°±0.3°, 28.9°±0.3°, 30.4°±0.3°, 31.4°±0.3°, and 32.0°±0.3° 2θ.

In certain embodiments, Form C of the mono-fumarate salt of the compound of formula (I) has an XRPD pattern comprising peaks at 5.2°±0.3°, 7.6°±0.3°, 10.6°±0.3°, 13.8°±0.3°, 14.4°±0.3°, 15.1°±0.3°, 15.8°±0.3°, 16.7°±0.3°, 17.3°±0.3°, 17.9°±0.3°, 19.4°±0.3°, 19.9°±0.3°, 20.6°±0.3°, 21.2°±0.3°, 22.7°±0.3°, 24.5°±0.3°, 25.0°±0.3°, 25.7°±0.3°, 26.6°±0.3°, 28.9°±0.3°, 30.4°±0.3°, 31.4°±0.3°, and 32.0°±0.3° 2θ.

In certain embodiments, Form C of the mono-fumarate salt of the compound of formula (I) has an XRPD pattern comprising a peak at 5.2°±0.2° 2θ.

In certain embodiments, the XRPD pattern further comprises one or more peaks at 7.6°±0.2°, 10.6°±0.2°, 13.8°±0.2°, and 14.4°±0.2° 2θ.

In certain embodiments, the XRPD pattern further comprises one or more peaks at 15.8°±0.2°, 21.2°±0.2°, 24.5°±0.2°, and 25.0°±0.2° 2θ.

In certain embodiments, the XRPD pattern further comprises one or more peaks at 15.1°±0.2°, 16.7°±0.2°, 17.3°±0.2°, 17.9°±0.2°, 19.4°±0.2°, 19.9°±0.2°, 20.6°±0.2°, and 22.7°±0.2° 2θ.

In certain embodiments, the XRPD pattern further comprises one or more peaks at 15.1°±0.2°, 15.8°±0.2°, 16.7°±0.2°, 17.3°±0.2°, 17.2°±0.2°, 19.2°±0.2°, 19.9°±0.2°, 20.6°±0.2°, 21.2°±0.2°, 22.7°±0.2°, 24.5°±0.2°, and 25.0°±0.2° 2θ.

In certain embodiments, the XRPD pattern further comprises one or more peaks at 25.7°±0.2°, 26.6°±0.2°, 28.9°±0.2°, 30.4°±0.2°, 31.4°±0.2°, and 32.0°±0.2° 2θ.

In certain embodiments, Form C of the mono-fumarate salt of the compound of formula (I) has an XRPD pattern comprising one or more peaks at 5.2°±0.2°, 7.6°±0.2°, 10.6°±0.2°, 13.8°±0.2°, 14.4°±0.2°, 15.1°±0.2°, 15.8°±0.2°, 16.7°±0.2°, 17.3°±0.2°, 17.9°±0.2°, 19.4°±0.2°, 19.9°±0.2°, 20.6°±0.2°, 21.2°±0.2°, 22.7°±0.2°, 24.5°±0.2°, 25.0°±0.2°, 25.7°±0.2°, 26.6°±0.2°, 28.9°±0.2°, 30.4°±0.2°, 31.4°±0.2°, and 32.0°±0.2° 2θ.

In certain embodiments, Form C of the mono-fumarate salt of the compound of formula (I) has an XRPD pattern comprising peaks at 5.2°±0.2°, 7.6°±0.2°, 10.6°±0.2°, 13.8°±0.2°, 14.4°±0.2°, 15.1°±0.2°, 15.8°±0.2°, 16.7°±0.2°, 17.3°±0.2°, 17.9°±0.2°, 19.4°±0.2°, 19.9°±0.2°, 20.6°±0.2°, 21.2°±0.2°, 22.7°±0.2°, 24.5°±0.2°, 25.0°±0.2°, 25.7°±0.2°, 26.6°±0.2°, 28.9°±0.2°, 30.4°±0.2°, 31.4°±0.2°, and 32.0°±0.2° 2θ.

In certain embodiments, Form C of the mono-fumarate salt of the compound of formula (I) has an XRPD pattern substantially the same as shown in FIG. 5. In certain embodiments, Form C of the mono-fumarate salt of the compound of formula (I) has an XRPD pattern comprising one or more diffraction peaks (2θ) disclosed in Table 9.

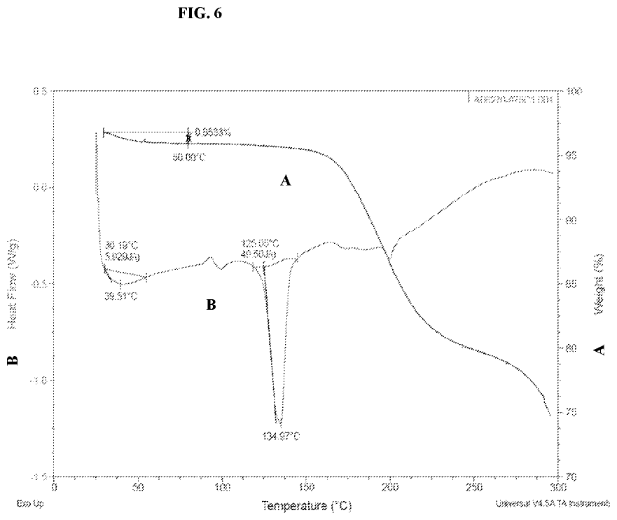

In certain embodiments, Form C of the mono-fumarate salt of the compound of formula (I) has a differential scanning calorimetry (DSC) thermogram comprising an endotherm with a peak onset at about 30° C. In certain embodiments, Form C of the mono-fumarate salt of the compound of formula (I) has a DSC thermogram comprising an endotherm with a peak onset at about 125° C. In certain embodiments, Form C of the mono-fumarate salt of the compound of formula (I) has a DSC thermogram comprising one or more endotherms with peak onsets at about 30° C., and about 125° C. In certain embodiments, Form C of the mono-fumarate salt of the compound of formula (I) has a DSC thermogram substantially the same as shown in FIG. 6.

In certain embodiments, Form C of the mono-fumarate salt of the compound of formula (I) exhibits a weight loss of less than or equal to about 0.9% wt upon heating Form C from about 25° C. to about 80° C. The weight loss exhibited by a crystalline form (e.g., Form C of the mono-fumarate salt of the compound of formula (I)) can be determined, for example, using thermogravimetric analysis (TGA). In certain embodiments, Form C of the mono-fumarate salt of the compound of formula (I) has a TGA thermogram substantially the same as shown in FIG. 6.

### (B) Crystalline Forms of the Hemi-Fumarate Salt

In another aspect, provided herein is a crystalline hemi-fumarate salt of the compound of formula (I)

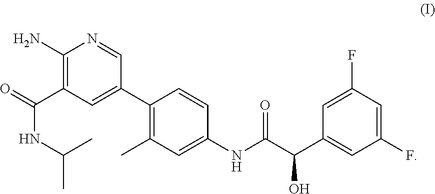 [SMILES: Cc1cc(NC(=O)[C@H](O)c2cc(F)cc(F)c2)ccc1-c1cnc(N)c(C(=O)NC(C)C)c1]

In certain embodiments, the crystalline hemi-fumarate salt has an XRPD pattern comprising one or more peaks at about 8.5°, about 11.0°, and about 12.0° 2θ.

In certain embodiments, the XRPD pattern further comprises one or more peaks at about 16.9°, about 17.9°, and about 24.1° 2θ.

In certain embodiments, the XRPD pattern further comprises one or more peaks at about 25.7°, about 27.7°, and about 34.1° 2θ.

In certain embodiments, the crystalline hemi-fumarate salt has an XRPD pattern comprising one or more peaks at about 8.5°, about 11.0°, about 12.0°, about 16.9°, about 17.9°, about 24.1°, about 25.7°, about 27.7°, and about 34.1° 2θ.

In certain embodiments, the crystalline hemi-fumarate salt has an XRPD pattern comprising peaks at about 8.5°, about 11.0°, about 12.0°, about 16.9°, about 17.9°, about 24.1°, about 25.7°, about 27.7°, and about 34.1° 2θ.

In certain embodiments, the crystalline hemi-fumarate salt has an XRPD pattern comprising one or more peaks at 8.5°±0.3°, 11.0°±0.3°, and 12.0°±0.3° 2θ.

In certain embodiments, the XRPD pattern further comprises one or more peaks at 16.9°±0.3°, 17.9°±0.3°, and 24.1°±0.3° 2θ.

In certain embodiments, the XRPD pattern further comprises one or more peaks at 25.7°±0.3°, 27.7°±0.3°, and 34.1°±0.3° 2θ.

In certain embodiments, the crystalline hemi-fumarate salt has an XRPD pattern comprising one or more peaks at 8.5°±0.3°, 11.0°±0.3°, 12.0°±0.3°, 16.9°±0.3°, 17.9°±0.3°, 24.1°±0.3°, 25.7°±0.3°, 27.7°±0.3°, and 34.1°±0.3° 2θ.

In certain embodiments, the crystalline hemi-fumarate salt has an XRPD pattern comprising peaks at 8.5°±0.3°, 11.0°±0.3°, 12.0°±0.3°, 16.9°±0.3°, 17.9°±0.3°, 24.1°±0.3°, 25.7°±0.3°, 27.7°±0.3°, and 34.1°±0.3° 2θ.

In certain embodiments, the crystalline hemi-fumarate salt has an XRPD pattern comprising one or more peaks at 8.5°±0.2°, 11.0°±0.2°, and 12.0°±0.2° 2θ.

In certain embodiments, the XRPD pattern further comprises one or more peaks at 16.9°±0.2°, 17.9°±0.2°, and 24.1°±0.2° 2θ.

In certain embodiments, the XRPD pattern further comprises one or more peaks at 25.7°±0.2°, 27.7°±0.2°, and 34.1°±0.2° 2θ.

In certain embodiments, the crystalline hemi-fumarate salt has an XRPD pattern comprising one or more peaks at 8.5°±0.2°, 11.0°±0.2°, 12.0°±0.2°, 16.9°±0.2°, 17.9°±0.2°, 24.1°±0.2°, 25.7°±0.2°, 27.7°±0.2°, and 34.1°±0.2° 2θ.

In certain embodiments, the crystalline hemi-fumarate salt has an XRPD pattern comprising peaks at 8.5°±0.2°, 11.0°±0.2°, 12.0°±0.2°, 16.9°±0.2°, 17.9°±0.2°, 24.1°±0.2°, 25.7°±0.2°, 27.7°±0.2°, and 34.1°±0.2° 2θ.

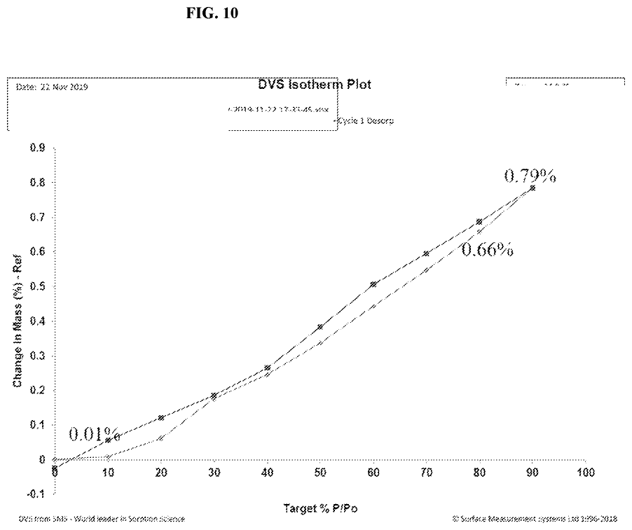

In certain embodiments, the crystalline hemi-fumarate salt of the compound of formula (I) is Form A of the hemi-fumarate salt of the compound of formula (I). In certain embodiments, the crystalline hemi-fumarate salt has an XRPD pattern substantially the same as shown in FIG. 7. In certain embodiments, the crystalline hemi-fumarate salt of the compound of formula (I) is Form B of the hemi-fumarate salt of the compound of formula (I). In certain embodiments, the crystalline hemi-fumarate salt has an XRPD pattern substantially the same as shown in FIG. 10.

In certain embodiments, the crystalline hemi-fumarate salt of the compound of formula (I) is an anhydrous crystalline form.

In certain embodiments, the crystalline hemi-fumarate salt of the compound of formula (I) is a crystalline hydrate.

### (i) Form A of the Hemi-Fumarate Salt

In various embodiments, provided herein is Form A of the hemi-fumarate salt of the compound of formula (I)

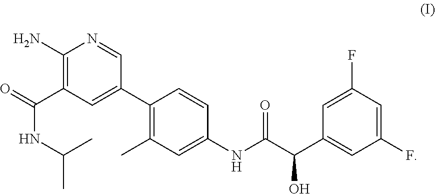 [SMILES: Cc1cc(NC(=O)[C@H](O)c2cc(F)cc(F)c2)ccc1-c1cnc(N)c(C(=O)NC(C)C)c1]

In certain embodiments, Form A of the hemi-fumarate salt of the compound of formula (I) has an X-ray powder diffraction (XRPD) pattern comprising one or more peaks at about 9.9° and about 11.0° 2θ.

In certain embodiments, the XRPD pattern further comprises one or more peaks at about 6.4°, about 8.3°, about 12.1°, about 13.2°, about 14.3°, and about 14.5° 2θ.

In certain embodiments, the XRPD pattern further comprises one or more peaks at about 16.9°, about 17.2°, about 17.8°, about 18.5°, about 20.2°, about 22.1°, and about 22.5° 2θ.

In certain embodiments, the XRPD pattern further comprises one or more peaks at about 15.1°, about 16.1°, about 19.5°, about 19.8°, about 21.2°, about 21.5°, about 23.3°, about 24.3°, about 24.5°, and about 25.0° 2θ.

In certain embodiments, the XRPD pattern further comprises one or more peaks at about 25.5°, about 26.3°, about 27.2°, about 28.9°, and about 31.1° 2θ.

In certain embodiments, the XRPD pattern further comprises one or more peaks at about 27.8°, about 28.0°, about 28.3°, about 29.5°, about 31.9°, about 32.5°, about 32.7°, about 33.6°, about 34.1°, and about 34.4° 2θ.

In certain embodiments, Form A of the hemi-fumarate salt of the compound of formula (I) has an XRPD pattern comprising one or more peaks at about 6.4°, about 8.3°, about 9.9°, about 11.0°, about 12.1°, about 13.2°, about 14.3°, about 14.5°, about 15.1°, about 16.1°, about 16.9°, about 17.2°, about 17.8°, about 18.5°, about 19.5°, about 19.8°, about 20.2°, about 21.2°, about 21.5°, about 22.1°, about 22.5°, about 23.3°, about 24.3°, about 24.5°, about 25.0°, about 25.5°, about 26.3°, about 27.2°, about 27.8°, about 28.0°, about 28.3°, about 28.9°, about 29.5°, about 31.1°, about 31.9°, about 32.5°, about 32.7°, about 33.6°, about 34.1°, and about 34.4° 2θ.

In certain embodiments, Form A of the hemi-fumarate salt of the compound of formula (I) has an XRPD pattern comprising peaks at about 6.4°, about 8.3°, about 9.9°, about 11.0°, about 12.1°, about 13.2°, about 14.3°, about 14.5°, about 15.1°, about 16.1°, about 16.9°, about 17.2°, about 17.8°, about 18.5°, about 19.5°, about 19.8°, about 20.2°, about 21.2°, about 21.5°, about 22.1°, about 22.5°, about 23.3°, about 24.3°, about 24.5°, about 25.0°, about 25.5°, about 26.3°, about 27.2°, about 27.8°, about 28.0°, about 28.3°, about 28.9°, about 29.5°, about 31.1°, about 31.9°, about 32.5°, about 32.7°, about 33.6°, about 34.1°, and about 34.4° 2θ.

In certain embodiments, Form A of the hemi-fumarate salt of the compound of formula (I) has an X-ray powder diffraction (XRPD) pattern comprising one or more peaks at 9.9°±0.3° and 11.0°±0.3° 2θ.

In certain embodiments, the XRPD pattern further comprises one or more peaks at 6.4°±0.3°, 8.3°±0.3°, 12.1°±0.3°, 13.2°±0.3°, 14.3°±0.3°, and 14.5°±0.3° 2θ.

In certain embodiments, the XRPD pattern further comprises one or more peaks at 16.9°±0.3°, 17.2°±0.3°, 17.8°±0.3°, 18.5°±0.3°, 20.2°±0.3°, 22.1°±0.3°, and 22.5°±0.3° 2θ.

In certain embodiments, the XRPD pattern further comprises one or more peaks at 15.1°±0.3°, 16.1°±0.3°, 19.5°±0.3°, 19.8°±0.3°, 21.2°±0.3°, 21.5°±0.3°, 23.3°±0.3°, 24.3°±0.3°, 24.5°±0.3°, and 25.0°±0.3° 2θ.

In certain embodiments, the XRPD pattern further comprises one or more peaks at 25.5°±0.3°, 26.3°±0.3°, 27.2°±0.3°, 28.9°±0.3°, and 31.1°±0.3° 2θ.

In certain embodiments, the XRPD pattern further comprises one or more peaks at 27.8°±0.3°, 28.0°±0.3°, 28.3°±0.3°, 29.5°±0.3°, 31.9°±0.3°, 32.5°±0.3°, 32.7°±0.3°, 33.6°±0.3°, 34.1°±0.3°, and 34.4°±0.3° 2θ.

In certain embodiments, Form A of the hemi-fumarate salt of the compound of formula (I) has an XRPD pattern comprising one or more peaks at 6.4°±0.3°, 8.3°±0.3°, 9.9°±0.3°, 11.0°±0.3°, 12.1°±0.3°, 13.2°±0.3°, 14.3°±0.3°, 14.5°±0.3°, 15.1°±0.3°, 16.1°±0.3°, 16.9°±0.3°, 17.2°±0.3°, 17.8°±0.3°, 18.5°±0.3°, 19.5°±0.3°, 19.8°±0.3°, 20.2°±0.3°, 21.2°±0.3°, 21.5°±0.3°, 22.1°±0.3°, 22.5°±0.3°, 23.3°±0.3°, 24.3°±0.3°, 24.5°±0.3°, 25.0°±0.3°, 25.5°±0.3°, 26.3°±0.3°, 27.2°±0.3°, 27.8°±0.3°, 28.0°±0.3°, 28.3°±0.3°, 28.9°±0.3°, 29.5°±0.3°, 31.1°±0.3°, 31.9°±0.3°, 32.5°±0.3°, 32.7°±0.3°, 33.6°±0.3°, 34.1°±0.3°, and 34.4°±0.3° 2θ.

In certain embodiments, Form A of the hemi-fumarate salt of the compound of formula (I) has an XRPD pattern comprising peaks at 6.4°±0.3°, 8.3°±0.3°, 9.9°±0.3°, 11.0°±0.3°, 12.1°±0.3°, 13.2°±0.3°, 14.3°±0.3°, 14.5°±0.3°, 15.1°±0.3°, 16.1°±0.3°, 16.9°±0.3°, 17.2°±0.3°, 17.8°±0.3°, 18.5°±0.3°, 19.5°±0.3°, 19.8°±0.3°, 20.2°±0.3°, 21.2°±0.3°, 21.5°±0.3°, 22.1°±0.3°, 22.5°±0.3°, 23.3°±0.3°, 24.3°±0.3°, 24.5°±0.3°, 25.0°±0.3°, 25.5°±0.3°, 26.3°±0.3°, 27.2°±0.3°, 27.8°±0.3°, 28.0°±0.3°, 28.3°±0.3°, 28.9°±0.3°, 29.5°±0.3°, 31.1°±0.3°, 31.9°±0.3°, 32.5°±0.3°, 32.7°±0.3°, 33.6°±0.3°, 34.1°±0.3°, and 34.4°±0.3° 2θ.

In certain embodiments, Form A of the hemi-fumarate salt of the compound of formula (I) has an X-ray powder diffraction (XRPD) pattern comprising one or more peaks at 9.9°±0.2° and 11.0°±0.2° 2θ.

In certain embodiments, the XRPD pattern further comprises one or more peaks at 6.4°±0.2°, 8.3°±0.2°, 12.1°±0.2°, 13.2°±0.2°, 14.3°±0.2°, and 14.5°±0.2° 2θ.

In certain embodiments, the XRPD pattern further comprises one or more peaks at 16.9°±0.2°, 17.2°±0.2°, 17.8°±0.2°, 18.5°±0.2°, 20.2°±0.2°, 22.1°±0.2°, and 22.5°±0.2° 2θ.

In certain embodiments, the XRPD pattern further comprises one or more peaks at 15.1°±0.2°, 16.1°±0.2°, 19.5°±0.2°, 19.8°±0.2°, 21.2°±0.2°, 21.5°±0.2°, 23.3°±0.2°, 24.3°±0.2°, 24.5°±0.2°, and 25.0°±0.2° 2θ.

In certain embodiments, the XRPD pattern further comprises one or more peaks at 25.5°±0.2°, 26.3°±0.2°, 27.2°±0.2°, 28.9°±0.2°, and 31.1°±0.2° 2θ.

In certain embodiments, the XRPD pattern further comprises one or more peaks at 27.8°±0.2°, 28.0°±0.2°, 28.3°±0.2°, 29.5°±0.2°, 31.9°±0.2°, 32.5°±0.2°, 32.7°±0.2°, 33.6°±0.2°, 34.1°±0.2°, and 34.4°±0.2° 2θ.

In certain embodiments, Form A of the hemi-fumarate salt of the compound of formula (I) has an XRPD pattern comprising one or more peaks at 6.4°±0.2°, 8.3°±0.2°, 9.9°±0.2°, 11.0°±0.2°, 12.1°±0.2°, 13.2°±0.2°, 14.3°±0.2°, 14.5°±0.2°, 15.1°±0.2°, 16.1°±0.2°, 16.9°±0.2°, 17.2°±0.2°, 17.8°±0.2°, 18.5°±0.2°, 19.5°±0.2°, 19.8°±0.2°, 20.2°±0.2°, 21.2°±0.2°, 21.5°±0.2°, 22.1°±0.2°, 22.5°±0.2°, 23.3°±0.2°, 24.3°±0.2°, 24.5°±0.2°, 25.0°±0.2°, 25.5°±0.2°, 26.3°±0.2°, 27.2°±0.2°, 27.8°±0.2°, 28.0°±0.2°, 28.3°±0.2°, 28.9°±0.2°, 29.5°±0.2°, 31.1°±0.2°, 31.9°±0.2°, 32.5°±0.2°, 32.7°±0.2°, 33.6°±0.2°, 34.1°±0.2°, and 34.4°±0.2° 2θ.

In certain embodiments, Form A of the hemi-fumarate salt of the compound of formula (I) has an XRPD pattern comprising peaks at 6.4°±0.2°, 8.3°±0.2°, 9.9°±0.2°, 11.0°±0.2°, 12.1°±0.2°, 13.2°±0.2°, 14.3°±0.2°, 14.5°±0.2°, 15.1°±0.2°, 16.1°±0.2°, 16.9°±0.2°, 17.2°±0.2°, 17.8°±0.2°, 18.5°±0.2°, 19.5°±0.2°, 19.8°±0.2°, 20.2°±0.2°, 21.2°±0.2°, 21.5°±0.2°, 22.1°±0.2°, 22.5°±0.2°, 23.3°±0.2°, 24.3°±0.2°, 24.5°±0.2°, 25.0°±0.2°, 25.5°±0.2°, 26.3°±0.2°, 27.2°±0.2°, 27.8°±0.2°, 28.0°±0.2°, 28.3°±0.2°, 28.9°±0.2°, 29.5°±0.2°, 31.1°±0.2°, 31.9°±0.2°, 32.5°±0.2°, 32.7°±0.2°, 33.6°±0.2°, 34.1°±0.2°, and 34.4°±0.2° 2θ.

In certain embodiments, Form A of the hemi-fumarate salt of the compound of formula (I) has an XRPD pattern substantially the same as shown in FIG. 8. In certain embodiments, Form A of the hemi-fumarate salt of the compound of formula (I) has an XRPD pattern comprising one or more diffraction peaks (2θ) disclosed in Table 11.

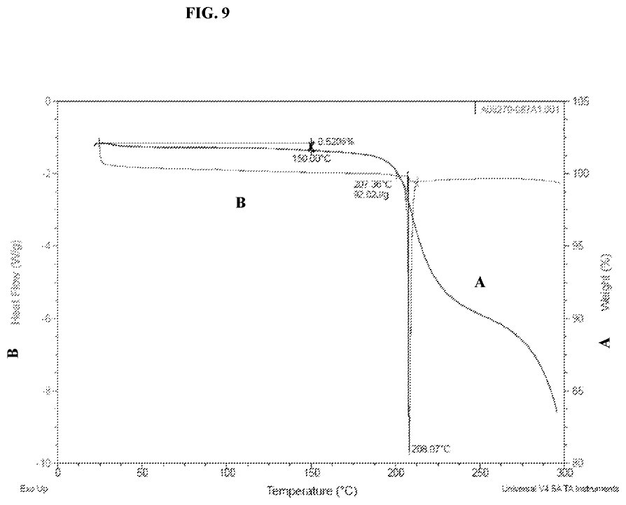

In certain embodiments, Form A of the hemi-fumarate salt of the compound of formula (I) has a differential scanning calorimetry (DSC) thermogram comprising an endotherm with a peak onset at about 206° C. In certain embodiments, Form B of the hemi-fumarate salt of the compound of formula (I) has a DSC thermogram substantially the same as shown in FIG. 9.

In certain embodiments, Form A of the hemi-fumarate salt of the compound of formula (I) exhibits a weight loss of less than or equal to about 0.5% wt upon heating Form A from about 25° C. to about 150° C. The weight loss exhibited by a crystalline form (e.g., Form A of the hemi-fumarate salt of the compound of formula (I)) can be determined, for example, using thermogravimetric analysis (TGA). In certain embodiments, Form A of the hemi-fumarate salt of the compound of formula (I) has a TGA thermogram substantially the same as shown in FIG. 9.

In certain embodiments, Form A of the hemi-fumarate salt of the compound of formula (I) exhibits a change in mass of less than or equal to about 0.7% wt when varying the relative humidity between 0% and about 80%, when measured at 25° C. The weight change exhibited by a crystalline form (e.g., Form A of the hemi-fumarate salt of the compound of formula (I)) can be determined, for example, using DVS. In certain embodiments, Form A of the hemi-fumarate salt of the compound of formula (I) has a water sorption isotherm substantially the same as shown in FIG. 10.

In certain embodiments, Form A of the hemi-fumarate salt of the compound of formula (I) is an anhydrous crystalline form.

### (ii) Form B of the Hemi-Fumarate Salt

In various embodiments, provided herein is Form B of the hemi-fumarate salt of the compound of formula (I)

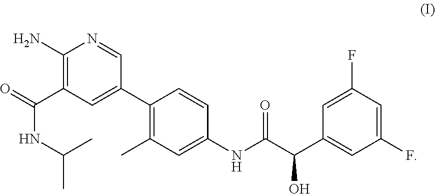 [SMILES: Cc1cc(NC(=O)[C@H](O)c2cc(F)cc(F)c2)ccc1-c1cnc(N)c(C(=O)NC(C)C)c1]

In certain embodiments, Form B of the hemi-fumarate salt of the compound of formula (I) has an X-ray powder diffraction (XRPD) pattern comprising a peak at about 5.4° 2θ. In certain embodiments, Form B of the hemi-fumarate salt of the compound of formula (I) has an XRPD pattern comprising a peak at about 8.7° 2θ. In certain embodiments, In certain embodiments, Form B of the hemi-fumarate salt of the compound of formula (I) has an XRPD pattern comprising one or more peaks at about 5.4° and 8.7° 2θ.

In certain embodiments, the XRPD pattern further comprises one or more peaks at about 10.4°, about 11.0°, about 11.9°, and about 12.6° 2θ.

In certain embodiments, the XRPD pattern further comprises one or more peaks at about 16.5°, about 16.9°, about 17.4°, about 17.9°, and about 23.9° 2θ.

In certain embodiments, the XRPD pattern further comprises one or more peaks at about 25.7°, about 27.7°, and about 34.1° 2θ.

In certain embodiments, Form B of the hemi-fumarate salt of the compound of formula (I) has an XRPD pattern comprising one or more peaks at about 5.4°, about 8.7°, about 10.4°, about 11.0°, about 11.9°, about 12.6°, about 16.5°, about 16.9°, about 17.4°, about 17.9°, and about 23.9°, about 25.7°, about 27.7°, and about 34.1° 2θ.

In certain embodiments, Form B of the hemi-fumarate salt of the compound of formula (I) has an XRPD pattern comprising peaks at about 5.4°, about 8.7°, about 10.4°, about 11.0°, about 11.9°, about 12.6°, about 16.5°, about 16.9°, about 17.4°, about 17.9°, and about 23.9°, about 25.7°, about 27.7°, and about 34.1° 2θ.

In certain embodiments, Form B of the hemi-fumarate salt of the compound of formula (I) has an XRPD pattern comprising a peak at 5.4°±0.3° 2θ. In certain embodiments, Form B of the hemi-fumarate salt of the compound of formula (I) has an XRPD pattern comprising a peak at about 8.7°±0.3° 2θ. In certain embodiments, Form B of the hemi-fumarate salt of the compound of formula (I) has an XRPD pattern comprising one or more peaks at about 5.4°±0.3° and 8.7°±0.3° 2θ.

In certain embodiments, the XRPD pattern further comprises one or more peaks at about 10.4°±0.3°, about 11.0°±0.3°, about 11.9°±0.3°, and about 12.6°±0.3° 2θ.

In certain embodiments, the XRPD pattern further comprises one or more peaks at about 16.5°±0.3°, about 16.9°±0.3°, about 17.4°±0.3°, about 17.9°±0.3°, and about 23.9°±0.3° 2θ.

In certain embodiments, the XRPD pattern further comprises one or more peaks at about 25.7°±0.3°, about 27.7°±0.3°, and about 34.1°±0.3° 2θ.

In certain embodiments, Form B of the hemi-fumarate salt of the compound of formula (I) has an XRPD pattern comprising one or more peaks at 5.4°±0.3°, 8.7°±0.3°, 10.4°±0.3°, 11.0°±0.3°, 11.9°±0.3°, 12.6°±0.3°, 16.5°±0.3°, 16.9°±0.3°, 17.4°±0.3°, 17.9°±0.3°, 23.9°±0.3°, 25.7°±0.3°, 27.7°±0.3°, and 34.1°±0.3° 2θ.

In certain embodiments, Form B of the hemi-fumarate salt of the compound of formula (I) has an XRPD pattern comprising peaks at 5.4°±0.3°, 8.7°±0.3°, 10.4°±0.3°, 11.0°±0.3°, 11.9°±0.3°, 12.6°±0.3°, 16.5°±0.3°, 16.9°±0.3°, 17.4°±0.3°, 17.9°±0.3°, 23.9°±0.3°, 25.7°±0.3°, 27.7°±0.3°, and 34.1°±0.3° 2θ.

In certain embodiments, Form B of the hemi-fumarate salt of the compound of formula (I) has an XRPD pattern comprising a peak at 5.4°±0.2° 2θ. In certain embodiments, Form B of the hemi-fumarate salt of the compound of formula (I) has an XRPD pattern comprising a peak at about 8.7°±0.2° 2θ. In certain embodiments, Form B of the hemi-fumarate salt of the compound of formula (I) has an XRPD pattern comprising one or more peaks at about 5.4°±0.2° and 8.7°±0.2° 2θ.

In certain embodiments, the XRPD pattern further comprises one or more peaks at about 10.4°±0.2°, about 11.0°±0.2°, about 11.9°±0.2°, and about 12.6°±0.2° 2θ.

In certain embodiments, the XRPD pattern further comprises one or more peaks at about 16.5°±0.2°, about 16.9°±0.2°, about 17.4°±0.2°, about 17.9°±0.2°, and about 23.9°±0.2° 2θ.

In certain embodiments, the XRPD pattern further comprises one or more peaks at about 25.7°±0.2°, about 27.7°±0.2°, and about 34.1°±0.2° 2θ.

In certain embodiments, Form B of the hemi-fumarate salt of the compound of formula (I) has an XRPD pattern comprising one or more peaks at 5.4°±0.2°, 8.7°±0.2°, 10.4°±0.2°, 11.0°±0.2°, 11.9°±0.2°, 12.6°±0.2°, 16.5°±0.2°, 16.9°±0.2°, 17.4°±0.2°, 17.9°±0.2°, 23.9°±0.2°, 25.7°±0.2°, 27.7°±0.2°, and 34.1°±0.2° 2θ.

In certain embodiments, Form B of the hemi-fumarate salt of the compound of formula (I) has an XRPD pattern comprising peaks at 5.4°±0.2°, 8.7°±0.2°, 10.4°±0.2°, 11.0°±0.2°, 11.9°±0.2°, 12.6°±0.2°, 16.5°±0.2°, 16.9°±0.2°, 17.4°±0.2°, 17.9°±0.2°, 23.9°±0.2°, 25.7°±0.2°, 27.7°±0.2°, and 34.1°±0.2° 2θ.

In certain embodiments, Form B of the hemi-fumarate salt of the compound of formula (I) has an XRPD pattern substantially the same as shown in FIG. 13. In certain embodiments, Form B of the hemi-fumarate salt of the compound of formula (I) has an XRPD pattern comprising one or more diffraction peaks (2θ) disclosed in Table 19.

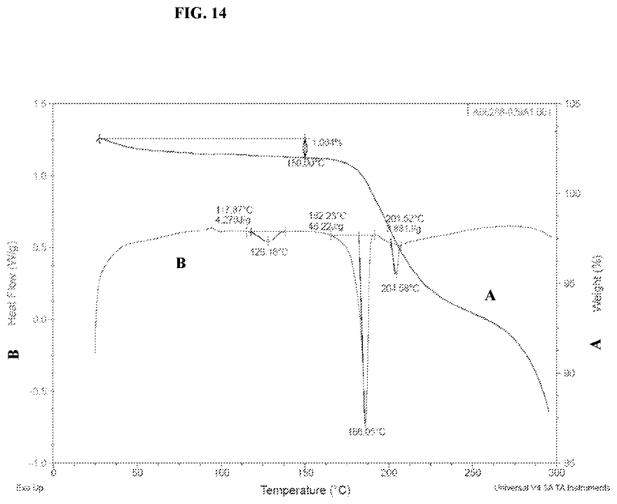

In certain embodiments, Form B of the hemi-fumarate salt of the compound of formula (I) has a differential scanning calorimetry (DSC) thermogram comprising an endotherm with a peak onset at about 118° C. In certain embodiments, Form B of the hemi-fumarate salt of the compound of formula (I) has a DSC thermogram comprising an endotherm with a peak onset at about 182° C. In certain embodiments, Form B of the hemi-fumarate salt of the compound of formula (I) has a DSC thermogram comprising an endotherm with a peak onset at about 202° C. In certain embodiments, Form B of the hemi-fumarate salt of the compound of formula (I) has a DSC thermogram comprising one or more endotherms with peak onsets at about 118° C., about 182° C., and about 202° C. In certain embodiments, Form B of the hemi-fumarate salt of the compound of formula (I) has a DSC thermogram substantially the same as shown in FIG. 14.

In certain embodiments, Form B of the hemi-fumarate salt of the compound of formula (I) exhibits a weight loss of less than or equal to about 1.1% wt upon heating Form B from about 25° C. to about 150° C. The weight loss exhibited by a crystalline form (e.g., Form B of the hemi-fumarate salt of the compound of formula (I)) can be determined, for example, using thermogravimetric analysis (TGA). In certain embodiments, Form B of the hemi-fumarate salt of the compound of formula (I) has a TGA thermogram substantially the same as shown in FIG. 14.

In certain embodiments, Form B of the hemi-fumarate salt of the compound of formula (I) is a crystalline hydrate.

### (2) L-Tartrate Salt Forms

In various embodiments, provided herein is an L-tartrate salt of the compound of formula (I)

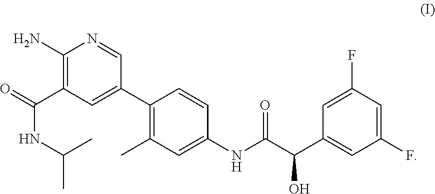 [SMILES: Cc1cc(NC(=O)[C@H](O)c2cc(F)cc(F)c2)ccc1-c1cnc(N)c(C(=O)NC(C)C)c1]

In certain embodiments, the L-tartrate salt of the compound of formula (I) is a crystalline salt form.

In various embodiments, provided herein is a crystalline L-tartrate salt of the compound of formula (I)

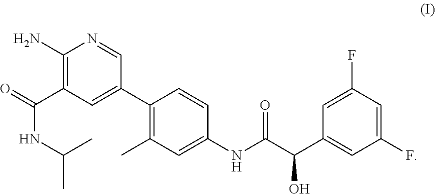 [SMILES: Cc1cc(NC(=O)[C@H](O)c2cc(F)cc(F)c2)ccc1-c1cnc(N)c(C(=O)NC(C)C)c1]

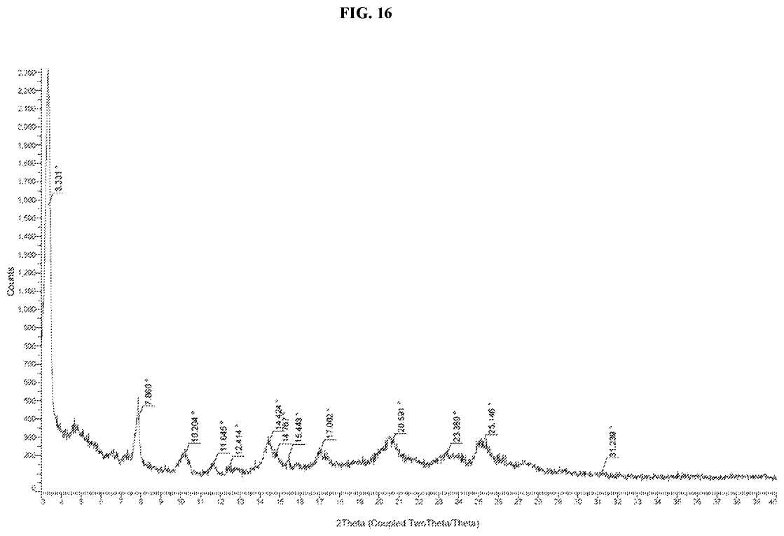

In certain embodiments, the crystalline L-tartrate salt has an XRPD pattern substantially the same as shown in FIG. 16.

In certain embodiments, the crystalline fumarate salt of the compound of formula (I) has an XRPD pattern comprising one or more diffraction peaks (2θ) disclosed in Table 21.

In certain embodiments, the L-tartrate salt of the compound of formula (I) is a solvated crystalline form. In certain embodiments, the L-tartrate salt of the compound of formula (I) is a crystalline MTBE solvate form.

In certain embodiments, the L-tartrate salt of the compound of formula (I) has an X-ray powder diffraction (XRPD) pattern comprising a peak at about 3.3° 2θ. In certain embodiments, the L-tartrate salt of the compound of formula (I) has an XRPD pattern comprising a peak at about 7.9° 2θ. In certain embodiments, the L-tartrate salt of the compound of formula (I) has an XRPD pattern comprising one or more peaks at about 3.3° and about 7.9° 2θ.

In certain embodiments, the XRPD pattern further comprises one or more peaks at about 10.2°, about 11.6°, about 12.4°, about 14.4°, and about 14.8° 2θ.

In certain embodiments, the XRPD pattern further comprises one or more peaks at about 15.4°, about 17.0°, about 20.6°, and about 23.4° 2θ.

In certain embodiments, the XRPD pattern further comprises one or more peaks at about 25.1°, and about 31.2° 2θ.

In certain embodiments, the L-tartrate salt of the compound of formula (I) has an XRPD pattern comprising one or more peaks at about 3.3°, about 7.9°, about 10.2°, about 11.6°, about 12.4°, about 14.4°, about 14.8°, about 15.4°, about 17.0°, about 20.6°, about 23.4°, about 25.1°, and about 31.2° 2θ.

In certain embodiments, the L-tartrate salt of the compound of formula (I) has an XRPD pattern comprising peaks at about 3.3°, about 7.9°, about 10.2°, about 11.6°, about 12.4°, about 14.4°, about 14.8°, about 15.4°, about 17.0°, about 20.6°, about 23.4°, about 25.1°, and about 31.2° 2θ.

In certain embodiments, the L-tartrate salt of the compound of formula (I) has an XRPD pattern comprising a peak at 3.3°±0.3° 2θ. In certain embodiments, the L-tartrate salt of the compound of formula (I) has an XRPD pattern comprising a peak at 7.9°±0.3° 2θ. In certain embodiments, the L-tartrate salt of the compound of formula (I) has an X-ray powder diffraction (XRPD) pattern comprising one or more peaks at 3.3°±0.3° and 7.9°±0.3° 2θ.

In certain embodiments, the XRPD pattern further comprises one or more peaks at 10.2°±0.3°, 11.6°±0.3°, 12.4°±0.3°, 14.4°±0.3°, and 14.8°±0.3° 2θ.

In certain embodiments, the XRPD pattern further comprises one or more peaks at 15.4°±0.3°, 17.0°±0.3°, 20.6°±0.3°, and 23.4°±0.3° 2θ.

In certain embodiments, the XRPD pattern further comprises one or more peaks at 25.1°±0.3°, and 31.2°±0.3° 2θ.

In certain embodiments, the L-tartrate salt of the compound of formula (I) has an XRPD pattern comprising one or more peaks at 3.3°±0.3°, 7.9°±0.3°, 10.2°±0.3°, 11.6°±0.3°, 12.4°±0.3°, 14.4°±0.3°, 14.8°±0.3°, 15.4°±0.3°, 17.0°±0.3°, 20.6°±0.3°, 23.4°±0.3°, 25.1°±0.3°, and 31.2°±0.3° 2θ.

In certain embodiments, the L-tartrate salt of the compound of formula (I) has an XRPD pattern comprising peaks at 3.3°±0.3°, 7.9°±0.3°, 10.2°±0.3°, 11.6°±0.3°, 12.4°±0.3°, 14.4°±0.3°, 14.8°±0.3°, 15.4°±0.3°, 17.0°±0.3°. 20.6°±0.3°, 23.4°±0.3°, 25.1°±0.3°, and 31.2°±0.3° 2θ.

In certain embodiments, the L-tartrate salt of the compound of formula (I) has an XRPD pattern comprising a peak at 3.3°±0.2° 2θ. In certain embodiments, the L-tartrate salt of the compound of formula (I) has an XRPD pattern comprising a peak at 7.9°±0.2° 2θ. In certain embodiments, the L-tartrate salt of the compound of formula (I) has an XRPD pattern comprising one or more peaks at 3.3°±0.2° and 7.9°±0.2° 2θ.

In certain embodiments, the XRPD pattern further comprises one or more peaks at 10.2°±0.2°, 11.6°±0.2°, 12.4°±0.2°, 14.4°±0.2°, and 14.8°±0.2° 2θ.

In certain embodiments, the XRPD pattern further comprises one or more peaks at 15.4°±0.2°, 17.0°±0.2°, 20.6°±0.2°, and 23.4°±0.2° 2θ.

In certain embodiments, the XRPD pattern further comprises one or more peaks at 25.1°±0.2°, and 31.2°±0.2° 2θ.

In certain embodiments, the L-tartrate salt of the compound of formula (I) has an XRPD pattern comprising one or more peaks at 3.3°±0.2°, 7.9°±0.2°, 10.2°±0.2°, 11.6°±0.2°, 12.4°±0.2°, 14.4°±0.2°, 14.8°±0.2°, 15.4°±0.2°, 17.0°±0.2°, 20.6°±0.2°, 23.4°±0.2°, 25.1°±0.2°, and 31.2°±0.2° 2θ.

In certain embodiments, the L-tartrate salt of the compound of formula (I) has an XRPD pattern comprising peaks at 3.3°±0.2°, 7.9°±0.2°, 10.2°±0.2°, 11.6°±0.2°, 12.4° 0.2°, 14.4°±0.2°, 14.8°±0.2°, 15.4°±0.2°, 17.0°±0.2°, 20.6°±0.2°, 23.4°±0.2°, 25.1°±0.2°, and 31.2°±0.2° 2θ.

In certain embodiments, the L-tartrate salt of the compound of formula (I) has an XRPD pattern substantially the same as shown in FIG. 16. In certain embodiments, the L-tartrate salt of the compound of formula (I) has an XRPD pattern comprising one or more diffraction peaks (2θ) disclosed in Table 21.

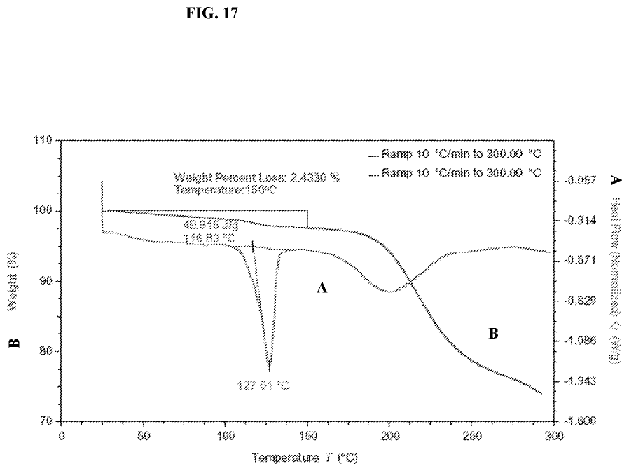

In certain embodiments, the L-tartrate salt of the compound of formula (I) has a differential scanning calorimetry (DSC) thermogram comprising an endotherm with a peak onset at about 117° C. In certain embodiments, the L-tartrate salt of the compound of formula (I) has a DSC thermogram substantially the same as shown in FIG. 17.

In certain embodiments, the L-tartrate of the compound of formula (I) exhibits a weight loss of less than or equal to about 2.4% wt. upon heating the L-tartrate salt from about 25° C. to about 150° C. The weight loss exhibited by a crystalline form (e.g., the L-tartrate salt of the compound of formula (I)) can be determined, for example, using thermogravimetric analysis (TGA). In certain embodiments, the L-tartrate salt of the compound of formula (I) has a TGA thermogram substantially the same as shown in FIG. 17.

### (3) Tosylate Salt Forms

In various embodiments, provided herein is a tosylate salt of the compound of formula (I)

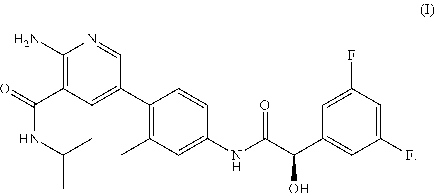 [SMILES: Cc1cc(NC(=O)[C@H](O)c2cc(F)cc(F)c2)ccc1-c1cnc(N)c(C(=O)NC(C)C)c1]

In certain embodiments, the tosylate salt of the compound of formula (I) is a crystalline salt form.

In various embodiments, provided herein is a crystalline tosylate salt of the compound of formula (I)

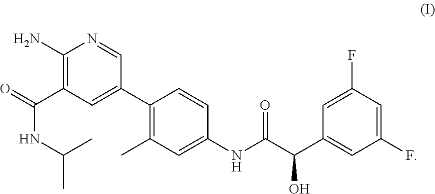 [SMILES: Cc1cc(NC(=O)[C@H](O)c2cc(F)cc(F)c2)ccc1-c1cnc(N)c(C(=O)NC(C)C)c1]

In certain embodiments, the crystalline tosylate salt of the compound of formula (I) is Form A of the tosylate salt of the compound of formula (I).

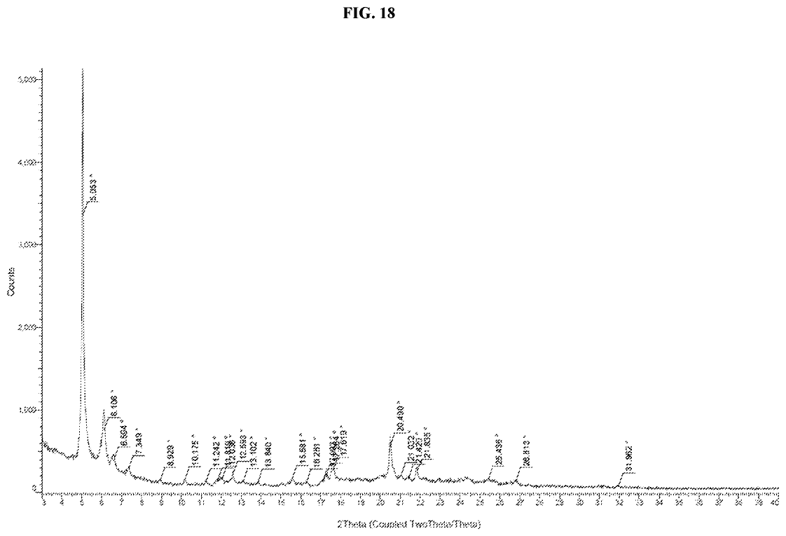

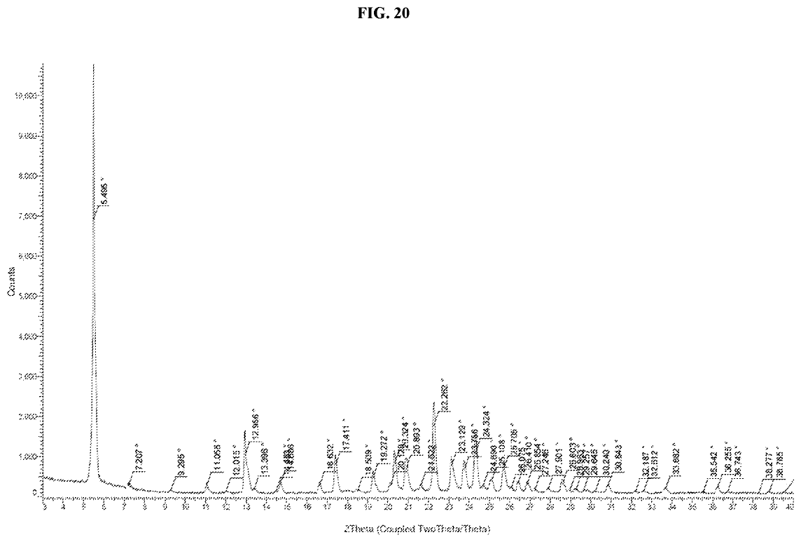

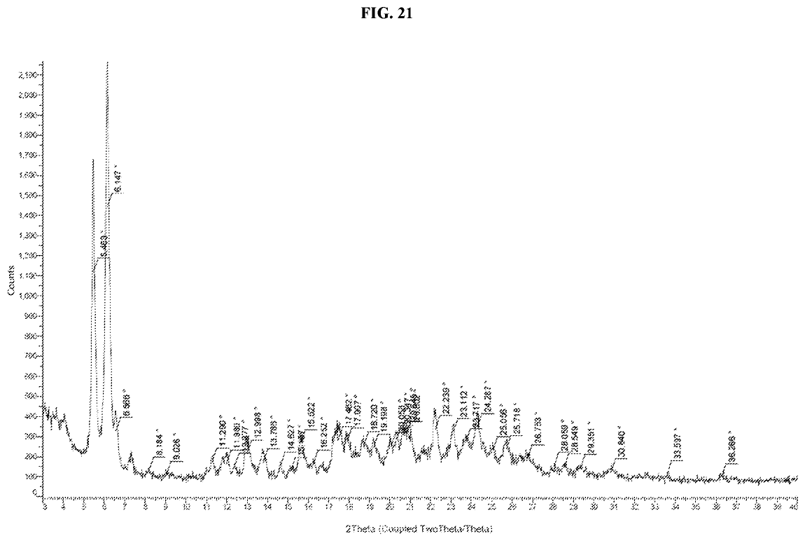

In certain embodiments, the crystalline tosylate salt has an XRPD pattern substantially the same as shown in FIG. 18. In certain embodiments, the crystalline tosylate salt has an XRPD pattern substantially the same as shown in FIG. 20. In certain embodiments, the crystalline tosylate salt has an XRPD pattern substantially the same as shown in FIG. 21.

In certain embodiments, the crystalline fumarate salt of the compound of formula (I) has an XRPD pattern comprising one or more diffraction peaks (2θ) disclosed in Table 22. In certain embodiments, the crystalline fumarate salt of the compound of formula (I) has an XRPD pattern comprising one or more diffraction peaks (2θ) disclosed in Table 23. In certain embodiments, the crystalline fumarate salt of the compound of formula (I) has an XRPD pattern comprising one or more diffraction peaks (2θ) disclosed in Table 24.

In certain embodiments, Form A of the tosylate salt of the compound of formula (I) is a solvated crystalline form. In certain embodiments, Form A of the tosylate salt of the compound of formula (I) is a crystalline IPAc solvate form.

### (A) Form A of the Tosylate Salt

In various embodiments, provided herein is Form A of the tosylate salt of the compound of formula (I)

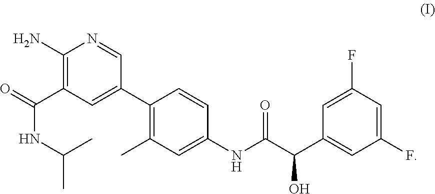 [SMILES: Cc1cc(NC(=O)[C@H](O)c2cc(F)cc(F)c2)ccc1-c1cnc(N)c(C(=O)NC(C)C)c1]

In certain embodiments, Form A of the tosylate salt of the compound of formula (I) has an X-ray powder diffraction (XRPD) pattern comprising a peak at about 5.1° 2θ.

In certain embodiments, the XRPD pattern further comprises one or more peaks at about 6.1°, about 6.6°, about 7.3°, about 8.9°, about 10.2°, about 11.2°, about 11.8°, about 12.0°, about 12.6°, about 13.1°, and about 13.8° 2θ.

In certain embodiments, the XRPD pattern further comprises one or more peaks at about 15.6°, about 16.3°, about 17.1°, about 17.3°, about 17.6°, about 20.5°, about 21.0°, and about 21.8° 2θ.

In certain embodiments, the XRPD pattern further comprises one or more peaks at about 25.4°, about 26.8°, and about 32.0° 2θ.

In certain embodiments, Form A of the tosylate salt of the compound of formula (I) has an XRPD pattern comprising one or more peaks at about 5.1°, about 6.1°, about 6.6°, about 7.3°, about 8.9°, about 10.2°, about 11.2°, about 11.8°, about 12.0°, about 12.6°, about 13.1°, about 13.8°, about 15.6°, about 16.3°, about 17.1°, about 17.3°, about 17.6°, about 20.5°, about 21.0°, about 21.8°, about 25.4°, about 26.8°, and about 32.0° 2θ.

In certain embodiments, Form A of the tosylate salt of the compound of formula (I) has an XRPD pattern comprising peaks at about 5.1°, about 6.1°, about 6.6°, about 7.3°, about 8.9°, about 10.2°, about 11.2°, about 11.8°, about 12.0°, about 12.6°, about 13.1°, about 13.8°, about 15.6°, about 16.3°, about 17.1°, about 17.3°, about 17.6°, about 20.5°, about 21.0°, about 21.8°, about 25.4°, about 26.8°, and about 32.0° 2θ.

In certain embodiments, Form A of the tosylate salt of the compound of formula (I) has an XRPD pattern comprising a peak at 5.1°±0.3° 2θ.

In certain embodiments, the XRPD pattern further comprises one or more peaks at 6.1°±0.3°, 6.6°±0.3°, 7.3°±0.3°, 8.9°±0.3°, 10.2°±0.3°, 11.2°±0.3°, 11.8°±0.3°, 12.0°±0.3°, 12.6°±0.3°, 13.1°±0.3°, and 13.8°±0.3° 2θ.

In certain embodiments, the XRPD pattern further comprises one or more peaks at 15.6°±0.3°, 16.3°±0.3°, 17.1°±0.3°, 17.3°±0.3°, 17.6°±0.3°, 20.5°±0.3°, 21.0°±0.3°, and 21.8°±0.3° 2θ.

In certain embodiments, the XRPD pattern further comprises one or more peaks at 25.4°±0.3°, 26.8°±0.3°, and 32.0°±0.3° 2θ.

In certain embodiments, Form A of the tosylate salt of the compound of formula (I) has XRPD pattern comprising one or more peaks at 5.1°±0.3°, 6.1°±0.3°, 6.6°±0.3°, 7.3°±0.3°, 8.9°±0.3°, 10.2°±0.3°, 11.2°±0.3°, 11.8°±0.3°, 12.0°±0.3°, 12.6°±0.3°, 13.1°±0.3°, 13.8=0.3°, 15.6°±0.3°, 16.3°±0.3°, 17.1°±0.3°, 17.3°±0.3°, 17.6°±0.3°, 20.5°±0.3°, 21.0±0.3°, 21.8°±0.3°, 25.4°±0.3°, 26.8°±0.3°, and 32.0°±0.3° 2θ.

In certain embodiments, Form A of the tosylate salt of the compound of formula (I) has XRPD pattern comprising peaks at 5.1°±0.3°, 6.1°±0.3°, 6.6°±0.3°, 7.3°±0.3°, 8.9°±0.3°, 10.2°±0.3°, 11.2°±0.3°, 11.8°±0.3°, 12.0°±0.3°, 12.6°±0.3°, 13.1°±0.3°, 13.8=0.3°, 15.6°±0.3°, 16.3°±0.3°, 17.1°±0.3°, 17.3°±0.3°, 17.6°±0.3°, 20.5°±0.3°, 21.0±0.3°, 21.8°±0.3°, 25.4°±0.3°, 26.8°±0.3°, and 32.0°±0.3° 2θ.

In certain embodiments, Form A of the tosylate salt of the compound of formula (I) has XRPD pattern comprising a peak at 5.1°±0.2° 2θ.

In certain embodiments, the XRPD pattern further comprises one or more peaks at 6.1°±0.2°, 6.6°±0.2°, 7.3°±0.2°, 8.9°±0.2°, 10.2°±0.2°, 11.2°±0.2°, 11.8°±0.2°, 12.0°±0.2°, 12.6°±0.2°, 13.1°±0.2°, and 13.8°±0.2° 2θ.

In certain embodiments, the XRPD pattern further comprises one or more peaks at 15.6°±0.2°, 16.3°±0.2°, 17.1°±0.2°, 17.3°±0.2°, 17.6°±0.2°, 20.5°±0.2°, 21.0°±0.2°, and 21.8°±0.2° 2θ.

In certain embodiments, the XRPD pattern further comprises one or more peaks at 25.4°±0.2°, 26.8°±0.2°, and 32.0°±0.2° 2θ.

In certain embodiments, Form A of the tosylate salt of the compound of formula (I) has XRPD pattern comprising one or more peaks at 5.1°±0.2°, 6.1°±0.2°, 6.6°±0.2°, 7.3°±0.2°, 8.9°±0.2°, 10.2°±0.2°, 11.2°±0.2°, 11.8°±0.2°, 12.0°±0.2°, 12.6°±0.2°, 13.1°±0.2°, 13.8=0.2°, 15.6°±0.2°, 16.3°±0.2°, 17.1°±0.2°, 17.3°±0.2°, 17.6°±0.2°, 20.5°±0.2°, 21.0=0.2°, 21.8°±0.2°, 25.4°±0.2°, 26.8°±0.2°, and 32.0°±0.2° 2θ.

In certain embodiments, Form A of the tosylate salt of the compound of formula (I) has XRPD pattern comprising peaks at 5.1°±0.2°, 6.1°±0.2°, 6.6°±0.2°, 7.3°±0.2°, 8.9°±0.2°, 10.2°±0.2°, 11.2°±0.2°, 11.8°±0.2°, 12.0°±0.2°, 12.6°±0.2°, 13.1°±0.2°, 13.8±0.2°, 15.6°±0.2°, 16.3°±0.2°, 17.1°±0.2°, 17.3°±0.2°, 17.6°±0.2°, 20.5°±0.2°, 21.0±0.2°, 21.8°±0.2°, 25.4°±0.2°, 26.8°±0.2°, and 32.0°±0.2° 2θ.

In certain embodiments, Form A of the tosylate salt of the compound of formula (I) has an XRPD pattern substantially the same as shown in FIG. 18. In certain embodiments, Form A of the tosylate salt of the compound of formula (I) has an XRPD pattern comprising one or more diffraction peaks (2θ) disclosed in Table 22.

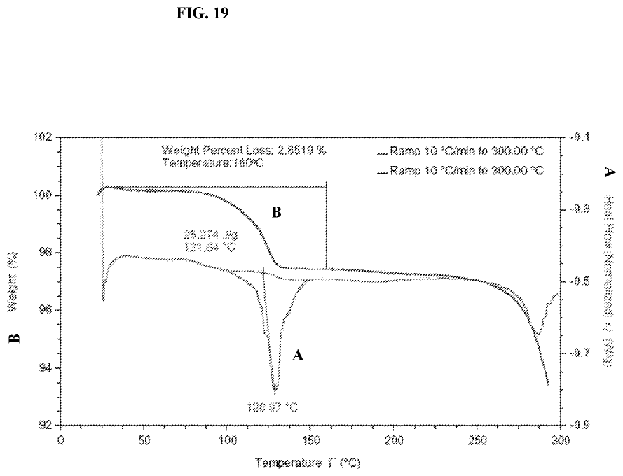

In certain embodiments, Form A of the tosylate salt of the compound of formula (I) has a differential scanning calorimetry (DSC) thermogram comprising an endotherm with a peak onset at about 126° C. In certain embodiments, Form A of the tosylate salt of the compound of formula (I) has a DSC thermogram substantially the same as shown in FIG. 19.

In certain embodiments, Form A of the tosylate salt of the compound of formula (I) exhibits a weight loss of less than or equal to about 5.1% wt. upon heating Form A from about 25° C. to about 170° C. The weight loss exhibited by a crystalline form (e.g., Form A of the tosylate salt of the compound of formula (I)) can be determined, for example, using thermogravimetric analysis (TGA). In certain embodiments, Form A of the tosylate salt of the compound of formula (I) has a TGA thermogram substantially the same as shown in FIG. 19.

### (4) Succinate Salt Forms

In various embodiments, provided herein is a succinate salt of the compound of formula (I)

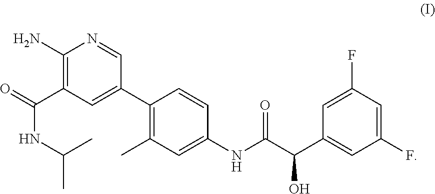 [SMILES: Cc1cc(NC(=O)[C@H](O)c2cc(F)cc(F)c2)ccc1-c1cnc(N)c(C(=O)NC(C)C)c1]

In certain embodiments, the succinate salt of the compound of formula (I) is a crystalline salt form.

In various embodiments, provided herein is a crystalline succinate salt of the compound of formula (I)

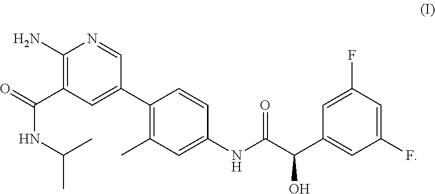 [SMILES: Cc1cc(NC(=O)[C@H](O)c2cc(F)cc(F)c2)ccc1-c1cnc(N)c(C(=O)NC(C)C)c1]

In certain embodiments, the crystalline succinate salt of the compound of formula (I) is Form A of the succinate salt of the compound of formula (I).

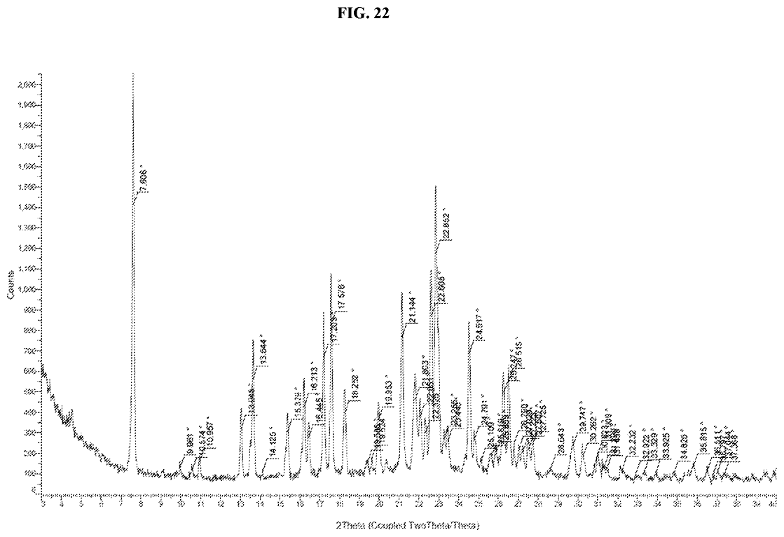

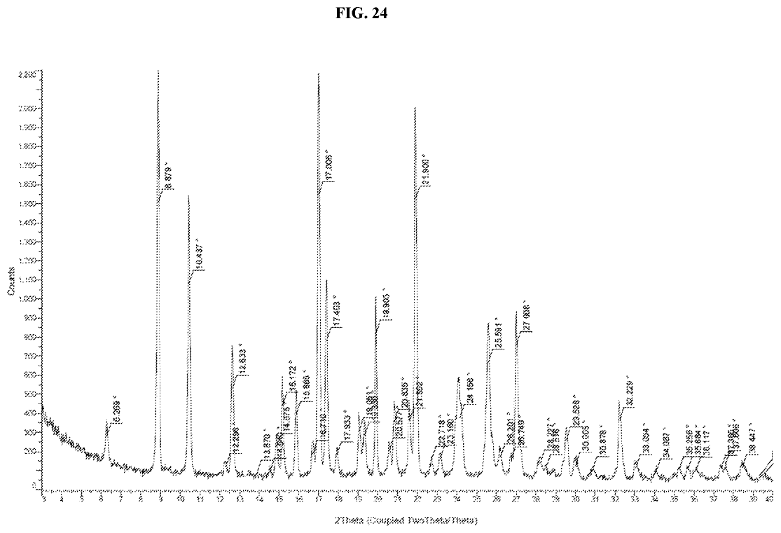

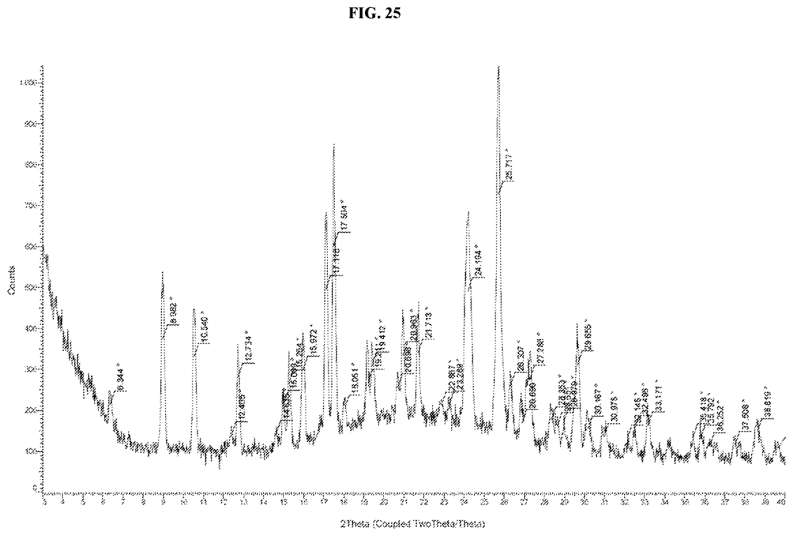

In certain embodiments, the crystalline succinate salt has an XRPD pattern substantially the same as shown in FIG. 22. In certain embodiments, the crystalline tosylate salt has an XRPD pattern substantially the same as shown in FIG. 24. In certain embodiments, the crystalline tosylate salt has an XRPD pattern substantially the same as shown in FIG. 25.

In certain embodiments, the crystalline fumarate salt of the compound of formula (I) has an XRPD pattern comprising one or more diffraction peaks (2θ) disclosed in Table 25. In certain embodiments, the crystalline fumarate salt of the compound of formula (I) has an XRPD pattern comprising one or more diffraction peaks (2θ) disclosed in Table 26. In certain embodiments, the crystalline fumarate salt of the compound of formula (I) has an XRPD pattern comprising one or more diffraction peaks (2θ) disclosed in Table 27.

### (A) Form A

In various embodiments, provided herein is Form A of the succinate salt of the compound of formula (I)

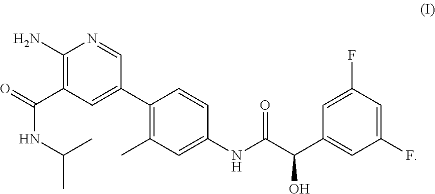 [SMILES: Cc1cc(NC(=O)[C@H](O)c2cc(F)cc(F)c2)ccc1-c1cnc(N)c(C(=O)NC(C)C)c1]

In certain embodiments, Form A of the succinate salt of the compound of formula (I) has an X-ray powder diffraction (XRPD) pattern comprising a peak at about 7.6° 2θ.

In certain embodiments, the XRPD pattern further comprises one or more peaks at about 5.6°, about 8.9°, about 10.0°, about 11.0°, about 12.7°, and about 13.1° 2θ.

In certain embodiments, the XRPD pattern further comprises one or more peaks at about 15.4°, about 16.2°, about 16.4°, about 17.2°, about 17.6°, about 18.3°, about 19.1°, about 21.2°, about 21.8°, about 22.1°, about 22.3°, about 22.6°, about 22.9°, about 23.4°, about 24.2°, about 24.5°, and about 24.8° 2θ.

In certain embodiments, the XRPD pattern further comprises one or more peaks at about 25.8°, about 26.3°, about 26.5°, about 27.0°, about 27.6°, about 27.7°, about 28.6°, about 29.8°, about 30.2°, about 31.0°, about 32.2°, about 32.5°, about 33.3°, and about 34.9° 2θ.

In certain embodiments, the XRPD pattern further comprises one or more peaks at about 35.8°, about 36.5°, about 37.1°, about 37.9°, about 38.8°, and about 39.5° 2θ.

In certain embodiments, Form A of the succinate salt of the compound of formula (I) has an XRPD pattern comprising one or more peaks at about 5.6°, about 7.6°, about 8.9°, about 10.0°, about 11.0°, about 12.7°, about 13.1°, about 15.4°, about 16.2°, about 16.4°, about 17.2°, about 17.6°, about 18.3°, about 19.1°, about 21.2°, about 21.8°, about 22.1°, about 22.3°, about 22.6°, about 22.9°, about 23.4°, about 24.2°, about 24.5°, about 24.8°, about 25.8°, about 26.3°, about 26.5°, about 27.0°, about 27.6°, about 27.7°, about 28.6°, about 29.8°, about 30.2°, about 31.0°, about 32.2°, about 32.5°, about 33.3°, about 34.9°, about 35.8°, about 36.5°, about 37.1°, about 37.9°, about 38.8°, and about 39.5° 2θ.

In certain embodiments, Form A of the succinate salt of the compound of formula (I) has an XRPD pattern comprising peaks at about 5.6°, about 7.6°, about 8.9°, about 10.0°, about 11.0°, about 12.7°, about 13.1°, about 15.4°, about 16.2°, about 16.4°, about 17.2°, about 17.6°, about 18.3°, about 19.1°, about 21.2°, about 21.8°, about 22.1°, about 22.3°, about 22.6°, about 22.9°, about 23.4°, about 24.2°, about 24.5°, about 24.8°, about 25.8°, about 26.3°, about 26.5°, about 27.0°, about 27.6°, about 27.7°, about 28.6°, about 29.8°, about 30.2°, about 31.0°, about 32.2°, about 32.5°, about 33.3°, about 34.9°, about 35.8°, about 36.5°, about 37.1°, about 37.9°, about 38.8°, and about 39.5° 2θ.

In certain embodiments, Form A of the succinate salt of the compound of formula (I) has an XRPD pattern comprising a peak at 7.6°±0.3° 2θ.

In certain embodiments, the XRPD pattern further comprises one or more peaks at 5.6°±0.3°, 8.9°±0.3°, 10.0°±0.3°, 11.0°±0.3°, 12.7°±0.3°, and 13.1°±0.3° 2θ.

In certain embodiments, the XRPD pattern further comprises one or more peaks at 15.4°±0.3°, 16.2°±0.3°, 16.4°±0.3°, 17.2°±0.3°, 17.6°±0.3°, 18.3°±0.3°, 19.1°±0.3°, 21.2°±0.3°, 21.8°±0.3°, 22.1°±0.3°, 22.3°±0.3°, 22.6°±0.3°, 22.9°±0.3°, 23.4°±0.3°, 24.2°±0.3°, 24.5°±0.3°, and 24.8°±0.3° 2θ.

In certain embodiments, the XRPD pattern further comprises one or more peaks at 25.8°±0.3°, 26.3°±0.3°, 26.5°±0.3°, 27.0°±0.3°, 27.6°±0.3°, 27.7°±0.3°, 28.6°±0.3°, 29.8°±0.3°, 30.2°±0.3°, 31.0°±0.3°, 32.2°±0.3°, 32.5°±0.3°, 33.3°±0.3°, and 34.9°±0.3° 2θ.

In certain embodiments, the XRPD pattern further comprises one or more peaks at 35.8°±0.3°, 36.5°, 37.1°±0.3°, 37.9°±0.3°, 38.8°±0.3°, and 39.5°±0.3° 2θ.

In certain embodiments, Form A of the succinate salt of the compound of formula (I) has an XRPD pattern comprising one or more peaks at 5.6°±0.3°, 7.6°±0.3°, 8.9°±0.3°, 10.0°±0.3°, 11.0°±0.3°, 12.7°±0.3°, 13.1°±0.3°, 15.4°±0.3°, 16.2°±0.3°, 16.4°±0.3°, 17.2°±0.3°, 17.6°±0.3°, 18.3°±0.3°, 19.1°±0.3°, 21.2°±0.3°, 21.8°±0.3°, 22.1°±0.3°, 22.3°±0.3°, 22.6°±0.3°, 22.9°±0.3°, 23.4°±0.3°, 24.2°±0.3°, 24.5°±0.3°, 24.8°±0.3°, 25.8°±0.3°, 26.3°±0.3°, 26.5°±0.3°, 27.0°±0.3°, 27.6°±0.3°, 27.7°±0.3°, 28.6°±0.3°, 29.8°±0.3°, 30.2°±0.3°, 31.0°±0.3°, 32.2°±0.3°, 32.5°±0.3°, 33.3°±0.3°, 34.9°±0.3°, 35.8°±0.3°, 36.5°±0.3°, 37.1°±0.3°, 37.9°±0.3°, 38.8°±0.3°, and 39.5°±0.3° 2θ.

In certain embodiments, Form A of the succinate salt of the compound of formula (I) has an XRPD pattern comprising peaks at 5.6°±0.3°, 7.6°±0.3°, 8.9°±0.3°, 10.0°±0.3°, 11.0°±0.3°, 12.7°±0.3°, 13.1°±0.3°, 15.4°±0.3°, 16.2°±0.3°, 16.4°±0.3°, 17.2°±0.3°, 17.6°±0.3°, 18.3°±0.3°, 19.1°±0.3°, 21.2°±0.3°, 21.8°±0.3°, 22.1°±0.3°, 22.3°±0.3°, 22.6°±0.3°, 22.9°±0.3°, 23.4°±0.3°, 24.2°±0.3°, 24.5°±0.3°, 24.8°±0.3°, 25.8°±0.3°, 26.3°±0.3°, 26.5°±0.3°, 27.0°±0.3°, 27.6°±0.3°, 27.7°±0.3°, 28.6°±0.3°, 29.8°±0.3°, 30.2°±0.3°, 31.0°±0.3°, 32.2°±0.3°, 32.5°±0.3°, 33.3°±0.3°, 34.9°±0.3°, 35.8°±0.3°, 36.5°±0.3°, 37.1°±0.3°, 37.9°±0.3°, 38.8°±0.3°, and 39.5°±0.3° 2θ.

In certain embodiments, Form A of the succinate salt of the compound of formula (I) has an XRPD pattern comprising a peak at 7.6°±0.2° 2θ.

In certain embodiments, the XRPD pattern further comprises one or more peaks at 5.6°±0.2°, 8.9°±0.2°, 10.0°±0.2°, 11.0°±0.2°, 12.7°±0.2°, and 13.1°±0.2° 2θ.

In certain embodiments, the XRPD pattern further comprises one or more peaks at 15.4°±0.2°, 16.2°±0.2°, 16.4°±0.2°, 17.2°±0.2°, 17.6°±0.2°, 18.3°±0.2°, 19.1°±0.2°, 21.2°±0.2°, 21.8°±0.2°, 22.1°±0.2°, 22.3°±0.2°, 22.6°±0.2°, 22.9°±0.2°, 23.4°±0.2°, 24.2°±0.2°, 24.5°±0.2°, and 24.8°±0.2° 2θ.

In certain embodiments, the XRPD pattern further comprises one or more peaks at 25.8°±0.2°, 26.3°±0.2°, 26.5°±0.2°, 27.0°±0.2°, 27.6°±0.2°, 27.7°±0.2°, 28.6°±0.2°, 29.8°±0.2°, 30.2°±0.2°, 31.0°±0.2°, 32.2°±0.2°, 32.5°±0.2°, 33.3°±0.2°, and 34.9°±0.2° 2θ.

In certain embodiments, the XRPD pattern further comprises one or more peaks at 35.8°±0.2°, 36.5°±0.2°, 37.1°±0.2°, 37.9°±0.2°, 38.8°±0.2°, and 39.5°±0.2° 2θ.

In certain embodiments, Form A of the succinate salt of the compound of formula (I) has an XRPD pattern comprising one or more peaks at 5.6°±0.2°, 7.6°±0.2°, 8.9°±0.2°, 10.0°±0.2°, 11.0°±0.2°, 12.7°±0.2°, 13.1°±0.2°, 15.4°±0.2°, 16.2°±0.2°, 16.4°±0.2°, 17.2°±0.2°, 17.6°±0.2°, 18.3°±0.2°, 19.1°±0.2°, 21.2°±0.2°, 21.8°±0.2°, 22.1°±0.2°, 22.3°±0.2°, 22.6°±0.2°, 22.9°±0.2°, 23.4°±0.2°, 24.2°±0.2°, 24.5°±0.2°, 24.8°±0.2°, 25.8°±0.2°, 26.3°±0.2°, 26.5°±0.2°, 27.0°±0.2°, 27.6°±0.2°, 27.7°±0.2°, 28.6°±0.2°, 29.8°±0.2°, 30.2°±0.2°, 31.0°±0.2°, 32.2°±0.2°, 32.5°±0.2°, 33.3°±0.2°, 34.9°±0.2°, 35.8°±0.2°, 36.5°±0.2°, 37.1°±0.2°, 37.9°±0.2°, 38.8°±0.2°, and 39.5°±0.2° 2θ.

In certain embodiments, Form A of the succinate salt of the compound of formula (I) has an XRPD pattern comprising peaks at 5.6°±0.2°, 7.6°±0.2°, 8.9°±0.2°, 10.0°±0.2°, 11.0°±0.2°, 12.7°±0.2°, 13.1°±0.2°, 15.4°±0.2°, 16.2°±0.2°, 16.4°±0.2°, 17.2°±0.2°, 17.6°±0.2°, 18.3°±0.2°, 19.1°±0.2°, 21.2°±0.2°, 21.8°±0.2°, 22.1°±0.2°, 22.3°±0.2°, 22.6°±0.2°, 22.9°±0.2°, 23.4°±0.2°, 24.2°±0.2°, 24.5°±0.2°, 24.8°±0.2°, 25.8°±0.2°, 26.3°±0.2°, 26.5°±0.2°, 27.0°±0.2°, 27.6°±0.2°, 27.7°±0.2°, 28.6°±0.2°, 29.8°±0.2°, 30.2°±0.2°, 31.0°±0.2°, 32.2°±0.2°, 32.5°±0.2°, 33.3°±0.2°, 34.9°±0.2°, 35.8°±0.2°, 36.5°±0.2°, 37.1°±0.2°, 37.9°±0.2°, 38.8°±0.2°, and 39.5°±0.2° 2θ.

In certain embodiments, Form A of the succinate salt of the compound of formula (I) has an XRPD pattern substantially the same as shown in FIG. 22. In certain embodiments, Form A of the succinate salt of the compound of formula (I) has an XRPD pattern comprising one or more diffraction peaks (2θ) disclosed in Table 25.

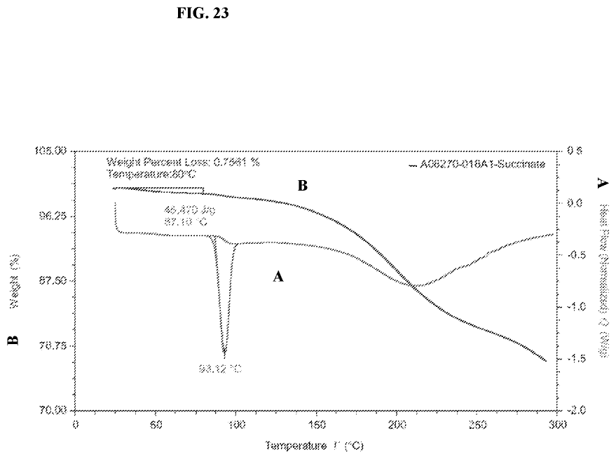

In certain embodiments, Form A of the succinate salt of the compound of formula (I) has a differential scanning calorimetry (DSC) thermogram comprising an endotherm with a peak onset at about 87° C. In certain embodiments, Form A of the succinate salt of the compound of formula (I) has a DSC thermogram substantially the same as shown in FIG. 23.

In certain embodiments, Form A of the succinate salt of the compound of formula (I) exhibits a weight loss of less than or equal to about 0.8% wt. upon heating Form A from about 25° C. to about 80° C. The weight loss exhibited by a crystalline form (e.g., Form A of the succinate salt of the compound of formula (I)) can be determined, for example, using thermogravimetric analysis (TGA). In certain embodiments, Form A of the succinate salt of the compound of formula (I) has a TGA thermogram substantially the same as shown in FIG. 23.

### (5) Camsylate Salt Form

In one aspect, provided herein is a crystalline camsylate salt of the compound of formula (I)

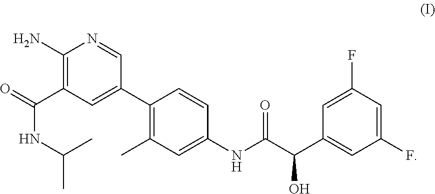 [SMILES: Cc1cc(NC(=O)[C@H](O)c2cc(F)cc(F)c2)ccc1-c1cnc(N)c(C(=O)NC(C)C)c1]

In certain embodiments, the crystalline camsylate salt of the compound of formula (I) has an XRPD pattern comprising a peak at about 5.2° 2θ. In certain embodiments, the crystalline camsylate salt of the compound of formula (I) has an XRPD pattern comprising a peak at about 13.2° 2θ. In certain embodiments, the crystalline camsylate salt of the compound of formula (I) has an XRPD pattern comprising a peak at about 14.7° 2θ. In certain embodiments, the crystalline camsylate salt of the compound of formula (I) has an XRPD pattern comprising one or more peaks at about 5.2°, about 13.2°, and about 14.7° 2θ.

In certain embodiments, the XRPD pattern further comprises one or more peaks at about 15.7°, about 17.8°, about 18.2°, about 19.3°, and about 21.8° 2θ.

In certain embodiments, the crystalline camsylate salt of the compound of formula (I) has an XRPD pattern comprising one or more peaks at about 5.2°, about 13.2°, about 14.7°, about 15.7°, about 17.8°, about 18.2°, about 19.3°, and about 21.8° 2θ.

In certain embodiments, the crystalline camsylate salt of the compound of formula (I) has an XRPD pattern comprising peaks at about 5.2°, about 13.2°, about 14.7°, about 15.7°, about 17.8°, about 18.2°, about 19.3°, and about 21.8° 2θ.

In certain embodiments, the crystalline camsylate salt of the compound of formula (I) has an XRPD pattern comprising one or more peaks at about 5.2°, about 13.2°, and about 14.7° 2θ.

In certain embodiments, the XRPD pattern further comprises one or more peaks at about 6.9°, about 8.5°, about 8.9°, about 10.5°, about 10.8°, about 12.3°, about 12.6°, about 13.4°, about 14.0°, about 14.1°, and about 14.7° 2θ.

In certain embodiments, the XRPD pattern further comprises one or more peaks at about 17.8°, about 18.2°, about 21.8°, and about 21.9° 2θ.

In certain embodiments, the XRPD pattern further comprises one or more peaks at about 15.1°, about 15.7°, about 17.0°, about 17.2, about 17.6°, about 18.1°, about 19.3°, about 19.8°, about 20.2°, about 20.6°, about 20.9°, about 21.1°, about 21.6°, about 22.5°, about 22.9°, about 23.9°, and about 24.9° 2θ.

In certain embodiments, the XRPD pattern further comprises peaks at about 25.4°, about 25.6°, and about 25.9° 2θ.

In certain embodiments, the XRPD pattern further comprises one or more peaks at about 26.3°, about 26.6°, about 27.1°, about 27.5°, about 28.5°, about 28.9°, about 29.4°, and about 30.1° 2θ.

In certain embodiments, the crystalline camsylate salt of the compound of formula (I) has an XRPD pattern comprising one or more peaks at about 5.2°, about 6.9°, about 8.5°, about 8.9°, about 10.5°, about 10.8°, about 12.3°, about 12.6°, about 13.2°, about 13.4°, about 14.0°, about 14.1°, about 14.7°, about 15.1°, about 15.7°, about 17.0°, about 17.2°, about 17.6°, about 17.8°, about 18.1°, about 18.2°, about 19.3°, about 19.8°, about 20.2°, about 20.6°, about 20.9°, about 21.1°, about 21.6°, about 21.8°, about 22.5°, about 22.9°, about 23.9°, about 24.9°, about 25.4°, about 25.6°, about 25.9°, about 26.3°, about 26.6°, about 27.1°, about 27.5°, about 28.5°, about 28.9°, about 29.4°, and about 30.1° 2θ.

In certain embodiments, the crystalline camsylate salt of the compound of formula (I) has an XRPD pattern comprising peaks at about 5.2°, about 6.9°, about 8.5°, about 8.9°, about 10.5°, about 10.8°, about 12.3°, about 12.6°, about 13.2°, about 13.4°, about 14.0°, about 14.1°, about 14.7°, about 15.1°, about 15.7°, about 17.0°, about 17.2°, about 17.6°, about 17.8°, about 18.1°, about 18.2°, about 19.3°, about 19.8°, about 20.2°, about 20.6°, about 20.9°, about 21.1°, about 21.6°, about 21.8°, about 22.5°, about 22.9°, about 23.9°, about 24.9°, about 25.4°, about 25.6°, about 25.9°, about 26.3°, about 26.6°, about 27.1°, about 27.5°, about 28.5°, about 28.9°, about 29.4°, and about 30.1° 2θ.

In certain embodiments, the crystalline camsylate salt of the compound of formula (I) having an XRPD pattern comprising peaks at about 5.2°, about 6.9°, about 8.5°, about 8.9°, about 10.5°, about 10.8°, about 12.3°, about 12.6°, about 13.2°, about 13.4°, about 14.0°, about 14.1°, about 14.7°, about 15.1°, about 15.7°, about 17.0°, about 17.2°, about 17.6°, about 17.8°, about 18.1°, about 18.2°, about 19.3°, about 19.8°, about 20.2°, about 20.6°, about 20.9°, about 21.1°, about 21.6°, about 21.8°, about 22.5°, about 22.9°, about 23.9°, about 24.9°, about 25.4°, about 25.6°, about 25.9°, about 26.3°, about 26.6°, about 27.1°, about 27.5°, about 28.5°, about 28.9°, about 29.4°, and about 30.1° 2θ may be referred to herein as “crystalline camsylate salt Form A”.

In certain embodiments, the crystalline camsylate salt of the compound of formula (I) has an XRPD pattern comprising a peak at 5.2°±0.3° 2θ. In certain embodiments, the crystalline camsylate salt of the compound of formula (I) has an XRPD pattern comprising a peak at 13.2°±0.3° 2θ. In certain embodiments, the crystalline camsylate salt of the compound of formula (I) has an XRPD pattern comprising a peak at 14.7°±0.3° 2θ. In certain embodiments, the crystalline camsylate salt of the compound of formula (I) has an XRPD pattern comprising one or more peaks at 5.2°±0.3°, 13.2°±0.3°, and 14.7°±0.3° 2θ.

In certain embodiments, the XRPD pattern further comprises one or more peaks at 15.7°±0.3°, 17.8°±0.3°, 18.2°±0.3°, 19.3°±0.3°, and 21.8°±0.3° 2θ.

In certain embodiments, the crystalline camsylate salt of the compound of formula (I) has an XRPD pattern comprising one or more peaks at 5.2°±0.3°, 13.2°±0.3°, 14.7°±0.3°, 15.7°±0.3°, 17.8°±0.3°, 18.2°±0.3°, 19.3°±0.3°, and 21.8°±0.3° 2θ.

In certain embodiments, the crystalline camsylate salt of the compound of formula (I) has an XRPD pattern comprising one or more peaks at 5.2°±0.3°, 13.2°±0.3°, and 14.7°±0.3° 2θ.

In certain embodiments, the XRPD pattern further comprises one or more peaks at 6.9°±0.3°, 8.5°±0.3°, 8.9°±0.3°, 10.5°±0.3°, 10.8°±0.3°, 12.3°±0.3°, 12.6°±0.3°, 13.4°±0.3°, 14.0°±0.3°, 14.1°±0.3°, and 14.7°±0.3° 2θ.

In certain embodiments, the XRPD pattern further comprises one or more peaks at 17.8°±0.3°, 18.2°±0.3°, 21.8°±0.3°, and 21.9°±0.3° 2θ.

In certain embodiments, the XRPD pattern further comprises one or more peaks at 15.1°±0.3°, 15.7°±0.3°, 17.0°±0.3°, 17.2°±0.3°, 17.6°±0.3°, 18.1°±0.3°, 19.3°±0.3°, 19.8°±0.3°, 20.2°±0.3°, 20.6°±0.3°, 20.9°±0.3°, 21.1°±0.3°, 21.6°±0.3°, 22.5°±0.3°, 22.9°±0.3°, 23.9°±0.3°, and 24.9°±0.3° 2θ.

In certain embodiments, the XRPD pattern further comprises peaks at 25.4°±0.3°, 25.6°±0.3°, and 25.9°±0.3° 2θ.

In certain embodiments, the XRPD pattern further comprises one or more peaks at 26.3°±0.3°, 26.6°±0.3°, 27.1°±0.3°, 27.5°±0.3°, 28.5°±0.3°, 28.9°±0.3°, 29.4°±0.3°, and 30.1°±0.3° 2θ.

In certain embodiments, the crystalline camsylate salt of the compound of formula (I) has an XRPD pattern comprising one or more peaks at 5.2°±0.3°, 6.9°±0.3°, 8.5°±0.3°, 8.9°±0.3°, 10.5°±0.3°, 10.8°±0.3°, 12.3°±0.3°, 12.6°±0.3°, 13.2°±0.3°, 13.4°±0.3°, 14.0°±0.3°, 14.1°±0.3°, 14.7°±0.3°, 15.1°±0.3°, 15.7°±0.3°, 17.0°±0.3°, 17.2°±0.3°, 17.6°±0.3°, 17.8°±0.3°, 18.1°±0.3°, 18.2°±0.3°, 19.3°±0.3°, 19.8°±0.3°, 20.2°±0.3°, 20.6°±0.3°, 20.9°±0.3°, 21.1°±0.3°, 21.6°±0.3°, 21.8°±0.3°, 22.5°±0.3°, 22.9°±0.3°, 23.9°±0.3°, 24.9°±0.3°, 25.4°±0.3°, 25.6°±0.3°, 25.9°±0.3°, 26.3°±0.3°, 26.6°±0.3°, 27.1°±0.3°, 27.5°±0.3°, 28.5°±0.3°, 28.9°±0.3°, 29.4°±0.3°, and 30.1°±0.3° 2θ.

In certain embodiments, the crystalline camsylate salt of the compound of formula (I) having an XRPD pattern comprising peaks at 5.2°±0.3°, 6.9°±0.3°, 8.5°±0.3°, 8.9°±0.3°, 10.5°±0.3°, 10.8°±0.3°, 12.3°±0.3°, 12.6°±0.3°, 13.2°±0.3°, 13.4°±0.3°, 14.0°±0.3°, 14.1°±0.3°, 14.7°±0.3°, 15.1°±0.3°, 15.7°±0.3°, 17.0°±0.3°, 17.2°±0.3°, 17.6°±0.3°, 17.8°±0.3°, 18.1°±0.3°, 18.2°±0.3°, 19.3°±0.3°, 19.8°±0.3°, 20.2°±0.3°, 20.6°±0.3°, 20.9°±0.3°, 21.1°±0.3°, 21.6°±0.3°, 21.8°±0.3°, 22.5°±0.3°, 22.9°±0.3°, 23.9°±0.3°, 24.9°±0.3°, 25.4°±0.3°, 25.6°±0.3°, 25.9°±0.3°, 26.3°±0.3°, 26.6°±0.3°, 27.1°±0.3°, 27.5°±0.3°, 28.5°±0.3°, 28.9°±0.3°, 29.4°±0.3°, and 30.1°±0.3° 2θ may be referred to herein as “crystalline camsylate salt Form A”.

In certain embodiments, the crystalline camsylate salt of the compound of formula (I) has an XRPD pattern comprising a peak at 5.2°±0.2° 2θ. In certain embodiments, the crystalline camsylate salt of the compound of formula (I) has an XRPD pattern comprising a peak at 13.2°±0.2° 2θ. In certain embodiments, the crystalline camsylate salt of the compound of formula (I) has an XRPD pattern comprising a peak at 14.7°±0.2° 2θ. In certain embodiments, the crystalline camsylate salt of the compound of formula (I) has an XRPD pattern comprising one or more peaks at 5.2°±0.2°, 13.2°±0.2°, and 14.7°±0.2° 2θ.

In certain embodiments, the XRPD pattern further comprises one or more peaks at 15.7°±0.2°, 17.8°±0.2°, 18.2°±0.2°, 19.3°±0.2°, and 21.8°±0.2° 2θ.

In certain embodiments, the crystalline camsylate salt of the compound of formula (I) has an XRPD pattern comprising one or more peaks at 5.2°±0.2°, 13.2°±0.2°, 14.7°±0.2°, 15.7°±0.2°, 17.8°±0.2°, 18.2°±0.2°, 19.3°±0.2°, and 21.8°±0.2° 2θ.

In certain embodiments, the crystalline camsylate salt of the compound of formula (I) has an XRPD pattern comprising peaks at 5.2°±0.2°, 13.2°±0.2°, 14.7°±0.2°, 15.7°±0.2°, 17.8°±0.2°, 18.2°±0.2°, 19.3°±0.2°, and 21.8°±0.2° 2θ.

In certain embodiments, the crystalline camsylate salt of the compound of formula (I) has an XRPD pattern comprising one or more peaks at 5.2°±0.2°, 13.2°±0.2°, and 14.7°±0.2° 2θ.

In certain embodiments, the XRPD pattern further comprises one or more peaks at 6.9°±0.2°, 8.5°±0.2°, 8.9°±0.2°, 10.5°±0.2°, 10.8°±0.2°, 12.3°±0.2°, 12.6°±0.2°, 13.4°±0.2°, 14.0°±0.2°, 14.1°±0.2°, and 14.7°±0.2° 2θ.

In certain embodiments, the XRPD pattern further comprises one or more peaks at 17.8°±0.2°, 18.2°±0.2°, 21.8°±0.2°, and 21.9°±0.2° 2θ.

In certain embodiments, the XRPD pattern further comprises one or more peaks at 15.1°±0.2°, 15.7°±0.2°, 17.0°±0.2°, 17.2°±0.2°, 17.6°±0.2°, 18.1°±0.2°, 19.3°±0.2°, 19.8°±0.2°, 20.2°±0.2°, 20.6°±0.2°, 20.9°±0.2°, 21.1°±0.2°, 21.6°±0.2°, 22.5°±0.2°, 22.9°±0.2°, 23.9°±0.2°, and 24.9°±0.2° 2θ.

In certain embodiments, the XRPD pattern further comprises peaks at 25.4°±0.2°, 25.6°±0.2°, and 25.9°±0.2° 2θ.

In certain embodiments, the XRPD pattern further comprises one or more peaks at 26.3°±0.2°, 26.6°±0.2°, 27.1°±0.2°, 27.5°±0.2°, 28.5°±0.2°, 28.9°±0.2°, 29.4°±0.2°, and 30.1°±0.2° 2θ.

In certain embodiments, the crystalline camsylate salt of the compound of formula (I) has an XRPD pattern comprising one or more peaks at 5.2°±0.2°, 6.9°±0.2°, 8.5°±0.2°, 8.9°±0.2°, 10.5°±0.2°, 10.8°±0.2°, 12.3°±0.2°, 12.6°±0.2°, 13.2°±0.2°, 13.4°±0.2°, 14.0°±0.2°, 14.1°±0.2°, 14.7°±0.2°, 15.1°±0.2°, 15.7°±0.2°, 17.0°±0.2°, 17.2°±0.2°, 17.6°±0.2°, 17.8°±0.2°, 18.1°±0.2°, 18.2°±0.2°, 19.3°±0.2°, 19.8°±0.2°, 20.2°±0.2°, 20.6°±0.2°, 20.9°±0.2°, 21.1°±0.2°, 21.6°±0.2°, 21.8°±0.2°, 22.5°±0.2°, 22.9°±0.2°, 23.9°±0.2°, 24.9°±0.2°, 25.4°±0.2°, 25.6°±0.2°, 25.9°±0.2°, 26.3°±0.2°, 26.6°±0.2°, 27.1°±0.2°, 27.5°±0.2°, 28.5°±0.2°, 28.9°±0.2°, 29.4°±0.2°, and 30.1°±0.2° 2θ.

In certain embodiments, the crystalline camsylate salt of the compound of formula (I) having an XRPD pattern comprising peaks at 5.2°±0.2°, 6.9°±0.2°, 8.5°±0.2°, 8.9°±0.2°, 10.5°±0.2°, 10.8°±0.2°, 12.3°±0.2°, 12.6°±0.2°, 13.2°±0.2°, 13.4°±0.2°, 14.0°±0.2°, 14.1°±0.2°, 14.7°±0.2°, 15.1°±0.2°, 15.7°±0.2°, 17.0°±0.2°, 17.2°±0.2°, 17.6°±0.2°, 17.8°±0.2°, 18.1°±0.2°, 18.2°±0.2°, 19.3°±0.2°, 19.8°±0.2°, 20.2°±0.2°. 20.6°±0.2°, 20.9°±0.2°, 21.1°±0.2°, 21.6°±0.2°, 21.8°±0.2°, 22.5°±0.2°, 22.9°±0.2°, 23.9°±0.2°, 24.9°±0.2°, 25.4°±0.2°, 25.6°±0.2°, 25.9°±0.2°, 26.3°±0.2°, 26.6°±0.2°, 27.1°±0.2°, 27.5°±0.2°, 28.5°±0.2°, 28.9°±0.2°, 29.4°±0.2°, and 30.1°±0.2° 2θ may be referred to herein as “crystalline camsylate salt Form A”.

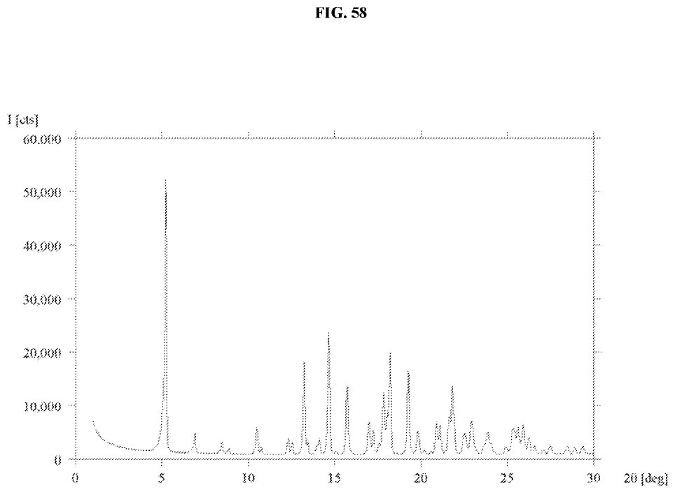

In certain embodiments, the crystalline camsylate salt of the compound of formula (I) (e.g., crystalline camsylate salt Form A) has an XRPD pattern substantially the same as shown in FIG. 58. In certain embodiments, the crystalline camsylate salt of the compound of formula (I) (e.g., crystalline camsylate salt Form A) has an XRPD pattern comprising one or more diffraction peaks (2θ) disclosed in Table 43.

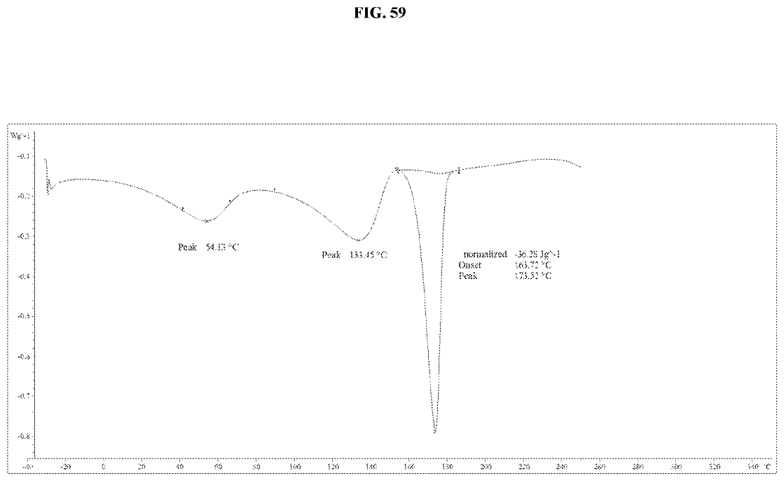

In certain embodiments, crystalline camsylate salt Form A of the compound of formula (I) has a DSC thermogram comprising an endotherm with a peak maximum at about 54° C. In certain embodiments, crystalline camsylate salt Form A of the compound of formula (I) has a DSC thermogram comprising an endotherm with a peak maximum at about 133° C. In certain embodiments, crystalline camsylate salt Form A of the compound of formula (I) has a DSC thermogram comprising an endotherm with a peak maximum at about 174° C. In certain embodiments, crystalline camsylate salt Form A of the compound of formula (I) has a DSC thermogram comprising one or more endotherms with peak onsets at about 54° C., about 133° C. and about 174° C. In certain embodiments, crystalline camsylate salt Form A of the compound of formula (I) has a DSC thermogram substantially the same as shown in FIG. 59.

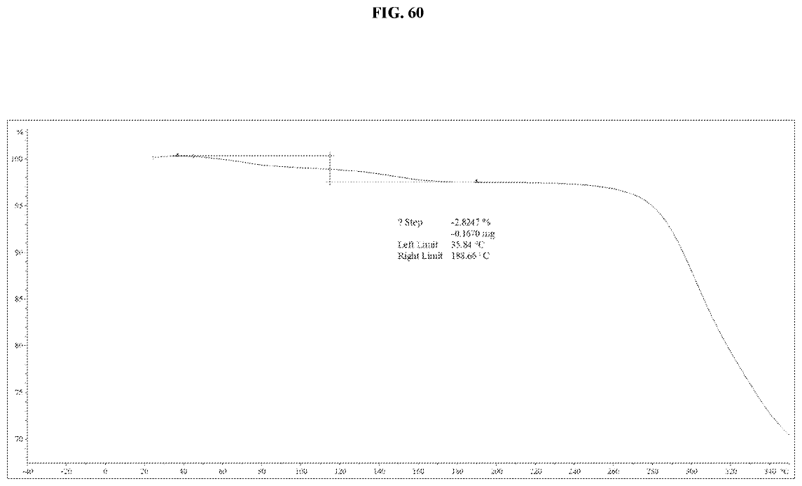

In certain embodiments, crystalline camsylate salt Form A of the compound of formula (I) exhibits a weight loss of less than or equal to about 2.8% wt. upon heating crystalline camsylate salt Form A of the compound of formula (I) from about 36° C. to about 189° C. The weight loss exhibited by a crystalline form (e.g., crystalline camsylate salt Form A of the compound of formula (I)) can be determined, for example, using TGA. In certain embodiments, the crystalline camsylate salt of the compound of formula (I) (e.g., crystalline camsylate salt Form A of the compound of formula (I)) has a TGA thermogram substantially the same as shown in FIG. 60.

In certain embodiments, crystalline camsylate salt Form A of the compound of formula (I) is a crystalline hydrate.

### (6) Phosphate Salt Forms

In one aspect, provided herein is a phosphate salt of a compound of formula (I)

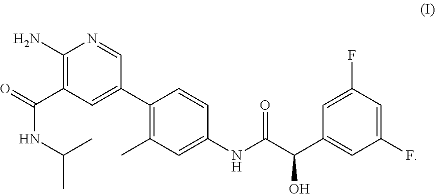 [SMILES: Cc1cc(NC(=O)[C@H](O)c2cc(F)cc(F)c2)ccc1-c1cnc(N)c(C(=O)NC(C)C)c1]

In certain embodiments, the phosphate salt of the compound of formula (I) is a crystalline phosphate salt. In certain embodiments, provided herein is a material (e.g., a pharmaceutical material) comprising a crystalline phosphate salt of the compound of formula (I).

### (A) Phosphate Salt Material A

In certain embodiments, the crystalline phosphate salt of the compound of formula (I) has an XRPD pattern comprising a peak at about 3.9° 2θ. In certain embodiments, the crystalline phosphate salt of the compound of formula (I) has an XRPD pattern comprising a peak at about 7.7° 2θ. In certain embodiments, the crystalline phosphate salt of the compound of formula (I) has an XRPD pattern comprising a peak at about 13.7° 2θ. In certain embodiments, the crystalline phosphate salt of the compound of formula (I) has an XRPD pattern comprising one or more peaks at about 3.9°, about 7.7°, and about 13.7° 2θ.

In certain embodiments, the crystalline phosphate salt of the compound of formula (I) has an XRPD pattern comprising peaks at about 3.9°, about 7.7°, and about 13.7° 2θ.

In certain embodiments, the material comprising a crystalline phosphate salt comprising XRPD peaks at about 3.9°, about 7.7°, and about 13.7° 2θ may be referred to herein as “phosphate salt Material A”.

In certain embodiments, the crystalline phosphate salt of the compound of formula (I) has an XRPD pattern comprising one or more peaks at about 3.9° 2θ.

In certain embodiments, the XRPD pattern further comprises one or more peaks at about 5.5°, about 7.0°, about 7.7°, about 13.7°, and about 14.0° 2θ.

In certain embodiments, the XRPD pattern further comprises one or more peaks at about 16.0°, about 16.3°, about 17.2°, and about 24.2° 2θ.

In certain embodiments, the crystalline phosphate salt of the compound of formula (I) has an XRPD pattern comprising one or more peaks at about 3.9°, about 5.5°, about 7.0°, about 7.7°, about 13.7°, about 14.0°, about 16.0°, about 16.3°, about 17.2°, and about 24.2° 2θ.

In certain embodiments, the crystalline phosphate salt of the compound of formula (I) has an XRPD pattern comprising peaks at about 3.9°, about 5.5°, about 7.0°, about 7.7°, about 13.7°, about 14.0°, about 16.0°, about 16.3°, about 17.2°, and about 24.2° 2θ.

In certain embodiments, the material comprising a crystalline phosphate salt comprising XRPD peaks at about 3.9°, about 5.5°, about 7.0°, about 7.7°, about 13.7°, about 14.0°, about 16.0°, about 16.3°, about 17.2°, and about 24.2° 2θ may be referred to herein as “phosphate salt Material A”.

In certain embodiments, the crystalline phosphate salt of the compound of formula (I) has an XRPD pattern comprising a peak at 3.9°±0.3° 2θ. In certain embodiments, the crystalline phosphate salt of the compound of formula (I) has an XRPD pattern comprising a peak at 7.7°±0.3° 2θ. In certain embodiments, the crystalline phosphate salt of the compound of formula (I) has an XRPD pattern comprising a peak at 13.7°±0.3° 2θ. In certain embodiments, the crystalline phosphate salt of the compound of formula (I) has an XRPD pattern comprising one or more peaks at 3.9°±0.3°, 7.7°±0.3°, and 13.7°±0.3° 2θ.

In certain embodiments, the crystalline phosphate salt of the compound of formula (I) has an XRPD pattern comprising peaks at 3.9°±0.3°, 7.7°±0.3°, and 13.7°±0.3° 2θ.

In certain embodiments, the material comprising a crystalline phosphate salt comprising XRPD peaks at 3.9°±0.3°, 7.7°±0.3°, and 13.7°±0.3° 2θ may be referred to herein as “phosphate salt Material A”.

In certain embodiments, the crystalline phosphate salt of the compound of formula (I) has an XRPD pattern comprising one or more peaks at 3.9°±0.3° 2θ.

In certain embodiments, the XRPD pattern further comprises one or more peaks at 5.5°±0.3°, 7.0°±0.3°, 7.7°±0.3°, 13.7°±0.3°, and 14.0°±0.3° 2θ.

In certain embodiments, the XRPD pattern further comprises one or more peaks at 16.0°±0.3°, 16.3°±0.3°, 17.2°±0.3°, and 24.2°±0.3° 2θ.

In certain embodiments, the crystalline phosphate salt of the compound of formula (I) has an XRPD pattern comprising one or more peaks at 3.9°±0.3°, 5.5°±0.3°, 7.0°±0.3°, 7.7°±0.3°, 13.7°±0.3°, 14.0°±0.3°, 16.0°±0.3°, 16.3°±0.3°, 17.2°±0.3°, and 24.2°±0.3° 2θ.

In certain embodiments, the crystalline phosphate salt of the compound of formula (I) has an XRPD pattern comprising peaks at 3.9°±0.3°, 5.5°±0.3°, 7.0°±0.3°, 7.7°±0.3°, 13.7°±0.3°, 14.0°±0.3°, 16.0°±0.3°, 16.3°±0.3°, 17.2°±0.3°, and 24.2°±0.3° 2θ.

In certain embodiments, the material comprising a crystalline phosphate salt comprising XRPD peaks at 3.9°±0.3°, 5.5°±0.3°, 7.0°±0.3°. 7.7°±0.3°, 13.7°±0.3°, 14.0°±0.3°, 16.0°±0.3°, 16.3°±0.3°, 17.2°±0.3°, and 24.2°±0.3° 2θ may be referred to herein as “phosphate salt Material A”.

In certain embodiments, the crystalline phosphate salt of the compound of formula (I) has an XRPD pattern comprising a peak at 3.9°±0.2° 2θ. In certain embodiments, the crystalline phosphate salt of the compound of formula (I) has an XRPD pattern comprising a peak at 7.7°±0.2° 2θ. In certain embodiments, the crystalline phosphate salt of the compound of formula (I) has an XRPD pattern comprising a peak at 13.7°±0.2° 2θ. In certain embodiments, the crystalline phosphate salt of the compound of formula (I) has an XRPD pattern comprising one or more peaks at 3.9°±0.2°, 7.7°±0.2°, and 13.7°±0.2° 2θ.

In certain embodiments, the crystalline phosphate salt of the compound of formula (I) has an XRPD pattern comprising peaks at 3.9°±0.2°, 7.7°±0.2° and 13.7°±0.2° 2θ.

In certain embodiments, the material comprising a crystalline phosphate salt comprising XRPD peaks at 3.9°±0.2°, 7.7°±0.2°, and 13.7°±0.2° 2θ may be referred to herein as “phosphate salt Material A”.

In certain embodiments, the crystalline phosphate salt of the compound of formula (I) has an XRPD pattern comprising one or more peaks at 3.9°±0.2° 2θ.

In certain embodiments, the XRPD pattern further comprises one or more peaks at 5.5°±0.2°, 7.0°±0.2°, 7.7°±0.2°, 13.7°±0.2°, and 14.0°±0.2° 2θ.

In certain embodiments, the XRPD pattern further comprises one or more peaks at 16.0°±0.2°, 16.3°±0.2°, 17.2°±0.2°, and 24.2°±0.2° 2θ.

In certain embodiments, the crystalline phosphate salt of the compound of formula (I) has an XRPD pattern comprising one or more peaks at 3.9°±0.2°, 5.5°±0.2°, 7.0°±0.2°, 7.7°±0.2°, 13.7°±0.2°, 14.0°±0.2°, 16.0°±0.2°, 16.3°±0.2°, 17.2°±0.2°, and 24.2°±0.2° 2θ.

In certain embodiments, the crystalline phosphate salt of the compound of formula (I) has an XRPD pattern comprising peaks at 3.9°±0.2°, 5.5°±0.2°, 7.0°±0.2°, 7.7°±0.2°, 13.7°±0.2°, 14.0°±0.2°, 16.0°±0.2°, 16.3°±0.2°, 17.2°±0.2°, and 24.2°±0.2° 2θ.

In certain embodiments, the material comprising a crystalline phosphate salt comprising XRPD peaks at 3.9°±0.2°, 5.5°±0.2°, 7.0°±0.2°. 7.7°±0.2°, 13.7°±0.2°, 14.0°±0.2°, 16.0°±0.2°, 16.3°±0.2°, 17.2°±0.2°, and 24.2°±0.2° 2θ may be referred to herein as “phosphate salt Material A”.

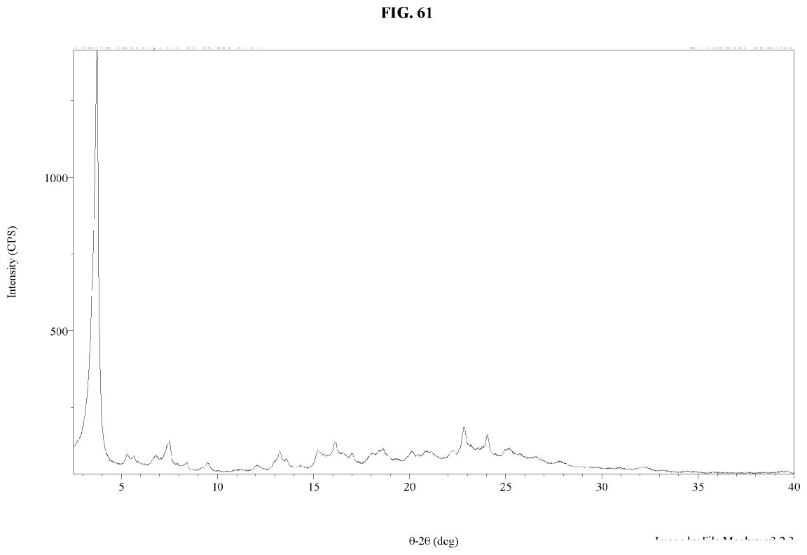

In certain embodiments, phosphate salt Material A of the compound of formula (I) has an XRPD pattern substantially the same as shown in FIG. 61. In certain embodiments, phosphate salt Material A of the compound of formula (I) has an XRPD pattern comprising one or more diffraction peaks (2θ) disclosed in Table 44.

### (B) Phosphate Salt Material B

In certain embodiments, the crystalline phosphate salt of the compound of formula (I) has an XRPD pattern comprising peaks at about 4.1°, about 6.9°, about 10.8°, and about 11.4° 2θ.

In certain embodiments, the XRPD pattern further comprises one or more peaks at about 7.8°, about 8.3°, about 8.5°, about 9.1°, about 9.3°, about 10.6°, about 12.3°, about 13.4°, and about 14.6° 2θ.

In certain embodiments, the XRPD pattern further comprises peaks at about 18.0°, about 20.0°, about 21.4°, and about 21.7° 2θ.

In certain embodiments, the XRPD pattern further comprises one or more peaks at about 15.8°, about 16.2°, about 16.4°, about 16.6°, about 16.7°, about 17.1°, about 18.4°, about 18.8°, about 19.8°, about 20.8°, about 22.0°, about 22.5°, about 22.9°, about 23.2°, and about 24.0° 2θ.

In certain embodiments, the XRPD pattern further comprises peaks at about 25.1°, about 26.1°, and about 28.0° 2θ.

In certain embodiments, the XRPD pattern further comprises one or more peaks at about 26.7°, about 27.3°, about 28.5°, and about 28.7° 2θ.

In certain embodiments, the crystalline phosphate salt of the compound of formula (I) has an XRPD pattern comprising one or more peaks at about 4.1°, about 6.9°, about 7.8°, about 8.3°, about 8.5°, about 9.1°, about 9.3°, about 10.6°, about 10.8°, about 11.4°, about 12.3°, about 13.4°, about 14.6°, about 15.8°, about 16.2°, about 16.4°, about 16.6°, about 16.7°, about 17.1°, about 18.0°, about 18.4°, about 18.8°, about 19.8°, about 20.0°, about 20.8°, about 21.4°, about 21.7°, about 22.0°, about 22.5°, about 22.9°, about 23.2°, about 24.0°, about 25.1°, about 26.1°, about 26.7°, about 27.3°, about 28.0°, about 28.5°, and about 28.7° 2θ.

In certain embodiments, the crystalline phosphate salt of the compound of formula (I) has an XRPD pattern comprising peaks at about 4.1°, about 6.9°, about 7.8°, about 8.3°, about 8.5°, about 9.1°, about 9.3°, about 10.6°, about 10.8°, about 11.4°, about 12.3°, about 13.4°, about 14.6°, about 15.8°, about 16.2°, about 16.4°, about 16.6°, about 16.7°, about 17.1°, about 18.0°, about 18.4°, about 18.8°, about 19.8°, about 20.0°, about 20.8°, about 21.4°, about 21.7°, about 22.0°, about 22.5°, about 22.9°, about 23.2°, about 24.0°, about 25.1°, about 26.1°, about 26.7°, about 27.3°, about 28.0°, about 28.5°, and about 28.7° 2θ.

In certain embodiments, the material comprising a crystalline phosphate salt comprising XRPD peaks at about 4.1°, about 6.9°, about 7.8°, about 8.3°, about 8.5°, about 9.1°, about 9.3°, about 10.6°, about 10.8°, about 11.4°, about 12.3°, about 13.4°, about 14.6°, about 15.8°, about 16.2°, about 16.4°, about 16.6°, about 16.7°, about 17.1°, about 18.0°, about 18.4°, about 18.8°, about 19.8°, about 20.0°, about 20.8°, about 21.4°, about 21.7°, about 22.0°, about 22.5°, about 22.9°, about 23.2°, about 24.0°, about 25.1°, about 26.1°, about 26.7°, about 27.3°, about 28.0°, about 28.5°, and about 28.7° 2θ may be referred to herein as “phosphate salt Material B”.

In certain embodiments, the crystalline phosphate salt of the compound of formula (I) has an XRPD pattern comprising peaks at 4.1°±0.3°, 6.9°±0.3°, 10.8°±0.3°, and 11.4°±0.3° 2θ.

In certain embodiments, the XRPD pattern further comprises one or more peaks at 7.8°±0.3°, 8.3°±0.3°, 8.5°±0.3°, 9.1°±0.3°, 9.3°±0.3°, 10.6°±0.3°, 12.3°±0.3°, 13.4°±0.3°, and 14.6°±0.3° 2θ.

In certain embodiments, the XRPD pattern further comprises peaks at 18.0°±0.3°, 20.0°±0.3°, 21.4°±0.3°, and 21.7°±0.3° 2θ.

In certain embodiments, the XRPD pattern further comprises one or more peaks at 15.8°±0.3°, 16.2°±0.3°, 16.4°±0.3°, 16.6°±0.3°, 16.7°±0.3°, 17.1°±0.3°, 18.4°±0.3°, 18.8°±0.3°, 19.8°±0.3°, 20.8°±0.3°, 22.0°±0.3°, 22.5°±0.3°, 22.9°±0.3°, 23.2°±0.3°, and 24.0°±0.3° 2θ.

In certain embodiments, the XRPD pattern further comprises peaks at 25.1°±0.3°, 26.1°±0.3°, and 28.0°±0.3° 2θ.

In certain embodiments, the XRPD pattern further comprises one or more peaks at 26.7°±0.3°, 27.3°±0.3°, 28.5°±0.3°, and 28.7°±0.3° 2θ.

In certain embodiments, the crystalline phosphate salt of the compound of formula (I) has an XRPD pattern comprising one or more peaks at 4.1°±0.3°, 6.9°±0.3°, 7.8°±0.3°, 8.3°±0.3°, 8.5°±0.3°, 9.1°±0.3°, 9.3°±0.3°, 10.6°±0.3°, 10.8°±0.3°, 11.4°±0.3°, 12.3°±0.3°, 13.4°±0.3°, 14.6°±0.3°, 15.8°±0.3°, 16.2°±0.3°, 16.4°±0.3°, 16.6°±0.3°, 16.7°±0.3°, 17.1°±0.3°, 18.0°±0.3°, 18.4°±0.3°, 18.8°±0.3°, 19.8°±0.3°, 20.0°±0.3°, 20.8°±0.3°, 21.4°±0.3°, 21.7°±0.3°, 22.0°±0.3°, 22.5°±0.3°, 22.9°±0.3°, 23.2°±0.3°, 24.0°±0.3°, 25.1°±0.3°, 26.1°±0.3°, 26.7°±0.3°, 27.3°±0.3°, 28.0°±0.3°, 28.5°±0.3°, and 28.7°±0.3° 2θ.

In certain embodiments, the crystalline phosphate salt of the compound of formula (I) has an XRPD pattern comprising peaks at 4.1°±0.3°, 6.9°±0.3°, 7.8°±0.3°, 8.3°±0.3°, 8.5°±0.3°, 9.1°±0.3°, 9.3°±0.3°, 10.6°±0.3°, 10.8°±0.3°, 11.4°±0.3°, 12.3°±0.3°, 13.4°±0.3°, 14.6°±0.3°, 15.8°±0.3°, 16.2°±0.3°, 16.4°±0.3°, 16.6°±0.3°, 16.7°±0.3°, 17.1°±0.3°, 18.0°±0.3°, 18.4°±0.3°, 18.8°±0.3°, 19.8°±0.3°, 20.0°±0.3°, 20.8°±0.3°, 21.4°±0.3°, 21.7°±0.3°, 22.0°±0.3°, 22.5°±0.3°, 22.9°±0.3°, 23.2°±0.3°, 24.0°±0.3°, 25.1°±0.3°, 26.1°±0.3°, 26.7°±0.3°, 27.3°±0.3°, 28.0°±0.3°, 28.5°±0.3°, and 28.7° 0.3° 2θ

In certain embodiments, the material comprising a crystalline phosphate salt comprising XRPD peaks at 4.1°±0.3°, 6.9°±0.3°, 7.8°±0.3°, 8.3°±0.3°, 8.5°±0.3°, 9.1°±0.3°, 9.3°±0.3°, 10.6°±0.3°, 10.8°±0.3°, 11.4°±0.3°, 12.3°±0.3°, 13.4°±0.3°, 14.6°±0.3°, 15.8°±0.3°, 16.2°±0.3°, 16.4°±0.3°, 16.6°±0.3°, 16.7°±0.3°, 17.1°±0.3°, 18.0°±0.3°, 18.4°±0.3°, 18.8°±0.3°, 19.8°±0.3°, 20.0°±0.3°, 20.8°±0.3°, 21.4°±0.3°. 21.7°±0.3°, 22.0°±0.3°, 22.5°±0.3°, 22.9°±0.3°, 23.2°±0.3°, 24.0°±0.3°, 25.1°±0.3°, 26.1°±0.3°, 26.7°±0.3°, 27.3°±0.3°, 28.0°±0.3°, 28.5°±0.3°, and 28.7°±0.3° 2θ may be referred to herein as “phosphate salt Material B”.

In certain embodiments, the crystalline phosphate salt of the compound of formula (I) has an XRPD pattern comprising peaks at 4.1°±0.2°, 6.9°±0.2°, 10.8°±0.2°, and 11.4°±0.2° 2θ.

In certain embodiments, the XRPD pattern further comprises one or more peaks at 7.8°±0.2°, 8.3°±0.2°, 8.5°±0.2°, 9.1°±0.2°, 9.3°±0.2°, 10.6°±0.2°, 12.3°±0.2°, 13.4°±0.2°, and 14.6°±0.2° 2θ.

In certain embodiments, the XRPD pattern further comprises peaks at 18.0°±0.2°, 20.0°±0.2°, 21.4°±0.2°, and 21.7°±0.2° 2θ.

In certain embodiments, the XRPD pattern further comprises one or more peaks at 15.8°±0.2°, 16.2°±0.2°, 16.4°±0.2°, 16.6°±0.2°, 16.7°±0.2°, 17.1°±0.2°, 18.4°±0.2°, 18.8°±0.2°, 19.8°±0.2°, 20.8°±0.2°, 22.0°±0.2°, 22.5°±0.2°, 22.9°±0.2°, 23.2°±0.2°, and 24.0°±0.2° 2θ.

In certain embodiments, the XRPD pattern further comprises peaks at 25.1°±0.2°, 26.1°±0.2°, and 28.0°±0.2° 2θ.

In certain embodiments, the XRPD pattern further comprises one or more peaks at 26.7°±0.2°, 27.3°±0.2°, 28.5°±0.2°, and 28.7°±0.2° 2θ.

In certain embodiments, the crystalline phosphate salt of the compound of formula (I) has an XRPD pattern comprising one or more peaks at 4.1°±0.2°, 6.9°±0.2°, 7.8°±0.2°, 8.3°±0.2°, 8.5°±0.2°, 9.1°±0.2°, 9.3°±0.2°, 10.6°±0.2°, 10.8°±0.2°, 11.4°±0.2°, 12.3°±0.2°, 13.4°±0.2°, 14.6°±0.2°, 15.8°±0.2°, 16.2°±0.2°, 16.4°±0.2°, 16.6°±0.2°, 16.7°±0.2°, 17.1°±0.2°, 18.0°±0.2°, 18.4°±0.2°, 18.8°±0.2°, 19.8°±0.2°, 20.0°±0.2°, 20.8°±0.2°, 21.4°±0.2°, 21.7°±0.2°, 22.0°±0.2°, 22.5°±0.2°, 22.9°±0.2°, 23.2°±0.2°, 24.0°±0.2°, 25.1°±0.2°, 26.1°±0.2°, 26.7°±0.2°, 27.3°±0.2°, 28.0°±0.2°, 28.5°±0.2°, and 28.7°±0.2° 2θ.

In certain embodiments, the crystalline phosphate salt of the compound of formula (I) has an XRPD pattern comprising peaks at 4.1°±0.2°, 6.9°±0.2°, 7.8°±0.2°, 8.3°±0.2°, 8.5°±0.2°, 9.1°±0.2°, 9.3°±0.2°, 10.6°±0.2°, 10.8°±0.2°, 11.4°±0.2°, 12.3°±0.2°, 13.4°±0.2°, 14.6°±0.2°, 15.8°±0.2°, 16.2°±0.2°, 16.4°±0.2°, 16.6°±0.2°, 16.7°±0.2°, 17.1°±0.2°, 18.0°±0.2°, 18.4°±0.2°, 18.8°±0.2°, 19.8°±0.2°, 20.0°±0.2°, 20.8°±0.2°, 21.4°±0.2°, 21.7°±0.2°, 22.0°±0.2°, 22.5°±0.2°, 22.9°±0.2°, 23.2°±0.2°, 24.0°±0.2°, 25.1°±0.2°, 26.1°±0.2°, 26.7°±0.2°, 27.3°±0.2°, 28.0°±0.2°, 28.5°±0.2°, and 28.7°±0.2° 2θ.

In certain embodiments, the material comprising a crystalline phosphate salt comprising XRPD peaks at 4.1°±0.2°, 6.9°±0.2°, 7.8°±0.2°, 8.3°±0.2°, 8.5°±0.2°, 9.1°±0.2°, 9.3°±0.2°, 10.6°±0.2°, 10.8°±0.2°, 11.4°±0.2°, 12.3°±0.2°, 13.4°±0.2°, 14.6°±0.2°, 15.8°±0.2°, 16.2°±0.2°, 16.4°±0.2°, 16.6°±0.2°, 16.7°±0.2°, 17.1°±0.2°, 18.0°±0.2°, 18.4°±0.2°, 18.8°±0.2°, 19.8°±0.2°, 20.0°±0.2°, 20.8°±0.2°, 21.4°±0.2°, 21.7°±0.2°, 22.0°±0.2°, 22.5°±0.2°, 22.9°±0.2°, 23.2°±0.2°, 24.0°±0.2°, 25.1°±0.2°, 26.1°±0.2°, 26.7°±0.2°, 27.3°±0.2°, 28.0°±0.2°, 28.5°±0.2°, and 28.7°±0.2° 2θ may be referred to herein as “phosphate salt Material B”.

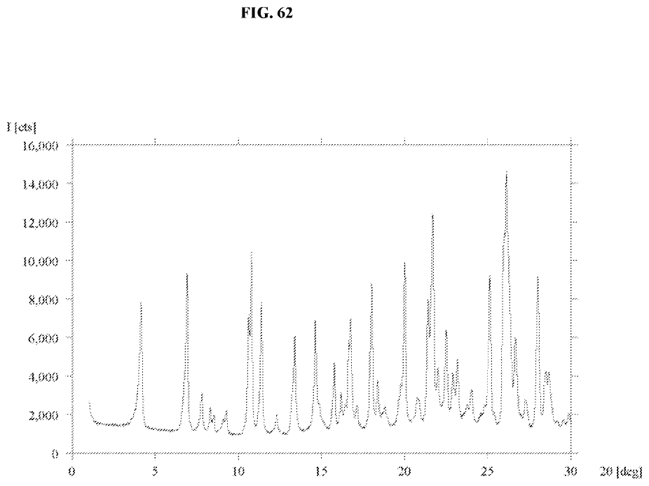

In certain embodiments, phosphate salt Material B of the compound of formula (I) has an XRPD pattern substantially the same as shown in FIG. 62. In certain embodiments, phosphate salt Material B of the compound of formula (I) has an XRPD pattern comprising one or more diffraction peaks (2θ) disclosed in Table 45.

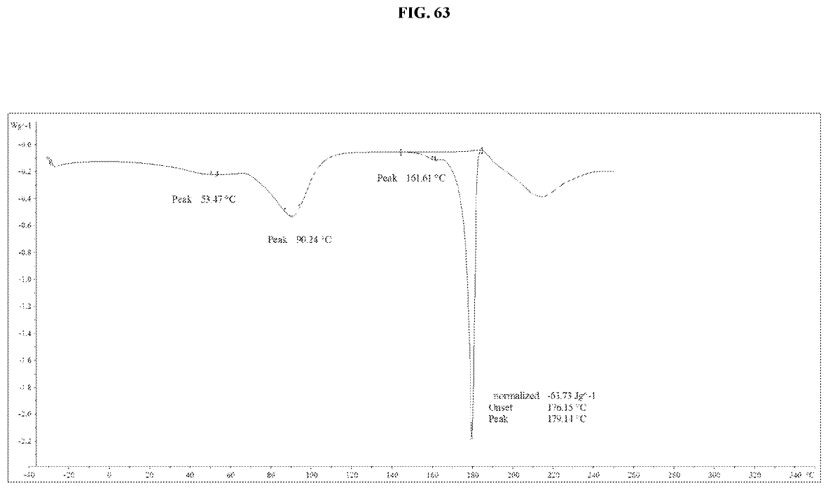

In certain embodiments, phosphate salt Material B of the compound of formula (I) has a DSC thermogram comprising an endotherm with a peak maximum at about 54° C. In certain embodiments, phosphate salt Material B of the compound of formula (I) has a DSC thermogram comprising an endotherm with a peak maximum at about 90° C. In certain embodiments, phosphate salt Material B of the compound of formula (I) has a DSC thermogram comprising one or more endotherms with peak maxima at about 54° C. and about 90° C. In certain embodiments, phosphate salt Material B of the compound of formula (I) has a DSC thermogram comprising an endotherm with a peak onset at about 176° C. In certain embodiments, phosphate salt Material B of the compound of formula (I) has a DSC thermogram substantially the same as shown in FIG. 63.

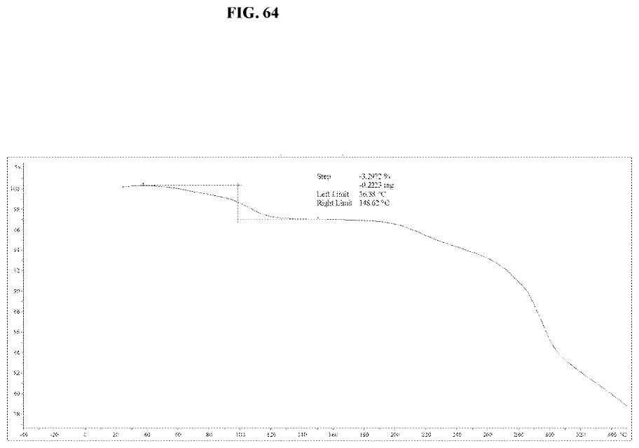

In certain embodiments, phosphate salt Material B of the compound of formula (I) exhibits a weight loss of less than or equal to about 3.3% wt. upon heating phosphate salt Material B of the compound of formula (I) from about 36° C. to about 149° C. The weight loss exhibited by a crystalline form (e.g., phosphate salt Material B of the compound of formula (I)) can be determined, for example, using TGA. In certain embodiments, phosphate salt Material B of the compound of formula (I) has a TGA thermogram substantially the same as shown in FIG. 64.

### Crystalline Free Base Forms of a Compound of Formula (I)

In one aspect, provided herein are crystalline forms of the compound of formula (I)

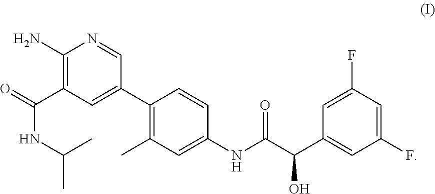 [SMILES: Cc1cc(NC(=O)[C@H](O)c2cc(F)cc(F)c2)ccc1-c1cnc(N)c(C(=O)NC(C)C)c1]

In certain embodiments, the crystalline form of the compound of formula (I) is a crystalline free base form. In certain embodiments, the crystalline form of the compound of formula (I) is an anhydrous crystalline form.

In certain embodiments, the crystalline form of the compound of formula (I) is Form A of the compound of formula (I). In certain embodiments, the crystalline form of the compound of formula (I) is Form B of the compound of formula (I). In certain embodiments, the crystalline form of the compound of formula (I) is Form C of the compound of formula (I). In certain embodiments, the crystalline form of the compound of formula (I) is Form D of the compound of formula (I). In certain embodiments, the crystalline form of the compound of formula (I) is Form E of the compound of formula (I). In certain embodiments, the crystalline form of the compound of formula (I) is Form H of the compound of formula (I).

In certain embodiments, the crystalline form of the compound of formula (I) is a crystalline solvate. In certain embodiments, the crystalline solvate is a crystalline acetonitrile solvate. In certain embodiments, the crystalline solvate is a crystalline acetone solvate. In certain embodiments, the crystalline solvate is a crystalline ethyl acetate solvate.

In certain embodiments, the crystalline form of the compound of formula (I) is a crystalline hydrate.

### Crystalline Free Base Forms

### (1) Form A

In various embodiments, provided herein is Form A of a compound of formula (I)

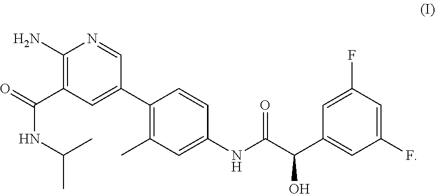 [SMILES: Cc1cc(NC(=O)[C@H](O)c2cc(F)cc(F)c2)ccc1-c1cnc(N)c(C(=O)NC(C)C)c1]

In certain embodiments, Form A of the compound of formula (I) has an X-ray powder diffraction (XRPD) pattern comprising a peak at about 8.1° 2θ. In certain embodiments, Form A of the compound of formula (I) has an XRPD pattern comprising a peak at about 9.4° 2θ. In certain embodiments, Form A of the compound of formula (I) has an XRPD pattern comprising a peak at about 9.8° 2θ. In certain embodiments, Form A of the compound of formula (I) has an XRPD pattern comprising one or more peaks at about 8.1°, about 9.4°, and about 9.8° 2θ.

In certain embodiments, the XRPD pattern further comprises one or more peaks at about 15.6°, about 16.0°, about 16.3°, about 16.9°, about 17.1°, about 18.4°, about 18.8°, about 20.0°, about 20.1°, about 20.3°, about 20.6°, about 21.4°, about 21.6°, and about 24.5° 2θ.

In certain embodiments, Form A of the compound of formula (I) has an XRPD pattern comprising one or more peaks at about 8.1°, about 9.4°, about 9.8°, about 15.6°, about 16.0°, about 16.3°, about 16.9°, about 17.1°, about 18.4°, about 18.8°, about 20.0°, about 20.1°, about 20.3°, about 20.6°, about 21.4°, about 21.6°, and about 24.5° 2θ.

In certain embodiments, Form A of the compound of formula (I) has an XRPD pattern comprising peaks at about 8.1°, about 9.4°, about 9.8°, about 15.6°, about 16.0°, about 16.3°, about 16.9°, about 17.1°, about 18.4°, about 18.8°, about 20.0°, about 20.1°, about 20.3°, about 20.6°, about 21.4°, about 21.6°, and about 24.5° 2θ.

In certain embodiments, Form A of the compound of formula (I) has an XRPD pattern comprising one or more peaks at about 8.1° and about 9.4° 2θ.

In certain embodiments, the XRPD pattern further comprises one or more peaks at about 5.3°, about 6.9°, about 9.8°, about 10.6°, about 10.7°, about 11.9°, about 12.2°, about 12.7°, about 12.9°, about 13.1°, about 13.6°, about 13.9°, about 14.5°, and about 14.8° 2θ.

In certain embodiments, the XRPD pattern further comprises one or more peaks at about 16.3°, about 17.1°, about 18.8°, about 20.0°, about 20.1°, about 20.6°, and about 24.5 2θ.

In certain embodiments, the XRPD pattern further comprises one or more peaks at about 15.6°, about 16.0°, about 16.9°, about 17.6°, about 18.4, about 19.1°, about 19.3°, about 19.6°, about 20.3°, about 20.8°, about 21.0°, about 21.1°, about 21.4°, about 21.6°, about 21.8°, about 22.0°, about 22.3°, about 22.7°, about 22.9°, about 23.3°, about 23.8°, about 24.1°, and about 24.9° 2θ.

In certain embodiments, the XRPD pattern further comprises one or more peaks at about 25.5°, about 26.1°, about 26.3°, about 26.4°, about 26.9°, about 27.1°, and about 29.1° 2θ.

In certain embodiments, the XRPD pattern further comprises one or more peaks at about 26.6°, about 27.7°, about 27.8°, about 28.3°, about 28.8°, about 29.8°, about 30.8°, about 31.2°, about 31.5°, about 31.7°, about 32.3°, about 33.1°, about 33.6°, and about 34.5° 2θ.

In certain embodiments, Form A of the compound of formula (I) has an XRPD pattern comprising one or more peaks at about 5.3°, about 6.9°, about 8.1°, about 9.4°, about 9.8°, about 10.6°, about 10.7°, about 11.9°, about 12.2°, about 12.7°, about 12.9°, about 13.1°, about 13.6°, about 13.9°, about 14.5°, about 14.8°, about 15.6°, about 16.0°, about 16.3°, about 16.9°, about 17.1°, about 17.6°, about 18.5°, about 18.8°, about 19.1°, about 19.3°, about 19.6°, about 20.0°, about 20.1°, about 20.3°, about 20.6°, about 20.8°, about 21.0°, about 21.1°, about 21.4°, about 21.6°, about 21.8°, about 22.0°, about 22.3°, about 22.7°, about 22.9°, about 23.3°, about 23.8°, about 24.1°, about 24.5°, about 24.9°, about 25.5°, about 26.1°, about 26.3°, about 26.4°, about 26.6°, about 26.9°, about 27.1°, about 27.7°, about 27.8°, about 28.3°, about 28.8°, about 29.1°, about 29.8°, about 30.8°, about 31.2°, about 31.5°, about 31.7°, about 32.3°, about 33.1°, about 33.6°, and about 34.5° 2θ.

In certain embodiments, Form A of the compound of formula (I) has an XRPD pattern comprising peaks at about 5.3°, about 6.9°, about 8.1°, about 9.4°, about 9.8°, about 10.6°, about 10.7°, about 11.9°, about 12.2°, about 12.7°, about 12.9°, about 13.1°, about 13.6°, about 13.9°, about 14.5°, about 14.8°, about 15.6°, about 16.0°, about 16.3°, about 16.9°, about 17.1°, about 17.6°, about 18.5°, about 18.8°, about 19.1°, about 19.3°, about 19.6°, about 20.0°, about 20.1°, about 20.3°, about 20.6°, about 20.8°, about 21.0°, about 21.1°, about 21.4°, about 21.6°, about 21.8°, about 22.0°, about 22.3°, about 22.7°, about 22.9°, about 23.3°, about 23.8°, about 24.1°, about 24.5°, about 24.9°, about 25.5°, about 26.1°, about 26.3°, about 26.4°, about 26.6°, about 26.9°, about 27.1°, about 27.7°, about 27.8°, about 28.3°, about 28.8°, about 29.1°, about 29.8°, about 30.8°, about 31.2°, about 31.5°, about 31.7°, about 32.3°, about 33.1°, about 33.6°, and about 34.5° 2θ.

In certain embodiments, Form A of the compound of formula (I) has an XRPD pattern comprising a peak at 8.1°±0.3° 2θ. In certain embodiments, Form A of the compound of formula (I) has an XRPD pattern comprising a peak at 9.4°±0.3° 2θ. In certain embodiments, Form A the compound of formula (I) has an XRPD pattern comprising a peak at 9.8°±0.3° 2θ. In certain embodiments, Form A of the compound of formula (I) has an XRPD pattern comprising one or more peaks at 8.1°±0.3°, 9.4°±0.3°, and 9.8°±0.3° 2θ.

In certain embodiments, the XRPD pattern further comprises one or more peaks at 15.6°±0.3°, 16.0°±0.3°, 16.3°±0.3°, 16.9°±0.3°, 17.1°±0.3°, 18.4°±0.3°, 18.8°±0.3°, 20.0°±0.3°, 20.1°±0.3°, 20.3°±0.3°, 20.6°±0.3°, 21.4°±0.3°, 21.6°±0.3°, and 24.5°±0.3° 2θ.

In certain embodiments, Form A of the compound of formula (I) has an XRPD pattern comprising one or more peaks at 8.1°±0.3°, 9.4°±0.3°, 9.8°±0.3°, 15.6°±0.3°, 16.0°±0.3°, 16.3°±0.3°, 16.9°±0.3°, 17.1°±0.3°, 18.4°±0.3°, 18.8°±0.3°, 20.0°±0.3°, 20.1°±0.3°, 20.3°±0.3°, 20.6°±0.3°, 21.4°±0.3°, 21.6°±0.3°, and 24.5°±0.3° 2θ.

In certain embodiments, Form A of the compound of formula (I) has an XRPD pattern comprising peaks at 8.1°±0.3°, 9.4°±0.3°, 9.8°±0.3°, 15.6°±0.3°, 16.0°±0.3°, 16.3°±0.3°, 16.9°±0.3°, 17.1°±0.3°, 18.4°±0.3°, 18.8°±0.3°, 20.0°±0.3°, 20.1°±0.3°, 20.3°±0.3°, 20.6°±0.3°, 21.4°±0.3°, 21.6°±0.3°, and 24.5°±0.3° 2θ.

In certain embodiments, Form A of the compound of formula (I) has an XRPD pattern comprising one or more peaks at 8.1°±0.3° and 9.4°±0.3° 2θ.

In certain embodiments, the XRPD pattern further comprises one or more peaks at 5.3°±0.3°, 6.9°±0.3°, 9.8°±0.3°, 10.6°±0.3°, 10.7°±0.3°, 11.9°±0.3°, 12.2°±0.3°, 12.7°±0.3°, 12.9°±0.3°, 13.1°±0.3°, 13.6°±0.3°, 13.9°±0.3°, 14.5°±0.3°, and 14.8°±0.3° 2θ.

In certain embodiments, the XRPD pattern further comprises one or more peaks at 16.3°±0.3°, 17.1°±0.3°, 18.8°±0.3°, 20.0°±0.3°, 20.1°±0.3°, 20.6°±0.3°, and 24.5°±0.3° 2θ.

In certain embodiments, the XRPD pattern further comprises one or more peaks at 15.6°±0.3°, 16.0°±0.3°, 16.9°±0.3°, 17.6°±0.3°, 18.4°±0.3°, 19.1°±0.3°, 19.3°±0.3°, 19.6°±0.3°, 20.3°±0.3°, 20.8°±0.3°, 21.0°±0.3°, 21.1°±0.3°, 21.4°±0.3°, 21.6°±0.3°, 21.8°±0.3°, 22.0°±0.3°, 22.3°±0.3°, 22.7°±0.3°, 22.9°±0.3°, 23.3°±0.3°, 23.8°±0.3°, 24.1°±0.3°, and 24.9°±0.3° 2θ.

In certain embodiments, the XRPD pattern further comprises one or more peaks at 25.5°±0.3°, 26.1°±0.3°, 26.3°±0.3°, 26.4°±0.3°, 26.9°±0.3°, 27.1°±0.3°, and 29.1°±0.3° 2θ.

In certain embodiments, the XRPD pattern further comprises one or more peaks at 26.6°±0.3°, 27.7°±0.3°, 27.8°±0.3°, 28.3°±0.3°, 28.8°±0.3°, 29.8°±0.3°, 30.8°±0.3°, 31.2°±0.3°, 31.5°±0.3°, 31.7°±0.3°, 32.3°±0.3°, 33.1°±0.3°, 33.6°±0.3°, and 34.5°±0.3° 2θ.

In certain embodiments, Form A of the compound of formula (I) has an XRPD pattern comprising one or more peaks at 5.3°±0.3°, 6.9°±0.3°, 8.1°±0.3°, 9.4°±0.3°, 9.8°±0.3°, 10.6°±0.3°, 10.7°±0.3°, 11.9°±0.3°, 12.2°±0.3°, 12.7°±0.3°, 12.9°±0.3°, 13.1°±0.3°, 13.6°±0.3°, 13.9°±0.3°, 14.5°±0.3°, 14.8°±0.3°, 15.6°±0.3°, 16.0°±0.3°, 16.3°±0.3°, 16.9°±0.3°, 17.1°±0.3°, 17.6°±0.3°, 18.5°±0.3°, 18.8°±0.3°, 19.1°±0.3°, 19.3°±0.3°, 19.6°±0.3°, 20.0°±0.3°, 20.1°±0.3°, 20.3°±0.3°, 20.6°±0.3°, 20.8°±0.3°, 21.0°±0.3°, 21.1°±0.3°, 21.4°±0.3°, 21.6°±0.3°, 21.8°±0.3°, 22.0°±0.3°, 22.3°±0.3°, 22.7°±0.3°, 22.9°±0.3°, 23.3°±0.3°, 23.8°±0.3°, 24.1°±0.3°, 24.5°±0.3°, 24.9°±0.3°, 25.5°±0.3°, 26.1°±0.3°, 26.3°±0.3°, 26.4°±0.3°, 26.6°±0.3°, 26.9°±0.3°, 27.1°±0.3°, 27.7°±0.3°, 27.8°±0.3°, 28.3°±0.3°, 28.8°±0.3°, 29.1°±0.3°, 29.8°±0.3°, 30.8°±0.3°, 31.2°±0.3°, 31.5°±0.3°, 31.7°±0.3°, 32.3°±0.3°, 33.1°±0.3°, 33.6°±0.3°, and 34.5°±0.3° 2θ.

In certain embodiments, Form A of the compound of formula (I) has an XRPD pattern comprising peaks at 5.3°±0.3°, 6.9°±0.3°, 8.1°±0.3°, 9.4°±0.3°, 9.8°±0.3°, 10.6°±0.3°, 10.7°±0.3°, 11.9°±0.3°, 12.2°±0.3°, 12.7°±0.3°, 12.9°±0.3°, 13.1°±0.3°, 13.6°±0.3°, 13.9°±0.3°, 14.5°±0.3°, 14.8°±0.3°, 15.6°±0.3°, 16.0°±0.3°, 16.3°±0.3°, 16.9°±0.3°, 17.1°±0.3°, 17.6°±0.3°, 18.5°±0.3°, 18.8°±0.3°, 19.1°±0.3°, 19.3°±0.3°, 19.6°±0.3°, 20.0°±0.3°, 20.1°±0.3°, 20.3°±0.3°, 20.6°±0.3°, 20.8°±0.3°, 21.0°±0.3°, 21.1°±0.3°, 21.4°±0.3°, 21.6°±0.3°, 21.8°±0.3°, 22.0°±0.3°, 22.3°±0.3°, 22.7°±0.3°, 22.9°±0.3°, 23.3°±0.3°, 23.8°±0.3°, 24.1°±0.3°, 24.5°±0.3°, 24.9°±0.3°, 25.5°±0.3°, 26.1°±0.3°, 26.3°±0.3°, 26.4°±0.3°, 26.6°±0.3°, 26.9°±0.3°, 27.1°±0.3°, 27.7°±0.3°, 27.8°±0.3°, 28.3°±0.3°, 28.8°±0.3°, 29.1°±0.3°, 29.8°±0.3°, 30.8°±0.3°, 31.2°±0.3°, 31.5°±0.3°, 31.7°±0.3°, 32.3°±0.3°, 33.1°±0.3°, 33.6°±0.3°, and 34.5°±0.3° 2θ.

In certain embodiments, Form A of the compound of formula (I) has an XRPD pattern comprising a peak at 8.1°±0.2° 2θ. In certain embodiments, Form A of the compound of formula (I) has an XRPD pattern comprising a peak at 9.4°±0.2° 2θ. In certain embodiments, Form A of the compound of formula (I) has an XRPD pattern comprising a peak at 9.8°±0.2° 2θ. In certain embodiments, Form A of the compound of formula (I) has an XRPD pattern comprising one or more peaks at 8.1°±0.2°, 9.4°±0.2°, and 9.8°±0.2° 2θ.

In certain embodiments, the XRPD pattern further comprises one or more peaks at 15.6°±0.2°, 16.0°±0.2°, 16.3°±0.2°, 16.9°±0.2°, 17.1°±0.2°, 18.4°±0.2°, 18.8°±0.2°, 20.0°±0.2°, 20.1°±0.2°, 20.3°±0.2°, 20.6°±0.2°, 21.4°±0.2°, 21.6°±0.2°, and 24.5°±0.2° 2θ.

In certain embodiments, Form A of the compound of formula (I) has an XRPD pattern comprising one or more peaks at 8.1°±0.2°, 9.4°±0.2°, 9.8°±0.2°, 15.6°±0.2°, 16.0°±0.2°, 16.3°±0.2°, 16.9°±0.2°, 17.1°±0.2°, 18.4°±0.2°, 18.8°±0.2°, 20.0°±0.2°, 20.1°±0.2°, 20.3°±0.2°, 20.6°±0.2°, 21.4°±0.2°, 21.6°±0.2°, and 24.5°±0.2° 2θ.

In certain embodiments, Form A of the compound of formula (I) has an XRPD pattern comprising peaks at 8.1°±0.2°, 9.4°±0.2°, 9.8°±0.2°, 15.6°±0.2°, 16.0°±0.2°, 16.3°±0.2°, 16.9°±0.2°, 17.1°±0.2°, 18.4°±0.2°, 18.8°±0.2°, 20.0°±0.2°, 20.1°±0.2°, 20.3°±0.2°, 20.6°±0.2°, 21.4°±0.2°, 21.6°±0.2°, and 24.5°±0.2° 2θ.

In certain embodiments, Form A of the compound of formula (I) has an XRPD pattern comprising one or more peaks at 8.1°±0.2° and 9.4°±0.2° 2θ.

In certain embodiments, the XRPD pattern further comprises one or more peaks at 5.3°±0.2°, 6.9°±0.2°, 9.8°±0.2°, 10.6°±0.2°, 10.7°±0.2°, 11.9°±0.2°, 12.2°±0.2°, 12.7°±0.2°, 12.9°±0.2°, 13.1°±0.2°, 13.6°±0.2°, 13.9°±0.2°, 14.5°±0.2°, and 14.8°±0.2° 2θ.

In certain embodiments, the XRPD pattern further comprises one or more peaks at 16.3°±0.2°, 17.1°±0.2°, 18.8°±0.2°, 20.0°±0.2°, 20.1°±0.2°, 20.6°±0.2°, and 24.5°±0.2° 2θ.

In certain embodiments, the XRPD pattern further comprises one or more peaks at 15.6°±0.2°, 16.0°±0.2°, 16.9°±0.2°, 17.6°±0.2°, 18.4°±0.2°, 19.1°±0.2°, 19.3°±0.2°, 19.6°±0.2°, 20.3°±0.2°, 20.8°±0.2°, 21.0°±0.2°, 21.1°±0.2°, 21.4°±0.2°, 21.6°±0.2°, 21.8°±0.2°, 22.0°±0.2°, 22.3°±0.2°, 22.7°±0.2°, 22.9°±0.2°, 23.3°±0.2°, 23.8°±0.2°, 24.1°±0.2° and 24.9°±0.2° 2θ.

In certain embodiments, the XRPD pattern further comprises one or more peaks at 25.5°±0.2°, 26.1°±0.2°, 26.3°±0.2°, 26.4°±0.2°, 26.9°±0.2°, 27.1°±0.2°, and 29.1°±0.2° 2θ.

In certain embodiments, the XRPD pattern further comprises one or more peaks at 26.6°±0.2°, 27.7°±0.2°, 27.8°±0.2°, 28.3°±0.2°, 28.8°±0.2°, 29.8°±0.2°, 30.8°±0.2°, 31.2°±0.2°, 31.5°±0.2°, 31.7°±0.2°, 32.3°±0.2°, 33.1°±0.2°, 33.6°±0.2°, and 34.5°±0.2° 2θ.

In certain embodiments, Form A of the compound of formula (I) has an XRPD pattern comprising one or more peaks at 5.3°±0.2°, 6.9°±0.2°, 8.1°±0.2°, 9.4°±0.2°, 9.8°±0.2°, 10.6°±0.2°, 10.7°±0.2°, 11.9°±0.2°, 12.2°±0.2°, 12.7°±0.2°, 12.9°±0.2°, 13.1°±0.2°, 13.6°±0.2°, 13.9°±0.2°, 14.5°±0.2°, 14.8°±0.2°, 15.6°±0.2°, 16.0°±0.2°, 16.3°±0.2°, 16.9°±0.2°, 17.1°±0.2°, 17.6°±0.2°, 18.5°±0.2°, 18.8°±0.2°, 19.1°±0.2°, 19.3°±0.2°, 19.6°±0.2°, 20.0°±0.2°, 20.1°±0.2°, 20.3°±0.2°, 20.6°±0.2°, 20.8°±0.2°, 21.0°±0.2°, 21.1°±0.2°, 21.4°±0.2°, 21.6°±0.2°, 21.8°±0.2°, 22.0°±0.2°, 22.3°±0.2°, 22.7°±0.2°, 22.9°±0.2°, 23.3°±0.2°, 23.8°±0.2°, 24.1°±0.2°, 24.5°±0.2°, 24.9°±0.2°, 25.5°±0.2°, 26.1°±0.2°, 26.3°±0.2°, 26.4°±0.2°, 26.6°±0.2°, 26.9°±0.2°, 27.1°±0.2°, 27.7°±0.2°, 27.8°±0.2°, 28.3°±0.2°, 28.8°±0.2°, 29.1°±0.2°, 29.8°±0.2°, 30.8°±0.2°, 31.2°±0.2°, 31.5°±0.2°, 31.7°±0.2°, 32.3°±0.2°, 33.1°±0.2°, 33.6°±0.2°, and 34.5°±0.2° 2θ.

In certain embodiments, Form A of the compound of formula (I) has an XRPD pattern comprising peaks at 5.3°±0.2°, 6.9°±0.2°, 8.1°±0.2°, 9.4°±0.2°, 9.8°±0.2°, 10.6°±0.2°, 10.7°±0.2°, 11.9°±0.2°, 12.2°±0.2°, 12.7°±0.2°, 12.9°±0.2°, 13.1°±0.2°, 13.6°±0.2°, 13.9°±0.2°, 14.5°±0.2°, 14.8°±0.2°, 15.6°±0.2°, 16.0°±0.2°, 16.3°±0.2°, 16.9°±0.2°, 17.1°±0.2°, 17.6°±0.2°, 18.5°±0.2°, 18.8°±0.2°, 19.1°±0.2°, 19.3°±0.2°, 19.6°±0.2°, 20.0°±0.2°, 20.1°±0.2°, 20.3°±0.2°, 20.6°±0.2°, 20.8°±0.2°, 21.0°±0.2°, 21.1°±0.2°, 21.4°±0.2°, 21.6°±0.2°, 21.8°±0.2°, 22.0°±0.2°, 22.3°±0.2°, 22.7°±0.2°, 22.9°±0.2°, 23.3°±0.2°, 23.8°±0.2°, 24.1°±0.2°, 24.5°±0.2°, 24.9°±0.2°, 25.5°±0.2°, 26.1°±0.2°, 26.3°±0.2°, 26.4°±0.2°, 26.6°±0.2°, 26.9°±0.2°, 27.1°±0.2°, 27.7°±0.2°, 27.8°±0.2°, 28.3°±0.2°, 28.8°±0.2°, 29.1°±0.2°, 29.8°±0.2°, 30.8°±0.2°, 31.2°±0.2°, 31.5°±0.2°, 31.7°±0.2°, 32.3°±0.2°, 33.1°±0.2°, 33.6°±0.2°, and 34.5°±0.2° 2θ.

In certain embodiments, Form A of the compound of formula (I) exists in a triclinic crystal system and has a P1 space group. In certain embodiments, Form A of the compound of formula (I) is characterized by the crystallographic unit cell parameters as set forth below.

### Unit Cell Parameters of Form A of the Compound of Formula I

| | | |
|---|---|---|
| Unit cell dimensions | a = 10.80853(12) Å | α = 79.7155(11)° |
| | b = 13.1889(2) Å | β = 80.3819(9)° |
| | c = 16.99402(19) Å | γ = 77.3515(11)° |

| | |
|---|---|
| Unit cell volume (Å³) | 2305.02(5) |
| Cell formula units, Z | 4 |
| Calculated density (g · cm⁻³) | 1.310 |

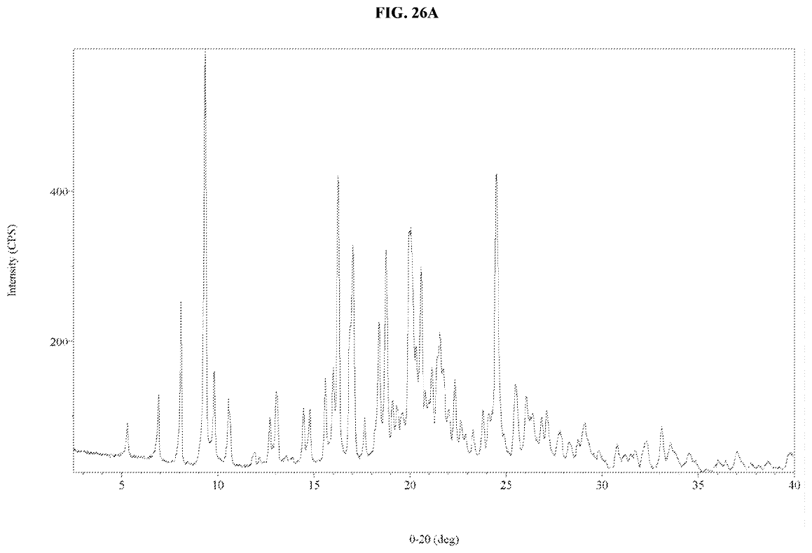

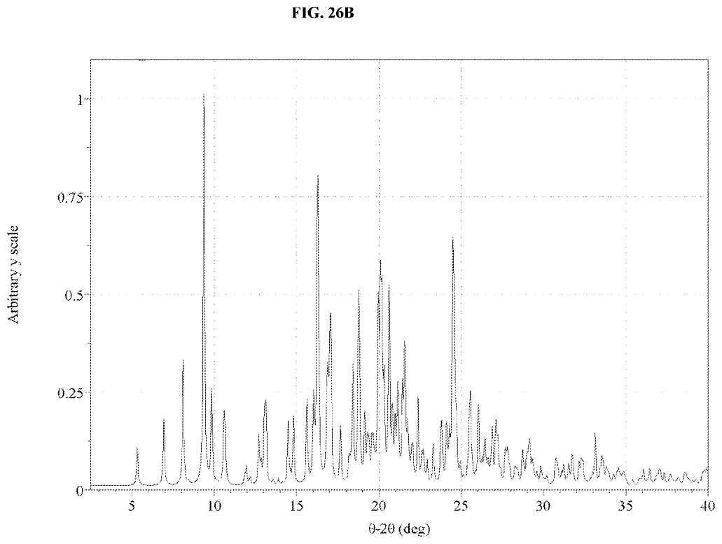

In certain embodiments, Form A of the compound of formula (I) has an XRPD pattern substantially the same as shown in FIG. 26A. In certain embodiments, Form A of the compound of formula (I) has an XRPD pattern comprising one or more diffraction peaks (2θ) disclosed in Table 28.

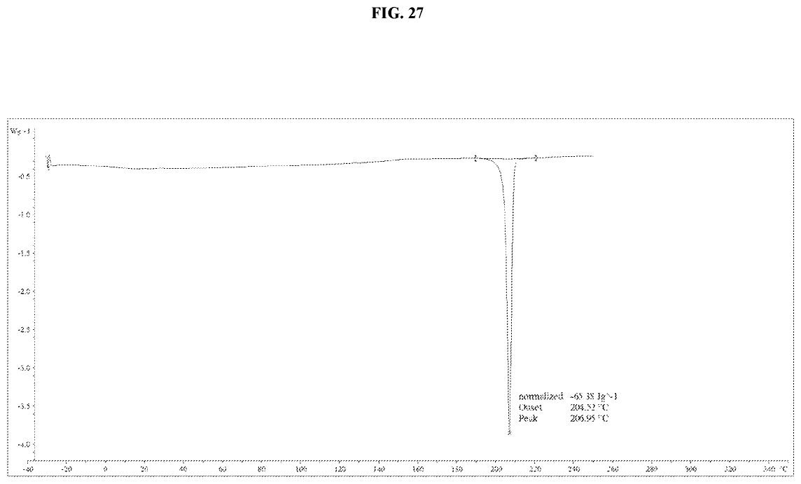

In certain embodiments, Form A of the compound of formula (I) has a differential scanning calorimetry (DSC) thermogram comprising an endotherm with a peak onset at about 205° C. In certain embodiments, Form A of the compound of formula (I) has a DSC thermogram substantially the same as shown in FIG. 27.

In certain embodiments, Form A of the compound of formula (I) is an anhydrous crystalline form.

### (2) Form B

In various embodiments, provided herein is Form B of a compound of formula (I)

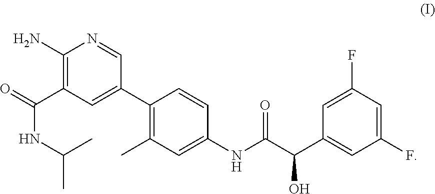 [SMILES: Cc1cc(NC(=O)[C@H](O)c2cc(F)cc(F)c2)ccc1-c1cnc(N)c(C(=O)NC(C)C)c1]

In certain embodiments, Form B of the compound of formula (I) has an XRPD pattern comprising one or more peaks at about 4.0°, about 11.1°, and about 12.9° 2θ.

In certain embodiments, the XRPD pattern further comprises one or more peaks at about 7.9°, about 9.1°, about 10.4°, and about 11.9° 2θ.

In certain embodiments, the XRPD pattern further comprises one or more peaks at about 15.9°, about 18.8°, about 22.4°, about 23.1°, and about 23.8° 2θ.

In certain embodiments, the XRPD pattern further comprises one or more peaks at about 18.2°, about 18.5°, about 19.4°, about 19.9°, about 20.2°, about 21.4°, about 24.4°, and about 24.7° 2θ.

In certain embodiments, the XRPD pattern further comprises one or more peaks at about 28.8° and about 31.5° 2θ.

In certain embodiments, the XRPD pattern further comprises one or more peaks at about 26.0°, about 26.3°, about 26.6°, about 27.5°, about 29.7°, about 30.1°, about 32.1°, about 32.3°, about 32.7°, about 33.0°, about 33.4°, about 33.9°, and about 33.4° 2θ.

In certain embodiments, Form B of the compound of formula (I) has an XRPD pattern comprising one or more peaks at about 4.0°, about 7.9°, about 9.1°, about 10.4°, about 11.1°, about 11.9°, about 12.9°, about 15.9°, about 18.2°, about 18.5°, about 18.8°, about 19.4°, about 19.9°, about 20.2°, about 21.4°, about 22.4°, about 23.1°, about 23.8°, about 24.4°, about 24.7°, about 26.0°, about 26.3°, about 26.6°, about 27.5°, about 28.8°, about 29.7°, about 30.1°, about 31.5°, about 32.1°, about 32.3°, about 32.7°, about 33.0°, about 33.4°, about 33.9°, and about 33.4° 2θ.

In certain embodiments, Form B of the compound of formula (I) has an XRPD pattern comprising peaks at about 4.0°, about 7.9°, about 9.1°, about 10.4°, about 11.1°, about 11.9°, about 12.9°, about 15.9°, about 18.2°, about 18.5°, about 18.8°, about 19.4°, about 19.9°, about 20.2°, about 21.4°, about 22.4°, about 23.1°, about 23.8°, about 24.4°, about 24.7°, about 26.0°, about 26.3°, about 26.6°, about 27.5°, about 28.8°, about 29.7°, about 30.1°, about 31.5°, about 32.1°, about 32.3°, about 32.7°, about 33.0°, about 33.4°, about 33.9°, and about 33.4° 2θ.

In certain embodiments, Form B of the compound of formula (I) has an XRPD pattern comprising one or more peaks at 4.0°±0.3°, 11.1°±0.3°, and 12.9°±0.3° 2θ.

In certain embodiments, the XRPD pattern further comprises one or more peaks at 7.9°±0.3°, 9.1°±0.3°, 10.4°±0.3°, and 11.9°±0.3° 2θ.

In certain embodiments, the XRPD pattern further comprises one or more peaks at 15.9°±0.3°, 18.8°±0.3°, 22.4°±0.3°, 23.1°±0.3°, and 23.8°±0.3° 2θ.

In certain embodiments, the XRPD pattern further comprises one or more peaks at 18.2°±0.3°, 18.5°±0.3°, 19.4°±0.3°, 19.9°±0.3°, 20.2°±0.3°, 21.4°±0.3°, 24.4°±0.3°, and 24.7°±0.3° 2θ.

In certain embodiments, the XRPD pattern further comprises one or more peaks at 28.8°±0.3° and 31.5°±0.3° 2θ.

In certain embodiments, the XRPD pattern further comprises one or more peaks at 26.0°±0.3°, 26.3°±0.3°, 26.6°±0.3°, 27.5°±0.3°, 29.7°±0.3°, 30.1°±0.3°, 32.1°±0.3°, 32.3°±0.3°, 32.7°±0.3°, 33.0°±0.3°, 33.4°±0.3°, 33.9°±0.3°, and 33.4°±0.3° 2θ.

In certain embodiments, Form B of the compound of formula (I) has an XRPD pattern comprising one or more peaks at 4.0°±0.3°, 7.9°±0.3°, 9.1°±0.3°, 10.4°±0.3°, 11.1°±0.3°, 11.9°±0.3°, 12.9°±0.3°, 15.9°±0.3°, 18.2°±0.3°, 18.5°±0.3°, 18.8°±0.3°, 19.4°±0.3°, 19.9°±0.3°, 20.2°±0.3°, 21.4°±0.3°, 22.4°±0.3°, 23.1°±0.3°, 23.8°±0.3°, 24.4°±0.3°, 24.7°±0.3°, 26.0°±0.3°, 26.3°±0.3°, 26.6°±0.3°, 27.5°±0.3°, 28.8°±0.3°, 29.7°±0.3°, 30.1°±0.3°, 31.5°±0.3°, 32.1°±0.3°, 32.3°±0.3°, 32.7°±0.3°, 33.0°±0.3°, 33.4°±0.3°, 33.9°±0.3°, and 33.4°±0.3° 2θ.

In certain embodiments, Form B of the compound of formula (I) has an XRPD pattern comprising peaks at 4.0°±0.3°, 7.9°±0.3°, 9.1°±0.3°, 10.4°±0.3°, 11.1°±0.3°, 11.9°±0.3°, 12.9°±0.3°, 15.9°±0.3°, 18.2°±0.3°, 18.5°±0.3°, 18.8°±0.3°, 19.4°±0.3°, 19.9°±0.3°, 20.2°±0.3°, 21.4°±0.3°, 22.4°±0.3°, 23.1°±0.3°, 23.8°±0.3°, 24.4°±0.3°, 24.7°±0.3°, 26.0°±0.3°, 26.3°±0.3°, 26.6°±0.3°, 27.5°±0.3°, 28.8°±0.3°, 29.7°±0.3°, 30.1°±0.3°, 31.5°±0.3°, 32.1°±0.3°, 32.3°±0.3°, 32.7°±0.3°, 33.0°±0.3°, 33.4°±0.3°, 33.9°±0.3°, and 33.4°±0.3° 2θ.

In certain embodiments, Form B of the compound of formula (I) has an XRPD pattern comprising one or more peaks at 4.0°±0.2°, 11.1°±0.2°, and 12.9°±0.2° 2θ.

In certain embodiments, the XRPD pattern further comprises one or more peaks at 7.9°±0.2°, 9.1°±0.2°, 10.4°±0.2°, and 11.9°±0.2° 2θ.

In certain embodiments, the XRPD pattern further comprises one or more peaks at 15.9°±0.2°, 18.8°±0.2°, 22.4°±0.2°, 23.1°±0.2°, and 23.8°±0.2° 2θ.

In certain embodiments, the XRPD pattern further comprises one or more peaks at 18.2°±0.2°, 18.5°±0.2°, 19.4°±0.2°, 19.9°±0.2°, 20.2°±0.2°, 21.4°±0.2°, 24.4°±0.2°, and 24.7°±0.2° 2θ.

In certain embodiments, the XRPD pattern further comprises one or more peaks at 28.8°±0.2° and 31.5°±0.2° 2θ.

In certain embodiments, the XRPD pattern further comprises one or more peaks at 26.0°±0.2°, 26.3°±0.2°, 26.6°±0.2°, 27.5°±0.2°, 29.7°±0.2°, 30.1°±0.2°, 32.1°±0.2°, 32.3°±0.2°, 32.7°±0.2°, 33.0°±0.2°, 33.4°±0.2°, 33.9°±0.2°, and 33.4°±0.2° 2θ.

In certain embodiments, Form B of the compound of formula (I) has an XRPD pattern comprising one or more peaks at 4.0°±0.2°, 7.9°±0.2°, 9.1°±0.2°, 10.4°±0.2°, 11.1°±0.2°, 11.9°±0.2°, 12.9°±0.2°, 15.9°±0.2°, 18.2°±0.2°, 18.5°±0.2°, 18.8°±0.2°, 19.4°±0.2°, 19.9°±0.2°, 20.2°±0.2°, 21.4°±0.2°, 22.4°±0.2°, 23.1°±0.2°, 23.8°±0.2°, 24.4°±0.2°, 24.7°±0.2°, 26.0°±0.2°, 26.3°±0.2°, 26.6°±0.2°, 27.5°±0.2°, 28.8°±0.2°, 29.7°±0.2°, 30.1°±0.2°, 31.5°±0.2°, 32.1°±0.2°, 32.3°±0.2°, 32.7°±0.2°, 33.0°±0.2°, 33.4°±0.2°, 33.9°±0.2°, and 33.4°±0.2° 2θ.

In certain embodiments, Form B of the compound of formula (I) has an XRPD pattern comprising peaks at 4.0°±0.2°, 7.9°±0.2°, 9.1°±0.2°, 10.4°±0.2°, 11.1°±0.2°, 11.9°±0.2°, 12.9°±0.2°, 15.9°±0.2°, 18.2°±0.2°, 18.5°±0.2°, 18.8°±0.2°, 19.4°±0.2°, 19.9°±0.2°, 20.2°±0.2°, 21.4°±0.2°, 22.4°±0.2°, 23.1°±0.2°, 23.8°±0.2°, 24.4°±0.2°, 24.7°±0.2°, 26.0°±0.2°, 26.3°±0.2°, 26.6°±0.2°, 27.5°±0.2°, 28.8°±0.2°, 29.7°±0.2°, 30.1°±0.2°, 31.5°±0.2°, 32.1°±0.2°, 32.3°±0.2°, 32.7°±0.2°, 33.0°±0.2°, 33.4°±0.2°, 33.9°±0.2°, and 33.4°±0.2° 2θ.

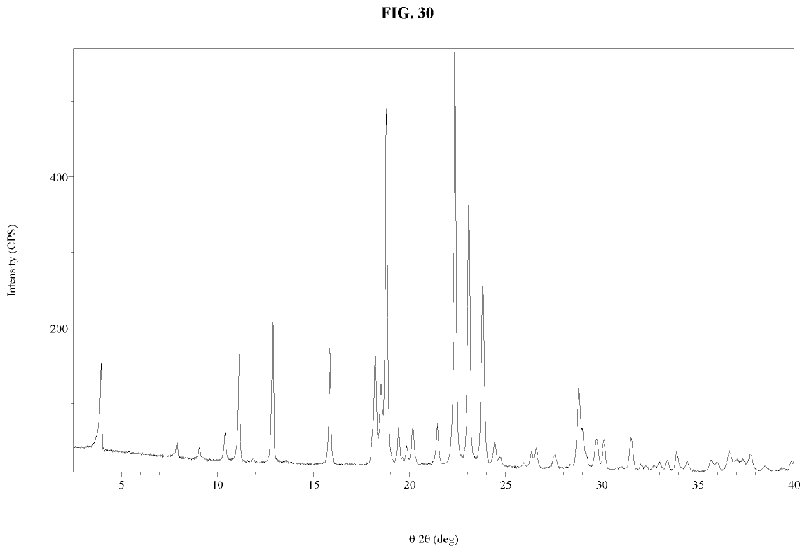

In certain embodiments, Form B of the compound of formula (I) has an XRPD pattern substantially the same as shown in FIG. 30. In certain embodiments, Form B of the compound of formula (I) has an XRPD pattern comprising one or more diffraction peaks (2θ) disclosed in Table 32.

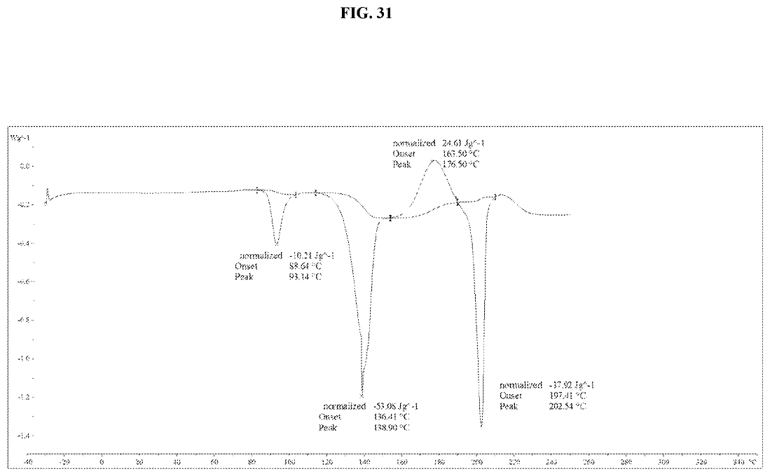

In certain embodiments, Form B of the compound of formula (I) has a DSC thermogram comprising an endotherm with a peak onset at about 89° C. In certain embodiments, Form B of the compound of formula (I) has a DSC thermogram comprising an endotherm with a peak onset at about 136° C. In certain embodiments, Form B of the compound of formula (I) has a DSC thermogram comprising an endotherm with a peak onset at about 197° C. In certain embodiments, Form B of the compound of formula (I) has a DSC thermogram comprising one or more endotherms with peak onsets at about 89° C., about 136° C. and about 197° C. In certain embodiments, Form B of the compound of formula (I) has a DSC thermogram comprising an exotherm with a peak onset at about 164° C. In certain embodiments, Form B of the compound of formula (I) has a DSC thermogram substantially the same as shown in FIG. 31.

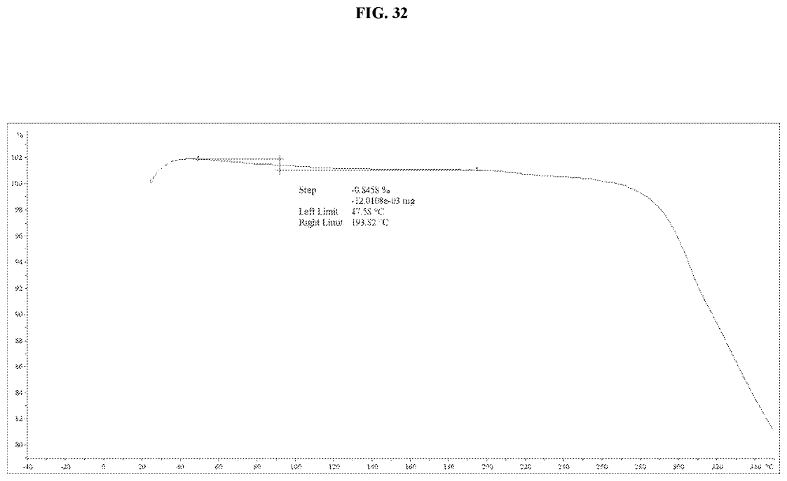

In certain embodiments, Form B of the compound of formula (I) exhibits a weight loss of less than or equal to about 0.8% wt. upon heating Form B from about 25° C. to about 194° C. The weight loss exhibited by a crystalline form (e.g., Form B of the compound of formula (I)) can be determined, for example, using thermogravimetric analysis (TGA). In certain embodiments, Form B of the compound of formula (I) has a TGA thermogram substantially the same as shown in FIG. 32.

In certain embodiments, Form B of the compound of formula (I) is an anhydrous crystalline form.

### (3) Form C

In various embodiments, provided herein is Form C of a compound of formula (I)

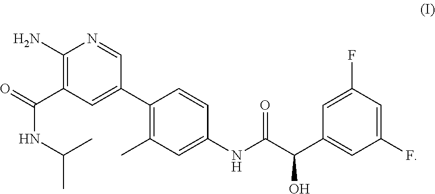 [SMILES: Cc1cc(NC(=O)[C@H](O)c2cc(F)cc(F)c2)ccc1-c1cnc(N)c(C(=O)NC(C)C)c1]

In certain embodiments, Form C of the compound of formula (I) has an XRPD pattern comprising one or more peaks at about 6.7° and about 12.0° 2θ.

In certain embodiments, the XRPD pattern further comprises one or more peaks at about 3.4°, about 9.3°, about 9.6°, about 10.2°, about 11.0°, about 13.1°, about 13.5°, and about 14.4° 2θ.

In certain embodiments, the XRPD pattern further comprises one or more peaks at about 18.9°, about 19.3°, about 22.6°, about 22.8°, and about 23.6° 2θ.

In certain embodiments, the XRPD pattern further comprises one or more peaks at about 15.7°, about 16.9°, about 17.1°, about 18.4°, about 18.7°, about 19.1°, about 20.1°, about 20.4°, about 21.6°, about 22.1°, about 23.2°, about 24.2°, about 24.7°, and about 25.0° 2θ.

In certain embodiments, the XRPD pattern further comprises one or more peaks at about 25.9° and about 30.1° 2θ.

In certain embodiments, the XRPD pattern further comprises one or more peaks at about 26.2°, about 26.6°, about 28.1°, about 28.5°, about 29.1°, about 29.4°, about 29.6°, and about 30.7° 2θ.

In certain embodiments, Form C of the compound of formula (I) has an XRPD pattern comprising one or more peaks at about 3.4°, about 6.7°, about 9.3°, about 9.6°, about 10.2°, about 11.0°, about 12.0°, about 13.1°, about 13.5°, about 14.4°, about 15.7°, about 16.9°, about 17.1°, about 18.4°, about 18.7°, about 18.9°, about 19.1°, about 19.3°, about 20.1°, about 20.4°, about 21.6°, about 22.1°, about 22.6°, about 22.8°, about 23.2°, about 23.6°, about 24.2°, about 24.7°, about 25.0°, about 25.9°, about 26.2°, about 26.6°, about 28.1°, about 28.5°, about 29.1°, about 29.4°, about 29.6°, about 30.1°, and about 30.7° 2θ.

In certain embodiments, Form C of the compound of formula (I) has an XRPD pattern comprising peaks at about 3.4°, about 6.7°, about 9.3°, about 9.6°, about 10.2°, about 11.0°, about 12.0°, about 13.1°, about 13.5°, about 14.4°, about 15.7°, about 16.9°, about 17.1°, about 18.4°, about 18.7°, about 18.9°, about 19.1°, about 19.3°, about 20.1°, about 20.4°, about 21.6°, about 22.1°, about 22.6°, about 22.8°, about 23.2°, about 23.6°, about 24.2°, about 24.7°, about 25.0°, about 25.9°, about 26.2°, about 26.6°, about 28.1°, about 28.5°, about 29.1°, about 29.4°, about 29.6°, about 30.1°, and about 30.7° 2θ.

In certain embodiments, Form C of the compound of formula (I) has an XRPD pattern comprising one or more peaks at 6.7°±0.3° and 12.0°±0.3° 2θ.

In certain embodiments, the XRPD pattern further comprises one or more peaks at 3.4°±0.3°, 9.3°±0.3°, 9.6°±0.3°, 10.2°±0.3°, 11.0°±0.3°, 13.1°±0.3°, 13.5°±0.3°, and 14.4°±0.3° 2θ.

In certain embodiments, the XRPD pattern further comprises one or more peaks at 18.9°±0.3°, 19.3°±0.3°, 22.6°±0.3°, 22.8°±0.3°, and 23.6°±0.3° 2θ.

In certain embodiments, the XRPD pattern further comprises one or more peaks at 15.7°±0.3°, 16.9°±0.3°, 17.1°±0.3°, 18.4°±0.3°, 18.7°±0.3°, 19.1°±0.3°, 20.1°±0.3°, 20.4°±0.3°, 21.6°±0.3°, 22.1°±0.3°, 23.2°±0.3°, 24.2°±0.3°, 24.7°±0.3°, and 25.0°±0.3° 2θ.

In certain embodiments, the XRPD pattern further comprises one or more peaks at 25.9°±0.3° and 30.1°±0.3° 2θ.

In certain embodiments, the XRPD pattern further comprises one or more peaks at 26.2°±0.3°, 26.6°±0.3°, 28.1°±0.3°, 28.5°±0.3°, 29.1°±0.3°, 29.4°±0.3°, 29.6°±0.3°, and 30.7°±0.3° 2θ.

In certain embodiments, Form C of the compound of formula (I) has an XRPD pattern comprising one or more peaks at 3.4°±0.3°, 6.7°±0.3°, 9.3°±0.3°, 9.6°±0.3°, 10.2°±0.3°, 11.0°±0.3°, 12.0°±0.3°, 13.1°±0.3°, 13.5°±0.3°, 14.4°±0.3°, 15.7°±0.3°, 16.9°±0.3°, 17.1°±0.3°, 18.4°±0.3°, 18.7°±0.3°, 18.9°±0.3°, 19.1°±0.3°, 19.3°±0.3°, 20.1°±0.3°, 20.4°±0.3°, 21.6°±0.3°, 22.1°±0.3°, 22.6°±0.3°, 22.8°±0.3°, 23.2°±0.3°, 23.6°±0.3°, 24.2°±0.3°, 24.7°±0.3°, 25.0°±0.3°, 25.9°±0.3°, 26.2°±0.3°, 26.6°±0.3°, 28.1°±0.3°, 28.5°±0.3°, 29.1°±0.3°, 29.4°±0.3°, 29.6°±0.3°, 30.1°±0.3°, and 30.7°±0.3° 2θ.

In certain embodiments, Form C of the compound of formula (I) has an XRPD pattern comprising peaks at 3.4°±0.3°, 6.7°±0.3°, 9.3°±0.3°, 9.6°±0.3°, 10.2°±0.3°, 11.0°±0.3°, 12.0°±0.3°, 13.1°±0.3°, 13.5°±0.3°, 14.4°±0.3°, 15.7°±0.3°, 16.9°±0.3°, 17.1°±0.3°, 18.4°±0.3°, 18.7°±0.3°, 18.9°±0.3°, 19.1°±0.3°, 19.3°±0.3°, 20.1°±0.3°, 20.4°±0.3°, 21.6°±0.3°, 22.1°±0.3°, 22.6°±0.3°, 22.8°±0.3°, 23.2°±0.3°, 23.6°±0.3°, 24.2°±0.3°, 24.7°±0.3°, 25.0°±0.3°, 25.9°±0.3°, 26.2°±0.3°, 26.6°±0.3°, 28.1°±0.3°, 28.5°±0.3°, 29.1°±0.3°, 29.4°±0.3°, 29.6°±0.3°, 30.1°±0.3°, and 30.7°±0.3° 2θ.

In certain embodiments, Form C of the compound of formula (I) has an XRPD pattern comprising one or more peaks at 6.7°±0.2° and 12.0°±0.2° 2θ.

In certain embodiments, the XRPD pattern further comprises one or more peaks at 3.4°±0.2°, 9.3°±0.2°, 9.6°±0.2°, 10.2°±0.2°, 11.0°±0.2°, 13.1°±0.2°, 13.5°±0.2°, and 14.4°±0.2° 2θ.

In certain embodiments, the XRPD pattern further comprises one or more peaks at 18.9°±0.2°, 19.3°±0.2°, 22.6°±0.2°, 22.8°±0.2°, and 23.6°±0.2° 2θ.

In certain embodiments, the XRPD pattern further comprises one or more peaks at 15.7°±0.2°, 16.9°±0.2°, 17.1°±0.2°, 18.4°±0.2°, 18.7°±0.2°, 19.1°±0.2°, 20.1°±0.2°, 20.4°±0.2°, 21.6°±0.2°, 22.1°±0.2°, 23.2°±0.2°, 24.2°±0.2°, 24.7°±0.2°, and 25.0°±0.2° 2θ.

In certain embodiments, the XRPD pattern further comprises one or more peaks at 25.9°±0.2° and 30.1°±0.2° 2θ.

In certain embodiments, the XRPD pattern further comprises one or more peaks at 26.2°±0.2°, 26.6°±0.2°, 28.1°±0.2°, 28.5°±0.2°, 29.1°±0.2°, 29.4°±0.2°, 29.6°±0.2°, and 30.7°±0.2° 2θ.

In certain embodiments, Form C of the compound of formula (I) has an XRPD pattern comprising one or more peaks at 3.4°±0.2°, 6.7°±0.2°, 9.3°±0.2°, 9.6°±0.2°, 10.2°±0.2°, 11.0°±0.2°, 12.0°±0.2°, 13.1°±0.2°, 13.5°±0.2°, 14.4°±0.2°, 15.7°±0.2°, 16.9°±0.2°, 17.1°±0.2°, 18.4°±0.2°, 18.7°±0.2°, 18.9°±0.2°, 19.1°±0.2°, 19.3°±0.2°, 20.1°±0.2°, 20.4°±0.2°, 21.6°±0.2°, 22.1°±0.2°, 22.6°±0.2°, 22.8°±0.2°, 23.2°±0.2°, 23.6°±0.2°, 24.2°±0.2°, 24.7°±0.2°, 25.0°±0.2°, 25.9°±0.2°, 26.2°±0.2°, 26.6°±0.2°, 28.1°±0.2°, 28.5°±0.2°, 29.1°±0.2°, 29.4°±0.2°, 29.6°±0.2°, 30.1°±0.2°, and 30.7°±0.2° 2θ.

In certain embodiments, Form C of the compound of formula (I) has an XRPD pattern comprising peaks at 3.4°±0.2°, 6.7°±0.2°, 9.3°±0.2°, 9.6°±0.2°, 10.2°±0.2°, 11.0°±0.2°, 12.0°±0.2°, 13.1°±0.2°, 13.5°±0.2°, 14.4°±0.2°, 15.7°±0.2°, 16.9°±0.2°, 17.1°±0.2°, 18.4°±0.2°, 18.7°±0.2°, 18.9°±0.2°, 19.1°±0.2°, 19.3°±0.2°, 20.1°±0.2°, 20.4°±0.2°, 21.6°±0.2°, 22.1°±0.2°, 22.6°±0.2°, 22.8°±0.2°, 23.2°±0.2°, 23.6°±0.2°, 24.2°±0.2°, 24.7°±0.2°, 25.0°±0.2°, 25.9°±0.2°, 26.2°±0.2°, 26.6°±0.2°, 28.1°±0.2°, 28.5°±0.2°, 29.1°±0.2°, 29.4°±0.2°, 29.6°±0.2°, 30.1°±0.2°, and 30.7°±0.2° 2θ.

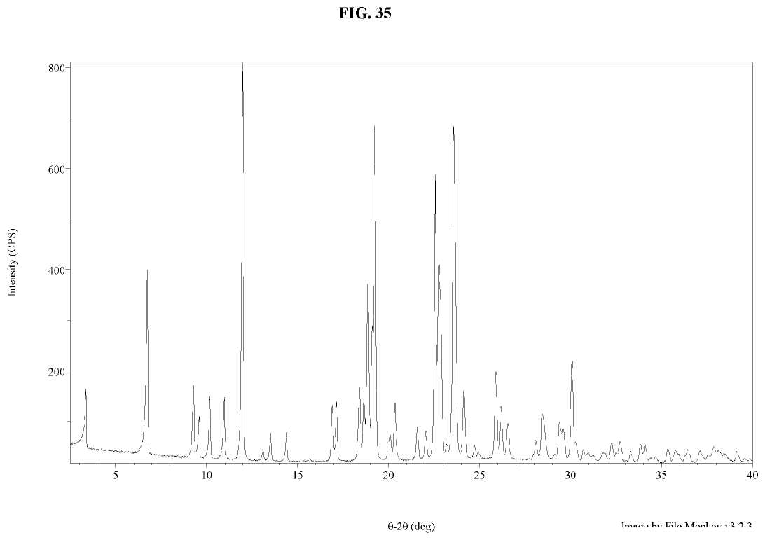

In certain embodiments, Form C of the compound of formula (I) has an XRPD pattern substantially the same as shown in FIG. 35. In certain embodiments, Form C of the compound of formula (I) has an XRPD pattern comprising one or more diffraction peaks (2θ) disclosed in Table 34.

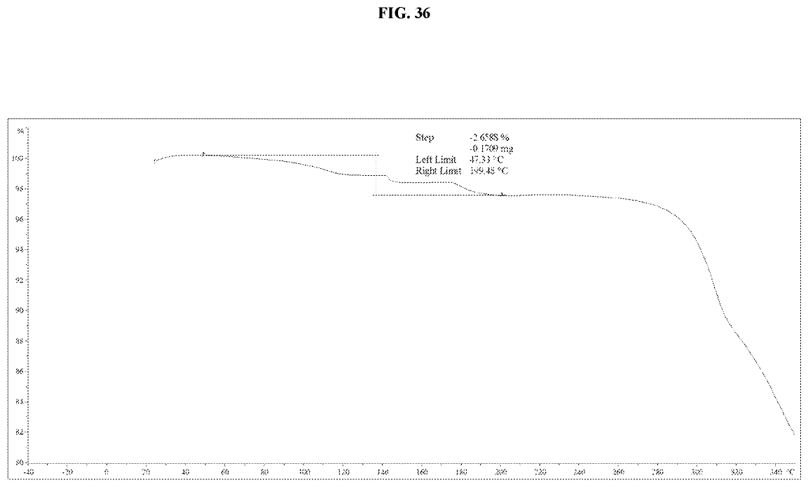

In certain embodiments, Form C of the compound of formula (I) has a DSC thermogram comprising an endotherm with a peak onset at about 91° C. In certain embodiments, Form C of the compound of formula (I) has a DSC thermogram comprising an endotherm with a peak onset at about 132° C. In certain embodiments, Form C of the compound of formula (I) has a DSC thermogram comprising an endotherm with a peak onset at about 203° C. In certain embodiments, Form C of the compound of formula (I) has a DSC thermogram comprising one or more endotherms with peak onsets at about 91° C., about 132° C., and about 203° C. In certain embodiments, Form C of the compound of formula (I) has a DSC thermogram comprising an endotherm with a peak maximum at about 65° C. In certain embodiments, Form C of the compound of formula (I) has a DSC thermogram comprising an exotherm with a peak onset at about 157° C. In certain embodiments, Form C of the compound of formula (I) has a DSC thermogram substantially the same as shown in FIG. 36.

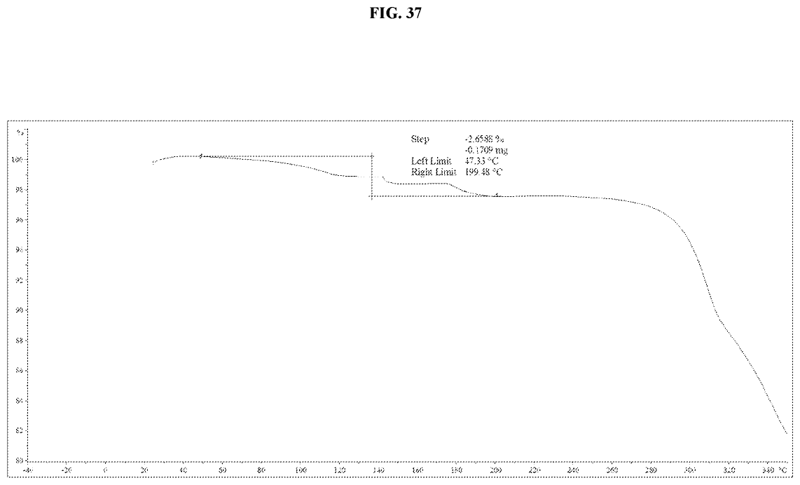

In certain embodiments, Form C of the compound of formula (I) exhibits a weight loss of less than or equal to about 2.7% wt. upon heating Form C from about 25° C. to about 199° C. The weight loss exhibited by a crystalline form (e.g., Form C of the compound of formula (I)) can be determined, for example, using TGA. In certain embodiments, Form C of the compound of formula (I) has a TGA thermogram substantially the same as shown in FIG. 37.

In certain embodiments, Form C of the compound of formula (I) is a crystalline solvate. In certain embodiments, Form C of the compound of formula (I) is a crystalline acetonitrile solvate. In certain embodiments, Form C of the compound of formula (I) is a crystalline ethyl acetate solvate.

### (4) Form D

In various embodiments, provided herein is Form D of a compound of formula (I)

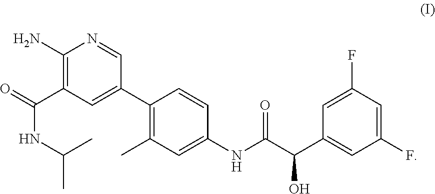 [SMILES: Cc1cc(NC(=O)[C@H](O)c2cc(F)cc(F)c2)ccc1-c1cnc(N)c(C(=O)NC(C)C)c1]

In certain embodiments, Form D of the compound of formula (I) has an XRPD pattern comprising one or more peaks at about 9.2°, about 10.3°, and about 10.6° 2θ.

In certain embodiments, the XRPD pattern further comprises one or more peaks at about 7.4°, about 7.6°, about 11.0°, about 12.0°, about 12.2°, about 12.8°, about 13.0°, and about 14.8° 2θ.

In certain embodiments, the XRPD pattern further comprises one or more peaks at about 16.4°, about 17.3°, about 21.3°, and about 22.6° 2θ.

In certain embodiments, the XRPD pattern further comprises one or more peaks at about 15.1°, about 16.0°, about 16.9°, about 17.6°, about 18.1°, about 18.5°, about 19.0°, about 19.5°, about 19.7°, about 20.3°, about 21.8°, about 22.0°, about 22.2°, about 22.9°, about 23.3°, about 23.6°, about 24.0°, about 24.4°, about 24.6°, and about 24.9° 2θ.

In certain embodiments, the XRPD pattern further comprises one or more peaks at about 26.6°, about 27.8°, about 28.7°, and about 32.2° 2θ.

In certain embodiments, the XRPD pattern further comprises one or more peaks at about 25.1°, about 25.8°, about 26.2°, about 26.7°, about 27.0°, about 27.6°, about 28.2°, about 29.0°, about 29.2°, about 30.2°, about 30.9°, about 31.5°, and about 32.6° 2θ.

In certain embodiments, Form D of the compound of formula (I) has an XRPD pattern comprising one or more peaks at about 7.4°, about 7.6°, about 9.2°, about 10.3°, about 10.6°, about 11.0°, about 12.0°, about 12.2°, about 12.8°, about 13.0°, about 14.8°, about 15.1°, about 16.0°, about 16.4°, about 16.9°, about 17.3°, about 17.6°, about 18.1°, about 18.5°, about 19.0°, about 19.5°, about 19.7°, about 20.3°, about 21.3°, about 21.8°, about 22.0°, about 22.2°, about 22.6°, about 22.9°, about 23.3°, about 23.6°, about 24.0°, about 24.4°, about 24.6°, about 24.9°, about 25.1°, about 25.8°, about 26.2°, about 26.6°, about 26.7°, about 27.0°, about 27.6°, about 27.8°, about 28.2°, about 28.7°, about 29.0°, about 29.2°, about 30.2°, about 30.9°, about 31.5°, about 32.2°, and about 32.6° 2θ.

In certain embodiments, Form D of the compound of formula (I) has an XRPD pattern comprising peaks at about 7.4°, about 7.6°, about 9.2°, about 10.3°, about 10.6°, about 11.0°, about 12.0°, about 12.2°, about 12.8°, about 13.0°, about 14.8°, about 15.1°, about 16.0°, about 16.4°, about 16.9°, about 17.3°, about 17.6°, about 18.1°, about 18.5°, about 19.0°, about 19.5°, about 19.7°, about 20.3°, about 21.3°, about 21.8°, about 22.0°, about 22.2°, about 22.6°, about 22.9°, about 23.3°, about 23.6°, about 24.0°, about 24.4°, about 24.6°, about 24.9°, about 25.1°, about 25.8°, about 26.2°, about 26.6°, about 26.7°, about 27.0°, about 27.6°, about 27.8°, about 28.2°, about 28.7°, about 29.0°, about 29.2°, about 30.2°, about 30.9°, about 31.5°, about 32.2°, and about 32.6° 2θ.

In certain embodiments, Form D of the compound of formula (I) has an XRPD pattern comprising one or more peaks at 9.2°±0.3°, 10.3°±0.3°, and 10.6°±0.3° 2θ.

In certain embodiments, the XRPD pattern further comprises one or more peaks at 7.4°±0.3°, 7.6°±0.3°, 11.0°±0.3°, 12.0°±0.3°, 12.2°±0.3°, 12.8°±0.3°, 13.0°±0.3°, and 14.8°±0.3° 2θ.

In certain embodiments, the XRPD pattern further comprises one or more peaks at 16.4°±0.3°, 17.3°±0.3°, 21.3°±0.3°, and 22.6°±0.3° 2θ.

In certain embodiments, the XRPD pattern further comprises one or more peaks at 15.1°±0.3°, 16.0°±0.3°, 16.9°±0.3°, 17.6°±0.3°, 18.1°±0.3°, 18.5°±0.3°, 19.0°±0.3°, 19.5°±0.3°, 19.7°±0.3°, 20.3°±0.3°, 21.8°±0.3°, 22.0°±0.3°, 22.2°±0.3°, 22.9°±0.3°, 23.3°±0.3°, 23.6°±0.3°, 24.0°±0.3°, 24.4°±0.3°, 24.6°±0.3°, and 24.9°±0.3° 2θ.

In certain embodiments, the XRPD pattern further comprises one or more peaks at 26.6°±0.3°, 27.8°±0.3°, 28.7°±0.3°, and 32.2°±0.3° 2θ.

In certain embodiments, the XRPD pattern further comprises one or more peaks at 25.1°±0.3°, 25.8°±0.3°, 26.2°±0.3°, 26.7°±0.3°, 27.0°±0.3°, 27.6°±0.3°, 28.2°±0.3°, 29.0°±0.3°, 29.2°±0.3°, 30.2°±0.3°, 30.9°±0.3°, 31.5°±0.3°, and 32.6°±0.3° 2θ.

In certain embodiments, Form D of the compound of formula (I) has an XRPD pattern comprising one or more peaks at 7.4°±0.3°, 7.6°±0.3°, 9.2°±0.3°, 10.3°±0.3°, 10.6°±0.3°, 11.0°±0.3°, 12.0°±0.3°, 12.2°±0.3°, 12.8°±0.3°, 13.0°±0.3°, 14.8°±0.3°, 15.1°±0.3°, 16.0°±0.3°, 16.4°±0.3°, 16.9°±0.3°, 17.3°±0.3°, 17.6°±0.3°, 18.1°±0.3°, 18.5°±0.3°, 19.0°±0.3°, 19.5°±0.3°, 19.7°±0.3°, 20.3°±0.3°, 21.3°±0.3°, 21.8°±0.3°, 22.0°±0.3°, 22.2°±0.3°, 22.6°±0.3°, 22.9°±0.3°, 23.3°±0.3°, 23.6°±0.3°, 24.0°±0.3°, 24.4°±0.3°, 24.6°±0.3°, 24.9°±0.3°, 25.1°±0.3°, 25.8°±0.3°, 26.2°±0.3°, 26.6°±0.3°, 26.7°±0.3°, 27.0°±0.3°, 27.6°±0.3°, 27.8°±0.3°, 28.2°±0.3°, 28.7°±0.3°, 29.0°±0.3°, 29.2°±0.3°, 30.2°±0.3°, 30.9°±0.3°, 31.5°±0.3°, 32.2°±0.3°, and 32.6°±0.3° 2θ.

In certain embodiments, Form D of the compound of formula (I) has an XRPD pattern comprising peaks at 7.4°±0.3°, 7.6°±0.3°, 9.2°±0.3°, 10.3°±0.3°, 10.6°±0.3°, 11.0°±0.3°, 12.0°±0.3°, 12.2°±0.3°, 12.8°±0.3°, 13.0°±0.3°, 14.8°±0.3°, 15.1°±0.3°, 16.0°±0.3°, 16.4°±0.3°, 16.9°±0.3°, 17.3°±0.3°, 17.6°±0.3°, 18.1°±0.3°, 18.5°±0.3°, 19.0°±0.3°, 19.5°±0.3°, 19.7°±0.3°, 20.3°±0.3°, 21.3°±0.3°, 21.8°±0.3°, 22.0°±0.3°, 22.2°±0.3°, 22.6°±0.3°, 22.9°±0.3°, 23.3°±0.3°, 23.6°±0.3°, 24.0°±0.3°, 24.4°±0.3°, 24.6°±0.3°, 24.9°±0.3°, 25.1°±0.3°, 25.8°±0.3°, 26.2°±0.3°, 26.6°±0.3°, 26.7°±0.3°, 27.0°±0.3°, 27.6°±0.3°, 27.8°±0.3°, 28.2°±0.3°, 28.7°±0.3°, 29.0°±0.3°, 29.2°±0.3°, 30.2°±0.3°, 30.9°±0.3°, 31.5°±0.3°, 32.2°±0.3°, and 32.6°±0.3° 2θ.

In certain embodiments, Form D of the compound of formula (I) has an XRPD pattern comprising one or more peaks at 9.2°±0.2°, 10.3°±0.2°, and 10.6°±0.2° 2θ.

In certain embodiments, the XRPD pattern further comprises one or more peaks at 7.4°±0.2°, 7.6°±0.2°, 11.0°±0.2°, 12.0°±0.2°, 12.2°±0.2°, 12.8°±0.2°, 13.0°±0.2°, and 14.8°±0.2° 2θ.

In certain embodiments, the XRPD pattern further comprises one or more peaks at 16.4°±0.2°, 17.3°±0.2°, 21.3°±0.2°, and 22.6°±0.2° 2θ.

In certain embodiments, the XRPD pattern further comprises one or more peaks at 15.1°±0.2°, 16.0°±0.2°, 16.9°±0.2°, 17.6°±0.2°, 18.1°±0.2°, 18.5°±0.2°, 19.0°±0.2°, 19.5°±0.2°, 19.7°±0.2°, 20.3°±0.2°, 21.8°±0.2°, 22.0°±0.2°, 22.2°±0.2°, 22.9°±0.2°, 23.3°±0.2°, 23.6°±0.2°, 24.0°±0.2°, 24.4°±0.2°, 24.6°±0.2°, and 24.9°±0.2° 2θ.

In certain embodiments, the XRPD pattern further comprises one or more peaks at 26.6°±0.2°, 27.8°±0.2°, 28.7°±0.2°, and 32.2°±0.2° 2θ.

In certain embodiments, the XRPD pattern further comprises one or more peaks at 25.1°±0.2°, 25.8°±0.2°, 26.2°±0.2°, 26.7°±0.2°, 27.0°±0.2°, 27.6°±0.2°, 28.2°±0.2°, 29.0°±0.2°, 29.2°±0.2°, 30.2°±0.2°, 30.9°±0.2°, 31.5°±0.2°, and 32.6°±0.2° 2θ.

In certain embodiments, Form D of the compound of formula (I) has an XRPD pattern comprising one or more peaks at 7.4°±0.2°, 7.6°±0.2°, 9.2°±0.2°, 10.3°±0.2°, 10.6°±0.2°, 11.0°±0.2°, 12.0°±0.2°, 12.2°±0.2°, 12.8°±0.2°, 13.0°±0.2°, 14.8°±0.2°, 15.1°±0.2°, 16.0°±0.2°, 16.4°±0.2°, 16.9°±0.2°, 17.3°±0.2°, 17.6°±0.2°, 18.1°±0.2°, 18.5°±0.2°, 19.0°±0.2°, 19.5°±0.2°, 19.7°±0.2°, 20.3°±0.2°, 21.3°±0.2°, 21.8°±0.2°, 22.0°±0.2°, 22.2°±0.2°, 22.6°±0.2°, 22.9°±0.2°, 23.3°±0.2°, 23.6°±0.2°, 24.0°±0.2°, 24.4°±0.2°, 24.6°±0.2°, 24.9°±0.2°, 25.1°±0.2°, 25.8°±0.2°, 26.2°±0.2°, 26.6°±0.2°, 26.7°±0.2°, 27.0°±0.2°, 27.6°±0.2°, 27.8°±0.2°, 28.2°±0.2°, 28.7°±0.2°, 29.0°±0.2°, 29.2°±0.2°, 30.2°±0.2°, 30.9°±0.2°, 31.5°±0.2°, 32.2°±0.2°, and 32.6°±0.2° 2θ.

In certain embodiments, Form D of the compound of formula (I) has an XRPD pattern comprising peaks at 7.4°±0.2°, 7.6°±0.2°, 9.2°±0.2°, 10.3°±0.2°, 10.6°±0.2°, 11.0°±0.2°, 12.0°±0.2°, 12.2°±0.2°, 12.8°±0.2°, 13.0°±0.2°, 14.8°±0.2°, 15.1°±0.2°, 16.0°±0.2°, 16.4°±0.2°, 16.9°±0.2°, 17.3°±0.2°, 17.6°±0.2°, 18.1°±0.2°, 18.5°±0.2°, 19.0°±0.2°, 19.5°±0.2°, 19.7°±0.2°, 20.3°±0.2°, 21.3°±0.2°, 21.8°±0.2°, 22.0°±0.2°, 22.2°±0.2°, 22.6°±0.2°, 22.9°±0.2°, 23.3°±0.2°, 23.6°±0.2°, 24.0°±0.2°, 24.4°±0.2°, 24.6°±0.2°, 24.9°±0.2°, 25.1°±0.2°, 25.8°±0.2°, 26.2°±0.2°, 26.6°±0.2°, 26.7°±0.2°, 27.0°±0.2°, 27.6°±0.2°, 27.8°±0.2°, 28.2°±0.2°, 28.7°±0.2°, 29.0°±0.2°, 29.2°±0.2°, 30.2°±0.2°, 30.9°±0.2°, 31.5°±0.2°, 32.2°±0.2°, and 32.6°±0.2° 2θ.

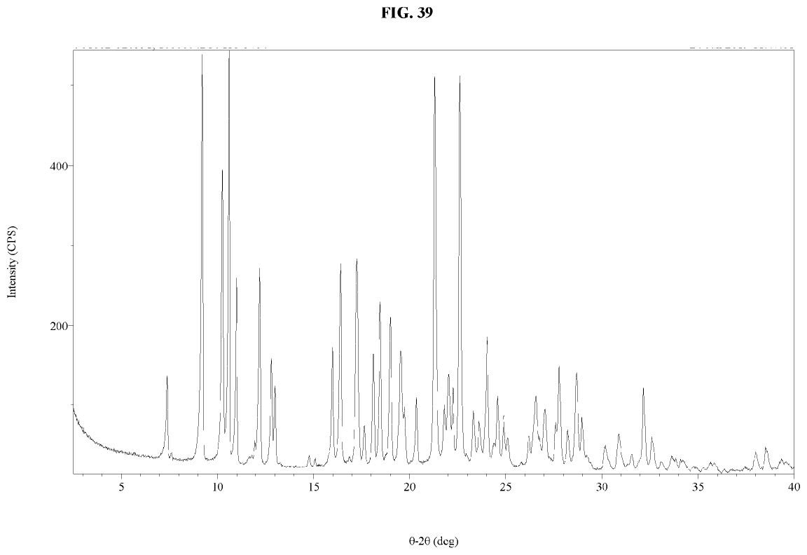

In certain embodiments, Form D of the compound of formula (I) has an XRPD pattern substantially the same as shown in FIG. 39. In certain embodiments, Form D of the compound of formula (I) has an XRPD pattern comprising one or more diffraction peaks (2θ) disclosed in Table 36.

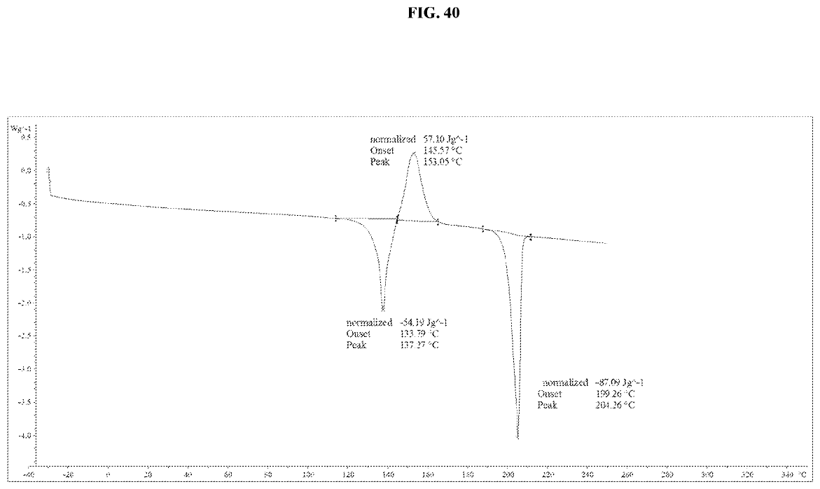

In certain embodiments, Form D of the compound of formula (I) has a DSC thermogram comprising an endotherm with a peak maximum at about 66° C. In certain embodiments, Form D of the compound of formula (I) has a DSC thermogram comprising an endotherm with a peak maximum at about 138° C. In certain embodiments, Form D of the compound of formula (I) has a DSC thermogram comprising one or more endotherms with peak maximums at about 66° C. and about 138° C. In certain embodiments, Form D of the compound of formula (I) has a DSC thermogram comprising an exotherm with a peak maximum at about 157° C. In certain embodiments, Form D of the compound of formula (I) has a DSC thermogram substantially the same as shown in FIG. 40.

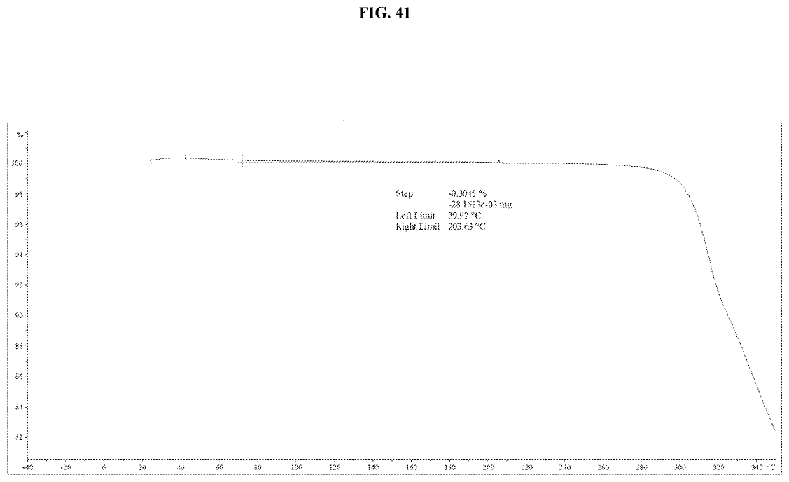

In certain embodiments, Form D of the compound of formula (I) exhibits a weight loss of less than or equal to about 1.0% wt. upon heating Form D from about 25° C. to about 108° C. The weight loss exhibited by a crystalline form (e.g., Form D of the compound of formula (I)) can be determined, for example, using TGA. In certain embodiments, Form D of the compound of formula (I) has a TGA thermogram substantially the same as shown in FIG. 41.

In certain embodiments, Form D of the compound of formula (I) is an anhydrous crystalline form.

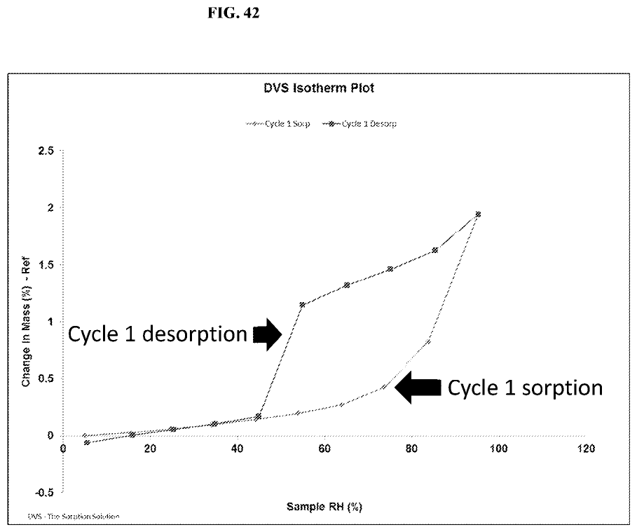

In certain embodiments, Form D of the compound of formula (I) exhibits a change in mass of less than or equal to about 1.9% wt when varying the relative humidity between about 5% to about 95%, when measured at ambient temperature. The weight change exhibited by a crystalline form (e.g., Form D of the compound of formula (I)) can be determined, for example, using dynamic vapor sorption/desorption (DVS). In certain embodiments, Form D of the compound of formula (I) has a water sorption isotherm substantially the same as shown in FIG. 42.

### (5) Form E

In various embodiments, provided herein is Form E of a compound of formula (I)

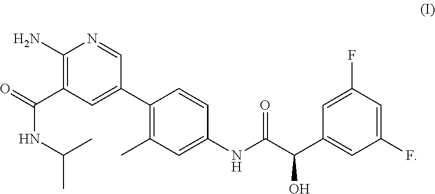 [SMILES: Cc1cc(NC(=O)[C@H](O)c2cc(F)cc(F)c2)ccc1-c1cnc(N)c(C(=O)NC(C)C)c1]

In certain embodiments, Form E of the compound of formula (I) has an XRPD pattern comprising one or more peaks at about 6.7°, about 8.5°, about 8.8°, and about 10.2° 2θ.

In certain embodiments, the XRPD pattern further comprises one or more peaks at about 7.2°, about 11.1°, about 11.4°, about 12.2°, about 13.5°, about 14.2°, and about 14.5° 2θ.

In certain embodiments, the XRPD pattern further comprises one or more peaks at about 16.1°, about 18.5°, about 19.0°, about 21.1°, about 22.8°, and about 23.4° 2θ.

In certain embodiments, the XRPD pattern further comprises one or more peaks at about 15.1°, about 16.0°, about 16.8°, about 17.0°, about 17.6°, about 17.7°, about 19.2°, about 19.7°, about 20.5°, about 21.0°, about 21.7°, about 22.2°, about 24.1°, and about 24.6° 2θ.

In certain embodiments, the XRPD pattern further comprises one or more peaks at about 25.7°, about 26.1°, and about 27.2° 2θ.

In certain embodiments, the XRPD pattern further comprises one or more peaks at about 25.1°, about 26.6°, about 27.7°, about 28.2°, about 29.2°, about 29.5°, about 30.1°, about 30.4°, about 30.9°, and about 31.4° 2θ.

In certain embodiments, Form E of the compound of formula (I) has an XRPD pattern comprising one or more peaks at about 6.7°, about 7.2°, about 8.5°, about 8.8°, about 10.2°, about 11.1°, about 11.4°, about 12.2°, about 13.5°, about 14.2°, about 14.5°, about 15.1°, about 16.0°, about 16.1°, about 16.8°, about 17.0°, about 17.6°, about 17.7°, about 18.5°, about 19.0°, about 19.2°, about 19.7°, about 20.5°, about 21.0°, about 21.1°, about 21.7°, about 22.2°, about 22.8°, about 23.4°, about 24.1°, about 24.6°, about 25.1°, about 25.7°, about 26.1°, about 26.6°, about 27.2°, about 27.7°, about 28.2°, about 29.2°, about 29.5°, about 30.1°, about 30.4°, about 30.9°, and about 31.4° 2θ.

In certain embodiments, Form E of the compound of formula (I) has an XRPD pattern comprising peaks at about 6.7°, about 7.2°, about 8.5°, about 8.8°, about 10.2°, about 11.1°, about 11.4°, about 12.2°, about 13.5°, about 14.2°, about 14.5°, about 15.1°, about 16.0°, about 16.1°, about 16.8°, about 17.0°, about 17.6°, about 17.7°, about 18.5°, about 19.0°, about 19.2°, about 19.7°, about 20.5°, about 21.0°, about 21.1°, about 21.7°, about 22.2°, about 22.8°, about 23.4°, about 24.1°, about 24.6°, about 25.1°, about 25.7°, about 26.1°, about 26.6°, about 27.2°, about 27.7°, about 28.2°, about 29.2°, about 29.5°, about 30.1°, about 30.4°, about 30.9°, and about 31.4° 2θ.

In certain embodiments, Form E of the compound of formula (I) has an XRPD pattern comprising one or more peaks at 6.7°±0.3°, 8.5°±0.3°, 8.8°±0.3°, and 10.2°±0.3° 2θ.

In certain embodiments, the XRPD pattern further comprises one or more peaks at 7.2°±0.3°, 11.1°±0.3°, 11.4°±0.3°, 12.2°±0.3°, 13.5°±0.3°, 14.2°±0.3°, and 14.5°±0.3° 2θ.

In certain embodiments, the XRPD pattern further comprises one or more peaks at 16.1°±0.3°, 18.5°±0.3°, 19.0°±0.3°, 21.1°±0.3°, 22.8°±0.3°, and 23.4°±0.3° 2θ.

In certain embodiments, the XRPD pattern further comprises one or more peaks at 15.1°±0.3°, 16.0°±0.3°, 16.8°±0.3°, 17.0°±0.3°, 17.6°±0.3°, 17.7°±0.3°, 19.2°±0.3°, 19.7°±0.3°, 20.5°±0.3°, 21.0°±0.3°, 21.7°±0.3°, 22.2°±0.3°, 24.1°±0.3°, and 24.6°±0.3° 2θ.

In certain embodiments, the XRPD pattern further comprises one or more peaks at 25.7°±0.3°, 26.1°±0.3°, and 27.2°±0.3° 2θ.

In certain embodiments, the XRPD pattern further comprises one or more peaks at 25.1°±0.3°, 26.6°±0.3°, 27.7°±0.3°, 28.2°±0.3°, 29.2°±0.3°, 29.5°±0.3°, 30.1°±0.3°, 30.4°±0.3°, 30.9°±0.3°, and 31.4°±0.3° 2θ.

In certain embodiments, Form E of the compound of formula (I) has an XRPD pattern comprising one or more peaks at 6.7°±0.3°, 7.2°±0.3°, 8.5°±0.3°, 8.8°±0.3°, 10.2°±0.3°, 11.1°±0.3°, 11.4°±0.3°, 12.2°±0.3°, 13.5°±0.3°, 14.2°±0.3°, 14.5°±0.3°, 15.1°±0.3°, 16.0°±0.3°, 16.1°±0.3°, 16.8°±0.3°, 17.0°±0.3°, 17.6°±0.3°, 17.7°±0.3°, 18.5°±0.3°, 19.0°±0.3°, 19.2°±0.3°, 19.7°±0.3°, 20.5°±0.3°, 21.0°±0.3°, 21.1°±0.3°, 21.7°±0.3°, 22.2°±0.3°, 22.8°±0.3°, 23.4°±0.3°, 24.1°±0.3°, 24.6°±0.3°, 25.1°±0.3°, 25.7°±0.3°, 26.1°±0.3°, 26.6°±0.3°, 27.2°±0.3°, 27.7°±0.3°, 28.2°±0.3°, 29.2°±0.3°, 29.5°±0.3°, 30.1°±0.3°, 30.4°±0.3°, 30.9°±0.3°, and 31.4°±0.3° 2θ.

In certain embodiments, Form E of the compound of formula (I) has an XRPD pattern comprising peaks at 6.7°±0.3°, 7.2°±0.3°, 8.5°±0.3°, 8.8°±0.3°, 10.2°±0.3°, 11.1°±0.3°, 11.4°±0.3°, 12.2°±0.3°, 13.5°±0.3°, 14.2°±0.3°, 14.5°±0.3°, 15.1°±0.3°, 16.0°±0.3°, 16.1°±0.3°, 16.8°±0.3°, 17.0°±0.3°, 17.6°±0.3°, 17.7°±0.3°, 18.5°±0.3°, 19.0°±0.3°, 19.2°±0.3°, 19.7°±0.3°, 20.5°±0.3°, 21.0°±0.3°, 21.1°±0.3°, 21.7°±0.3°, 22.2°±0.3°, 22.8°±0.3°, 23.4°±0.3°, 24.1°±0.3°, 24.6°±0.3°, 25.1°±0.3°, 25.7°±0.3°, 26.1°±0.3°, 26.6°±0.3°, 27.2°±0.3°, 27.7°±0.3°, 28.2°±0.3°, 29.2°±0.3°, 29.5°±0.3°, 30.1°±0.3°, 30.4°±0.3°, 30.9°±0.3°, and 31.4°±0.3° 2θ.

In certain embodiments, Form E of the compound of formula (I) has an XRPD pattern comprising one or more peaks at 6.7°±0.2°, 8.5°±0.2°, 8.8°±0.2°, and 10.2°±0.2° 2θ.

In certain embodiments, the XRPD pattern further comprises one or more peaks at 7.2°±0.2°, 11.1°±0.2°, 11.4°±0.2°, 12.2°±0.2°, 13.5°±0.2°, 14.2°±0.2°, and 14.5°±0.2° 2θ.

In certain embodiments, the XRPD pattern further comprises one or more peaks at 16.1°±0.2°, 18.5°±0.2°, 19.0°±0.2°, 21.1°±0.2°, 22.8°±0.2°, and 23.4°±0.2° 2θ.

In certain embodiments, the XRPD pattern further comprises one or more peaks at 15.1°±0.2°, 16.0°±0.2°, 16.8°±0.2°, 17.0°±0.2°, 17.6°±0.2°, 17.7°±0.2°, 19.2°±0.2°, 19.7°±0.2°, 20.5°±0.2°, 21.0°±0.2°, 21.7°±0.2°, 22.2°±0.2°, 24.1°±0.2°, and 24.6°±0.2° 2θ.

In certain embodiments, the XRPD pattern further comprises one or more peaks at 25.7°±0.2°, 26.1°±0.2°, and 27.2°±0.2° 2θ.

In certain embodiments, the XRPD pattern further comprises one or more peaks at 25.1°±0.2°, 26.6°±0.2°, 27.7°±0.2°, 28.2°±0.2°, 29.2°±0.2°, 29.5°±0.2°, 30.1°±0.2°, 30.4°±0.2°, 30.9°±0.2°, and 31.4°±0.2° 2θ.

In certain embodiments, Form E of the compound of formula (I) has an XRPD pattern comprising one or more peaks at 6.7°±0.2°, 7.2°±0.2°, 8.5°±0.2°, 8.8°±0.2°, 10.2°±0.2°, 11.1°±0.2°, 11.4°±0.2°, 12.2°±0.2°, 13.5°±0.2°, 14.2°±0.2°, 14.5°±0.2°, 15.1°±0.2°, 16.0°±0.2°, 16.1°±0.2°, 16.8°±0.2°, 17.0°±0.2°, 17.6°±0.2°, 17.7°±0.2°, 18.5°±0.2°, 19.0°±0.2°, 19.2°±0.2°, 19.7°±0.2°, 20.5°±0.2°, 21.0°±0.2°, 21.1°±0.2°, 21.7°±0.2°, 22.2°±0.2°, 22.8°±0.2°, 23.4°±0.2°, 24.1°±0.2°, 24.6°±0.2°, 25.1°±0.2°, 25.7°±0.2°, 26.1°±0.2°, 26.6°±0.2°, 27.2°±0.2°, 27.7°±0.2°, 28.2°±0.2°, 29.2°±0.2°, 29.5°±0.2°, 30.1°±0.2°, 30.4°±0.2°, 30.9°±0.2°, and 31.4°±0.2° 2θ.

In certain embodiments, Form E of the compound of formula (I) has an XRPD pattern comprising peaks at 6.7°±0.2°, 7.2°±0.2°, 8.5°±0.2°, 8.8°±0.2°, 10.2°±0.2°, 11.1°±0.2°, 11.4°±0.2°, 12.2°±0.2°, 13.5°±0.2°, 14.2°±0.2°, 14.5°±0.2°, 15.1°±0.2°, 16.0°±0.2°, 16.1°±0.2°, 16.8°±0.2°, 17.0°±0.2°, 17.6°±0.2°, 17.7°±0.2°, 18.5°±0.2°, 19.0°±0.2°, 19.2°±0.2°, 19.7°±0.2°, 20.5°±0.2°, 21.0°±0.2°, 21.1°±0.2°, 21.7°±0.2°, 22.2°±0.2°, 22.8°±0.2°, 23.4°±0.2°, 24.1°±0.2°, 24.6°±0.2°, 25.1°±0.2°, 25.7°±0.2°, 26.1°±0.2°, 26.6°±0.2°, 27.2°±0.2°, 27.7°±0.2°, 28.2°±0.2°, 29.2°±0.2°, 29.5°±0.2°, 30.1°±0.2°, 30.4°±0.2°, 30.9°±0.2°, and 31.4°±0.2° 2θ.

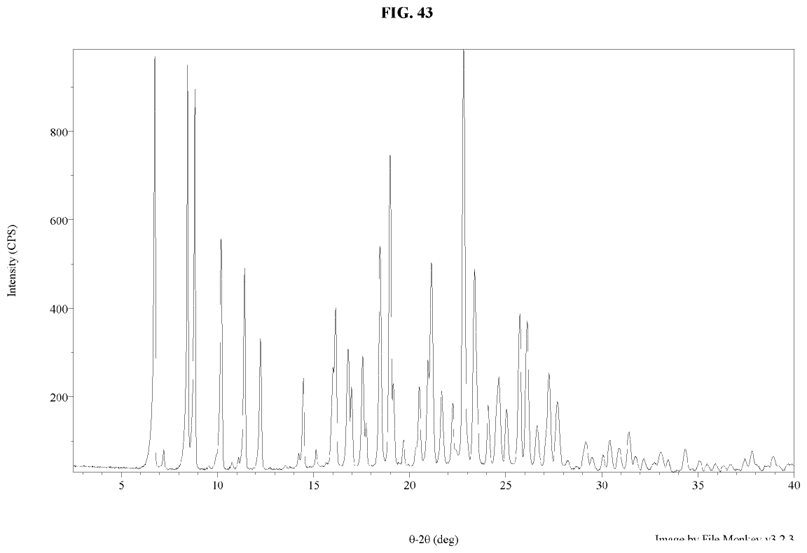

In certain embodiments, Form E of the compound of formula (I) has an XRPD pattern substantially the same as shown in FIG. 43. In certain embodiments, Form E of the compound of formula (I) has an XRPD pattern comprising one or more diffraction peaks (2θ) disclosed in Table 38.

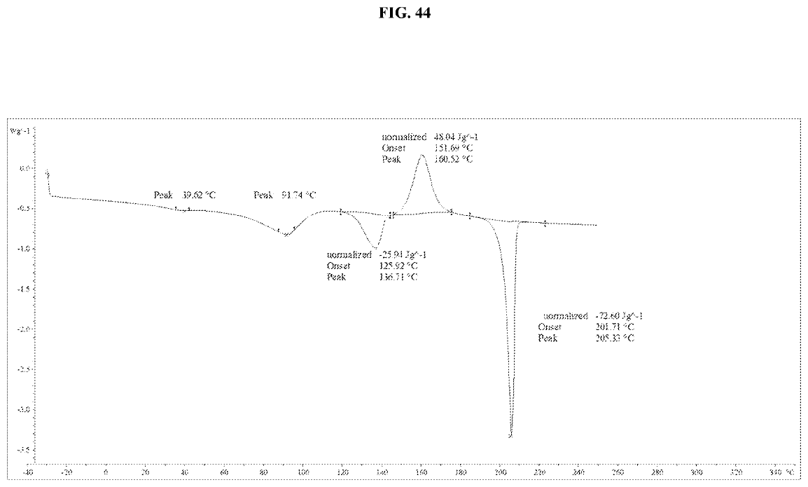

In certain embodiments, Form E of the compound of formula (I) has a DSC thermogram comprising an endotherm with a peak maximum at about 40° C. In certain embodiments, Form E of the compound of formula (I) has a DSC thermogram comprising an endotherm with a peak maximum at about 92° C. In certain embodiments, Form E of the compound of formula (I) has a DSC thermogram comprising an endotherm with a peak maximum at about 137° C. In certain embodiments, Form E of the compound of formula (I) has a DSC thermogram comprising one or more endotherms with peak maximums at about 40° C., about 92° C., and about 137° C. In certain embodiments, Form E of the compound of formula (I) has a DSC thermogram comprising an endotherm with a peak onset at about 202° C. In certain embodiments, Form E of the compound of formula (I) has a DSC thermogram comprising an exotherm with a peak maximum at about 161° C. In certain embodiments, Form E of the compound of formula (I) has a DSC thermogram substantially the same as shown in FIG. 44.

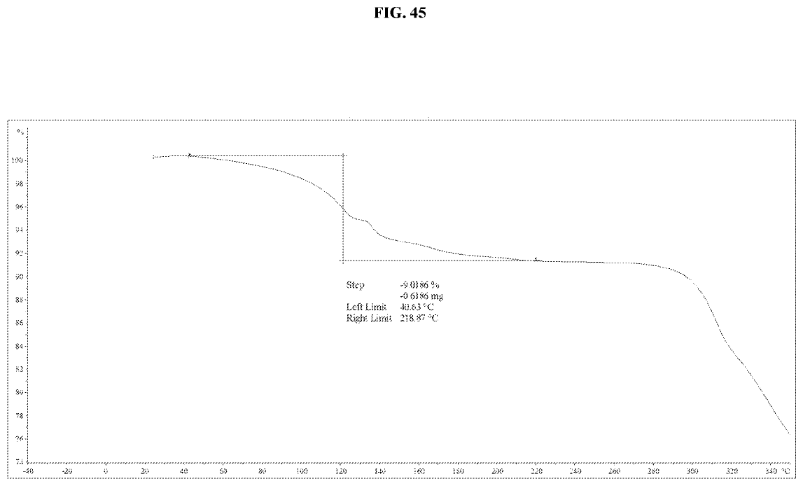

In certain embodiments, Form E of the compound of formula (I) exhibits a weight loss of less than or equal to about 9.0% wt. upon heating Form E from about 25° C. to about 219° C. The weight loss exhibited by a crystalline form (e.g., Form E of the compound of formula (I)) can be determined, for example, using TGA. In certain embodiments, Form E of the compound of formula (I) has a TGA thermogram substantially the same as shown in FIG. 45.

In certain embodiments, Form E of the compound of formula (I) is a crystalline solvate. In certain embodiments, Form E of the compound of formula (I) is a crystalline ethyl acetate solvate.

### (6) Form H

In various embodiments, provided herein is Form H of a compound of formula (I)

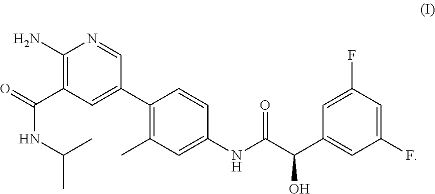 [SMILES: Cc1cc(NC(=O)[C@H](O)c2cc(F)cc(F)c2)ccc1-c1cnc(N)c(C(=O)NC(C)C)c1]

In certain embodiments, Form H of the compound of formula (I) has an XRPD pattern comprising a peak at about 8.1° 2θ. In certain embodiments, Form H of a compound of formula (I) has an XRPD pattern comprising a peak at about 9.4° 2θ. In certain embodiments, Form H of the compound of formula (I) has an XRPD pattern comprising one or more peaks at about 8.1° and about 9.4° 2θ.

In certain embodiments, the XRPD pattern further comprises one or more peaks at about 15.6°, about 16.3°, about 16.9°, about 17.0°, about 18.4°, about 18.8°, about 19.9°, about 20.2°, about 20.5°, about 21.5°, about 21.6°, about 21.8°, about 24.5°, and about 26.3° 2θ.

In certain embodiments, Form H of the compound of formula (I) has an XRPD pattern comprising one or more peaks at about 8.1°, about 9.4°, about 15.6°, about 16.3°, about 16.9°, about 17.0°, about 18.4°, about 18.8°, about 19.9°, about 20.2°, about 20.5°, about 21.5°, about 21.6°, about 21.8°, about 24.5°, and about 26.3° 2θ.

In certain embodiments, Form H of the compound of formula (I) has an XRPD pattern comprising peaks at about 8.1°, about 9.4°, about 15.6°, about 16.3°, about 16.9°, about 17.0°, about 18.4°, about 18.8°, about 19.9°, about 20.2°, about 20.5°, about 21.5°, about 21.6°, about 21.8°, about 24.5°, and about 26.3° 2θ.

In certain embodiments, Form H of the compound of formula (I) has an XRPD pattern comprising one or more peaks at about 8.1° and about 9.4° 2θ.

In certain embodiments, the XRPD pattern further comprises one or more peaks at about 5.3°, about 6.9°, about 8.1°, about 9.4°, about 9.7°, about 10.7°, about 11.6°, about 12.3°, about 12.4°, about 13.5°, about 13.8°, and about 14.7° 2θ.

In certain embodiments, the XRPD pattern further comprises one or more peaks at about 16.3°, about 16.9°, about 17.0°, about 18.4°, about 18.8°, about 19.9°, about 20.2°, about 20.5°, about 21.6°, and 24.5° 2θ.

In certain embodiments, the XRPD pattern further comprises one or more peaks at about 15.6°, about 17.7°, about 19.4°, about 20.8°, about 21.2°, about 21.5°, about 21.8°, about 22.4°, about 22.8°, about 23.1°, about 24.3°, and about 24.9° 2θ.

In certain embodiments, the XRPD pattern further comprises one or more peaks at about 26.3° 2θ.

In certain embodiments, Form H of the compound of formula (I) has an XRPD pattern comprising one or more peaks at about 5.3°, about 6.9°, about 8.1°, about 9.4°, about 9.7°, about 10.7°, about 11.6°, about 12.3°, about 12.4°, about 13.5°, about 13.8°, about 14.7°, about 15.6°, about 16.0°, about 16.3°, about 16.9°, about 17.0°, about 17.7°, about 18.4°, about 18.8°, about 19.4°, about 19.9°, about 20.2°, about 20.5°, about 20.8°, about 21.2°, about 21.5°, about 21.6°, about 21.8°, about 22.4°, about 22.8°, about 23.1°, about 24.3°, about 24.5°, about 24.9°, about 25.6°, about 26.3°, about 26.9°, about 28.2°, about 29.0°, about 29.3°, and about 30.0° 2θ.

In certain embodiments, Form H of the compound of formula (I) has an XRPD pattern comprising peaks at about 5.3°, about 6.9°, about 8.1°, about 9.4°, about 9.7°, about 10.7°, about 11.6°, about 12.3°, about 12.4°, about 13.5°, about 13.8°, about 14.7°, about 15.6°, about 16.0°, about 16.3°, about 16.9°, about 17.0°, about 17.7°, about 18.4°, about 18.8°, about 19.4°, about 19.9°, about 20.2°, about 20.5°, about 20.8°, about 21.2°, about 21.5°, about 21.6°, about 21.8°, about 22.4°, about 22.8°, about 23.1°, about 24.3°, about 24.5°, about 24.9°, about 25.6°, about 26.3°, about 26.9°, about 28.2°, about 29.0°, about 29.3°, and about 30.0° 2θ.

In certain embodiments, Form H of the compound of formula (I) has an XRPD pattern comprising a peak at 8.1°±0.3° 2θ. In certain embodiments, Form H of the compound of formula (I) has an XRPD pattern comprising a peak at 9.4°±0.3° 2θ. In certain embodiments, Form H of the compound of formula (I) has an XRPD pattern comprising one or more peaks at 8.1°±0.3° and 9.4°±0.3° 2θ.

In certain embodiments, the XRPD pattern further comprises one or more peaks at 15.6°±0.3°, 16.3°±0.3°, 16.9°±0.3°, 17.0°±0.3°, 18.4°±0.3°, 18.8°±0.3°, 19.9°±0.3°, 20.2°±0.3°, 20.5°±0.3°, 21.5°±0.3°, 21.6°±0.3°, 21.8°±0.3°, 24.5°±0.3°, and 26.3° 0.3° 2θ.

In certain embodiments, Form H of the compound of formula (I) has an XRPD pattern comprising one or more peaks at 8.1°±0.3°, 9.4°±0.3°, 15.6°±0.3°, 16.3°±0.3°, 16.9°±0.3°, 17.0°±0.3°, 18.4°±0.3°, 18.8°±0.3°, 19.9°±0.3°, 20.2°±0.3°, 20.5°±0.3°, 21.5°±0.3°, 21.6°±0.3°, 21.8°±0.3°, 24.5°±0.3°, and 26.3°±0.3° 2θ.

In certain embodiments, Form H of the compound of formula (I) has an XRPD pattern comprising peaks at 8.1°±0.3°, 9.4°±0.3°, 15.6°±0.3°, 16.3°±0.3°, 16.9°±0.3°, 17.0°±0.3°, 18.4°±0.3°, 18.8°±0.3°, 19.9°±0.3°, 20.2°±0.3°, 20.5°±0.3°, 21.5°±0.3°, 21.6°±0.3°, 21.8°±0.3°, 24.5°±0.3°, and 26.3°±0.3° 2θ.

In certain embodiments, Form H of the compound of formula (I) has an XRPD pattern comprising one or more peaks at 8.1°±0.3° and 9.4°±0.3° 2θ.

In certain embodiments, the XRPD pattern further comprises one or more peaks at 5.3°±0.3°, 6.9°±0.3°, 8.1°±0.3°, 9.4°±0.3°, 9.7°±0.3°, 10.7°±0.3°, 11.6°±0.3°, 12.3°±0.3°, 12.4°±0.3°, 13.5°±0.3°, 13.8°±0.3°, and 14.7°±0.3° 2θ.

In certain embodiments, the XRPD pattern further comprises one or more peaks at 16.3°±0.3°, 16.9°±0.3°, 17.0°±0.3°, 18.4°±0.3°, 18.8°±0.3°, 19.9°±0.3°, 20.2°±0.3°, 20.5°±0.3°, 21.6°±0.3°, and 24.5°±0.3° 2θ.

In certain embodiments, the XRPD pattern further comprises one or more peaks at 15.6°±0.3°, 17.7°±0.3°, 19.4°±0.3°, 20.8°±0.3°, 21.2°±0.3°, 21.5°±0.3°, 21.8°±0.3°, 22.4°±0.3°, 22.8°±0.3°, 23.1°±0.3°, 24.3°±0.3°, and 24.9°±0.3° 2θ.

In certain embodiments, the XRPD pattern further comprises one or more peaks at 26.3°±0.3° 2θ.

In certain embodiments, Form H of the compound of formula (I) has an XRPD pattern comprising one or more peaks at 5.3°±0.3°, 6.9°±0.3°, 8.1°±0.3°, 9.4°±0.3°, 9.7°±0.3°, 10.7°±0.3°, 11.6°±0.3°, 12.3°±0.3°, 12.4°±0.3°, 13.5°±0.3°, 13.8°±0.3°, 14.7°±0.3°, 15.6°±0.3°, 16.0°±0.3°, 16.3°±0.3°, 16.9°±0.3°, 17.0°±0.3°, 17.7°±0.3°, 18.4°±0.3°, 18.8°±0.3°, 19.4°±0.3°, 19.9°±0.3°, 20.2°±0.3°, 20.5°±0.3°, 20.8°±0.3°, 21.2°±0.3°, 21.5°±0.3°, 21.6°±0.3°, 21.8°±0.3°, 22.4°±0.3°, 22.8°±0.3°, 23.1°±0.3°, 24.3°±0.3°, 24.5°±0.3°, 24.9°±0.3°, 25.6°±0.3°, 26.3°±0.3°, 26.9°±0.3°, 28.2°±0.3°, 29.0°±0.3°, 29.3°±0.3°, and 30.0°±0.3° 2θ.

In certain embodiments, Form H of the compound of formula (I) has an XRPD pattern comprising peaks at 5.3°±0.3°, 6.9°±0.3°, 8.1°±0.3°, 9.4°±0.3°, 9.7°±0.3°, 10.7°±0.3°, 11.6°±0.3°, 12.3°±0.3°, 12.4°±0.3°, 13.5°±0.3°, 13.8°±0.3°, 14.7°±0.3°, 15.6°±0.3°, 16.0°±0.3°, 16.3°±0.3°, 16.9°±0.3°, 17.0°±0.3°, 17.7°±0.3°, 18.4°±0.3°, 18.8°±0.3°, 19.4°±0.3°, 19.9°±0.3°, 20.2°±0.3°, 20.5°±0.3°, 20.8°±0.3°, 21.2°±0.3°, 21.5°±0.3°, 21.6°±0.3°, 21.8°±0.3°, 22.4°±0.3°, 22.8°±0.3°, 23.1°±0.3°, 24.3°±0.3°, 24.5°±0.3°, 24.9°±0.3°, 25.6°±0.3°, 26.3°±0.3°, 26.9°±0.3°, 28.2°±0.3°, 29.0°±0.3°, 29.3°±0.3°, and 30.0°±0.3° 2θ.

In certain embodiments, Form H of the compound of formula (I) has an XRPD pattern comprising a peak at 8.1°±0.2° 2θ. In certain embodiments, Form H of the compound of formula (I) has an XRPD pattern comprising a peak at 9.4°±0.2° 2θ. In certain embodiments, Form H of the compound of formula (I) has an XRPD pattern comprising one or more peaks at 8.1°±0.2° and 9.4°±0.2° 2θ.

In certain embodiments, the XRPD pattern further comprises one or more peaks at 15.6°±0.2°, 16.3°±0.2°, 16.9°±0.2°, 17.0°±0.2°, 18.4°±0.2°, 18.8°±0.2°, 19.9°±0.2°, 20.2°±0.2°, 20.5°±0.2°, 21.5°±0.2°, 21.6°±0.2°, 21.8°±0.2°, 24.5°±0.2°, and 26.3°±0.2° 2θ.

In certain embodiments, Form H of the compound of formula (I) has an XRPD pattern comprising one or more peaks at 8.1°±0.2°, 9.4°±0.2°, 15.6°±0.2°, 16.3°±0.2°, 16.9°±0.2°, 17.0°±0.2°, 18.4°±0.2°, 18.8°±0.2°, 19.9°±0.2°, 20.2°±0.2°, 20.5°±0.2°, 21.5°±0.2°, 21.6°±0.2°, 21.8°±0.2°, 24.5°±0.2°, and 26.3°±0.2° 2θ.

In certain embodiments, Form H of the compound of formula (I) has an XRPD pattern comprising peaks at 8.1°±0.2°, 9.4°±0.2°, 15.6°±0.2°, 16.3°±0.2°, 16.9°±0.2°, 17.0°±0.2°, 18.4°±0.2°, 18.8°±0.2°, 19.9°±0.2°, 20.2°±0.2°, 20.5°±0.2°, 21.5°±0.2°, 21.6°±0.2°, 21.8°±0.2°, 24.5°±0.2°, and 26.3°±0.2° 2θ.

In certain embodiments, Form H of the compound of formula (I) has an XRPD pattern comprising one or more peaks at 8.1°±0.2° and 9.4°±0.2° 2θ.

In certain embodiments, the XRPD pattern further comprises one or more peaks at 5.3°±0.2°, 6.9°±0.2°, 8.1°±0.2°, 9.4°±0.2°, 9.7°±0.2°, 10.7°±0.2°, 11.6°±0.2°, 12.3°±0.2°, 12.4°±0.2°, 13.5°±0.2°, 13.8°±0.2°, and 14.7°±0.2° 2θ.

In certain embodiments, the XRPD pattern further comprises one or more peaks at 16.3°±0.2°, 16.9°±0.2°, 17.0°±0.2°, 18.4°±0.2°, 18.8°±0.2°, 19.9°±0.2°, 20.2°±0.2°, 20.5°±0.2°, 21.6°±0.2°, and 24.5°±0.2° 2θ.

In certain embodiments, the XRPD pattern further comprises one or more peaks at 15.6°±0.2°, 17.7°±0.2°, 19.4°±0.2°, 20.8°±0.2°, 21.2°±0.2°, 21.5°±0.2°, 21.8°±0.2°, 22.4°±0.2°, 22.8°±0.2°, 23.1°±0.2°, 24.3°±0.2°, and 24.9°±0.2° 2θ.

In certain embodiments, the XRPD pattern further comprises one or more peaks at 26.3°±0.2° 2θ.

In certain embodiments, Form H of the compound of formula (I) has an XRPD pattern comprising one or more peaks at 5.3°±0.2°, 6.9°±0.2°, 8.1°±0.2°, 9.4°±0.2°, 9.7°±0.2°, 10.7°±0.2°, 11.6°±0.2°, 12.3°±0.2°, 12.4°±0.2°, 13.5°±0.2°, 13.8°±0.2°, 14.7°±0.2°, 15.6°±0.2°, 16.0°±0.2°, 16.3°±0.2°, 16.9°±0.2°, 17.0°±0.2°, 17.7°±0.2°, 18.4°±0.2°, 18.8°±0.2°, 19.4°±0.2°, 19.9°±0.2°, 20.2°±0.2°, 20.5°±0.2°, 20.8°±0.2°, 21.2°±0.2°, 21.5°±0.2°, 21.6°±0.2°, 21.8°±0.2°, 22.4°±0.2°, 22.8°±0.2°, 23.1°±0.2°, 24.3°±0.2°, 24.5°±0.2°, 24.9°±0.2°, 25.6°±0.2°, 26.3°±0.2°, 26.9°±0.2°, 28.2°±0.2°, 29.0°±0.2°, 29.3°±0.2°, and 30.0°±0.2° 2θ.

In certain embodiments, Form H of the compound of formula (I) has an XRPD pattern comprising peaks at 5.3°±0.2°, 6.9°±0.2°, 8.1°±0.2°, 9.4°±0.2°, 9.7°±0.2°, 10.7°±0.2°, 11.6°±0.2°, 12.3°±0.2°, 12.4°±0.2°, 13.5°±0.2°, 13.8°±0.2°, 14.7°±0.2°, 15.6°±0.2°, 16.0°±0.2°, 16.3°±0.2°, 16.9°±0.2°, 17.0°±0.2°, 17.7°±0.2°, 18.4°±0.2°, 18.8°±0.2°, 19.4°±0.2°, 19.9°±0.2°, 20.2°±0.2°, 20.5°±0.2°, 20.8°±0.2°, 21.2°±0.2°, 21.5°±0.2°, 21.6°±0.2°, 21.8°±0.2°, 22.4°±0.2°, 22.8°±0.2°, 23.1°±0.2°, 24.3°±0.2°, 24.5°±0.2°, 24.9°±0.2°, 25.6°±0.2°, 26.3°±0.2°, 26.9°±0.2°, 28.2°±0.2°, 29.0°±0.2°, 29.3°±0.2°, and 30.0°±0.2° 2θ.

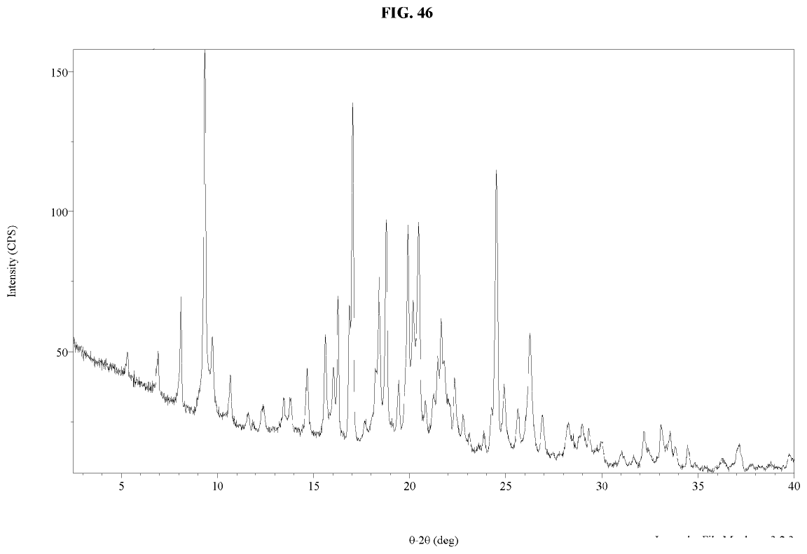

In certain embodiments, Form H of the compound of formula (I) has an XRPD pattern substantially the same as shown in FIG. 46. In certain embodiments, Form H of the compound of formula (I) has an XRPD pattern comprising one or more diffraction peaks (2θ) disclosed in Table 39.

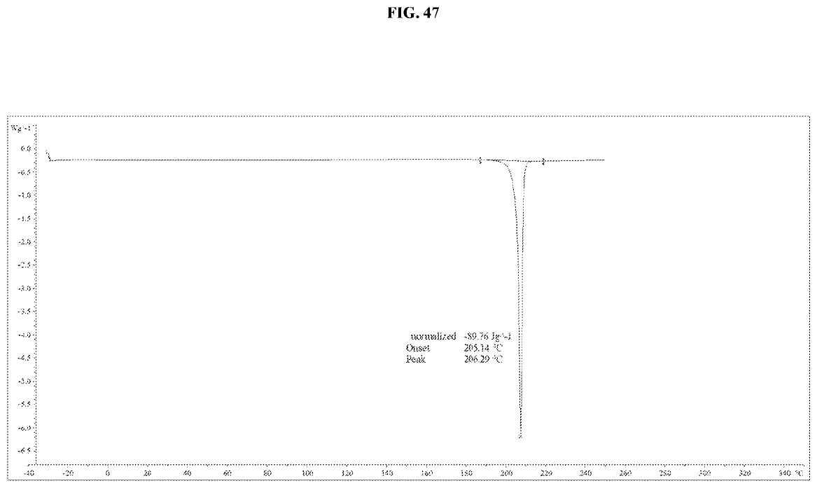

In certain embodiments, Form H of the compound of formula (I) has a DSC thermogram comprising an endotherm with a peak onset at about 205° C. In certain embodiments, Form H of the compound of formula (I) has a DSC thermogram substantially the same as shown in FIG. 47.

In certain embodiments, Form H of the compound of formula (I) is an anhydrous crystalline form.

### (7) Material F

In various embodiments, provided herein is a material (e.g., a pharmaceutical material) comprising a crystalline form of a compound of formula (I)

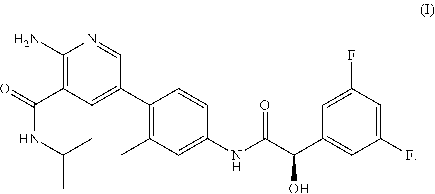 [SMILES: Cc1cc(NC(=O)[C@H](O)c2cc(F)cc(F)c2)ccc1-c1cnc(N)c(C(=O)NC(C)C)c1]

In certain embodiments, the crystalline form of the compound of formula (I) has an XRPD pattern comprising one or more peaks at about 12.9° and about 13.6° 2θ.

In certain embodiments, the XRPD pattern further comprises one or more peaks at about 4.0°, about 7.7°, about 9.1°, about 9.4°, about 10.5°, about 11.1°, and about 11.9° 2θ.

In certain embodiments, the XRPD pattern further comprises one or more peaks at about 15.4°, about 15.8°, about 18.4°, about 18.8°, about 22.4°, about 23.1°, and about 23.8° 2θ.

In certain embodiments, the XRPD pattern further comprises one or more peaks at about 16.7°, about 19.4°, about 19.9°, about 20.2°, about 21.0°, about 21.4°, about 22.7°, and about 24.7° 2θ.

In certain embodiments, the XRPD pattern further comprises one or more peaks at about 25.8° and about 28.8° 2θ.

In certain embodiments, the XRPD pattern further comprises one or more peaks at about 25.3°, about 26.5°, about 27.6°, and about 29.6° 2θ.

In certain embodiments, the crystalline form of the compound of formula (I) has an XRPD pattern comprising one or more peaks at about 4.0°, about 7.7°, about 9.1°, about 9.4°, about 10.5°, about 11.1°, about 11.9°, about 12.9°, about 13.6°, about 15.4°, about 15.8°, about 16.7°, about 18.4°, about 18.8°, about 19.4°, about 19.9°, about 20.2°, about 21.0°, about 21.4°, about 22.4°, about 22.7°, about 23.1°, about 23.8°, about 24.7°, about 25.3°, about 25.8°, about 26.5°, about 27.6°, about 28.8°, and about 29.6° 2θ.

In certain embodiments, the crystalline form of the compound of formula (I) has an XRPD pattern comprising peaks at about 4.0°, about 7.7°, about 9.1°, about 9.4°, about 10.5°, about 11.1°, about 11.9°, about 12.9°, about 13.6°, about 15.4°, about 15.8°, about 16.7°, about 18.4°, about 18.8°, about 19.4°, about 19.9°, about 20.2°, about 21.0°, about 21.4°, about 22.4°, about 22.7°, about 23.1°, about 23.8°, about 24.7°, about 25.3°, about 25.8°, about 26.5°, about 27.6°, about 28.8°, and about 29.6° 2θ.

In certain embodiments, the material comprising a crystalline form of the compound of formula (I) comprising XRPD peaks at about 4.0°, about 7.7°, about 9.1°, about 9.4°, about 10.5°, about 11.1°, about 11.9°, about 12.9°, about 13.6°, about 15.4°, about 15.8°, about 16.7°, about 18.4°, about 18.8°, about 19.4°, about 19.9°, about 20.2°, about 21.0°, about 21.4°, about 22.4°, about 22.7°, about 23.1°, about 23.8°, about 24.7°, about 25.3°, about 25.8°, about 26.5°, about 27.6°, about 28.8°, and about 29.6° 2θ may be referred to herein as “Material F”.

In certain embodiments, the crystalline form of the compound of formula (I) has an XRPD pattern comprising one or more peaks at 12.9°±0.3° and 13.6°±0.3° 2θ.

In certain embodiments, the XRPD pattern further comprises one or more peaks at 4.0°±0.3°, 7.7°±0.3°, 9.1°±0.3°, 9.4°±0.3° 10.5°±0.3°, 11.1°±0.3°, and 11.9°±0.3° 2θ.

In certain embodiments, the XRPD pattern further comprises one or more peaks at 15.4°±0.3°, 15.8°±0.3°, 18.4°±0.3°, 18.8°±0.3°, 22.4°±0.3°, 23.1°±0.3°, and 23.8°±0.3° 2θ.

In certain embodiments, the XRPD pattern further comprises one or more peaks at 16.7°±0.3°, 19.4°±0.3°, 19.9°±0.3°, 20.2°±0.3°, 21.0°±0.3°, 21.4°±0.3°, 22.7°±0.3°, and 24.7°±0.3° 2θ.

In certain embodiments, the XRPD pattern further comprises one or more peaks at 25.8°±0.3° and 28.8°±0.3° 2θ.

In certain embodiments, the XRPD pattern further comprises one or more peaks at 25.3°±0.3°, 26.5°±0.3°, 27.6°±0.3°, and 29.6°±0.3° 2θ.

In certain embodiments, the crystalline form of the compound of formula (I) has an XRPD pattern comprising one or more peaks at 4.0°±0.3°, 7.7°±0.3°, 9.1°±0.3°, 9.4°±0.3°, 10.5°±0.3°, 11.1°±0.3°, 11.9°±0.3°, 12.9°±0.3°, 13.6°±0.3°, 15.4°±0.3°, 15.8°±0.3°, 16.7°±0.3°, 18.4°±0.3°, 18.8°±0.3°, 19.4°±0.3°, 19.9°±0.3°, 20.2°±0.3°, 21.0°±0.3°, 21.4°±0.3°, 22.4°±0.3°, 22.7°±0.3°, 23.1°±0.3°, 23.8°±0.3°, 24.7°±0.3°, 25.3°±0.3°, 25.8°±0.3°, 26.5°±0.3°, 27.6°±0.3°, 28.8°±0.3°, and 29.6°±0.3° 2θ.

In certain embodiments, the crystalline form of the compound of formula (I) has an XRPD pattern comprising peaks at 4.0°±0.3°, 7.7°±0.3°, 9.1°±0.3°, 9.4°±0.3°, 10.5°±0.3°, 11.1°±0.3°, 11.9°±0.3°, 12.9°±0.3°, 13.6°±0.3°, 15.4°±0.3°, 15.8°±0.3°, 16.7°±0.3°, 18.4°±0.3°, 18.8°±0.3°, 19.4°±0.3°, 19.9°±0.3°, 20.2°±0.3°, 21.0°±0.3°, 21.4°±0.3°, 22.4°±0.3°, 22.7°±0.3°, 23.1°±0.3°, 23.8°±0.3°, 24.7°±0.3°, 25.3°±0.3°, 25.8°±0.3°, 26.5°±0.3°, 27.6°±0.3°, 28.8°±0.3°, and 29.6°±0.3° 2θ.

In certain embodiments, the material comprising a crystalline form of the compound of formula (I) comprising XRPD peaks at 4.0°±0.3°, 7.7°±0.3°, 9.1°±0.3°, 9.4°±0.3°, 10.5°±0.3°, 11.1°±0.3°, 11.9°±0.3°, 12.9°±0.3°, 13.6°±0.3°, 15.4°±0.3°, 15.8°±0.3°, 16.7°±0.3°, 18.4°±0.3°, 18.8°±0.3°, 19.4°±0.3°, 19.9°±0.3°, 20.2°±0.3°, 21.0°±0.3°, 21.4°±0.3°, 22.4°±0.3°, 22.7°±0.3°, 23.1°±0.3°, 23.8°±0.3°, 24.7°±0.3°, 25.3°±0.3°, 25.8°±0.3°, 26.5°±0.3°, 27.6°±0.3°, 28.8°±0.3°, and 29.6°±0.3° 2θ may be referred to herein as “Material F”.

In certain embodiments, the crystalline form of the compound of formula (I) has an XRPD pattern comprising one or more peaks at 12.9°±0.2° and 13.6°±0.2° 2θ.

In certain embodiments, the XRPD pattern further comprises one or more peaks at 4.0°±0.2°, 7.7°±0.2°, 9.1°±0.2°, 9.4°±0.2°, 10.5°±0.2°, 11.1°±0.2°, and 11.9°±0.2° 2θ.

In certain embodiments, the XRPD pattern further comprises one or more peaks at 15.4°±0.2°, 15.8°±0.2°, 18.4°±0.2°, 18.8°±0.2°, 22.4°±0.2°, 23.1°±0.2°, and 23.8°±0.2° 2θ.

In certain embodiments, the XRPD pattern further comprises one or more peaks at 16.7°±0.2°, 19.4°±0.2°, 19.9°±0.2°, 20.2°±0.2°, 21.0°±0.2°, 21.4°±0.2°, 22.7°±0.2°, and 24.7°±0.2° 2θ.

In certain embodiments, the XRPD pattern further comprises one or more peaks at 25.8°±0.2° and 28.8°±0.2° 2θ.

In certain embodiments, the XRPD pattern further comprises one or more peaks at 25.3°±0.2°, 26.5°±0.2°, 27.6°±0.2°, and 29.6°±0.2° 2θ.

In certain embodiments, the crystalline form of the compound of formula (I) has an XRPD pattern comprising one or more peaks at 4.0°±0.2°, 7.7°±0.2°, 9.1°±0.2°, 9.4°±0.2°, 10.5°±0.2°, 11.1°±0.2°, 11.9°±0.2°, 12.9°±0.2°, 13.6°±0.2°, 15.4°±0.2°. 15.8°±0.2°, 16.7°±0.2°, 18.4°±0.2°, 18.8°±0.2°, 19.4°±0.2°, 19.9°±0.2°, 20.2°±0.2°, 21.0°±0.2°, 21.4°±0.2°, 22.4°±0.2°, 22.7°±0.2°, 23.1°±0.2°, 23.8°±0.2°, 24.7°±0.2°, 25.3°±0.2°, 25.8°±0.2°, 26.5°±0.2°, 27.6°±0.2°, 28.8°±0.2°, and 29.6°±0.2° 2θ.

In certain embodiments, the crystalline form of the compound of formula (I) has an XRPD pattern comprising peaks at 4.0°±0.2°, 7.7°±0.2°, 9.1°±0.2°, 9.4°±0.2°, 10.5°±0.2°, 11.1°±0.2°, 11.9°±0.2°, 12.9°±0.2°, 13.6°±0.2°, 15.4°±0.2°, 15.8°±0.2°, 16.7°±0.2°, 18.4°±0.2°, 18.8°±0.2°, 19.4°±0.2°, 19.9°±0.2°, 20.2°±0.2°, 21.0°±0.2°, 21.4°±0.2°, 22.4°±0.2°, 22.7°±0.2°, 23.1°±0.2°, 23.8°±0.2°, 24.7°±0.2°, 25.3°±0.2°. 25.8°±0.2°, 26.5°±0.2°, 27.6°±0.2°, 28.8°±0.2°, and 29.6°±0.2° 2θ.

In certain embodiments, the material comprising a crystalline form of the compound of formula (I) comprising XRPD peaks at 4.0°±0.2°, 7.7°±0.2°, 9.1°±0.2°, 9.4°±0.2°, 10.5°±0.2°, 11.1°±0.2°, 11.9°±0.2°, 12.9°±0.2°, 13.6°±0.2°, 15.4°±0.2°, 15.8°±0.2°, 16.7°±0.2°, 18.4°±0.2°, 18.8°±0.2°, 19.4°±0.2°, 19.9°±0.2°, 20.2°±0.2°, 21.0°±0.2°, 21.4°±0.2°, 22.4°±0.2°, 22.7°±0.2°, 23.1°±0.2°, 23.8°±0.2°, 24.7°±0.2°, 25.3°±0.2°, 25.8°±0.2°, 26.5°±0.2°, 27.6°±0.2°, 28.8°±0.2°, and 29.6°±0.2° may be referred to herein as “Material F”.

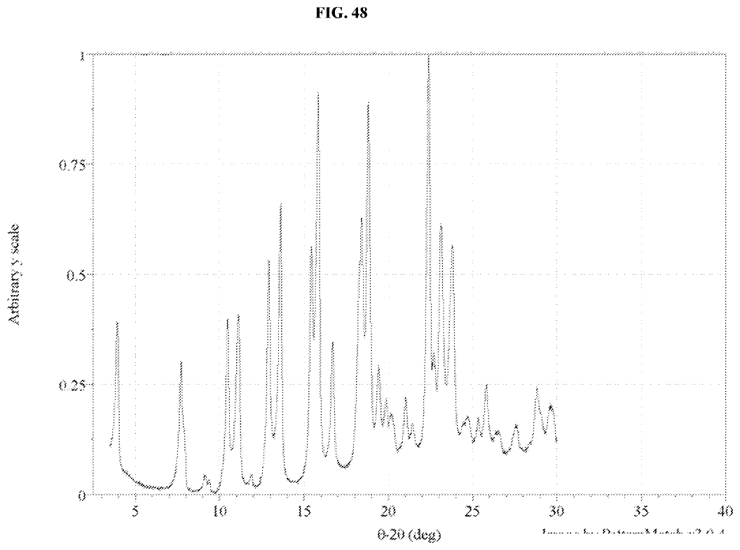

In certain embodiments, Material F of the compound of formula (I) has an XRPD pattern substantially the same as shown in FIG. 48. In certain embodiments, Material F of the compound of formula (I) has an XRPD pattern comprising one or more diffraction peaks (2θ) disclosed in Table 40.

In certain embodiments, Material F of the compound of formula (I) comprises an anhydrous crystalline form of the compound of formula (I).

### (8) Material G

In various embodiments, provided herein is a material (e.g., a pharmaceutical material) comprising a crystalline form of a compound of formula (I)

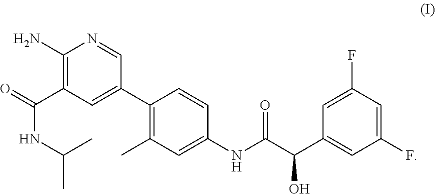 [SMILES: Cc1cc(NC(=O)[C@H](O)c2cc(F)cc(F)c2)ccc1-c1cnc(N)c(C(=O)NC(C)C)c1]

In certain embodiments, the crystalline form of the compound of formula (I) has an XRPD pattern comprising one or more peaks at about 8.7°, about 9.9°, and about 10.7° 2θ.

In certain embodiments, the XRPD pattern further comprises one or more peaks at about 6.9°, about 7.3°, about 8.9°, about 9.5°, about 9.9°, about 10.4°, about 11.0°, about 11.3°, about 11.5°, about 11.7°, about 12.3°, about 12.8°, about 13.8°, and about 14.8° 2θ.

In certain embodiments, the XRPD pattern further comprises one or more peaks at about 17.5°, about 18.6°, about 21.3°, about 21.5°, about 22.3°, and about 22.6° 2θ.

In certain embodiments, the XRPD pattern further comprises one or more peaks at about 15.2°, about 15.3°, about 15.7°, about 16.5°, about 16.8°, about 17.8°, about 19.0°, about 19.4°, about 19.8°, about 20.5°, about 20.8°, about 22.1°, about 23.7°, about 24.1°, about 24.4°, about 24.7°, and about 25.0° 2θ.

In certain embodiments, the XRPD pattern further comprises one or more peaks at about 25.3°, about 25.6°, and about 26.1° 2θ.

In certain embodiments, the XRPD pattern further comprises one or more peaks at about 26.7°, about 27.0°, about 27.5°, about 27.9°, about 28.3°, and about 28.9° 2θ.

In certain embodiments, the crystalline form of the compound of formula (I) has an XRPD pattern comprising one or more peaks at about 6.9°, about 7.3°, about 8.7°, about 8.9°, about 9.5°, about 9.9°, about 10.4°, about 10.7°, about 11.0°, about 11.3°, about 11.5°, about 11.7°, about 12.3°, about 12.8°, about 13.8°, about 14.8°, about 15.2°, about 15.3°, about 15.7°, about 16.5°, about 16.8°, about 17.5°, about 17.8°, about 18.6°, about 19.0°, about 19.4°, about 19.8°, about 20.5°, about 20.8°, about 21.3°, about 21.5°, about 22.1°, about 22.3°, about 22.6°, about 23.7°, about 24.1°, about 24.4°, about 24.7°, about 25.0°, about 25.3°, about 25.6°, about 26.1°, about 26.7°, about 27.0°, about 27.5°, about 27.9°, about 28.3°, and about 28.9° 2θ.

In certain embodiments, the crystalline form of the compound of formula (I) has an XRPD pattern comprising peaks at about 6.9°, about 7.3°, about 8.7°, about 8.9°, about 9.5°, about 9.9°, about 10.4°, about 10.7°, about 11.0°, about 11.3°, about 11.5°, about 11.7°, about 12.3°, about 12.8°, about 13.8°, about 14.8°, about 15.2°, about 15.3°, about 15.7°, about 16.5°, about 16.8°, about 17.5°, about 17.8°, about 18.6°, about 19.0°, about 19.4°, about 19.8°, about 20.5°, about 20.8°, about 21.3°, about 21.5°, about 22.1°, about 22.3°, about 22.6°, about 23.7°, about 24.1°, about 24.4°, about 24.7°, about 25.0°, about 25.3°, about 25.6°, about 26.1°, about 26.7°, about 27.0°, about 27.5°, about 27.9°, about 28.3°, and about 28.9° 2θ.

In certain embodiments, the material comprising a crystalline form of the compound of formula (I) comprising XRPD peaks at about 6.9°, about 7.3°, about 8.7°, about 8.9°, about 9.5°, about 9.9°, about 10.4°, about 10.7°, about 11.0°, about 11.3°, about 11.5°, about 11.7°, about 12.3°, about 12.8°, about 13.8°, about 14.8°, about 15.2°, about 15.3°, about 15.7°, about 16.5°, about 16.8°, about 17.5°, about 17.8°, about 18.6°, about 19.0°, about 19.4°, about 19.8°, about 20.5°, about 20.8°, about 21.3°, about 21.5°, about 22.1°, about 22.3°, about 22.6°, about 23.7°, about 24.1°, about 24.4°, about 24.7°, about 25.0°, about 25.3°, about 25.6°, about 26.1°, about 26.7°, about 27.0°, about 27.5°, about 27.9°, about 28.3°, and about 28.9° 2θ may be referred to herein as “Material G”.

In certain embodiments, the crystalline form of the compound of formula (I) has an XRPD pattern comprising one or more peaks at 8.7°±0.3°, 9.9°±0.3°, and 10.7°±0.3° 2θ.

In certain embodiments, the XRPD pattern further comprises one or more peaks at 6.9°±0.3°, 7.3°±0.3°, 8.9°±0.3°, 9.5°±0.3°, 9.9°±0.3°, 10.4°±0.3°, 11.0°±0.3°, 11.3°±0.3°, 11.5°±0.3°, 11.7°±0.3°, 12.3°±0.3°, 12.8°±0.3°, 13.8°±0.3°, and 14.8°±0.3° 2θ.

In certain embodiments, the XRPD pattern further comprises one or more peaks at 17.5°±0.3°, 18.6°±0.3°, 21.3°±0.3°, 21.5°±0.3°, 22.3°±0.3°, and 22.6°±0.3° 2θ.

In certain embodiments, the XRPD pattern further comprises one or more peaks at 15.2°±0.3°, 15.3°±0.3°, 15.7°±0.3°, 16.5°±0.3°, 16.8°±0.3°, 17.8°±0.3°, 19.0°±0.3°, 19.4°±0.3°, 19.8°±0.3°, 20.5°±0.3°, 20.8°±0.3°, 22.1°±0.3°, 23.7°±0.3°, 24.1°±0.3°, 24.4°±0.3°, 24.7°±0.3°, and 25.0°±0.3° 2θ.

In certain embodiments, the XRPD pattern further comprises one or more peaks at 25.3°±0.3°, 25.6°±0.3°, and 26.1°±0.3° 2θ.

In certain embodiments, the XRPD pattern further comprises one or more peaks at 26.7°±0.3°, 27.0°±0.3°, 27.5°±0.3°, 27.9°±0.3°, 28.3°±0.3°, and 28.9°±0.3° 2θ.

In certain embodiments, the crystalline form of the compound of formula (I) has an XRPD pattern comprising one or more peaks at 6.9°±0.3°, 7.3°±0.3°, 8.7°±0.3°, 8.9°±0.3°, 9.5°±0.3°, 9.9°±0.3°, 10.4°±0.3°, 10.7°±0.3°, 11.0°±0.3°, 11.3°±0.3°, 11.5°±0.3°, 11.7°±0.3°, 12.3°±0.3°, 12.8°±0.3°, 13.8°±0.3°, 14.8°±0.3°, 15.2°±0.3°, 15.3°±0.3°, 15.7°±0.3°, 16.5°±0.3°, 16.8°±0.3°, 17.5°±0.3°, 17.8°±0.3°, 18.6°±0.3°, 19.0°±0.3°, 19.4°±0.3°, 19.8°±0.3°, 20.5°±0.3°, 20.8°±0.3°, 21.3°±0.3°, 21.5°±0.3°, 22.1°±0.3°, 22.3°±0.3°, 22.6°±0.3°, 23.7°±0.3°, 24.1°±0.3°, 24.4°±0.3°, 24.7°±0.3°, 25.0°±0.3°, 25.3°±0.3°, 25.6°±0.3°, 26.1°±0.3°, 26.7°±0.3°, 27.0°±0.3°, 27.5°±0.3°, 27.9°±0.3°, 28.3°±0.3°, and 28.9°±0.3° 2θ.

In certain embodiments, the crystalline form of the compound of formula (I) has an XRPD pattern comprising peaks at 6.9°±0.3°, 7.3°±0.3°, 8.7°±0.3°, 8.9°±0.3°, 9.5°±0.3°, 9.9°±0.3°, 10.4°±0.3°, 10.7°±0.3°, 11.0°±0.3°, 11.3°±0.3°, 11.5°±0.3°, 11.7°±0.3°, 12.3°±0.3°, 12.8°±0.3°, 13.8°±0.3°, 14.8°±0.3°, 15.2°±0.3°, 15.3°±0.3°, 15.7°±0.3°, 16.5°±0.3°, 16.8°±0.3°, 17.5°±0.3°, 17.8°±0.3°, 18.6°±0.3°, 19.0°±0.3°, 19.4°±0.3°, 19.8°±0.3°, 20.5°±0.3°, 20.8°±0.3°, 21.3°±0.3°, 21.5°±0.3°, 22.1°±0.3°, 22.3°±0.3°, 22.6°±0.3°, 23.7°±0.3°, 24.1°±0.3°, 24.4°±0.3°, 24.7°±0.3°, 25.0°±0.3°, 25.3°±0.3°, 25.6°±0.3°, 26.1°±0.3°, 26.7°±0.3°, 27.0°±0.3°, 27.5°±0.3°, 27.9°±0.3°, 28.3°±0.3°, and 28.9°±0.3° 2θ.

In certain embodiments, the material comprising a crystalline form of the compound of formula (I) comprising XRPD peaks at 6.9°±0.3°, 7.3°±0.3°, 8.7°±0.3°, 8.9°±0.3°, 9.5°±0.3°, 9.9°±0.3°, 10.4°±0.3°, 10.7°±0.3°, 11.0°±0.3°, 11.3°±0.3°, 11.5°±0.3°, 11.7°±0.3°, 12.3°±0.3°, 12.8°±0.3°, 13.8°±0.3°, 14.8°±0.3°, 15.2°±0.3°, 15.3°±0.3°, 15.7°±0.3°, 16.5°±0.3°, 16.8°±0.3°, 17.5°±0.3°, 17.8°±0.3°, 18.6°±0.3°, 19.0°±0.3°, 19.4°±0.3°, 19.8°±0.3°, 20.5°±0.3°, 20.8°±0.3°, 21.3°±0.3°, 21.5°±0.3°, 22.1°±0.3°, 22.3°±0.3°, 22.6°±0.3°, 23.7°±0.3°, 24.1°±0.3°, 24.4°±0.3°, 24.7°±0.3°, 25.0°±0.3°, 25.3°±0.3°, 25.6°±0.3°, 26.1°±0.3°, 26.7°±0.3°, 27.0°±0.3°, 27.5°±0.3°, 27.9°±0.3°, 28.3°±0.3°, and 28.9°±0.3° 2θ may be referred to herein as “Material G”.

In certain embodiments, the crystalline form of the compound of formula (I) has an XRPD pattern comprising one or more peaks at 8.7°±0.2°, 9.9°±0.2°, and 10.7°±0.2° 2θ.

In certain embodiments, the XRPD pattern further comprises one or more peaks at 6.9°±0.2°, 7.3°±0.2°, 8.9°±0.2°, 9.5°±0.2°, 9.9°±0.2°, 10.4°±0.2°, 11.0°±0.2°, 11.3°±0.2°, 11.5°±0.2°, 11.7°±0.2°, 12.3°±0.2°, 12.8°±0.2°, 13.8°±0.2°, and 14.8°±0.2° 2θ.

In certain embodiments, the XRPD pattern further comprises one or more peaks at 17.5°±0.2°, 18.6°±0.2°, 21.3°±0.2°, 21.5°±0.2°, 22.3°±0.2°, and 22.6°±0.2° 2θ.

In certain embodiments, the XRPD pattern further comprises one or more peaks at 15.2°±0.2°, 15.3°±0.2°, 15.7°±0.2°, 16.5°±0.2°, 16.8°±0.2°, 17.8°±0.2°, 19.0°±0.2°, 19.4°±0.2°, 19.8°±0.2°, 20.5°±0.2°, 20.8°±0.2°, 22.1°±0.2°, 23.7°±0.2°, 24.1°±0.2°, 24.4°±0.2°, 24.7°±0.2°, and 25.0°±0.2° 2θ.

In certain embodiments, the XRPD pattern further comprises one or more peaks at 25.3°±0.2°, 25.6°±0.2°, and 26.1°±0.2° 2θ.

In certain embodiments, the XRPD pattern further comprises one or more peaks at 26.7°±0.2°, 27.0°±0.2°, 27.5°±0.2°, 27.9°±0.2°, 28.3°±0.2°, and 28.9°±0.2° 2θ.

In certain embodiments, the crystalline form of the compound of formula (I) has an XRPD pattern comprising one or more peaks at 6.9°±0.2°, 7.3°±0.2°, 8.7°±0.2°, 8.9°±0.2°, 9.5°±0.2°, 9.9°±0.2°, 10.4°±0.2°, 10.7°±0.2°, 11.0°±0.2°, 11.3°±0.2°, 11.5°±0.2°, 11.7°±0.2°, 12.3°±0.2°, 12.8°±0.2°, 13.8°±0.2°, 14.8°±0.2°, 15.2°±0.2°, 15.3°±0.2°, 15.7°±0.2°, 16.5°±0.2±°, 16.8°±0.2°, 17.5°±0.2°, 17.8°±0.2°, 18.6°±0.2°, 19.0°±0.2°, 19.4°±0.2°, 19.8°±0.2°, 20.5°±0.2°, 20.8°±0.2°, 21.3°±0.2°, 21.5°±0.2°, 22.1°±0.2°, 22.3°±0.2°, 22.6°±0.2°, 23.7°±0.2°, 24.1°±0.2°, 24.4°±0.2°, 24.7°±0.2°, 25.0°±0.2°, 25.3°±0.2°, 25.6°±0.2°, 26.1°±0.2°, 26.7°±0.2°, 27.0°±0.2°, 27.5°±0.2°, 27.9°±0.2°, 28.3°±0.2°, and 28.9°±0.2° 2θ.

In certain embodiments, the crystalline form of the compound of formula (I) has an XRPD pattern comprising peaks at 6.9°±0.2°, 7.3°±0.2°, 8.7°±0.2°, 8.9°±0.2°, 9.5°±0.2°, 9.9°±0.2°, 10.4°±0.2°, 10.7°±0.2°, 11.0°±0.2°, 11.3°±0.2°, 11.5°±0.2°, 11.7°±0.2°, 12.3°±0.2°, 12.8°±0.2°, 13.8°±0.2°, 14.8°±0.2°, 15.2°±0.2°, 15.3°±0.2°. 15.7°±0.2°, 16.5°±0.2±°, 16.8°±0.2°, 17.5°±0.2°, 17.8°±0.2°, 18.6°±0.2°, 19.0°±0.2°, 19.4°±0.2°, 19.8°±0.2°, 20.5°±0.2°, 20.8°±0.2°, 21.3°±0.2°, 21.5°±0.2°, 22.1°±0.2°, 22.3°±0.2°, 22.6°±0.2°, 23.7°±0.2°, 24.1°±0.2°, 24.4°±0.2°, 24.7°±0.2°, 25.0°±0.2°, 25.3°±0.2°, 25.6°±0.2°, 26.1°±0.2°, 26.7°±0.2°, 27.0°±0.2°, 27.5°±0.2°, 27.9°±0.2°, 28.3°±0.2°, and 28.9°±0.2° 2θ.

In certain embodiments, the material comprising a crystalline form of the compound of formula (I) comprising XRPD peaks at 6.9°±0.2°, 7.3°±0.2°, 8.7°±0.2°, 8.9°±0.2°, 9.5°±0.2°, 9.9°±0.2°, 10.4°±0.2°, 10.7°±0.2°, 11.0°±0.2°, 11.3°±0.2°, 11.5°±0.2°, 11.7°±0.2°, 12.3°±0.2°, 12.8°±0.2°, 13.8°±0.2°, 14.8°±0.2°, 15.2°±0.2°, 15.3°±0.2°, 15.7°±0.2°, 16.5°±0.2°, 16.8°±0.2°, 17.5°±0.2°, 17.8°±0.2°, 18.6°±0.2°, 19.0°±0.2°, 19.4°±0.2°, 19.8°±0.2°, 20.5°±0.2°, 20.8°±0.2°, 21.3°±0.2°, 21.5°±0.2°, 22.1° 0.2°, 22.3°±0.2°, 22.6°±0.2°, 23.7°±0.2°, 24.1°±0.2°, 24.4°±0.2°, 24.7°±0.2°, 25.0°±0.2°, 25.3°±0.2°, 25.6°±0.2°, 26.1°±0.2°, 26.7°±0.2°, 27.0°±0.2°, 27.5°±0.2°. 27.9°±0.2°, 28.3°±0.2°, and 28.9°±0.2° 2θ may be referred to herein as “Material G”.

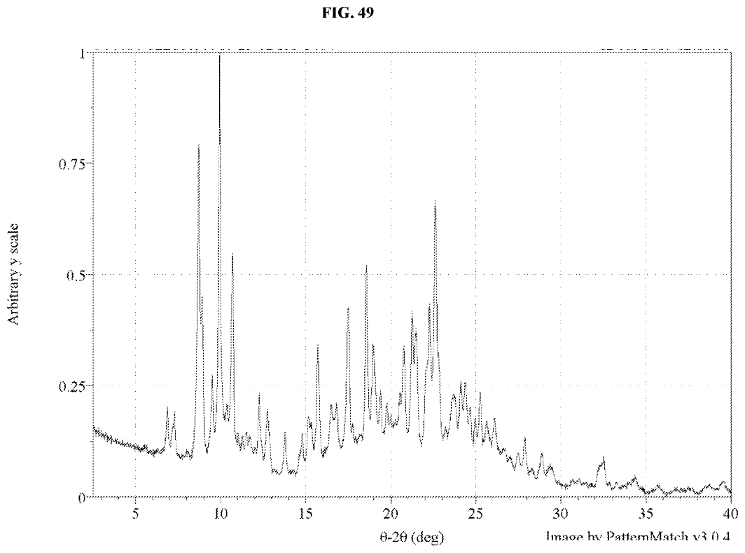

In certain embodiments, Material G of the compound of formula (I) has an XRPD pattern substantially the same as shown in FIG. 49. In certain embodiments, Material G of the compound of formula (I) has an XRPD pattern comprising one or more diffraction peaks (2θ) disclosed in Table 41.

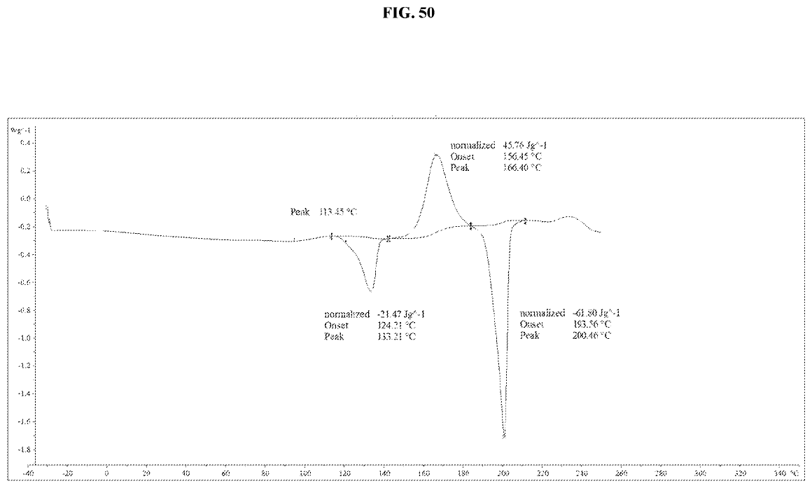

In certain embodiments, Material G of the compound of formula (I) has a DSC thermogram comprising an endotherm with a peak onset at about 124° C. In certain embodiments, Material G of the compound of formula (I) has a DSC thermogram comprising an endotherm with a peak onset at about 194° C. In certain embodiments, Material G of the compound of formula (I) has a DSC thermogram comprising one or more endotherms with peak onsets at about 124° C. and about 194° C. In certain embodiments, Material G of the compound of formula (I) has a DSC thermogram comprising an exotherm with a peak onset at about 124° C. In certain embodiments, Material G of the compound of formula (I) has a DSC thermogram substantially the same as shown in FIG. 50.

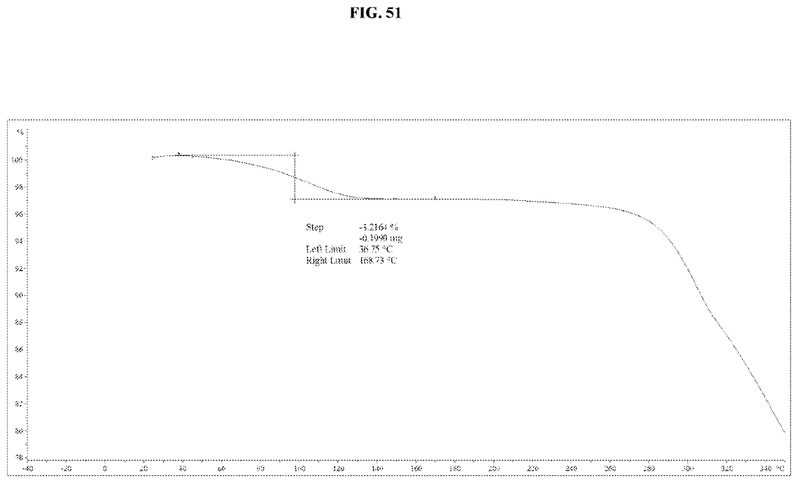

In certain embodiments, Material G of the compound of formula (I) exhibits a weight loss of less than or equal to about 3.2% wt. upon heating Material G of the compound of formula (I) from about 25° C. to about 169° C. The weight loss exhibited by a crystalline form (e.g., Material G of the compound of formula (I)) can be determined, for example, using TGA. In certain embodiments, Material G of the compound of formula (I) has a TGA thermogram substantially the same as shown in FIG. 51.

In certain embodiments, Material G of the compound of formula (I) comprises an anhydrous crystalline form of the compound of formula (I).

### Pharmaceutical Compositions

In one aspect, provided herein are pharmaceutical compositions generally comprising crystalline forms (e.g., crystalline free base forms and crystalline salt forms) of a compound of formula (I) described herein.

In one aspect, provided herein are pharmaceutical compositions generally comprising a salt form of the compound of formula (I) described herein (e.g., a fumarate salt form described herein, a L-tartrate salt form described herein, a tosylate salt form described herein, or a succinate salt form described herein).

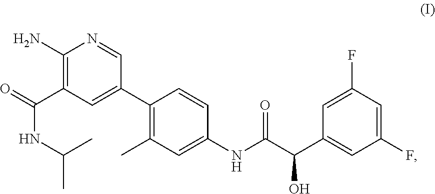 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions generally comprising:
(i) a fumarate salt of the compound of formula (I)

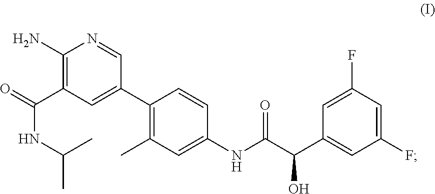 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In various embodiments, provided herein are pharmaceutical compositions generally comprising:
(i) a crystalline fumarate salt of the compound of formula (I)

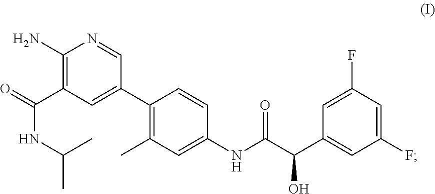 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In certain embodiments, the crystalline fumarate salt is a crystalline fumarate salt described herein.

In certain embodiments, the crystalline fumarate salt of the compound of formula (I) is a crystalline mono-fumarate salt of the compound of formula (I) described herein. In certain embodiments, the crystalline fumarate salt of the compound of formula (I) is the Form A of a mono-fumarate salt of the compound of formula (I) described herein. In certain embodiments, the crystalline fumarate salt of the compound of formula (I) is the Form B of a mono-fumarate of the compound of formula (I) described herein. In certain embodiments, the crystalline fumarate salt of the compound of formula (I) is the Form C of a mono-fumarate salt of the compound of formula (I) described herein.

In certain embodiments, the crystalline fumarate salt of the compound of formula (I) is a crystalline hemi-fumarate salt of the compound of formula (I) described herein. In certain embodiments, the crystalline fumarate salt of the compound of formula (I) is the Form A of the hemi-fumarate salt of the compound of formula (I) described herein. In certain embodiments, the crystalline fumarate salt of the compound of formula (I) is the Form B of the hemi-fumarate salt of the compound of formula (I) described herein.

In various embodiments, provided herein are pharmaceutical compositions comprising:
(i) an L-tartrate salt of the compound of formula (I)

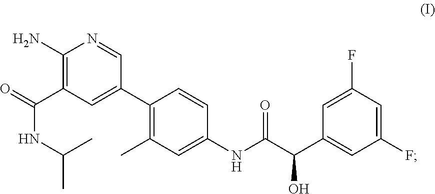 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In various embodiments, provided herein are pharmaceutical compositions comprising:
(i) a crystalline L-tartrate salt of the compound of formula (I)

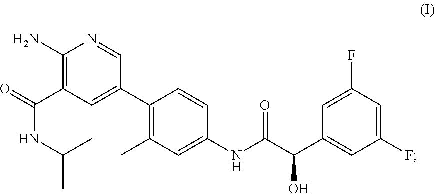 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In certain embodiments, the crystalline L-tartrate salt of the compound of formula (I) is a crystalline L-tartrate salt of the compound of formula (I) described herein.

In various embodiments, provided herein are pharmaceutical compositions comprising:
(i) a tosylate salt of the compound of formula (I)

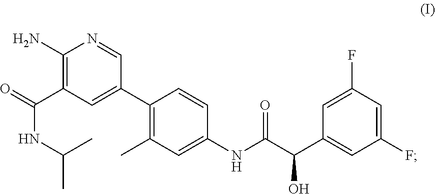 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In various embodiments, provided herein are pharmaceutical compositions comprising:
(i) a crystalline tosylate salt of the compound of formula (I)

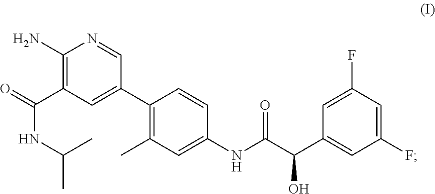 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In certain embodiments, the crystalline tosylate salt of the compound of formula (I) is a crystalline tosylate salt of the compound of formula (I) described herein. In certain embodiments, the crystalline tosylate salt of the compound of formula (I) is the Form A of a tosylate salt of the compound of formula (I) described herein.

In various embodiments, provided herein are pharmaceutical compositions comprising:
(i) a succinate salt of the compound of formula (I)

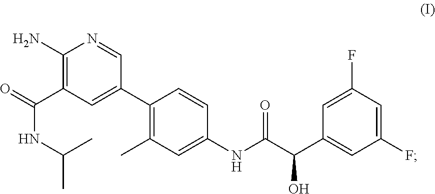 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In various embodiments, provided herein are pharmaceutical compositions comprising:
(i) a crystalline succinate salt of the compound of formula (I)

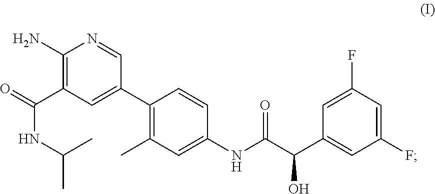 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In certain embodiments, the crystalline succinate salt of the compound of formula (I) is a crystalline succinate salt of the compound of formula (I) described herein. In certain embodiments, the crystalline succinate salt of the compound of formula (I) is the Form A of a succinate salt of the compound of formula (I) described herein.

In various embodiments, provided herein are pharmaceutical compositions comprising a crystalline mono-fumarate salt of the compound of formula (I) described herein (e.g., Form A, Form B, or Form C of the mono-fumarate salt of the compound of formula (I))

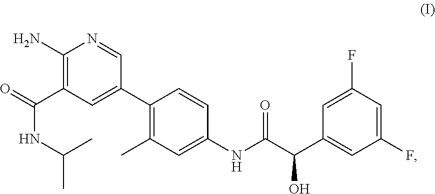 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising a crystalline hemi-fumarate salt of the compound of formula (I) described herein (e.g., Form A or Form B of the hemi-fumarate salt of the compound of formula (I))

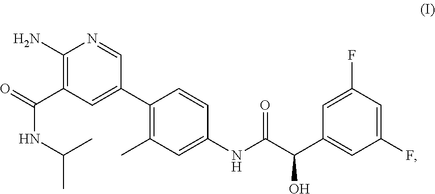 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Form A of the mono-fumarate salt of the compound of formula (I) described herein

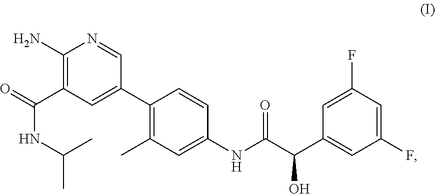 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Form B of the mono-fumarate salt of the compound of formula (I) described herein

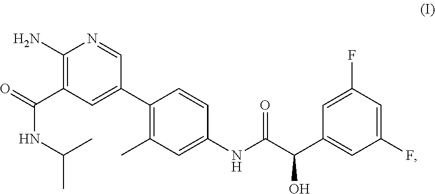 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Form C of the mono-fumarate salt of the compound of formula (I) described herein

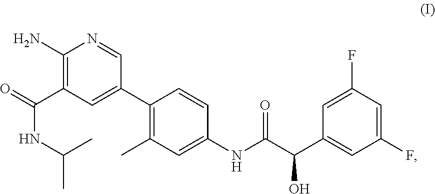 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Form A of the hemi-fumarate salt of the compound of formula (I) described herein

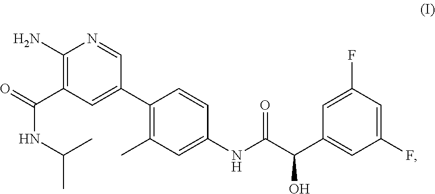 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Form B of the hemi-fumarate salt of the compound of formula (I) described herein

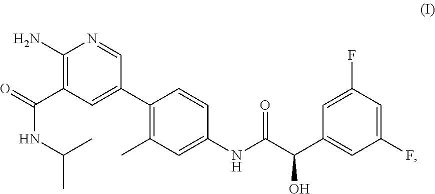 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising:
(i) a camsylate salt of the compound of formula (I)

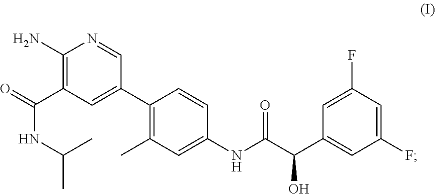 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In various embodiments, provided herein are pharmaceutical compositions comprising:
(i) a phosphate salt of the compound of formula (I)

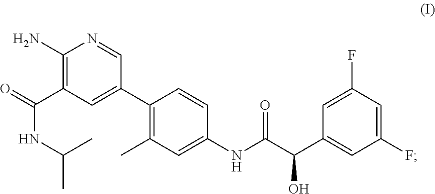 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In various embodiments, provided herein are pharmaceutical compositions comprising:
(iii) a crystalline camsylate salt of the compound of formula (I)

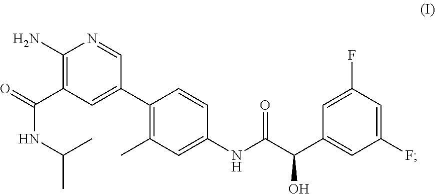 [SMILES: Cc1cc(NC(=O)[C@H](O)c2cc(F)cc(F)c2)ccc1-c1cnc(N)c(C(=O)NC(C)C)c1]

and
(iv) a pharmaceutically acceptable excipient.

In various embodiments, provided herein are pharmaceutical compositions generally comprising:
(i) a crystalline phosphate salt of the compound of formula (I)

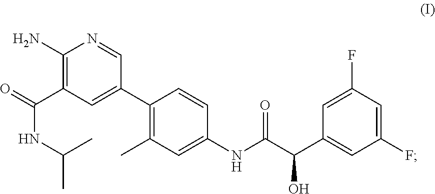 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In various embodiments, provided herein are pharmaceutical compositions generally comprising:
(i) phosphate salt Material A of the compound of formula (I) described herein

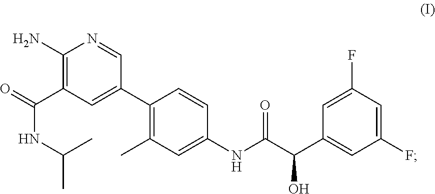 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In various embodiments, provided herein are pharmaceutical compositions generally comprising:
(i) phosphate salt Material B of the compound of formula (I) described herein

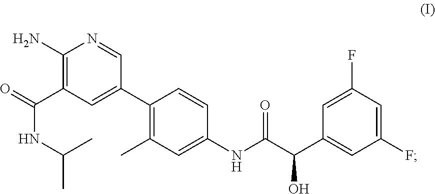 [SMILES: Cc1cc(NC(=O)[C@H](O)c2cc(F)cc(F)c2)ccc1-c1cnc(N)c(C(=O)NC(C)C)c1]

and
(ii) a pharmaceutically acceptable excipient.

In certain embodiments, the crystalline camsylate salt is a crystalline camsylate salt described herein.

In certain embodiments, the crystalline phosphate salt is a crystalline phosphate salt described herein.

In certain embodiments, the crystalline camsylate salt of the compound of formula (I) is crystalline camsylate salt Form A of the compound of formula (I) described herein.

In various embodiments, provided herein are pharmaceutical compositions comprising crystalline camsylate salt Form A of the compound of formula (I) described herein

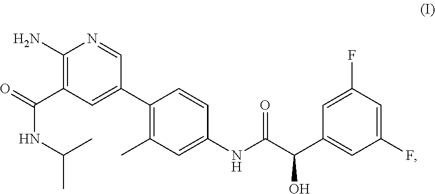 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In another aspect, provided herein are pharmaceutical compositions comprising crystalline camsylate salt Form A of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising phosphate salt Material A of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising phosphate salt Material B of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising a salt form of the compound of formula (I) described herein (e.g., a crystalline mono-fumarate salt of the compound of formula (I) described herein, a crystalline hemi-fumarate salt of the compound of formula (I) described herein, a crystalline L-tartrate salt of the compound of formula (I) described herein, a crystalline tosylate salt of the compound of formula (I) described herein, or a crystalline succinate salt of the compound of formula (I) described herein), and a pharmaceutically acceptable excipient, for the treatment of a condition, disease, or disorder described herein (e.g., a cancer) in a subject in need thereof.

In another aspect, provided herein are pharmaceutical compositions comprising a crystalline mono-fumarate salt of the compound of formula (I) described herein (e.g., Form A, Form B, or Form C of the mono-fumarate salt of the compound of formula (I)), and a pharmaceutically acceptable excipient, for the treatment of a condition, disease, or disorder described herein (e.g., a cancer) in a subject in need thereof.

In another aspect, provided herein are pharmaceutical compositions comprising a crystalline hemi-fumarate salt of the compound of formula (I) described herein (e.g., Form A or Form B of the hemi-fumarate salt of the compound of formula (I)), and a pharmaceutically acceptable excipient, for the treatment of a condition, disease, or disorder described herein (e.g., a cancer) in a subject in need thereof.

In another aspect, provided herein are pharmaceutical compositions comprising a crystalline L-tartrate salt of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising a crystalline tosylate salt of the compound of formula (I) described herein (e.g., Form A of the tosylate salt of the compound of formula (I)), and a pharmaceutically acceptable excipient, for the treatment of a condition, disease, or disorder described herein (e.g., a cancer) in a subject in need thereof.

In another aspect, provided herein are pharmaceutical compositions comprising a crystalline succinate salt of the compound of formula (I) described herein (e.g., Form A of the succinate salt of the compound of formula (I)), and a pharmaceutically acceptable excipient, for the treatment of a condition, disease, or disorder described herein (e.g., a cancer) in a subject in need thereof.

In another aspect, provided herein are pharmaceutical compositions comprising Form A of the mono-fumarate salt of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising Form B of the mono-fumarate salt of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising Form C of the mono-fumarate salt of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising Form A of the hemi-fumarate salt of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising Form B of the hemi-fumarate salt of the compound of formula (I) described herein, and a pharmaceutically acceptable excipient, for the treatment of a condition, disease, or disorder described herein (e.g., a cancer) in a subject in need thereof.

In one aspect, provided herein are pharmaceutical compositions generally comprising a crystalline free base of a compound of formula (I) described herein

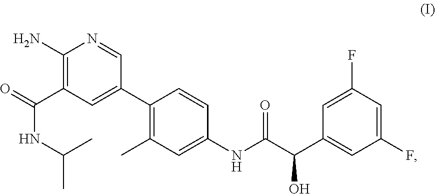 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In certain embodiments, the crystalline form of the compound of formula (I) is a crystalline free base form. In certain embodiments, the crystalline form of the compound of formula (I) is an anhydrous crystalline form. In certain embodiments, the crystalline form of the compound of formula (I) is Form A of the compound of formula (I). In certain embodiments, the crystalline form of the compound of formula (I) is Form B of the compound of formula (I). In certain embodiments, the crystalline form of the compound of formula (I) is Form C of the compound of formula (I). In certain embodiments, the crystalline form of the compound of formula (I) is Form D of the compound of formula (I). In certain embodiments, the crystalline form of the compound of formula (I) is Form E of the compound of formula (I). In certain embodiments, the crystalline form of the compound of formula (I) is Form H of the compound of formula (I). In certain embodiments, the crystalline form of the compound of formula (I) is a crystalline solvate. In certain embodiments, the crystalline solvate is a crystalline acetonitrile solvate. In certain embodiments, the crystalline solvate is a crystalline acetone solvate. In certain embodiments, the crystalline solvate is a crystalline ethyl acetate solvate. In certain embodiments, the crystalline form of the compound of formula (I) is a crystalline hydrate.

In certain embodiments, a pharmaceutical composition described herein comprises Material F of the compound of formula (I). In certain embodiments, a pharmaceutical composition described herein comprises Material G of the compound of formula (I).

In various embodiments, provided herein are pharmaceutical compositions comprising Form A of the compound of formula (I) described herein

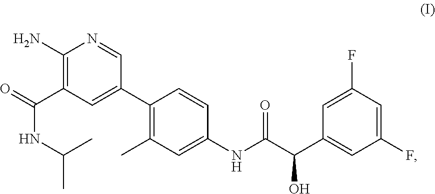 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Form B of the compound of formula (I) described herein

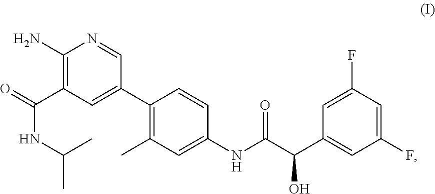 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Form C of the compound of formula (I) described herein

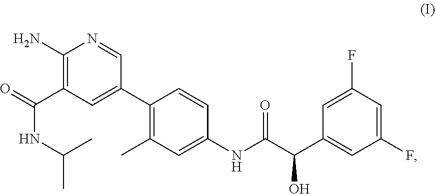 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Form D of the compound of formula (I) described herein

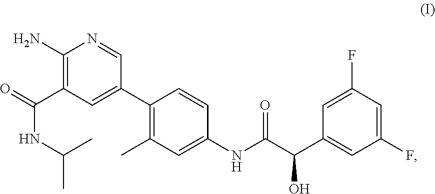 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Form E of the compound of formula (I) described herein

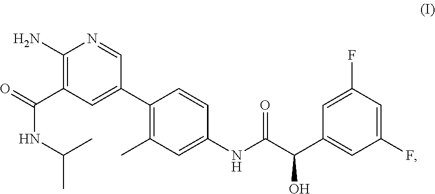 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Form H of the compound of formula (I) described herein

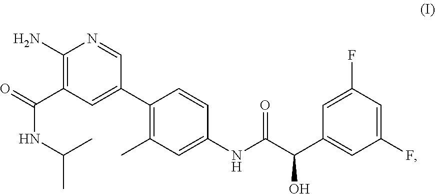 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Material F of the compound of formula (I) described herein

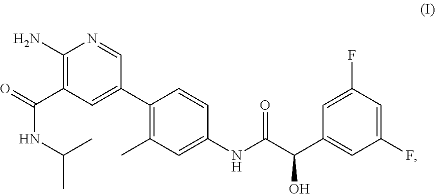 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In various embodiments, provided herein are pharmaceutical compositions comprising Material G of the compound of formula (I) described herein

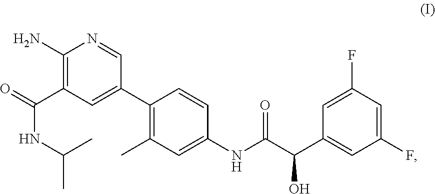 [SMILES: Cc1cc(NC(=O)[C@H](O)c2cc(F)cc(F)c2)ccc1-c1cnc(N)c(C(=O)NC(C)C)c1]

and a pharmaceutically acceptable excipient.

In another aspect, provided herein are pharmaceutical compositions comprising a crystalline form of the compound of formula (I) described herein (e.g., a crystalline free base form described herein and/or a crystalline salt form described herein), and a pharmaceutically acceptable excipient, for the treatment of a condition, disease, or disorder described herein (e.g., a cancer) in a subject in need thereof.

In another aspect, provided herein are pharmaceutical compositions comprising a crystalline free base form of the compound of formula (I) described herein (e.g., Form A, Form B, Form C, Form D, Form E, or Form H), and a pharmaceutically acceptable excipient, for the treatment of a condition, disease, or disorder described herein (e.g., a cancer) in a subject in need thereof.

In another aspect, provided herein are pharmaceutical compositions comprising a crystalline camsylate salt of the compound of formula (I) described herein (e.g., crystalline camsylate salt Form A of the compound of formula (I)), and a pharmaceutically acceptable excipient, for the treatment of a condition, disease, or disorder described herein (e.g., a cancer) in a subject in need thereof.

In another aspect, provided herein are pharmaceutical compositions comprising a material comprising a crystalline phosphate salt of the compound of formula (I) described herein (e.g., phosphate salt Material A of the compound of formula (I) or phosphate salt Material B of the compound of formula (I)), and a pharmaceutically acceptable excipient, for the treatment of a condition, disease, or disorder described herein (e.g., a cancer) in a subject in need thereof.

In another aspect, provided herein are pharmaceutical compositions comprising Form A of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising Form B of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising Form C of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising Form D of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising Form E of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising Form H of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising Material F of the compound of formula (I) described herein, and a pharmaceutically acceptable excipient, for the treatment of a condition, disease, or disorder described herein (e.g., a cancer) in a subject in need thereof.

In another aspect, provided herein are pharmaceutical compositions comprising Material G of the compound of formula (I) described herein, and a pharmaceutically acceptable excipient, for the treatment of a condition, disease, or disorder described herein (e.g., a cancer) in a subject in need thereof.

In certain embodiments, the amount of the compound of formula (I), or a pharmaceutically acceptable salt thereof, in a pharmaceutical composition described herein is about 22 mg to about 226 mg, about 35 mg to about 226 mg, about 45 mg to about 226 mg, about 55 mg to about 226 mg, about 65 mg to about 226 mg, about 75 mg to about 226 mg, about 85 mg to about 226 mg, about 95 mg to about 226 mg, about 105 mg to about 226 mg, about 115 mg to about 226 mg, about 125 mg to 226 mg, about 135 mg to about 226 mg, about 145 mg to about 226 mg, about 155 mg to about 226 mg, about 165 mg to about 226 mg, about 175 mg to about 226 mg, about 185 mg to about 226 mg, about 195 mg to about 226 mg, about 22 mg to about to about 190 mg, about 22 mg to about 180 mg, about 22 mg to about 170 mg, about 22 mg to about 160 mg, about 22 mg to about 150 mg, about 22 mg to about 140 mg, about 22 mg to about 130 mg, about 22 mg to about 120 mg, about 22 mg to about 110 mg, about 22 mg to about 100 mg, about 22 mg to about 90 mg, about 22 mg to about 80 mg, about 22 mg to about 70 mg, about 22 mg to about 60 mg, about 22 mg to about 50 mg, about 22 mg to about 40 mg, about 22 mg to about 30 mg, about 35 mg to about 185 mg, about 45 mg to about 175 mg, about 55 mg to about 165 mg, about 65 mg to about 155 mg, about 75 mg to about 145 mg, about 85 mg to about 135 mg, about 95 mg to about 125 mg, or about 105 mg to about 115 mg.

In certain embodiments, the amount of the compound of formula (I), or a pharmaceutically acceptable salt thereof, in a pharmaceutical composition described herein is about 177 mg to about 900 mg, about 250 mg to about 900 mg, about 300 mg to about 900 mg, about 350 mg to about 900 mg, about 400 mg to about 900 mg, about 450 mg to about 900 mg, about 500 mg to about 900 mg, about 550 mg to about 900 mg, about 600 mg to about 900 mg, about 650 mg to about 900 mg, about 700 mg to about 900 mg, about 750 mg to about 900 mg, about 800 mg to about 900 mg, about 850 mg to about 900 mg, about 177 mg to about 850 mg, about 177 mg to about 800 mg, about 177 mg to about 750 mg, about 177 mg to about 700 mg, about 177 mg to about 650 mg, about 177 mg to about 600 mg, about 177 mg to about 550 mg, about 177 mg to about 500 mg, about 177 mg to about 450 mg, about 177 mg to about 400 mg, about 177 mg to about 350 mg, about 177 mg to about 300 mg, about 177 mg to about 250 mg, about 250 mg to about 850 mg, about 300 mg to about 800 mg, about 350 mg to about 750 mg, about 400 mg to about 700 mg, about 450 mg to about 650 mg, about 500 mg to about 600 mg, or about 550 mg to about 600 mg.

In certain embodiments, the amount of the compound of formula (I), or a pharmaceutically acceptable salt thereof, in a pharmaceutical composition described herein is about 22 mg to about 451 mg, about 50 mg to about 451 mg, about 75 mg to about 451 mg, about 100 mg to about 451 mg, about 125 mg to about 451 mg, about 150 mg to about 451 mg, about 175 mg to about 451 mg, about 200 mg to about 451 mg, about 225 mg to about 451 mg, about 250 mg to about 451 mg, about 275 mg to about 451 mg, about 300 mg to about 451 mg, about 325 mg to about 451 mg, about 350 mg to about 451 mg, about 375 mg to about 451 mg, about 22 mg to about 375 mg, about 22 mg to about 350 mg, about 22 mg to about 325 mg, about 22 mg to about 300 mg, about 22 mg to about 275 mg, about 22 mg to about 250 mg, about 22 mg to about 225 mg, about 22 mg to about 200 mg, about 22 mg to about 175 mg, about 22 mg to about 150 mg, about 22 mg to about 125 mg, about 22 mg to about 100 mg, about 22 mg to about 75 mg, about 22 mg to about 50 mg, about 50 mg to about 375 mg, about 75 mg to about 350 mg, about 100 mg to about 325 mg, about 125 mg to about 300 mg, about 150 mg to about 275 mg, about 175 mg to about 250 mg, about 200 mg to about 225 mg, about 50 mg to about 100 mg, about 100 mg to about 150 mg, about 150 mg to about 200 mg, about 200 mg to about 250 mg, about 250 mg to about 300 mg, about 300 mg to about 350 mg, or about 350 mg to about 451 mg.

In certain embodiments, the amount of the compound of formula (I), or a pharmaceutically acceptable salt thereof, in a pharmaceutical composition described herein is about 22 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 m, about 160 mg, about 165 mg, about 170 mf, about 175 mg, about 180 mg, about 185 mg, about 190 mg, about 195 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, or about 900 mg.

In certain embodiments, the amount of the compound of formula (I), or a pharmaceutically acceptable salt thereof, in a pharmaceutical composition described herein is about 22 mg. In certain embodiments, the amount of the compound of formula (I), or a pharmaceutically acceptable salt thereof, in a pharmaceutical composition described herein is about 50 mg. In certain embodiments, the amount of the compound of formula (I), or a pharmaceutically acceptable salt thereof, in a pharmaceutical composition described herein is about 75 mg. In certain embodiments, the amount of the compound of formula (I), or a pharmaceutically acceptable salt thereof, in a pharmaceutical composition described herein is about 100 mg. In certain embodiments, the amount of the compound of formula (I), or a pharmaceutically acceptable salt thereof, in a pharmaceutical composition described herein is about 125 mg. In certain embodiments, the amount of the compound of formula (I), or a pharmaceutically acceptable salt thereof, in a pharmaceutical composition described herein is about 150 mg. In certain embodiments, the amount of the compound of formula (I), or a pharmaceutically acceptable salt thereof, in a pharmaceutical composition described herein is about 175 mg. In certain embodiments, the amount of the compound of formula (I), or a pharmaceutically acceptable salt thereof, in a pharmaceutical composition described herein is about 200 mg. In certain embodiments, the amount of the compound of formula (I), or a pharmaceutically acceptable salt thereof, in a pharmaceutical composition described herein is about 250 mg. In certain embodiments, the amount of the compound of formula (I), or a pharmaceutically acceptable salt thereof, in a pharmaceutical composition described herein is about 300 mg. In certain embodiments, the amount of the compound of formula (I), or a pharmaceutically acceptable salt thereof, in a pharmaceutical composition described herein is about 350 mg. In certain embodiments, the amount of the compound of formula (I), or a pharmaceutically acceptable salt thereof, in a pharmaceutical composition described herein is about 400 mg. In certain embodiments, the amount of the compound of formula (I), or a pharmaceutically acceptable salt thereof, in a pharmaceutical composition described herein is about 450 mg. In certain embodiments, the amount of the compound of formula (I), or a pharmaceutically acceptable salt thereof, in a pharmaceutical composition described herein is about 500 mg. In certain embodiments, the amount of the compound of formula (I), or a pharmaceutically acceptable salt thereof, in a pharmaceutical composition described herein is about 550 mg. In certain embodiments, the amount of the compound of formula (I), or a pharmaceutically acceptable salt thereof, in a pharmaceutical composition described herein is about 600 mg. In certain embodiments, the amount of the compound of formula (I), or a pharmaceutically acceptable salt thereof, in a pharmaceutical composition described herein is about 650 mg. In certain embodiments, the amount of the compound of formula (I), or a pharmaceutically acceptable salt thereof, in a pharmaceutical composition described herein is about 700 mg. In certain embodiments, the amount of the compound of formula (I), or a pharmaceutically acceptable salt thereof, in a pharmaceutical composition described herein is about 750 mg. In certain embodiments, the amount of the compound of formula (I), or a pharmaceutically acceptable salt thereof, in a pharmaceutical composition described herein is about 800 mg. In certain embodiments, the amount of the compound of formula (I), or a pharmaceutically acceptable salt thereof, in a pharmaceutical composition described herein is about 850 mg. In certain embodiments, the amount of the compound of formula (I), or a pharmaceutically acceptable salt thereof, in a pharmaceutical composition described herein is about 900 mg.

In certain embodiments, the amount of the pharmaceutically acceptable salt of a compound of formula (I) in a pharmaceutical composition described herein is about 22 mg to about 226 mg, about 35 mg to about 226 mg, about 45 mg to about 226 mg, about 55 mg to about 226 mg, about 65 mg to about 226 mg, about 75 mg to about 226 mg, about 85 mg to about 226 mg, about 95 mg to about 226 mg, about 105 mg to about 226 mg, about 115 mg to about 226 mg, about 125 mg to about 226 mg, about 135 mg to about 226 mg, about 145 mg to about 226 mg, about 155 mg to about 226 mg, about 165 mg to about 226 mg, about 175 mg to about 226 mg, about 185 mg to about 226 mg, about 195 mg to about 226 mg, about 22 mg to about to about 190 mg, about 22 mg to about 180 mg, about 22 mg to about 170 mg, about 22 mg to about 160 mg, about 22 mg to about 150 mg, about 22 mg to about 140 mg, about 22 mg to about 130 mg, about 22 mg to about 120 mg, about 22 mg to about 110 mg, about 22 mg to about 100 mg, about 22 mg to about 90 mg, about 22 mg to about 80 mg, about 22 mg to about 70 mg, about 22 mg to about 60 mg, about 22 mg to about 50 mg, about 22 mg to about 40 mg, about 22 mg to about 30 mg, about 35 mg to about 185 mg, about 45 mg to about 175 mg, about 55 mg to about 165 mg, about 65 mg to about 155 mg, about 75 mg to about 145 mg, about 85 mg to about 135 mg, about 95 mg to about 125 mg, or about 105 mg to about 115 mg.

In certain embodiments, the amount of the pharmaceutically acceptable salt of a compound of formula (I) in a pharmaceutical composition described herein is about 177 mg to about 900 mg, about 250 mg to about 900 mg, about 300 mg to about 900 mg, about 350 mg to about 900 mg, about 400 mg to about 900 mg, about 450 mg to about 900 mg, about 500 mg to about 900 mg, about 550 mg to about 900 mg, about 600 mg to about 900 mg, about 650 mg to about 900 mg, about 700 mg to about 900 mg, about 750 mg to about 900 mg, about 800 mg to about 900 mg, about 850 mg to about 900 mg, about 177 mg to about 850 mg, about 177 mg to about 800 mg, about 177 mg to about 750 mg, about 177 mg to about 700 mg, about 177 mg to about 650 mg, about 177 mg to about 600 mg, about 177 mg to about 550 mg, about 177 mg to about 500 mg, about 177 mg to about 450 mg, about 177 mg to about 400 mg, about 177 mg to about 350 mg, about 177 mg to about 300 mg, about 177 mg to about 250 mg, about 250 mg to about 850 mg, about 300 mg to about 800 mg, about 350 mg to about 750 mg, about 400 mg to about 700 mg, about 450 mg to about 650 mg, about 500 mg to about 600 mg, or about 550 mg to about 600 mg.

In certain embodiments, the amount of the pharmaceutically acceptable salt of a compound of formula (I) in a pharmaceutical composition described herein is about 22 mg to about 451 mg, about 50 mg to about 451 mg, about 75 mg to about 451 mg, about 100 mg to about 451 mg, about 125 mg to about 451 mg, about 150 mg to about 451 mg, about 175 mg to about 451 mg, about 200 mg to about 451 mg, about 225 mg to about 451 mg, about 250 mg to about 451 mg, about 275 mg to about 451 mg, about 300 mg to about 451 mg, about 325 mg to about 451 mg, about 350 mg to about 451 mg, about 375 mg to about 451 mg, about 22 mg to about 375 mg, about 22 mg to about 350 mg, about 22 mg to about 325 mg, about 22 mg to about 300 mg, about 22 mg to about 275 mg, about 22 mg to about 250 mg, about 22 mg to about 225 mg, about 22 mg to about 200 mg, about 22 mg to about 175 mg, about 22 mg to about 150 mg, about 22 mg to about 125 mg, about 22 mg to about 100 mg, about 22 mg to about 75 mg, about 22 mg to about 50 mg, about 50 mg to about 375 mg, about 75 mg to about 350 mg, about 100 mg to about 325 mg, about 125 mg to about 300 mg, about 150 mg to about 275 mg, about 175 mg to about 250 mg, about 200 mg to about 225 mg, about 50 mg to about 100 mg, about 100 mg to about 150 mg, about 150 mg to about 200 mg, about 200 mg to about 250 mg, about 250 mg to about 300 mg, about 300 mg to about 350 mg, or about 350 mg to about 451 mg.

In certain embodiments, the amount of the pharmaceutically acceptable salt of a compound of formula (I) in a pharmaceutical composition described herein is about 22 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 m, about 160 mg, about 165 mg, about 170 mf, about 175 mg, about 180 mg, about 185 mg, about 190 mg, about 195 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, or about 900 mg.

In certain embodiments, the amount of the pharmaceutically acceptable salt of a compound of formula (I) in a pharmaceutical composition described herein is about 22 mg. In certain embodiments, the amount of the pharmaceutically acceptable salt of a compound of formula (I) in a pharmaceutical composition described herein is about 50 mg. In certain embodiments, the amount of the pharmaceutically acceptable salt of a compound of formula (I) in a pharmaceutical composition described herein is about 75 mg. In certain embodiments, the amount of the pharmaceutically acceptable salt of a compound of formula (I) in a pharmaceutical composition described herein is about 100 mg. In certain embodiments, the amount of the pharmaceutically acceptable salt of a compound of formula (I) in a pharmaceutical composition described herein is about 125 mg. In certain embodiments, the amount of the pharmaceutically acceptable salt of a compound of formula (I) in a pharmaceutical composition described herein is about 150 mg. In certain embodiments, the amount of the pharmaceutically acceptable salt of a compound of formula (I) in a pharmaceutical composition described herein is about 175 mg. In certain embodiments, the amount of the pharmaceutically acceptable salt of a compound of formula (I) in a pharmaceutical composition described herein is about 200 mg. In certain embodiments, the amount of the pharmaceutically acceptable salt of a compound of formula (I) in a pharmaceutical composition described herein is about 250 mg. In certain embodiments, the amount of the pharmaceutically acceptable salt of a compound of formula (I) in a pharmaceutical composition described herein is about 300 mg. In certain embodiments, the amount of the pharmaceutically acceptable salt of a compound of formula (I) in a pharmaceutical composition described herein is about 350 mg. In certain embodiments, the amount of the pharmaceutically acceptable salt of a compound of formula (I) in a pharmaceutical composition described herein is about 400 mg. In certain embodiments, the amount of the pharmaceutically acceptable salt of a compound of formula (I) in a pharmaceutical composition described herein is about 450 mg. In certain embodiments, the amount of the pharmaceutically acceptable salt of a compound of formula (I) in a pharmaceutical composition described herein is about 500 mg. In certain embodiments, the amount of the pharmaceutically acceptable salt of a compound of formula (I) in a pharmaceutical composition described herein is about 550 mg. In certain embodiments, the amount of the pharmaceutically acceptable salt of a compound of formula (I) in a pharmaceutical composition described herein is about 600 mg. In certain embodiments, the amount of the pharmaceutically acceptable salt of a compound of formula (I) in a pharmaceutical composition described herein is about 650 mg. In certain embodiments, the amount of the pharmaceutically acceptable salt of a compound of formula (I) in a pharmaceutical composition described herein is about 700 mg. In certain embodiments, the amount of the pharmaceutically acceptable salt of a compound of formula (I) in a pharmaceutical composition described herein is about 750. In certain embodiments, the amount of the pharmaceutically acceptable salt of a compound of formula (I) in a pharmaceutical composition described herein is about 800 mg. In certain embodiments, the amount of the pharmaceutically acceptable salt of a compound of formula (I) in a pharmaceutical composition described herein is about 850 mg. In certain embodiments, the amount of the pharmaceutically acceptable salt of a compound of formula (I) in a pharmaceutical composition described herein is about 900 mg.

In various embodiments, provided herein are pharmaceutical compositions comprising:
(i) about 22 mg to about 451 mg of a compound of formula (I), or a pharmaceutically acceptable salt thereof; and
(ii) one or more pharmaceutically acceptable excipients.

In various embodiments, provided herein are pharmaceutical compositions comprising:
(i) about 177 mg to about 900 mg of a pharmaceutically acceptable salt of the compound of formula (I); and
(ii) one or more pharmaceutically acceptable excipients.

In another aspect, provided herein are pharmaceutical compositions comprising about 22 mg to about 177 mg of a compound of formula (I), or a pharmaceutically acceptable salt thereof, and one or more pharmaceutically acceptable excipients, for the treatment of a cancer described herein (e.g., a bladder cancer, a breast cancer, a carcinoma, a cervical cancer, a colorectal cancer, a gastric cancer, a hepatocellular cancer, a kidney cancer, a lung cancer, a neuroendocrine cancer, an ovarian cancer, a pancreatic cancer, a prostate cancer, a skin cancer, and a thyroid cancer) in a subject in need thereof.

In another aspect, provided herein are pharmaceutical compositions comprising about 177 mg to about 900 mg of a compound of formula (I), or a pharmaceutically acceptable salt thereof, and one or more pharmaceutically acceptable excipients, for the treatment of a cancer described herein (e.g., a bladder cancer, a breast cancer, a carcinoma, a cervical cancer, a colorectal cancer, a gastric cancer, a hepatocellular cancer, a kidney cancer, a lung cancer, a neuroendocrine cancer, an ovarian cancer, a pancreatic cancer, a prostate cancer, a skin cancer, and a thyroid cancer) in a subject in need thereof.

In another aspect, provided herein are pharmaceutical compositions comprising about 22 mg to about 226 mg of a pharmaceutically acceptable salt of the compound of formula (I), and one or more pharmaceutically acceptable excipients, for the treatment of a cancer described herein (e.g., a bladder cancer, a breast cancer, a carcinoma, a cervical cancer, a colorectal cancer, a gastric cancer, a hepatocellular cancer, a kidney cancer, a lung cancer, a neuroendocrine cancer, an ovarian cancer, a pancreatic cancer, a prostate cancer, a skin cancer, and a thyroid cancer) in a subject in need thereof.

In another aspect, provided herein are pharmaceutical compositions comprising about 177 mg to about 900 mg of a pharmaceutically acceptable salt of the compound of formula (I), and one or more pharmaceutically acceptable excipients, for the treatment of a cancer described herein (e.g., a bladder cancer, a breast cancer, a carcinoma, a cervical cancer, a colorectal cancer, a gastric cancer, a hepatocellular cancer, a kidney cancer, a lung cancer, a neuroendocrine cancer, an ovarian cancer, a pancreatic cancer, a prostate cancer, a skin cancer, and a thyroid cancer) in a subject in need thereof.

In certain embodiments, the pharmaceutically acceptable salt of the compound of formula (I) is a fumarate salt. In certain embodiments, the pharmaceutically acceptable salt of the compound of formula (I) is a mono-fumarate salt (e.g., a crystalline mono-fumarate salt described herein). In certain embodiments, the pharmaceutically acceptable salt of the compound of formula (I) is a hemi-fumarate salt (e.g., a crystalline hemi-fumarate salt described herein).

The pharmaceutical compositions described herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In certain embodiments, the pharmaceutical compositions described herein are administered orally.

The pharmaceutical compositions described herein may also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions described herein may be presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions.

In certain embodiments, the pharmaceutical compositions provided herein are administered to the patient as a solid dosage form. In certain embodiments, the solid dosage form is a capsule.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Methods of Use and Treatment

It is contemplated that the salt forms of the compound of formula (I) and pharmaceutical compositions described herein provide therapeutic benefits to subjects suffering from a cancer. Accordingly, one aspect of the invention provides therapeutic methods for treating the foregoing diseases and conditions using the salt forms of the compound of formula (I) and pharmaceutical compositions described herein. Various aspects and embodiments of the therapeutic methods are described below.

It is contemplated that the crystalline forms of the compound of formula (I) (e.g., crystalline salt forms and crystalline free base forms) and pharmaceutical compositions described herein provide therapeutic benefits to subjects suffering from a cancer. Accordingly, one aspect of the disclosure provides therapeutic methods for treating the foregoing diseases and conditions using the crystalline forms of the compound of formula (I) and pharmaceutical compositions described herein. Various aspects and embodiments of the therapeutic methods are described below.

### (1) Cancer

In one aspect, provided herein are methods of treating a cancer in a subject in need thereof. The methods generally comprise administering an effective amount of a salt form of the compound of formula (I) (e.g., a fumarate salt described herein, a tosylate salt described herein, a L-tartrate salt described herein, or a succinate salt described herein) or a pharmaceutical composition described herein to the subject to treat the cancer.

In another aspect, provided herein are methods of treating a solid tumor in a subject in need thereof. The methods generally comprise administering to the subject an effective amount of a salt form of the compound of formula (I) (e.g., a fumarate salt described herein, a tosylate salt described herein, a L-tartrate salt described herein, or a succinate salt described herein) or a pharmaceutical composition described herein. In certain embodiments, the solid tumor is an advanced solid tumor.

In another aspect, provided herein are methods of treating a blood cancer in a subject in need thereof. The methods generally comprise administering to the subject an effective amount of a salt form of the compound of formula (I) (e.g., a fumarate salt described herein, a tosylate salt described herein, a L-tartrate salt described herein, or a succinate salt described herein) or a pharmaceutical composition described herein.

In various embodiments, the methods comprise administering an effective amount of a crystalline mono-fumarate salt of the compound of formula (I) (e.g., Form A, Form B, or Form C of the mono-fumarate salt of the compound of formula (I)) to the subject.

In various embodiments, the methods comprise administering an effective amount of a crystalline hemi-fumarate salt of the compound of formula (I) (e.g., Form A or Form B of the hemi-fumarate salt of the compound of formula (I)) to the subject.

In various embodiments, the methods comprise administering an effective amount of a crystalline L-tartrate salt of the compound of formula (I) to the subject.

In various embodiments, the methods comprise administering an effective amount of a crystalline tosylate salt of the compound of formula (I) (e.g., Form A of the tosylate salt of the compound of formula (I)) to the subject.

In various embodiments, the methods comprise administering an effective amount of a crystalline succinate salt of the compound of formula (I) (e.g., Form A of the succinate salt of the compound of formula (I)) to the subject.

In various embodiments, the methods comprise administering an effective amount of Form A of the hemi-fumarate salt of the compound of formula (I)) to the subject.

In various embodiments, the methods comprise administering an effective amount of Form B of the hemi-fumarate salt of the compound of formula (I)) to the subject.

In various embodiments, the methods comprise administering an effective amount of a pharmaceutical composition described herein to the subject.

In certain embodiments, the salt form of the compound of formula (I) is administered orally to the subject once daily for at least 1 year. In some embodiments, the salt form of the compound of formula (I) is administered orally to the subject once daily for at least 2 years.

In one aspect, provided herein are methods of treating a cancer in a subject in need thereof. The methods generally comprise administering an effective amount of a crystalline form of the compound of formula (I) (e.g., a crystalline free base form described herein and/or a crystalline salt form described herein) or a pharmaceutical composition described herein to the subject to treat the cancer.

In another aspect, provided herein are methods of treating a solid tumor in a subject in need thereof. The methods generally comprise administering to the subject an effective amount of a crystalline form of the compound of formula (I) (e.g., a crystalline free base form described herein and/or a crystalline salt form described herein) or a pharmaceutical composition described herein. In certain embodiments, the solid tumor is an advanced solid tumor.

In another aspect, provided herein are methods of treating a blood cancer in a subject in need thereof. The methods generally comprise administering to the subject an effective amount of a crystalline form of the compound of formula (I) (e.g., a crystalline free base form described herein, or a crystalline salt form described herein) or a pharmaceutical composition described herein.

In various embodiments, the methods comprise administering an effective amount of a crystalline free base form of the compound of formula (I) (e.g., Form A, Form B, Form C, Form D, Form E, and/or Form H) to the subject.

In various embodiments, the methods comprise administering an effective amount of a material comprising a crystalline free base form of the compound of formula (I) (e.g., Material F and/or Material G) to the subject.

In various embodiments, the methods comprise administering an effective amount of a crystalline salt form of the compound of formula (I) (e.g., a crystalline camsylate salt of the compound of formula (I) described herein) to the subject.

In various embodiments, the methods comprise administering an effective amount of a material comprising a crystalline salt form of the compound of formula (I) (e.g., phosphate salt Material A and/or phosphate salt Material B) to the subject.

In various embodiments, the methods comprise administering an effective amount of a crystalline camsylate salt of the compound of formula (I) (e.g., crystalline camsylate salt Form A of the compound of formula (I)) to the subject.

In various embodiments, the methods comprise administering an effective amount of Form A of the compound of formula (I) to the subject.

In various embodiments, the methods comprise administering an effective amount of Form B of the compound of formula (I) to the subject.

In various embodiments, the methods comprise administering an effective amount of Form C of the compound of formula (I) to the subject.

In various embodiments, the methods comprise administering an effective amount of Form D of the compound of formula (I) to the subject.

In various embodiments, the methods comprise administering an effective amount of Form E of the compound of formula (I) to the subject.

In various embodiments, the methods comprise administering an effective amount of Form H of the compound of formula (I) to the subject.

In various embodiments, the methods comprise administering an effective amount of Material F of the compound of formula (I) to the subject.

In various embodiments, the methods comprise administering an effective amount of Material G of the compound of formula (I) to the subject.

In various embodiments, the methods comprise administering an effective amount of crystalline camsylate salt Form A of the compound of formula (I) to the subject.

In various embodiments, the methods comprise administering an effective amount of phosphate salt Material A of the compound of formula (I) to the subject.

In various embodiments, the methods comprise administering an effective amount of phosphate salt Material B of the compound of formula (I) to the subject.

In various embodiments, the methods comprise administering an effective amount of a pharmaceutical composition described herein to the subject.

In certain embodiments, a crystalline form of the compound of formula (I) described herein is administered orally to the subject.

In certain embodiments, a crystalline form of the compound of formula (I) described herein is administered to the subject once, twice, three, four, or five times daily. In certain embodiments, a crystalline form of the compound of formula (I) described herein is administered to the subject once daily. In certain embodiments, a crystalline form of the compound of formula (I) described herein is administered to the subject twice daily.

In certain embodiments, a crystalline form of the compound of formula (I) described herein is administered to the subject for at least 1, 2, 3, 4, 5, 6, 7, 8, 9, or 10 years. In certain embodiments, a crystalline form of the compound of formula (I) described herein is administered to the subject for the duration of the subject's life span.

In certain embodiments, a crystalline form of the compound of formula (I) described herein is administered orally to the subject once, twice, three, four, or five times daily for at least 1 year. In certain embodiments, a crystalline form of the compound of formula (I) described herein is administered orally to the subject once, twice, three, four, or five times daily for at least 2 years.

In certain embodiments, administering a therapeutically effective amount of the compound of formula (I), or a pharmaceutically acceptable salt thereof, comprises administering to the subject about 22 mg to about 451 mg of the compound of formula (I), or a pharmaceutically acceptable salt thereof.

In certain embodiments, administering a therapeutically effective amount of the compound of formula (I), or a pharmaceutically acceptable salt thereof, comprises administering orally to the subject about 22 mg to about 451 mg of the compound of formula (I), or a pharmaceutically acceptable salt thereof.

In certain embodiments, administering a therapeutically effective amount of the compound of formula (I), or a pharmaceutically acceptable salt thereof, comprises administering orally to the subject about 22 mg to about 451 mg of the compound of formula (I), or a pharmaceutically acceptable salt thereof, daily.

In certain embodiments, administering a therapeutically effective amount of the compound of formula (I), or a pharmaceutically acceptable salt thereof, comprises administering orally to the subject about 22 mg to about 451 mg of the compound of formula (I), or a pharmaceutically acceptable salt thereof, once daily.

In certain embodiments, administering a therapeutically effective amount of the compound of formula (I), or a pharmaceutically acceptable salt thereof, comprises administering orally to the subject about 22 mg to about 451 mg of the compound of formula (I), or a pharmaceutically acceptable salt thereof, twice daily.

In certain embodiments, the subject is in a fasting state. In certain embodiments, the subject is not in a fasting state. In certain embodiments, administering a therapeutically effective amount of the compound of formula (I), or a pharmaceutically acceptable salt thereof, comprises administering to the subject the effective amount within about 30 minutes of completing a meal.

In certain embodiments, administering a therapeutically effective amount of the compound of formula (I), or a pharmaceutically acceptable salt thereof, comprises administering to the subject about 22 mg to about 451 mg of the compound of formula (I), or a pharmaceutically acceptable salt thereof, within about 30 minutes of completing a meal.

In certain embodiments, administering a therapeutically effective amount of the compound of formula (I), or a pharmaceutically acceptable salt thereof, comprises administering to the subject about 177 mg to about 900 mg of the compound of formula (I), or a pharmaceutically acceptable salt thereof.

In certain embodiments, administering a therapeutically effective amount of the compound of formula (I), or a pharmaceutically acceptable salt thereof, comprises administering orally to the subject about 177 mg to about 900 mg of the compound of formula (I), or a pharmaceutically acceptable salt thereof.

In certain embodiments, administering a therapeutically effective amount of the compound of formula (I), or a pharmaceutically acceptable salt thereof, comprises administering orally to the subject about 177 mg to about 900 mg of the compound of formula (I), or a pharmaceutically acceptable salt thereof, daily.

In certain embodiments, administering a therapeutically effective amount of the compound of formula (I), or a pharmaceutically acceptable salt thereof, comprises administering orally to the subject about 177 mg to about 900 mg of the compound of formula (I), or a pharmaceutically acceptable salt thereof, once daily.

In certain embodiments, administering a therapeutically effective amount of the compound of formula (I), or a pharmaceutically acceptable salt thereof, comprises administering orally to the subject about 177 mg to about 900 mg of the compound of formula (I), or a pharmaceutically acceptable salt thereof, twice daily.

In certain embodiments, the subject is in a fasting state. In certain embodiments, the subject is not in a fasting state. In certain embodiments, administering a therapeutically effective amount of the compound of formula (I), or a pharmaceutically acceptable salt thereof, comprises administering to the subject the effective amount within about 30 minutes of completing a meal.

In certain embodiments, administering a therapeutically effective amount of the compound of formula (I), or a pharmaceutically acceptable salt thereof, comprises administering to the subject about 177 mg to about 900 mg of the compound of formula (I), or a pharmaceutically acceptable salt thereof, within about 30 minutes of completing a meal.

The compound of formula (I), or a pharmaceutically acceptable salt thereof, and the solid forms described herein are contemplated to be useful in treating cancers including, but not limited to, pancreatic cancer; bladder cancer; colorectal cancer; breast cancer, including metastatic breast cancer; prostate cancer, including androgen-dependent and androgen-independent prostate cancer; kidney or renal cancer, including, e.g., metastatic renal cell carcinoma; hepatocellular cancer; lung cancer, including, e.g., NSCLC, bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma, hepatic carcinoma, rectal cancer, penile carcinoma, vulval cancer, thyroid cancer, salivary gland carcinoma, endometrial or uterine carcinoma, hepatoma, hepatocellular cancer, liver cancer, gastric or stomach cancer including gastrointestinal cancer, cancer of the peritoneum, squamous carcinoma of the lung, gastroesophageal cancer, biliary tract cancer, gall bladder cancer, colorectal/appendiceal cancer, and squamous cell cancer (e.g., epithelial squamous cell cancer).

In certain embodiments, the cancer is a bladder cancer, a breast cancer, a carcinoma, a cervical cancer, a colorectal cancer, a gastric cancer, a hepatocellular cancer, a kidney cancer, a lung cancer, a neuroendocrine cancer, an ovarian cancer, a pancreatic cancer, a prostate cancer, a skin cancer, and a thyroid cancer.

In certain embodiments, the breast cancer is triple negative breast cancer or metastatic breast cancer; the carcinoma is carcinoma of unknown primary (CUP), endometrial carcinoma, head and neck squamous cell carcinoma, Merkel cell carcinoma, squamous cell carcinoma, or urothelial carcinoma; the gastric cancer is adenocarcinoma or gastrointestinal stromal tumor; the kidney cancer is a renal cell carcinoma (RCC); the lung cancer is small cell lung cancer (SCLC) or non-small cell lung cancer; the hepatocellular cancer is a hepatocellular carcinoma; or the skin cancer is malignant melanoma.

In certain embodiments, the cancer is selected from the group consisting of a renal cell carcinoma, a gastric cancer, a breast cancer, and a small-cell lung cancer.

In certain embodiments, the cancer is selected from the group consisting of Classical Hodgkin's lymphoma, primary thymic mediastinal lymphoma, multiple myeloma, and a B cell malignancy.

In certain embodiments, the B cell malignancy is non-Hodgkin lymphoma or chronic lymphocytic leukemia.

In certain embodiments, the subject is a human.

### EXAMPLES

In order that the disclosure described herein may be more fully understood, the following examples are set forth. The synthetic and biological examples described in this application are offered to illustrate the compounds, pharmaceutical compositions, and methods provided herein and are not to be construed in any way as limiting their scope.

### Abbreviations and Definitions

| | |
|---|---|
| API | Active pharmaceutical ingredient |
| a_{w} | Water activity |
| CD₃OD | Deuterated methanol |
| CO₂ | Carbon dioxide |
| DCM | Dichloromethane |
| DMSO | Dimethyl sulfoxide |
| DMSO-d₆ | Deuterated dimethyl sulfoxide |
| dm/dt | Mass change over time |
| DSC | Differential scanning calorimeter |
| DVS | Dynamic vapor sorption |
| ee | Enantiomeric excess |
| Equiv | Equivalence |
| ES | Electrospray ionization |
| Et₂O | Diethyl ether |
| EtOAc | Ethyl acetate |
| EtOH | Ethanol |
| FaSSIF | Fasted state simulated intestinal fluid |
| FeSSIF | Fed state simulated intestinal fluid |
| g | Gram |
| h | Hour(s) |
| HBr | Hydrobromic acid |
| HCl | Hydrochloric acid |
| H₂O | Water |
| HPLC | High performance liquid chromatography |
| H₃PO₄ | Phosphoric acid |
| H₂SO₄ | Sulfuric acid |
| IPA | Isopropanol |
| IPAc | Isopropyl acetate |
| J | Joules |
| K₂CO₃ | Potassium carbonate |
| KF | Karl Fisher |
| KV | Kilovolt |
| L | Liter |
| mA | Milliampere |
| mbar | Millibar |
| MeCN | Acetonitrile |
| MEK | Methyl ethyl ketone |
| MeOH | Methanol |
| 2-Me-THF | 2-methyltetrahydrofuran |
| mg | Milligram |
| min | Minute(s) |
| mol | Molar |
| MS | Mass spectrometry |
| MsOH | Methanesulfonic acid |
| MTBE | Methyl tert-butyl ether |
| m/z | Mass-to-charge ratio |
| NaCl | Sodium chloride |
| nm | Nanometer |
| NMR | Nuclear magnetic resonance |
| Pd(dppf)₂Cl₂ | [1,1′- |
| | Bis(diphenylphosphino)ferrocene]dichloropalladium(II) |
| p-TsOH | para-Toluenesulfonic acid |
| RH | Relative humidity |
| Rt | Retention time |
| RT | Room temperature |
| s | Second(s) |
| sccm | Standard cubic centimeters per minute |
| scXRD | Single crystal x-ray diffraction |
| SFC | Supercritical fluid chromatography |
| SGF | Simulated gastric fluid |
| T | Temperature |
| TFA | Trifluoroacetic acid |
| TGA | Thermogravimetric analysis |
| THF | Tetrahydrofuran |
| UV | Ultraviolet |
| V | Volume |
| v/v | Volume per volume |
| VT-XRPD | Variable temperature X-ray powder diffraction |
| XRPD | X-ray powder diffraction |
| μL | Microliter |
| 2θ | Diffraction angle(s) |

### Example 1: Analytical Methods for Characterizing Mono-Fumarate Salt, Hemi-Fumarate Salt, L-Tartrate Salt, Tosylate Salt, and Succinate Salt Forms of the Compound of Formula (I)

### (1) X-ray Powder Diffraction (XRPD)

XRPD patterns of samples were collected using with an X-ray diffractometer (Bruker D8 advance). The system was equipped with LynxEye detector. Samples were scanned from 3 to 40° 2θ, at a step size 0.02° 2θ. The tube voltage and current were 40 KV and 40 mA, respectively.

### (2) Differential Scanning Calorimeter (DSC)

DSC thermograms of samples were collected using a DSC Q200 or Discovery DSC 250 (TA Instruments, US). The sample was placed into an aluminum pin-hole hermetic pan and the weight was accurately recorded. The sample was heated at a rate of 10° C./min from 25° C. to the final temperature.

### (3) Thermogravimetric Analysis (TGA)

TGA thermograms of samples were collected using a TGA Q500 or Discovery TGA 55 (TA Instruments, US). The sample was placed into an open tared aluminum pan, automatically weighed, and inserted into the TGA furnace. The sample was heated at a rate of 10° C./min from ambient temperature to the final temperature.

### (4) Dynamic Vapor Sorption (DVS)

Moisture sorption/desorption data were collected on a DVS Intrinsic (SMS, UK). The sample was placed into a tared sample chamber and automatically weighed. The sample was dried at 40° C. until the dm/dt was less than 0.002% and cooled to 25° C. Set the instrument parameters as below.
Step time (min): 60 min
Sample temperature: 25° C.
Cycle: Full cycle
Adsorption: 0, 10, 20, 30, 40, 50, 60, 70, 80, 90
Desorption: 80, 70, 60, 50, 40, 30, 20, 10, 0
Save Data Rate: 5 s
Total flow rate: 200 sccm
Post experiment total flow: 200 sccm

### (5) Proton Nuclear Magnetic Resonance (¹H NMR)

¹H NMR data were collected using a Bruker Advance 300 equipped with automated sample (B-ACS 120).

### (6) High-Performance Liquid Chromatography (HPLC)

HPLC analysis was performed with an Agilent HPLC 1260 series instrument. HPLC method for solubility and stability testing is listed below.
Instrument Agilent 1260 series
Column Ascentis Express C18 4.6×100 mm, 2.7 μm
Column temperature 40° C.
Mobile phase A: 0.1% TFA in water
   B: 0.1% TFA in MeCN
Gradient condition 0 min: 15%
(% of B) 4.0 min: 50%
   8.0 min: 70%
   10.0 min: 90%
Flow rate 1.0 mL/min
Injection volume 4 μL
UV wavelength 268 nm
Post time 3 min
Diluent MeCN

### Example 2: Synthesis of (R)-2-amino-5-(4-(2-(3,5-difluorophenyl)-2-hydroxyacetamido)-2-methylphenyl)-N-isopropylnicotinamide (Compound of Formula (I))

### Step 1: Isolation of (2R)-2-(3,5-difluorophenyl)-2-hydroxy-acetic acid

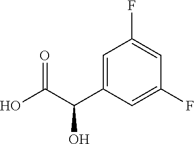 [SMILES: O=C(O)[C@H](O)c1cc(F)cc(F)c1]

Lipase PS Amano (see Mendiola, J. et al, Org. Process Res. Dev. 2012, 16, 1312-1316) was supported in diatomaceous earth prior to use by mixing 200 g of diatomaceous earth and 200 g of lipase PS Amano SD. H₂O was added to cover the solid and the mixture was stirred. H₂O was removed in an oven at 4 mbar and 40° C. for 16 h. H₂O was below 1% through Karl Fischer titration for water determination.

Supported lipase PS amano SD (250 g) and vinyl acetate (312 mL; 3.36 mol) were added to a suspension of racemic 2-(3,5-difluorophenyl)-2-hydroxyacetic acid (125 g, 664 mmol) in methyl tert-butyl ether (2.50 L), and the mixture was stirred at 26° C. for 72 h. After this time, the solid was rinsed and filtered with methyl tert-butyl ether (1.50 L), and combined filtrates were concentrated under reduced pressure. The residue was slurried in dichloromethane (160 mL) at 23° C. for 4 h. The mixture was filtered, and the solid was washed with petroleum ether (150 mL), and dried to give the title compound (47.0 g, 36%). ¹H NMR (DMSO-d₆) δ 5.11 (s, 1H), 6.20 (br s, 1H), 7.11-7.21 (m, 3H), 12.8 (br s, 1H). The absolute configuration of the title compound was determined by vibrational circular dichroism (see Freedman T. B et al, Chirality, 2003 Nov. 15 (9), 743-758). Chiral HPLC: Rt=7.39 min (UV); Column: Chiralpak® AD 4.6×150 mm 5 μm; 5% EtOH in n-hexane (0.05% TFA) isocratic; Flow Rate: 1.5 mL/min, ee>98%.

### Step 2: Synthesis of 2-Amino-5-(4-amino-2-methyl-phenyl)-N-isopropyl-pyridine-3-carboxamide

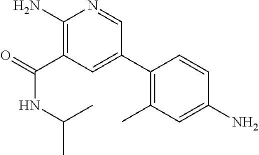 [SMILES: Cc1cc(N)ccc1-c1cnc(N)c(C(=O)NC(C)C)c1]

3-methyl-4-(4,4,5,5-tetramethyl-1,3,2-dioxaborolan-2-yl)aniline (93.6 g, 0.401 mol), K₂CO₃ (119 g, 0.860 mol), and Pd(dppf)₂Cl₂ (10.6 g, 140 mmol) were added to a solution of 2-amino-5-bromo-N-isopropyl-pyridine-3-carboxamide (74.0 g, 0.287 mol) in dioxane (888 mL) and H₂O (296 mL), and the mixture was heated at 55° C. overnight. The mixture was cooled to 23° C., ethyl acetate (150 mL) was added, the resulting suspension was filtered through diatomaceous earth, and the solid was rinsed with ethyl acetate (50 mL). The combined filtrates were washed with H₂O (30 mL) and saturated aqueous NaCl (300 mL), and concentrated under reduced pressure to give the title compound (78.0 g, 96%). ES/MS m/z 285.1 (M+H).

### Step 3: Synthesis of (R)-2-amino-5-(4-(2-(3,5-difluorophenyl)-2-hydroxyacetamido)-2-methylphenyl)-N-isopropylnicotinamide

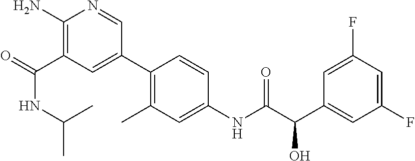 [SMILES: Cc1cc(NC(=O)[C@H](O)c2cc(F)cc(F)c2)ccc1-c1cnc(N)c(C(=O)NC(C)C)c1]

A mixture of (2R)-2-(3,5-difluorophenyl)-2-hydroxyacetic acid (29.0 g, 0.154 mol), 2-amino-5-(4-amino-2-methyl-phenyl)-N-isopropyl-pyridine-3-carboxamide (43.83 g, 0.154 mol), and N,N-diisopropylethylamine (39.8 g, 0.308 mol) in tetrahydrofuran (960 mL) was treated with (1-[bis(dimethylamino)methylene]-1H-1,2,3-triazolo[4,5-b]pyridinium 3-oxide hexafluorophosphate) (87.9 g, 0.231 mol) at 0° C. for 30 min, and the mixture was warmed to 20° C. and stirred for 2 h. Ethyl acetate (50 mL) was added, and the mixture was filtered. The filtrate was concentrated under reduced pressure, and the residue was purified by chromatography (eluent: 2:1 petroleum ether/ethyl acetate) and then by SFC (Column: Chiralpak® IC 30×250 mm 5 μm (Daicel); MeOH/CO₂=30:70 isocratic; Flow rate: 80 g/min; Back pressure: 100 Bar; Column temperature: 40° C.) to give the title compound (27.5 g, 39%) as a colorless solid. ES/MS m/z 455.2 (M+H).

### Example 3: Preparation and Characterization of the Form A of the Mono-Fumarate Salt of the Compound of Formula (I)

The compound of formula (I) (~2.96 g) was dissolved into 25 mL of EtOH at 50° C. and fumaric acid (832 mg, 1.0 equiv) was added. After stirring for 30 minutes, the solution was cooled to room temperature and precipitation appeared during cooling. After stirring at room temperature for one day, the sample was collected by filtration and dried under vacuum at 50° C. overnight. The XRPD pattern of the Form A of the mono-fumarate salt is depicted in FIG. 1. The XRPD pattern is indexed in Table 1, which tabulates diffraction angles (2θ), intensity, and relative intensity (expressed as a percentage with respect to the most intense peak).

TABLE 1
Indexed XRPD Peak Table For the Form A of the Mono-
Fumarate Salt of the Compound of Formula (I)

| Diffraction Angles (2θ, °) | Intensity | Relative Intensity (%) |
|---|---|---|
| 5.0 | 626 | 20.5 |
| 7.4 | 1003 | 46 |
| 8.2 | 765 | 33.2 |
| 9.7 | 279 | 7.5 |
| 10.0 | 321 | 9.5 |
| 10.8 | 238 | 5 |
| 11.4 | 356 | 11.4 |
| 11.9 | 269 | 7 |
| 13.2 | 298 | 8.4 |
| 13.8 | 211 | 3.4 |
| 14.5 | 270 | 5.8 |
| 14.9 | 447 | 14.2 |
| 15.2 | 698 | 26.9 |
| 16.1 | 721 | 26.8 |
| 16.5 | 767 | 28.8 |
| 17.5 | 712 | 25.1 |
| 17.9 | 344 | 5.6 |
| 18.5 | 295 | 2.8 |
| 19.3 | 468 | 11.5 |
| 19.5 | 562 | 16.4 |
| 19.9 | 502 | 13.2 |
| 20.3 | 505 | 13.4 |
| 20.8 | 575 | 16.7 |
| 21.1 | 721 | 23.8 |
| 21.9 | 606 | 16.5 |
| 22.6 | 476 | 8.9 |
| 23.1 | 676 | 19 |
| 23.5 | 745 | 22.5 |
| 24.1 | 1071 | 39.6 |
| 24.8 | 558 | 12.6 |
| 25.1 | 1493 | 62.1 |
| 25.4 | 2207 | 100 |
| 26.2 | 776 | 25.2 |
| 27.4 | 450 | 9.7 |
| 27.8 | 368 | 6.2 |
| 28.9 | 294 | 3.8 |
| 30.0 | 278 | 3.1 |
| 30.5 | 310 | 5.1 |
| 31.1 | 295 | 4.9 |
| 31.5 | 270 | 4.1 |
| 32.8 | 259 | 5 |
| 33.6 | 233 | 4 |
| 35.2 | 205 | 2.7 |
| 35.9 | 199 | 2.3 |
| 39.6 | 195 | 2.2 |

DSC/TGA thermograms of Form A of the mono-fumarate salt are depicted in FIG. 2.

The TGA thermogram of Form A of the mono-fumarate salt showed approximately 1.2% weight loss at 100° C., which was attributed to dehydration and the salt existed as a hydrate.

The DSC thermogram of Form A of the mono-fumarate salt showed broad endothermic peaks two broad endothermic peaks at 63° C. (20 J/g) and 143° C. (55 J/g).

¹H NMR analysis of Form A of the mono-fumarate salt confirmed 1:1 stoichiometry of the acid and base, with approximately 0.1% of EtOH remaining in the sample.

### Example 4: Crystallization Behavior of the Fumarate Salt from Binary Solvent Systems

Evaporative crystallization was performed in a 96-well plate using binary solvents with a permutation of the following solvents: MeOH, EtOH, IPA, isobutanol, MEK, THF, MeCN, MTBE, acetone, water, toluene, EtOAc, and IPAc. The drug solution was prepared by the following protocol: Appropriate amounts of mono-fumarate Form A were added into 2 mL of the solvents to make suspensions or solutions respectively. After stirring, each of the drug suspensions or solutions was filtered. Filtrates were then used for binary solvent screening in the 96-well plate. The well-plate was prepared by the following protocol: The saturated drug solutions (filtrates) were distributed in a 96-well plate. Each well contained two different filtrates and the volume of each filtrate was 100 μL. The plate was covered by sealing film with pin holes and allowed to evaporate in an operating laboratory fume hood under ambient conditions. The solid states of samples in the 96-well plate are summarized in Table 2.

TABLE 2
Result of the Evaporative Crystallization in Binary Solvents of the
Form A of the Mono-Fumarate Salt of the Compound of Formula (I)

| Binary Solvent | Result | Binary Solvent | Result |
|---|---|---|---|
| MeOH/water | Solid | THF/IPAc | Solid |
| EtOH/isobutanol | Form A | MeCN/acetone | Form A |
| EtOH/water | Solid | MTBE/acetone | Form A |
| EtOH/toluene | Form A | MTBE/water | Solid |
| IPA/water | Solid | MTBE/EtOAc | Solid |
| MEK/MeCN | Form C | Acetone/acetone | Form A |
| MEK/MTBE | Form C | Acetone/water | Solid |
| MEK/acetone | Form C | Acetone/toluene | Form C |
| MEK/water | Solid | Acetone/EtOAc | Form C |
| MEK/toluene | Form C | Acetone/IPAc | Form C |
| MEK/EtOAc | Solid | EtO Ac/EtOAc | Form C |
| MEK/IPAc | Form A | EtOAc/IPAc | Form C |
| THF/EtOAc | Solid | | |

“Solid” indicates a formation of crystalline solid but provided inadequate quantity for XRPD analysis.
Solids in other wells were in glassy state.

### Evaporative Crystallization in Single Solvent of the Form A of the Mono-Fumarate Salt of the Compound of Formula (I)

After the preparation of the 96-well plate, the residual filtrates from the evaporative crystallization study in binary solvents were used for slow evaporation study in a single solvent system. The Form C of the mono-fumarate salt of the compound of formula (I) was obtained in 2-butanone, MeCN, and EtOAc. A mixture of patterns was obtained in MeOH, and a low crystalline solid was obtained in EtOH and acetone. Other samples were all glassy or amorphous. The result of the study is summarized in Table 3.

TABLE 3
Result of the Evaporative Crystallization in
a Single Solvents of the Form A of the Mono-
Fumarate Salt of the Compound of Formula (I)

| Solvent | Result |
|---|---|
| MeOH | Mixed Forms |
| EtOH | Low crystalline solid |
| IPA | Glassy state |
| Isobutanol | Glassy state |
| 2-Butanone | Form C |
| THF | Solid |
| MeCN | Form C |
| MTBE | Amorphous |
| Acetone | Low crystalline solid |
| Water | Solid |
| Toluene | Solid |
| EtOAc | Form C |
| IPAc | Amorphous |

“Solid” indicates a formation of crystalline solid but provided inadequate quantity for XRPD analysis.

### Example 5: Slurry Study of the Form A of the Mono-Fumarate Salt of the Compound of Formula (I)

Slurry studies were conducted using 12 solvents at RT or 50° C. Solid samples were collected by filtration and analyzed by XRPD at a specific time. The results of the slurry study is summarized in Table 4.

TABLE 4
Result of the Slurry Study of the Form A of the
Mono-Fumarate Salt of the Compound of Formula (I)

| | Slurry at room | Slurry at 50° C., |
|---|---|---|
| Solvent | temperature, 5 days | 2 days |
| EtOH | Solid | Solid material comprising |
| | | the Mono-fumarate salt |
| IPA | Hemi-fumarate Form A | Hemi-fumarate Form A |
| Isobutanol | Hemi-fumarate Form A | Hemi-fumarate Form A |
| 2-Butanone | Mono-fumarate Form A | Mono-fumarate Form A |
| MeCN | Low crystallinity | Hemi-fumarate Form |
| | | A + additional peaks |
| MTBE | Low crystallinity | Hemi-fumarate Form A |
| Acetone | Mono-fumarate Form A | Mono-fumarate Form A |
| Water | Alternative pattern | Alternative pattern |
| Toluene | Low crystallinity | Hemi-fumarate Form A |
| EtOAc | Low crystallinity | Low crystallinity |
| IPAc | Low crystallinity | Hemi-fumarate Form A |
| Heptane | Mono-fumarate Form A | Mono-fumarate Form A |

“Solid” indicates a formation of crystalline solid but provided inadequate quantity for XRPD analysis.

### Solubility Testing of Form A of the Mono-Fumarate Salt of the Compound of Formula (I)

The solubility of Form A of the mono-fumarate salt of the compound of formula (I) was estimated using solvent addition method via visual assessment of the samples. Mono-fumarate Form A has the highest solubility in MeOH and THF (>50 mg/mL). The results are summarized in Table 5.

TABLE 5
Solubility of the Form A of the Mono-Fumarate
Salt of the Compound of Formula (I)

| Solvent | Result |
|---|---|
| MeOH | >53.2 |
| EtOH | >22.5 |
| IPA | >23.4 |
| Isobutanol | >11.1 |
| MEK | >11.7 |
| THF | >52.6 |
| MeCN | >2.2 |
| MTBE | >4.2 |
| Acetone | >13.4 |
| Water | <1.1 |
| Toluene | <1.1 |
| EtOAc | >3.0 |
| IPAc | >2.1 |
| Heptane | <1.0 |

“<” if dissolution was not observed.
“>” if dissolution occurred after addition of first aliquot of solvent.

### Example 6: Crystallization Behavior of Fumarate Salt by Anti-Solvent Precipitation

The Form A of the mono-fumarate salt (~20 mg) was dissolved into a solvent at room temperature, and the anti-solvent was added slowly until precipitation appeared or up to specific volume. If precipitation occurred, products were characterized accordingly by XRPD (Example 1, XRPD). According to the solubility of mono-fumarate Form A, total seven experiments were conducted using two solvents and four anti-solvents. As summarized in Table 6, Pattern 1, hemi-fumarate Form A and mono-fumarate Form C were obtained.

TABLE 6
Result of the Anti-Solvent Precipitation of the Form A of
the Mono-Fumarate Salt of the Compound of Formula (I)

| Solvent/Anti- | | | |
|---|---|---|---|
| Solvent | Ratio | Observation | Result |
| MeOH/water | 3:2 | Precipitation | Fumarate salt |
| | | occurred immediately | (Pattern 1) |
| MeOH/MTBE | 1:5 | No precipitation occurred after | Clear solution |
| | | stirring overnight | |
| MeOH/EtOAc | 1:10 | No precipitation occurred after | Clear solution |
| | | stirring overnight | |
| THF/water | 1:1 | Precipitation | Not enough |
| | | occurred immediately | solid |
| THF/heptane | 1:1 | Precipitation | Hemi-fumarate |
| | | occurred immediately | Form A |
| THF/MTBE | 1:10 | No precipitation occurred after | Clear solution |
| | | stirring overnight | |
| THF/EtOAc | 1:10 | Precipitation | Hemi-fumarate |
| | | occurred immediately | Form C |

### Example 7: Crystallization Behavior of the Fumarate Salt Via Cooling Crystallization

The Form A of the mono-fumarate salt (20-25 mg) was dissolved in different solvents at 60° C., then the solutions were cooled slowly to room temperature to induce crystallization. Solid samples were analyzed by XRPD (Example 1, XRPD). Total three experiments were conducted using cooling crystallization. As summarized in Table 7, the samples prepared in isobutanol and IPA were the Form A of the hemi-fumarate salt and the sample from 2-butanone was the Form C of the mono-fumarate salt.

TABLE 7
Result of the Cooling Crystallization Study of the Form A
of the Mono-Fumarate Salt of the Compound of Formula (I)

| | Solvent | | |
|---|---|---|---|
| Solvent | Volume (V) | Observation | Result |
| 2-Butanone | 15.6 | Precipitation occurred | Mono-fumarate |
| | | after stirring for 10 min | Form C |
| Isobutanol | 20.2 | Precipitation occurred | Hemi-fumarate |
| | | after stirring overnight | Form A |
| IPA | 10 | Precipitation occurred | Hemi-fumarate |
| | | after stirring for 10 min | Form A |

### Example 8: Preparation and Characterization of the Form B of the Mono-Fumarate Salt of the Compound of Formula (I)

The Form B of the mono-fumarate salt was obtained during the course of polymorph screening. The XRPD pattern of the Form B of the mono-fumarate salt is depicted in FIG. 3. The XRPD pattern is indexed in Table 8, which tabulates diffraction angles (2θ), intensity, and relative intensity (expressed as a percentage with respect to the most intense peak).

TABLE 8
Indexed XRPD Peak Table For the Form B of the Mono-
Fumarate Salt of the Compound of Formula (I)

| Diffraction Angles (2θ, °) | Intensity | Relative Intensity (%) |
|---|---|---|
| 4.7 | 355 | 5.1 |
| 7.5 | 1302 | 100 |
| 9.7 | 116 | 2.9 |
| 13.3 | 120 | 3.1 |
| 13.8 | 159 | 6 |
| 14.0 | 208 | 10 |
| 14.2 | 242 | 12.8 |
| 15.0 | 138 | 4.3 |
| 15.4 | 154 | 5.8 |
| 15.8 | 174 | 7.4 |
| 16.6 | 121 | 2.9 |
| 17.0 | 197 | 8.6 |
| 17.4 | 327 | 19.2 |
| 17.8 | 168 | 5.9 |
| 18.5 | 170 | 6 |
| 18.8 | 117 | 1.8 |
| 19.5 | 129 | 2.8 |
| 19.8 | 185 | 7.4 |
| 20.3 | 162 | 5.7 |
| 20.6 | 120 | 2.4 |
| 21.5 | 448 | 28.4 |
| 22.2 | 278 | 13.4 |
| 22.6 | 245 | 10.2 |
| 23.1 | 541 | 34.4 |
| 23.4 | 554 | 35.4 |
| 24.1 | 173 | 3.7 |
| 24.4 | 274 | 12.2 |
| 24.7 | 311 | 15.4 |
| 25.3 | 173 | 4.5 |
| 26.0 | 143 | 2.6 |
| 26.8 | 473 | 30.3 |
| 27.6 | 143 | 3.7 |
| 28.1 | 171 | 6.7 |
| 29.7 | 149 | 5.6 |
| 30.2 | 112 | 2.4 |
| 30.8 | 121 | 3.3 |
| 31.5 | 113 | 3.1 |
| 33.5 | 93 | 2.5 |
| 38.7 | 78 | 1.9 |

DSC/TGA thermograms of Form B of the mono-fumarate salt are depicted in FIG. 4.

The TGA thermogram of Form B of the mono-fumarate salt showed approximately 4.6% weight loss before 155° C., which was attributed to dehydration and the salt existed as a hydrate.

The DSC thermogram of Form B of the mono-fumarate salt showed broad endothermic peaks at 74° C., 116° C., and 126° C.

¹H NMR analysis of Form B of the mono-fumarate salt confirmed 1:1 stoichiometry of the acid and the base.

### Example 9: Preparation and Characterization of the Form C of the Mono-Fumarate Salt of the Compound of Formula (I)

The Form C of the mono-fumarate salt was obtained during the course of polymorph screening. The XRPD pattern of the Form C of the mono-fumarate salt is depicted in FIG. 5. The XRPD pattern is indexed in Table 9, which tabulates diffraction angles (2θ), intensity, and relative intensity (expressed as a percentage with respect to the most intense peak).

TABLE 9
Indexed XRPD Peak Table For the Form C of the Mono-
Fumarate Salt of the Compound of Formula (I)

| Diffraction Angles (2θ, °) | Intensity | Relative Intensity (%) |
|---|---|---|
| 5.2 | 1569 | 100.0 |
| 7.6 | 337 | 18.1 |
| 10.6 | 107 | 2.2 |
| 13.8 | 122 | 3.3 |
| 14.4 | 116 | 2.3 |
| 15.1 | 169 | 5.4 |
| 15.8 | 1321 | 91.4 |
| 16.7 | 224 | 9.0 |
| 17.3 | 150 | 3.7 |
| 17.9 | 133 | 3.0 |
| 19.4 | 237 | 9.2 |
| 19.9 | 171 | 3.1 |
| 20.6 | 199 | 4.2 |
| 21.2 | 1348 | 89.8 |
| 22.7 | 243 | 6.4 |
| 24.5 | 491 | 24.9 |
| 25.0 | 900 | 55.5 |
| 25.7 | 274 | 8.7 |
| 26.6 | 980 | 62.5 |
| 28.9 | 188 | 5.5 |
| 30.4 | 135 | 2.6 |
| 31.4 | 115 | 2.1 |
| 32.0 | 100 | 1.5 |
| 35.5 | 172 | 7.4 |

DSC/TGA thermograms of Form C of the mono-fumarate salt are depicted in FIG. 6.

The TGA thermogram of Form C of the mono-fumarate salt showed approximately 0.85% weight loss before 80° C., which was attributed to dehydration and the salt existed as a hydrate.

The DSC thermogram of Form C of the mono-fumarate salt showed broad endothermic peaks two broad endothermic peaks at 40° C. and 135° C.

¹H NMR analysis of Form C of the mono-fumarate salt confirmed 1:1 stoichiometry of the acid and the base.

### Example 10: Characterization of a Solid Material Comprising the Mono-Fumarate Salt of the Compound of Formula (I)

A solid material comprising the mono-fumarate salt of the compound of formula (I) was obtained during the solid screening of the mono-fumarate salt of the compound of formula (I) when 0.5 or 1.1 equiv of fumaric acid in MeOH at room temperature. The XRPD pattern of the solid material is depicted in FIG. 7. The XRPD pattern is indexed in Table 10, which tabulates diffraction angles (2θ), intensity, and relative intensity (expressed as a percentage with respect to the most intense peak).

TABLE 10
Indexed XRPD Peak Table For the Alternative Pattern of
the Mono-Fumarate Salt of the Compound of Formula (I)

| Diffraction Angles (2θ, °) | Intensity | Relative Intensity (%) |
|---|---|---|
| 5.4 | 451 | 35.0 |
| 10.9 | 107 | 3.3 |
| 16.4 | 844 | 100.0 |
| 16.8 | 259 | 19.0 |
| 23.9 | 244 | 11.7 |
| 27.5 | 343 | 26.8 |
| 28.7 | 222 | 13.2 |
| 38.9 | 87 | 2.4 |

### Example 11: Preparation and Characterization of the Form A of the Hemi-Fumarate Salt of the Compound of Formula (I)

The compound of formula (I) (1.5 g) was dissolved in 15 mL of IPA at 50° C. and 6 mL of fumaric acid/IPA solution (31.98 mg/mL) was added. The suspension became clear and no precipitation occurred after being cooled to room temperature. Precipitation was induced by introducing the Form A of the hemi-fumarate salt seeds. The resulting suspension was stirred at room temperature for one day and the salt was collected by filtration. The XRPD pattern of the Form A of the hemi-fumarate salt is depicted in FIG. 8. The XRPD pattern is indexed in Table 11, which tabulates diffraction angles (2θ), intensity, and relative intensity (expressed as a percentage with respect to the most intense peak).

Alternatively, the Form A of the hemi-fumarate salt was prepared by the following procedure: fumaric acid solid (1.265 g, 0.5 equiv) was dissolved into 63 mL IPA at 70° C. To this solution was added ~9 g of the compound of formula (I). The mixture became clear after stirring for approximately 10 minutes. The solution was stirred at 70° C. for further 20 minutes and cooled. A suspension of the Form A of the hemi-fumarate salt seeds in IPA (prepared by suspending 32 mg of hemi-fumarate salt Form A in 0.5 mL of IPA) was added at 49° C., which induced precipitation. After 2.5 hours, the suspension was finally cooled to room temperature and 63 mL of MTBE was added. The resulting suspension was stirred at room temperature for one day and the solid was collected by filtration. The wet cake was washed with 6 V IPA/MTBE (1:1). The filtrate was recovered, and dried at 50° C. under vacuum overnight.

TABLE 11
Indexed XRPD Peak Table For the
Form A of the Hemi-Fumarate Salt

| Diffraction Angles (2θ, °) | Intensity | Relative Intensity (%) |
|---|---|---|
| 6.4 | 293 | 3.8 |
| 8.3 | 623 | 14.0 |
| 9.9 | 1913 | 50.0 |
| 11.0 | 1195 | 30.0 |
| 12.1 | 255 | 3.8 |
| 13.2 | 566 | 12.6 |
| 14.3 | 201 | 2.4 |
| 14.5 | 175 | 1.6 |
| 15.1 | 152 | 1.0 |
| 16.1 | 387 | 7.1 |
| 16.9 | 816 | 18.6 |
| 17.2 | 3715 | 100.0 |
| 17.8 | 597 | 12.2 |
| 18.5 | 1121 | 27.0 |
| 19.5 | 421 | 7.3 |
| 19.8 | 415 | 7.1 |
| 20.2 | 539 | 10.6 |
| 21.2 | 197 | 1.0 |
| 21.5 | 338 | 4.9 |
| 22.1 | 629 | 13.0 |
| 22.5 | 712 | 15.4 |
| 23.3 | 359 | 5.7 |
| 24.3 | 440 | 7.9 |
| 24.5 | 295 | 3.7 |
| 25.0 | 419 | 7.2 |
| 25.5 | 569 | 11.4 |
| 26.3 | 448 | 8.0 |
| 27.2 | 1216 | 29.5 |
| 27.8 | 257 | 2.6 |
| 28.0 | 231 | 1.9 |
| 28.3 | 229 | 2.0 |
| 28.9 | 309 | 4.6 |
| 29.5 | 261 | 3.7 |
| 31.1 | 328 | 5.9 |
| 31.9 | 238 | 3.3 |
| 32.5 | 196 | 2.2 |
| 32.7 | 193 | 2.2 |
| 33.6 | 184 | 2.1 |
| 34.1 | 124 | 0.6 |
| 34.4 | 132 | 1.0 |
| 35.1 | 148 | 1.4 |
| 37.2 | 113 | 0.6 |
| 38.7 | 114 | 0.6 |
| 39.5 | 129 | 0.9 |

DSC/TGA thermograms of Form A of the hemi-fumarate salt are depicted in FIG. 9.

The TGA thermogram of Form A of the hemi-fumarate salt showed approximately 0.52% weight loss prior to 150° C. The batch of the Form A of the hemi-fumarate salt prepared by the alternative procedure exhibited approximately 0.2% weight loss prior to 130° C.

The DSC thermogram of Form A of the hemi-fumarate salt showed a broad endothermic peak at 207° C. (92 J/g) due to melting. The batch of the Form A of the hemi-fumarate salt prepared by the alternative procedure exhibited a melting point at 208° C. (98.2 J/g).

¹H NMR analysis of Form A of the hemi-fumarate salt confirmed 1:0.5 stoichiometry of the acid and the base, with approximately 0.35% of residual IPA in the sample. The batch of the Form A of the hemi-fumarate salt prepared by the alternative procedure existed as 1:0.5 stoichiometry of the acid and the base, with approximately 0.39% of residual IPA in the sample.

DVS analysis of Form A of the hemi-fumarate salt indicated slightly hygroscopic profile with 0.66% water uptake at 80% RH. The crystal form did not change after DVS analysis. The associated DVS isotherm plot of Form A of the hemi-fumarate salt is depicted in FIG. 10.

### Solubility Testing of Form A of the Hemi-Fumarate Salt of the Compound of Formula (I)

The solubility of the Form A of the hemi-fumarate salt was evaluated by visual assessment of samples upon solvent addition, employing organic solvents and water. Among the solvents, the Form A of the hemi-fumarate salt had the highest solubility in THF, MeOH, EtOH and acetone (>50 mg/mL). The results of the solubility testing are summarized in Table 12.

TABLE 12
Solubility of the Form A of the Hemi-Fumarate Salt
of the Compound of Formula (I) in Various Solvents

| Solvents | Solubility (mg/mL) |
|---|---|
| MeOH | >62.2 |
| EtOH | >53.4 |
| IPA | >17.8 |
| Isobutanol | >5.8 |
| 2-butanone | >27.5 |
| THF | >81.8 |
| MeCN | >3.2 |
| MTBE | <2.2 |
| Acetone | >50 |
| Water | <0.83 |
| Toluene | <0.96 |
| EtOAc | >9.8 |
| IPAc | >2.1 |
| Heptane | <0.63 |

“<” if dissolution was not observed.
“>” if dissolution occurred after addition of first aliquot of solvent.

### Example 12: Crystallization Behavior of the Hemi-Fumarate Salt of the Compound of Formula (I) from Single Solvent Systems

Slow evaporation of the Form A of the hemi-fumarate salt was carried out in organic solvents and water. The Form A of the hemi-fumarate salt (~10 mg) was added into 1 mL of different solvents respectively. Then the solutions or suspensions were filtered, and the filtrates were evaporated at ambient conditions. Most samples obtained by slow evaporation were glassy state. The results of the evaporation crystallization experiment are summarized in Table 13.

TABLE 13
Results of Evaporation Crystallization of the Form A of the Hemi-
Fumarate Salt of the Compound of Formula (I) in Single Solvent

| Solvents | Results |
|---|---|
| MeOH | Glassy state |
| EtOH | Glassy state |
| IPA | Glassy state |
| Isobutanol | Glassy state |
| 2-butanone | Glassy state |
| THF | Glassy state |
| MeCN | A little solid |
| MTBE | Amorphous |
| Acetone | Glassy state |
| Water | A little solid |
| Toluene | A little solid |
| EtOAc | New pattern |
| IPAc | Glassy state |
| Heptane | A little solid |

### Example 13: Crystallization Behavior of the Hemi-Fumarate Salt of the Compound of Formula (I) by Anti-Solvent Precipitation

Based on the solubility of the Form A of the hemi-fumarate salt, solvents and anti-solvents were chosen and nine experiments were conducted by precipitation method as shown in Table 14. The Form A of the hemi-fumarate salt (15-20 mg) was first dissolved in THF, MeOH or acetone to make a solution at room temperature, and then anti-solvents were added slowly. If precipitation occurred, resultant solids were characterized by XRPD (Example 1, XRPD). New crystal forms were obtained by precipitation in MeOH/water or THF/MTBE, which were respectively assigned as the Form B of the hemi-fumarate salt of the compound of formula (I) (Form B) and the free base form of the compound of formula (I) (FBF). The Form A of the hemi-fumarate salt easily dissociated in these conditions.

TABLE 14
Results of the Anti-Solvent Precipitation of the Form A
of the Hemi-Fumarate Salt of the Compound of Formula (I)

| | Solvent | Anti- | | | |
|---|---|---|---|---|---|
| Solvent | volume (V) | solvent | Ratio | Observation | Result |
| THF | 6.5 | Heptane | 1:2 | I | Form A |
| THF | 6.3 | Water | 1:8 | I | Form B + FBF |
| THF | 6.8 | MTBE | 1:15 | II | Low crystallinity |
| MeOH | 9.5 | MTBE | 1:20 | III | Clear solution |
| MeOH | 10.9 | Water | 1:1 | IV | Low crystallinity |
| Acetone | 16.7 | Water | 1:1 | IV | Form B + FBF |
| Acetone | 16.4 | Heptane | 4:5 | IV | Low crystallinity |
| Acetone | 16.1 | MTBE | 1:12 | III | Clear solution |
| Acetone | 17.1 | IPAc | 1:10 | III | Clear solution |

I = Precipitation appeared after stirring at room temperature for 20 minutes.
II = Precipitation appeared after stirring overnight.
III = The solution was still clear after stirring overnight.
IV = Precipitation appeared immediately.

### Example 14: Slurry Study of the Hemi-Fumarate Salt of the Compound of the Formula (I)

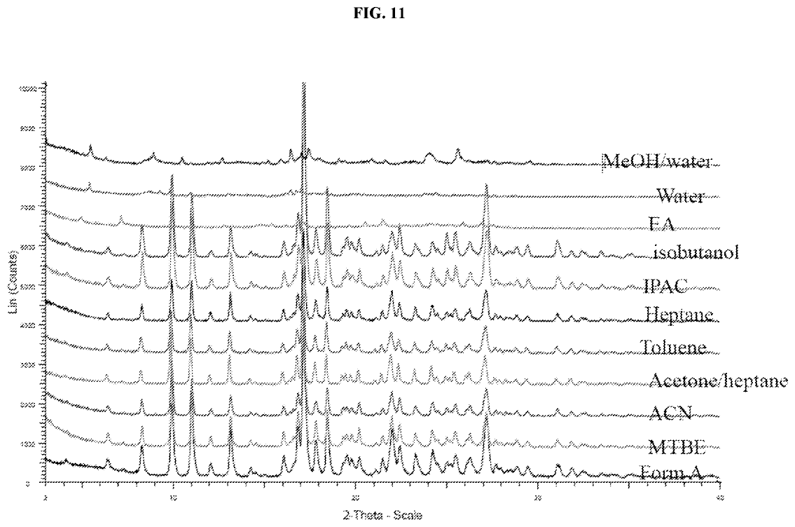

Slurry studies of the Form A of the hemi-fumarate salt were conducted at room temperature for four days and at 50° C. for one day. About 10 mg to 35 mg of the Form A of the hemi-fumarate salt was slurried in different solvents at RT or 50° C. for one to four days. Solid samples were collected by filtration and analyzed by XRPD (Example 1, XRPD) at a specific time. Most of the samples obtained by slurry study remained unchanged as the Form A. The samples obtained slurry study with water and MeOH/water at 50° C. were a mixture of the Form B of the hemi-fumarate salt of the compound of formula (I) (Form B) and the free base Form of the compound of formula (I) (FBF). The results are summarized in Table 15. The overlay of XRPD patterns from the slurry study of the Form A of the hemi-fumarate salt of the compound of formula (I) is depicted in FIG. 11.

TABLE 15
Slurry Study of the Form A of the Hemi-Fumarate
Salt of the Compound of Formula (I)

| | Slurry at room temperature | | Slurry at 50° C. | |
|---|---|---|---|---|
| | (4 days) | | (1 day) | |
| | Solvent | | Solvent | XRPD |
| Solvent | Volume | XRPD Result | Volume | Result |
| Water | 49.5 V | Low | 18.5 V | Form B + |
| | | crystallinity | | FBF |
| MTBE | 42.4 V | Form A | 18.3 V | Form A |
| MeCN | 39.4 V | Form A | 20.3 V | Form A |
| Heptane | 37.3 V | Form A* | 17.6 V | Form A |
| Toluene | 38.2 V | Form A* | 16.8 V | Form A |
| IPAc | 28.7 V | Form A* | 19.1 V | Form A |
| EtOAc | 38.2 V | Low | 17.6 V | Form A |
| | | crystallinity | | |
| Isobutanol | 19.6 V | Form A* | 8.4 V | Form A |
| Acetone/heptane | 48.5 V | Form A | 13.7 V | Form A |
| (1:4) | | | | |
| MeOH/water (1:4) | 43.9 V | Low | 16.2 V | Form B + |
| | | crystallinity | | FBF |

*The samples were slurried at room temperature for three days.

### Example 15: Crystallization Behavior of the Hemi-Fumarate Salt of the Compound of Formula (I) Via Cooling Crystallization

Cooling crystallizations of the Form A of the hemi-fumarate salt were carried out in MeCN, EtOAc, and isobutanol at 50° C. The Form A of the hemi-fumarate salt (~20 mg) was dissolved in MeCN, EtOAc or isobutanol at 50° C. Then the clear solution was cooled to room temperature, slowly with stirring. Any obtained solid was characterized accordingly with XRPD (Example 1, XRPD). New pattern was obtained by crystallization in EtOAc, which exhibited low crystallinity. The results of the cooling crystallization experiments are summarized in Table 16.

TABLE 16
Results of the Cooling Crystallization Experiment of the For A
of the Hemi-Fumarate Salt of the Compound of the Formula (I)

| | Solvent | | |
|---|---|---|---|
| Solvent | Volume | Observation | Result |
| MeCN | 103 V | Precipitation appeared | Form A |
| | | after stirring at room | |
| | | temperature for 1 hour | |
| EtOAc | 59 V | Precipitation appeared | Low |
| | | after stirring overnight | crystallinity |
| | | and concentration | |
| Isobutanol | 38 V | The solution was still clear | Clear solution |
| | | after stirring overnight | |

### Mechanical Treatment of the Form A of the Hemi-Fumarate Salt of the Compound of Formula (I)

Appropriate amount of the Form A of the hemi-fumarate salt was ground for about two and five minutes manually and the samples were analyzed by XRPD (Example 1, XRPD). The Form A of the hemi-fumarate salt remained unchanged and no new form was obtained by grinding. The crystallinity became low after grinding for five minutes. Solubility of the Form A of the Hemi-Fumarate Salt of the Compound of Formula (I) in Biologically Relevant Media

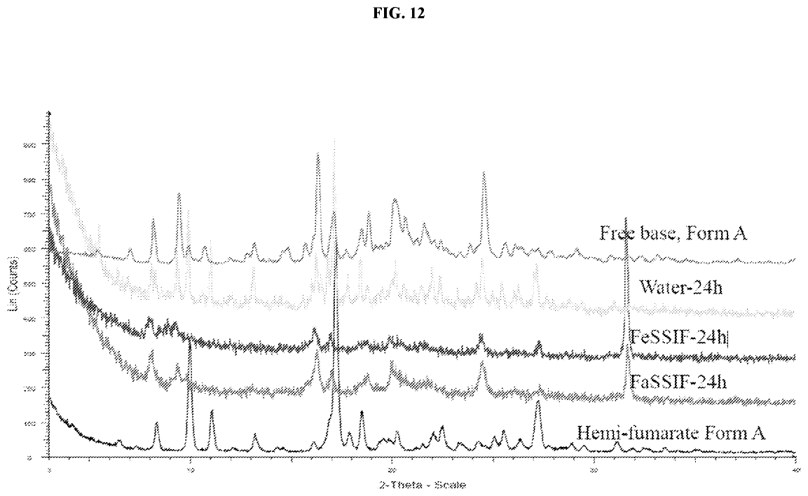

The solubility of the Form A of the hemi-fumarate salt was evaluated in water and biologically relevant media, SGF, FeSSIF, and FaSSIF. About 10 mg of the sample was weighed into sample vials and then 2 mL of three bio-relevant media and water were added to each respective sample to make suspensions. All suspensions were shaken at 37° C. with 200 rpm. At 0.5, 2 and 24 hours, about 600 μL of each suspension was filtered and the filtrate was analyzed by HPLC (Example 1, HPLC) to test the solubility. pH of the filtrate was measured, and the filter cake was analyzed by XRPD (Example 1, XRPD). The Form A of the hemi-fumarate salt was more soluble in SGF than in FeSSIF, FaSSIF, and water. Furthermore, the Form A of the hemi-fumarate salt in the latter three solvents exhibited dissociation into the free base Form, which was confirmed by XRPD analysis. No XRPD pattern change was observed with the salt in SGF, which suggested no dissociation into the free base Form. The summary of the solubility test is tabulated in Table 17. The XRPD pattern from the solubility test of the Form A of the hemi-fumarate salt of the compound of formula (I) is depicted in FIG. 12.

TABLE 17
Results of the Solubility Test of the Form A of the Hemi-Fumarate Salt of the
Compound of Formula (I)

| | Solubility (mg/mL) | | | pH | | | XRPD |
|---|---|---|---|---|---|---|---|
| Media | 0.5 | 2 | 24 | 0.5 | 2 | 24 | 24 h |
| SGF | 4.294 | 4.227 | 4.368 | 1.12 | 1.10 | 1.36 | N/A |
| FeSSIF | 0.214 | 0.149 | 0.041 | 5.14 | 4.88 | 4.71 | Changed |
| FaSSIF | 0.022 | 0.016 | 0.029 | 6.39 | 6.36 | 5.57 | Changed |
| Water | 0.010 | 0.081 | 0.081 | 3.77 | 2.97 | 2.91 | Changed |

### Stability of the Form A of the Hemi-Fumarate Salt of the Compound of Formula (I)

The physical stability of the Form A of the hemi-fumarate salt was evaluated under high humidity (92.5% RH) for 11 days. No form change was detected by XRPD analysis, suggesting that the Form A of the hemi-fumarate salt was physically stable under high humidity.

Appropriate amount of the Form A of the hemi-fumarate salt was placed at 40° C. (75% RH) and 60° C. for several days. The solids were analyzed by XRPD (Example 1, XRPD) and HPLC (Example 1, HPLC) at appropriate time points. The physical and chemical stabilities of the Form A of the hemi-fumarate salt was conducted at 60° C. and at 40° C. (75% RH) for 29 days. No obvious degradation occurred and no form change was observed under testing conditions. Thus, the Form A of the hemi-fumarate salt was considered physically and chemically stable at 60° C. and at 40° C. (75% RH) for 29 days. The results of the stability test is summarized in Table 18.

TABLE 18
Results of the Stability Evaluation of the Form A of
the Hemi-Fumarate Salt of the Compound of Formula (I)

| Condition | XRPD - 29 days | Purity (%) - 8 days/29 days |
|---|---|---|
| Starting material | Form A | 99.87 |
| 40° C. (75% RH) | No Change | 99.86/99.92 |
| 60° C. | No Change | 99.86/99.91 |

### Example 16: Preparation and Characterization of the Form B of the Hemi-Fumarate Salt of the Compound of Formula (I)

The Form B of the hemi-fumarate salt was obtained during the course of the polymorph screening via precipitation in MeOH/water. The crystallinity of the Form B of the hemi-fumarate salt was very low. The XRPD pattern of the Form B of the hemi-fumarate salt is depicted in FIG. 13. The XRPD pattern is indexed in Table 19, which tabulates diffraction angles (2θ), intensity, and relative intensity (expressed as a percentage with respect to the most intense peak).

TABLE 19
Indexed XRPD Peak Table For the Form B of the Hemi-
Fumarate Salt of the Compound of Formula (I)

| Diffraction Angles (2θ, °) | Intensity | Relative Intensity (%) |
|---|---|---|
| 5.4 | 651 | 100 |
| 8.7 | 315 | 44.8 |
| 10.4 | 161 | 9.1 |
| 11.0 | 155 | 8.4 |
| 11.9 | 170 | 10.8 |
| 12.6 | 181 | 12.8 |
| 16.5 | 449 | 77 |
| 16.9 | 374 | 57 |
| 17.4 | 366 | 53.1 |
| 17.9 | 306 | 37 |
| 23.9 | 337 | 40.1 |
| 25.7 | 280 | 25.2 |
| 27.7 | 193 | 8.6 |
| 34.1 | 128 | 8.2 |

DSC/TGA thermograms of Form B of the hemi-fumarate salt are depicted in FIG. 14.

The TGA thermogram of Form B of the hemi-fumarate salt showed approximately 1% weight loss prior to 150° C.

The DSC thermogram of Form B of the hemi-fumarate salt showed three endothermic peak at 128° C. due to dehydration, and 186° C. and 202° C. due to melting.

¹H NMR analysis of Form B of the hemi-fumarate salt showed no residual sample.

### Example 17: Inter-conversion Study of the Hemi-Fumarate Salt of the Compound of Formula (I)

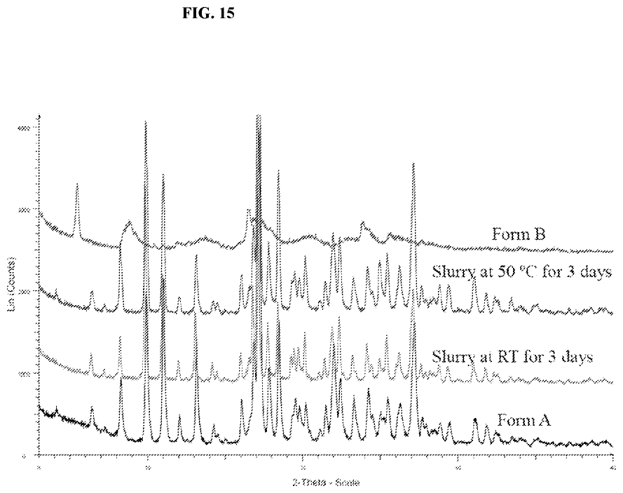

Equal amount of the Form A of the hemi-fumarate salt and the Form B of the hemi-fumarate salt were added into IPA, and the suspensions were stirred at RT or 50° C. for 3 days, respectively. Residual solids were obtained and characterized. The competitive slurry of the Form A and the Form B of the hemi-fumarate salts showed that all mixtures transformed to Form A at 50° C. and room temperature in IPA. Thus, the Form A of the hemi-fumarate salt was the more stable form. The overlay of the XRPD pattern from the inter-conversion study of the hemi-fumarate salt is depicted in FIG. 15.

### Example 18: Salt Screening of the Compound of Formula (I) in the 96-Well Plate

The salt screening of the compound of formula (I) was carried out by first preparing twelve acid solutions, in which the acids were dissolved in MeOH to prepare 0.1 M acid/methanolic solution. Separately, the compound of formula (I) (330.6 mg) was dissolved in 11 mL of MeOH/DCM (1:1) to prepare the drug solution at ~30 mg/mL. The resulting drug solution was distributed into the 96-well plate. Each well contained 100 μL of drug solution. Then, each of the prepared acid solution (1.1 equiv) was added into the well according to the acid/solvent matrix depicted in Table 20. After evaporation to dryness, 200 μL of selected solvents were added into each well, and the wells were covered with a film that has been puncture to make pinhole, then evaporated under ambient conditions. The solid samples with sufficient quantity were analyzed by XRPD (Example 1, XRPD) and ¹H NMR (Example 1, 1H NMR).

The compound of formula (I) formed salts with 11 acids and almost no chemical shifts were detected in the sample containing succinic acid. The XRPD analysis of the resulting salts showed that crystalline samples were obtained with p-TsOH, oxalic acid, fumaric acid, tartaric acid, H₃PO₄ acid and succinic acid.

TABLE 20
The 96-Well Plate Salt Screening Acid/Solvent Matrix

| | | Solvent | | | | | | | |
|---|---|---|---|---|---|---|---|---|---|
| | | A | B | C | D | E | F | G | H |
| Acid | | MeOH | IPA | THF | MeCN | MTBE | Acetone | H₂O | EtOAc |
| 1 | HCl | | | | | | | | |
| 2 | HBr | | | | | | | | |
| 3 | H₂SO₄ | | | | | | | | |
| 4 | p-TsOH | | | | | | | | S |
| 5 | MsOH | | | | | | | | |
| 6 | Oxalic acid | | | | | | S | | S |
| 7 | H₃PO₄ | S | S | S | S | S | S | S | S |
| 8 | Maleic acid | | | | | | | | |
| 9 | L-tartaric acid | | S | S | S | S | S | S | S |
| 10 | Fumaric acid | S | | S | S | S | S | S | S |
| 11 | Citric acid | | | | | | | | |
| 12 | Succinic acid | S | | | | | | | |

“'S” represents solid. Samples obtained in other wells were glassy state.

### Example 19: Preparation and Characterization of the L-Tartrate Salt of the Compound of Formula (I)

The L-tartrate salt was prepared using the salt preparation procedure outlined in Example 18. The acid was added at 50° C. and approximately 13 V MTBE was added. The solution was still clear after cooling to room temperature. The solution was stirred at room temperature for 2 hours and the seeds from the 96-well experiment (Example 9) were added. A suspension was immediately formed. The resulting suspension was stirred for additional 2 hours and the solid was collected by filtration. The XRPD pattern of the L-tartrate salt is depicted in FIG. 16. The XRPD pattern is indexed in Table 21, which tabulates diffraction angles (2θ), intensity, and relative intensity (expressed as a percentage with respect to the most intense peak).

TABLE 21
Indexed XRPD Peak Table For the L-Tartrate
Salt of the Compound of Formula (I)

| Diffraction Angles (2θ, °) | Intensity | Relative Intensity (%) |
|---|---|---|
| 3.3 | 1565 | 100 |
| 7.9 | 397 | 26.1 |
| 10.2 | 192 | 8.9 |
| 11.6 | 139 | 4.2 |
| 12.4 | 121 | 1.9 |
| 14.4 | 259 | 14.1 |
| 14.8 | 190 | 6.5 |
| 15.4 | 188 | 6 |
| 17.0 | 212 | 7.4 |
| 20.6 | 246 | 8.9 |
| 23.4 | 194 | 4 |
| 25.1 | 239 | 8.6 |
| 31.2 | 112 | 2.4 |

DSC/TGA thermograms of the L-tartrate salt are depicted in FIG. 17.

The TGA thermogram of the L-tartrate salt showed approximately 2.4% weight loss prior to 150° C.

The DSC thermogram of the L-tartrate salt showed an endothermic peak at 117° C.

¹H NMR analysis of the L-tartrate salt showed 1:0.75 stoichiometry of the base to acid, and the sample contained 1.7% MTBE and 0.5% acetone. Thus, the sample was considered a solvate.

### Example 20: Preparation and Characterization of the Form A of the Tosylate Salt of the Compound of Formula (I)

The Form A of the tosylate salt was prepared using the salt preparation procedure outlined in Example 18. IPAc was used the anti-solvent. after concentrating the mixture at room temperature and adding 9 V of IPAc, little precipitation was observed. After stirring for four hours, the solid was collected by filtration. The XRPD pattern of the Form A of the tosylate salt is depicted in FIG. 20. The XRPD pattern is indexed in Table 22, which tabulates diffraction angles (2θ), intensity, and relative intensity (expressed as a percentage with respect to the most intense peak).

TABLE 22
Indexed XRPD Peak Table For the Form A of the
Tosylate Salt of the Compound of Formula (I)

| Diffraction Angles (2θ, °) | Intensity | Relative Intensity (%) |
|---|---|---|
| 5.053 | 3358 | 100 |
| 6.106 | 747 | 14.3 |
| 6.594 | 388 | 3.4 |
| 7.349 | 289 | 2.2 |
| 8.929 | 146 | 1 |
| 10.175 | 150 | 1.8 |
| 11.242 | 133 | 1.3 |
| 11.819 | 138 | 1.4 |
| 12.036 | 156 | 1.9 |
| 12.593 | 218 | 3.9 |
| 13.102 | 130 | 1 |
| 13.84 | 123 | 1 |
| 15.581 | 171 | 2.7 |
| 16.281 | 120 | 1 |
| 17.093 | 133 | 0.9 |
| 17.264 | 198 | 2.9 |
| 17.619 | 266 | 4.9 |
| 20.49 | 560 | 13.7 |
| 21.032 | 204 | 1.7 |
| 21.42 | 180 | 1 |
| 21.835 | 242 | 3.1 |
| 25.436 | 163 | 1.6 |
| 26.813 | 145 | 1.6 |
| 31.962 | 78 | 0.5 |

DSC/TGA thermograms of Form A of Form A of the tosylate salt are depicted in FIG. 19.

The TGA thermogram of Form A of the tosylate salt showed approximately 5.1% weight loss prior to 170° C.

The DSC thermogram of Form A of the tosylate salt showed an endothermic peak at 126° C.

¹H NMR analysis of the Form A of the tosylate salt showed 1:1 stoichiometry of the base to acid and the sample contained 7% IPAc. Thus, the sample was considered an IPAc solvate.

### Example 21: Characterization of a Solid Material Comprising the Tosylate Salt of the Compound of Formula (I)

A solid material comprising the tosylate salt of the compound of formula (I) was found during the course of the salt-screening experiment (Example 18), wherein 1.1 equiv of 0.1 M p-toluenesulfonic acid in MeOH was added to a solution of the compound of formula (I) in 1:1 solution of MeOH/DCM, prepared at ~30 mg/mL. The XRPD pattern of the tosylate salt of the compound of formula (I) is depicted in FIG. 20. The XRPD pattern is indexed in Table 23, which tabulates diffraction angles (2θ), intensity, and relative intensity (expressed as a percentage with respect to the most intense peak).

TABLE 23
Indexed XRPD Peak Table For the First Pattern of
the Tosylate Salt of the Compound of Formula (I)

| Diffraction Angles (2θ, °) | Intensity | Relative Intensity (%) |
|---|---|---|
| 5.495 | 6921 | 100.0 |
| 7.207 | 290 | 1.0 |
| 9.295 | 157 | 0.7 |
| 11.058 | 280 | 2.9 |
| 12.015 | 137 | 0.6 |
| 12.956 | 1044 | 14.4 |
| 13.396 | 183 | 1.3 |
| 14.483 | 155 | 1.0 |
| 14.686 | 301 | 3.2 |
| 16.632 | 289 | 2.8 |
| 17.411 | 784 | 10.1 |
| 18.509 | 150 | 0.5 |
| 19.272 | 497 | 5.7 |
| 20.128 | 287 | 2.2 |
| 20.324 | 838 | 10.5 |
| 20.893 | 696 | 8.2 |
| 21.622 | 281 | 1.7 |
| 22.262 | 1784 | 24.5 |
| 23.129 | 767 | 9.0 |
| 23.756 | 670 | 7.4 |
| 24.324 | 1117 | 14.2 |
| 24.69 | 270 | 1.4 |
| 25.108 | 398 | 3.5 |
| 25.705 | 675 | 7.8 |
| 26.075 | 223 | 1.0 |
| 26.41 | 345 | 2.9 |
| 26.854 | 310 | 2.5 |
| 27.246 | 178 | 0.7 |
| 27.902 | 197 | 1.1 |
| 28.603 | 320 | 2.9 |
| 28.963 | 158 | 0.4 |
| 29.257 | 168 | 0.6 |
| 29.645 | 169 | 0.6 |
| 30.24 | 162 | 0.7 |
| 30.843 | 275 | 2.5 |
| 32.187 | 112 | 0.3 |
| 32.612 | 139 | 0.7 |
| 33.682 | 183 | 1.4 |
| 35.542 | 115 | 0.4 |
| 36.255 | 227 | 2.1 |
| 36.743 | 116 | 0.4 |
| 38.277 | 94 | 0.4 |
| 38.785 | 106 | 0.4 |
| 39.537 | 108 | 0.4 |

### Example 22: Characterization of a Solid Material Comprising the Tosylate Salt of the

### Compound of Formula (I)

Another solid material comprising the tosylate salt of the compound of formula (I) was isolated when 14 V of EtOAc anti-solvent and a seed of the tosylate salt obtained from the salt screening experiment (Example 10) were added to a methanolic solution of the compound of formula (I) and p-toluenesulfonic acid. The XRPD pattern of this pattern of the tosylate salt of the compound of formula (I) is depicted in FIG. 21. The XRPD pattern is indexed in Table 24, which tabulates diffraction angles (2θ), intensity, and relative intensity (expressed as a percentage with respect to the most intense peak).

TABLE 24
Indexed XRPD Peak Table For the Second Alternative Pattern
of the Tosylate Salt of the Compound of Formula (I)

| Diffraction Angles (2θ, °) | Intensity | Relative Intensity (%) |
|---|---|---|
| 5.5 | 1119 | 72.2 |
| 6.1 | 1444 | 100.0 |
| 6.6 | 327 | 12.9 |
| 8.2 | 129 | 2.1 |
| 9.0 | 109 | 1.7 |
| 11.3 | 175 | 5.7 |
| 12.0 | 164 | 4.4 |
| 12.4 | 148 | 3.0 |
| 13.0 | 215 | 8.4 |
| 13.8 | 171 | 5.5 |
| 14.6 | 156 | 4.5 |
| 15.1 | 128 | 1.6 |
| 15.6 | 266 | 11.8 |
| 16.3 | 164 | 2.9 |
| 17.5 | 281 | 10.5 |
| 17.9 | 275 | 9.3 |
| 18.7 | 254 | 6.9 |
| 19.2 | 244 | 5.8 |
| 20.1 | 251 | 5.7 |
| 20.3 | 287 | 8.2 |
| 20.6 | 308 | 9.6 |
| 20.8 | 304 | 9.2 |
| 22.2 | 338 | 10.9 |
| 23.1 | 323 | 9.5 |
| 23.7 | 270 | 5.4 |
| 24.3 | 347 | 11.9 |
| 25.1 | 232 | 3.6 |
| 25.7 | 237 | 5.1 |
| 26.8 | 191 | 3.8 |
| 28.1 | 154 | 3.0 |
| 28.5 | 147 | 2.8 |
| 29.4 | 141 | 2.7 |
| 30.8 | 121 | 1.5 |
| 33.6 | 111 | 2.1 |
| 36.3 | 96 | 1.4 |

### Example 23: Preparation and Characterization of the Form A of the Succinate Salt of the Compound of Formula (I)

The Form A of the succinate salt was prepared using the salt preparation procedure outlined in Example 18, using 1 or 2 equiv of acid. Precipitation occurred immediately after adding the seed from the 96-well plate experiment (Example 18). After stirring for one hour, the solid was collected by filtration. The XRPD pattern of Form A of the succinate salt is depicted in FIG. 22. The XRPD pattern is indexed in Table 25, which tabulates diffraction angles (2θ), intensity, and relative intensity (expressed as a percentage with respect to the most intense peak).

TABLE 25
Indexed XRPD Peak Table For the Form A of the
Succinate Salt of the Compound of Formula (I)

| Diffraction Angles (2θ, °) | Intensity | Relative Intensity (%) |
|---|---|---|
| 7.606 | 1411 | 100.0 |
| 9.961 | 127 | 3.4 |
| 10.574 | 116 | 2.9 |
| 10.957 | 160 | 6.3 |
| 13.045 | 321 | 18.8 |
| 13.644 | 561 | 37.0 |
| 14.125 | 109 | 2.2 |
| 15.379 | 304 | 17.0 |
| 16.213 | 427 | 25.9 |
| 16.445 | 276 | 14.0 |
| 17.203 | 668 | 43.7 |
| 17.576 | 829 | 55.8 |
| 18.252 | 391 | 22.1 |
| 19.385 | 150 | 3.2 |
| 19.624 | 176 | 5.0 |
| 19.953 | 361 | 19.0 |
| 21.144 | 768 | 49.3 |
| 21.803 | 457 | 25.1 |
| 22.051 | 377 | 18.9 |
| 22.326 | 297 | 12.8 |
| 22.608 | 868 | 56.7 |
| 22.852 | 1177 | 80.6 |
| 23.255 | 268 | 11.0 |
| 23.443 | 256 | 10.2 |
| 24.517 | 685 | 43.7 |
| 24.791 | 266 | 11.4 |
| 25.109 | 158 | 3.0 |
| 25.619 | 175 | 4.2 |
| 25.859 | 193 | 5.6 |
| 26.247 | 488 | 28.4 |
| 26.515 | 550 | 33.4 |
| 26.79 | 246 | 10.2 |
| 27.029 | 218 | 8.2 |
| 27.223 | 206 | 7.5 |
| 27.507 | 218 | 8.8 |
| 27.725 | 240 | 10.8 |
| 28.643 | 127 | 2.8 |
| 29.747 | 235 | 11.2 |
| 30.262 | 183 | 7.2 |
| 30.823 | 141 | 4.2 |
| 31.009 | 198 | 8.7 |
| 31.29 | 126 | 3.5 |
| 31.458 | 124 | 3.5 |
| 32.232 | 120 | 3.8 |
| 32.922 | 99 | 2.6 |
| 33.329 | 103 | 2.8 |
| 33.925 | 105 | 2.8 |
| 34.826 | 98 | 2.4 |
| 35.815 | 133 | 5.0 |
| 36.511 | 115 | 3.7 |
| 36.797 | 95 | 2.3 |
| 37.044 | 114 | 3.6 |
| 37.368 | 94 | 2.0 |
| 39.86 | 104 | 2.2 |

DSC/TGA thermograms of Form A of the succinate salt are depicted in FIG. 23.

The TGA thermogram of Form A of the succinate salt showed approximately 0.8% weight loss prior to 80° C.

The DSC thermogram of Form A of the succinate salt showed an endothermic peak at 87° C.

¹H NMR analysis of the Form A of the succinate salt showed likely less than 1:2 stoichiometry of the base to acid.

### Example 24: Characterization of a Solid Material Comprising the Succinate Salt of the Compound of Formula (I)

A solid material comprising the succinate salt of the compound of formula (I) was found during the course of the salt-screening experiment (Example 18), wherein 1.1 equiv of 0.1 M succinic acid solution in MeOH was added to a solution of the compound of formula (I) in 1:1 mixture of MeOH/DCM, prepared at ~30 mg/mL. The XRPD pattern of this pattern of the succinate salt of the compound of formula (I) is depicted in FIG. 24. The XRPD pattern is indexed in Table 26, which tabulates diffraction angles (2θ), intensity, and relative intensity (expressed as a percentage with respect to the most intense peak).

TABLE 26
Indexed XRPD Peak Table For the First Pattern Comprising
the Succinate Salt of the Compound of Formula (I)

| Diffraction Angles (2θ, °) | Intensity | Relative Intensity (%) |
|---|---|---|
| 6.3 | 280 | 9.2 |
| 8.9 | 1507 | 96.1 |
| 10.4 | 1080 | 67.7 |
| 12.3 | 118 | 3.0 |
| 12.6 | 529 | 31.1 |
| 13.9 | 83 | 1.2 |
| 14.5 | 93 | 1.7 |
| 14.9 | 225 | 10.5 |
| 15.2 | 439 | 25.0 |
| 15.9 | 392 | 21.8 |
| 16.7 | 189 | 7.8 |
| 17.0 | 1547 | 100.0 |
| 17.4 | 781 | 47.9 |
| 17.9 | 181 | 7.2 |
| 19.1 | 307 | 15.5 |
| 19.3 | 284 | 13.8 |
| 19.9 | 820 | 50.1 |
| 20.6 | 203 | 8.0 |
| 20.8 | 353 | 18.1 |
| 21.6 | 359 | 18.4 |
| 21.9 | 1525 | 97.8 |
| 22.7 | 156 | 5.0 |
| 23.2 | 163 | 5.6 |
| 24.2 | 377 | 20.3 |
| 25.6 | 668 | 40.2 |
| 26.2 | 170 | 6.3 |
| 26.7 | 155 | 5.4 |
| 27.0 | 759 | 46.7 |
| 28.2 | 140 | 4.9 |
| 28.5 | 113 | 3.0 |
| 29.5 | 259 | 13.2 |
| 30.0 | 127 | 4.6 |
| 30.9 | 105 | 3.4 |
| 32.2 | 362 | 20.6 |
| 33.1 | 114 | 3.7 |
| 34.1 | 92 | 2.5 |
| 35.3 | 103 | 3.1 |
| 35.7 | 105 | 3.1 |
| 36.1 | 102 | 2.9 |
| 37.4 | 108 | 3.5 |
| 37.6 | 136 | 5.3 |
| 38.4 | 113 | 3.8 |
| 39.4 | 83 | 2.0 |
| 39.6 | 81 | 2.0 |

### Example 25: Characterization of a Solid Material Comprising the Succinate Salt of the Compound of Formula (I)

Another solid material comprising the succinate salt of the compound of formula (I) was found during the course of the salt-screening experiment (Example 9), wherein 1.05 equiv of 0.1 M succinic acid solution in MeOH was added to a solution of the compound of formula (I) in 1:1 mixture of MeOH/DCM, prepared at ~30 mg/mL, then adding the seed of a succinate salt from the salt-screening experiment. The mixture was slurried overnight and the solid was collected by filtration. The XRPD pattern of this pattern of the succinate salt of the compound of formula (I) is depicted in FIG. 25. The XRPD pattern is indexed in Table 27, which tabulates diffraction angles (2θ), intensity, and relative intensity (expressed as a percentage with respect to the most intense peak).

TABLE 27
Indexed XRPD Peak Table For the Second Pattern Comprising
the Succinate Salt of the Compound of Formula (I)

| Diffraction Angles (2θ, °) | Intensity | Relative Intensity (%) |
|---|---|---|
| 6.3 | 214 | 7.6 |
| 9.0 | 376 | 47.4 |
| 10.5 | 330 | 39.6 |
| 12.4 | 139 | 7.4 |
| 12.7 | 282 | 32.2 |
| 14.6 | 143 | 7.1 |
| 15.0 | 216 | 18.7 |
| 15.3 | 266 | 26.7 |
| 16.0 | 300 | 30.8 |
| 17.1 | 497 | 61.8 |
| 17.5 | 602 | 79.1 |
| 18.1 | 205 | 8.6 |
| 19.2 | 267 | 16.6 |
| 19.4 | 309 | 23.5 |
| 20.7 | 256 | 12.6 |
| 21.0 | 335 | 26.1 |
| 21.7 | 341 | 27.2 |
| 22.9 | 212 | 5.6 |
| 23.3 | 216 | 6.5 |
| 24.2 | 492 | 55.9 |
| 25.7 | 727 | 100.0 |
| 26.3 | 260 | 20.1 |
| 26.9 | 169 | 6.2 |
| 27.3 | 278 | 26.5 |
| 28.4 | 176 | 10.7 |
| 28.7 | 162 | 8.3 |
| 29.0 | 173 | 10.5 |
| 29.7 | 312 | 35.5 |
| 30.2 | 153 | 8.9 |
| 31.0 | 135 | 7.0 |
| 32.1 | 132 | 6.9 |
| 32.5 | 155 | 10.9 |
| 33.2 | 163 | 12.4 |
| 35.4 | 135 | 8.5 |
| 35.8 | 132 | 7.9 |
| 36.3 | 113 | 4.7 |
| 37.5 | 115 | 5.8 |
| 38.6 | 147 | 11.6 |
| 39.6 | 109 | 4.8 |

### Example 26: Analytical Methods and General Procedures for Characterizing and Preparing Forms A, B, C, D, E and H, Materials F and G of the Compound of Formula (I), Crystalline Camsylate Salt Form A of the Compound of Formula (I), and Phosphate Salt Materials A and B of the Compound of Formula (I)

### Analytical Methods

### (1) X-ray Powder Diffraction (XRPD)

### Transmission Mode

XRPD patterns were collected with a PANalytical X'Pert PRO MPD or a PANalytical Empyrean diffractometer using an incident beam of Cu radiation produced by an Optix long, fine-focus source. An elliptically graded multilayer mirror was used to focus Cu Kα X-rays through the specimen and onto the detector. Prior to the analysis, a silicon specimen (NIST SRM 640e) was analyzed to verify that the observed position of the Si 111 peak is consistent with the NIST-certified position. A specimen of the sample was sandwiched between 3 μm-thick films and analyzed in transmission geometry. A beam-stop, short anti-scatter extension, and anti-scatter knife edge were used to minimize the background generated by air. Soller slits for the incident and diffracted beams were used to minimize broadening and asymmetry from axial divergence. Diffraction patterns were collected using a scanning position-sensitive detector (X'Celerator) located 240 mm from the specimen and Data Collector software v. 5.5.

### Reflection Geometry Mode

For samples in limited quantity, the following protocol was implemented: XRPD patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify that observed position of the Si 111 peak is consistent with the NIST-certified position. A specimen of the sample was prepared as a thin, circular layer centered on a silicon zero-background substrate. Anti-scatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 5.5. The data acquisition parameters for each pattern are disclosed, including the DS and the incident-beam SS.

### (2) Variable Temperature X-ray Powder Diffraction (VT-XRPD)

XRPD patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Data were collected and analyzed using Data Collector software v. 5.5. Prior to the analysis, a silicon specimen (NIST SRM 640e) was analyzed to verify the observed position of the Si 111 peak is consistent with the NIST-certified position. A specimen of the sample was packed in a nickel-coated copper well. Anti-scatter slits (SS) were used to minimize the background generated by air scattering. Soller slits for the incident and diffracted beams were used to minimize broadening from axial divergence. Diffraction patterns were collected using a scanning position-sensitive detector (X'Celerator) located 240 mm from the sample. The data acquisition parameters for each pattern are disclosed, including the divergence slit (DS) and the incident-beam SS.

An Anton Paar TTK 450 stage was used to collect in-situ XRPD patterns as a function of temperature. The sample was heated with a resistance heater located directly under the sample holder, and the temperature was monitored with a platinum-100 resistance sensor located in the specimen holder. The heater was powered and controlled by an Anton Paar TCU 100 interfaced with Data Collector.

### (3) Single Crystal X-Ray Diffraction

### Data Collection

A colorless plate having approximate dimensions of 0.2×0.14×0.02 mm³ was mounted on a polymer loop in random orientation. Preliminary examination and data collection were performed on a Rigaku SuperNova diffractometer, equipped with a copper anode microfocus sealed X-ray tube (Cu Kα λ=1.54184 Δ) and a Dectris Pilatus3 R 200K hybrid pixel array detector.

Cell constants and an orientation matrix for data collection were obtained from least-squares refinement using the setting angles of 20050 reflections in the range 4.0390°<θ<76.6740°. The space group was determined by the program CRYSALISPRO to be P1 (international tables no. 1).

The data were collected to a maximum diffraction angle (20) of 155.556° at room temperature.

### Data Reduction

Frames were integrated with CRYSALISPRO. A total of 44259 reflections were collected, of which 15541 were unique. Lorentz and polarization corrections were applied to the data. The linear absorption coefficient is 0.828 mm 1 for Cu Kα radiation. An empirical absorption correction using CRYSALISPRO was applied. Transmission coefficients ranged from 0.956 to 1.000. Intensities of equivalent reflections were averaged. The agreement factor for the averaging was 3% based on intensity.

### Structure Solution and Refinement

The structure was solved by direct methods using SHELXT. The remaining atoms were located in succeeding difference Fourier syntheses. The structure was refined using SHELXL-2014. Hydrogen atoms were refined independently. The structure was refined in full-matrix least-squares by minimizing the function:

$$\\ \sum {w ( {\vert {F}_{o} \vert}^{2} - {\vert {F}_{c} \vert}^{2} )}^{2}$$

where the weight, w, is defined as 1/[σ²(Fₒ²)+(0.1151P)²+(0.0941P)], where P=(Fₒ²+2F_{c}²)/3.

Scattering factors were taken from the “International Tables for Crystallography”. Of the 15541 reflections used in the refinements, only the reflections with intensities larger than twice their uncertainty [I>2σ(I)], 13233, were used in calculating the fit residual, R. The final cycle of refinement included 1230 variable parameters, 3 restraints, and converged with respective unweighted and weighted agreement factors of:

$$\\ R = \sum \vert {F}_{o} - {F}_{c} \vert / \sum {F}_{o} = 0.0574 \\ {R}_{w} = \sqrt{( \sum {w ( {F}_{o}^{2} - {F}_{c}^{2} )}^{2} / \sum {w ( {F}_{o}^{2} )}^{2} )} = 0.155$$

The standard deviation of an observation of unit weight (goodness of fit) was 1.03. The highest peak in the final difference Fourier had an electron density of 0.335e/Å³. The minimum negative peak had a value of −0.266 e/Å³.

### (4) Differential Scanning Calorimeter (DSC)

DSC was performed using a Mettler-Toledo DSC3+ differential scanning calorimeter. A tau lag adjustment was performed with indium, tin, and zinc. The temperature and enthalpy were adjusted with octane, phenyl salicylate, indium, tin, and zinc. The adjustment was then verified with octane, phenyl salicylate, indium, tin, and zinc. The sample was placed into a hermetically sealed aluminum DSC pan, and the weight was accurately recorded. The pan was then inserted into the DSC cell. A weighed aluminum pan configured as the sample pan was placed on the reference side of the cell. The pan lid was pierced prior to sample analysis. Samples were analyzed from −30° C. to 250° C. @ 10°/min. The cyclic DSC method heated from −30° C. to 100° C., returned to −30° C., then heated to 250° C. at 10°/min.

### (5) Thermogravimetric Analysis (TGA)

TGA was performed using a Mettler-Toledo TGA/DSC3 analyzer. Temperature and enthalpy adjustments were performed using phenyl salicylate, indium, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen. Each sample was heated from ambient temperature to 350° C. at 10° C./min. Although thermograms are plotted by reference temperature (x-axis), results are reported according to sample temperatures.

### (6) Dynamic Vapor Sorption (DVS)

Automated vapor sorption (VS) data were collected on a Surface Measurement System DVS Intrinsic instrument, or a VTI SGA-100 instrument. Samples were not dried prior to analysis. Sorption and desorption data were collected over a range from 5% to 95% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples.

### (7) Proton Nuclear Magnetic Resonance (¹H NMR)

The solution ¹H NMR spectra were obtained using an Avance 600 MHz NMR spectrometer.

### General Procedures

### (1) Cooling and Slow Cooling Crystallization

Solutions of the compound of formula (I) were prepared in various solvents or solvent/anti-solvent systems. These solutions were then chilled below room temperature using a refrigerator for varying lengths of time in an attempt to induce nucleation. Upon observation of solids, in quantities sufficient for analysis, the solid material was isolated. If insufficient quantities of solid material for analysis were observed the solutions were further cooled using a freezer until further crystallization occurred. Samples were then either isolated for analysis as wet or dry powders.

### (2) Crystallization by Fast Evaporation

Solutions of the compound of formula (I) were prepared by adding aliquots of various solvents to quantities of the compound of formula (I). The mixtures were agitated between aliquot additions to assist dissolution. Once complete dissolution occurred, as judged by visual observation, the solution was filtered through a 0.2-μm nylon filter and allowed to evaporate at ambient temperature in an uncapped vial or at ambient temperature under nitrogen. The solids that formed were isolated for evaluation.

### Example 27: Preparation and Characterization of Form A of the Compound of Formula (I)

Form A may be prepared using any of the procedures described below.

Method 1: a sample of Material F or Form D, prepared according to Example 37 or Example 34, respectively, was heated to its melting point (approx. 131° C. or approx. 134° C., respectively). Once the entire sample was determined to have melted (by visual inspection), the sample was cooled to ambient temperature until crystallization occurred. The crystals were then isolated and analyzed by XRPD and determined to be Form A.

Method 2: to a sample of the compound of formula (I) was added EtOH to form a solution. The solution was stored in ambient conditions for 3 days, upon which time Et₂O was added. The solution was then slow cooled according to General Procedure 1 (refrigerator: 1 day, freezer: 2 days). Thereafter, the solution was removed from the freezer, warmed to ambient temperature, and crystallization by fast evaporation of the solvent was performed according to General Procedure 2. The resulting solids were then isolated and analyzed by XRPD and determined to be a mixture of Form A (as single crystals) and an amorphous solid.

A representative XRPD pattern of Form A is shown in FIG. 26A. Tabulated characteristics of the XRPD pattern of Form A in FIG. 26A are provided in Table 28, which lists diffraction angles (2θ) and relative intensity (expressed as a percentage with respect to the most intense peak).

The XRPD acquisition parameters used to collect the XRPD data presented in Table 28 are as follows: X-ray tube: Cu (1.54059 Å); voltage: 45 kV; amperage: 40 mA; scan range: 1.1-40.0° 2θ; step size: 0.017° 2θ; collection time: 719 s; scan speed: 3.2° per min; slit: DS: fixed slit ½°; SS: null; revolution time: 1.0 s; and mode: transmission.

TABLE 28
XRPD Pattern Data of Form A of the Compound of Formula (I)

| Diffraction Angles (2θ, °) | Relative Intensity (%) |
|---|---|
| 5.3 | 15 |
| 6.9 | 22 |
| 8.1 | 42 |
| 9.4 | 100 |
| 9.8 | 27 |
| 10.6 | 21 |
| 10.7 | 17 |
| 11.9 | 9 |
| 12.2 | 8 |
| 12.7 | 17 |
| 12.9 | 10 |
| 13.1 | 22 |
| 13.6 | 8 |
| 13.9 | 8 |
| 14.5 | 19 |
| 14.8 | 19 |
| 15.6 | 26 |
| 16.0 | 28 |
| 16.3 | 72 |
| 16.9 | 37 |
| 17.1 | 55 |
| 17.6 | 17 |
| 18.4 | 38 |
| 18.8 | 55 |
| 19.1 | 21 |
| 19.3 | 19 |
| 19.6 | 18 |
| 20.0 | 59 |
| 20.1 | 57 |
| 20.3 | 33 |
| 20.6 | 50 |
| 20.8 | 23 |
| 21.0 | 21 |
| 21.1 | 28 |
| 21.4 | 31 |
| 21.6 | 36 |
| 21.8 | 27 |
| 22.0 | 19 |
| 22.3 | 25 |
| 22.7 | 16 |
| 22.9 | 13 |
| 23.3 | 14 |
| 23.8 | 18 |
| 24.1 | 18 |
| 24.5 | 72 |
| 24.9 | 13 |
| 25.5 | 24 |
| 26.1 | 21 |
| 26.3 | 17 |
| 26.4 | 18 |
| 26.6 | 12 |
| 26.9 | 17 |
| 27.1 | 18 |
| 27.7 | 13 |
| 27.8 | 13 |
| 28.3 | 11 |
| 28.8 | 12 |
| 29.1 | 16 |
| 29.8 | 9 |
| 30.8 | 10 |
| 31.2 | 8 |
| 31.5 | 8 |
| 31.7 | 9 |
| 32.3 | 12 |
| 33.1 | 14 |
| 33.6 | 11 |
| 34.5 | 9 |

A DSC thermogram of Form A is shown in FIG. 27

The DSC thermogram of Form A shows an endotherm with a peak onset temperature at about 205° C.

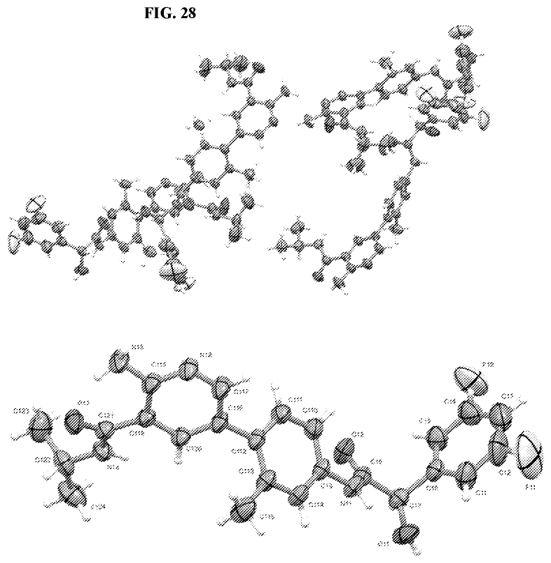

Single crystals of Form A were grown using Method 2 and scXRD data were collected using the method described in Example 26. A visual representation of the crystal structure of Form A is provided by the atomic displacement ellipsoid diagram shown in FIG. 28. Form A exists in a triclinic crystal system and has a P1 space group. Form A may be characterized by the crystallographic unit cell parameters as set forth in Table 29.

TABLE 29
Single Crystal X-ray Diffraction Data for Form A of the
Compound of Formula (I) and Data Collection Parameters

| | |
|---|---|
| Empirical formula | C₂₄H₂₄F₂N₄O₃ |
| Formula weight (g mol⁻¹) | 454.47 |
| Temperature (K) | 299.7(4) |
| Wavelength (Å) | 1.54184 |
| Crystal system | triclinic |
| Space group | P1 |
| Unit cell parameters | a = 10.80853(12) Å |
| | b = 13.1889(2) Å |
| | c = 16.99402(19) Å |
| | α = 79.7155(11)° |
| | β = 80.3819(9)° |
| | γ = 77.3515(11)° |
| Unit cell volume (Å³) | 2305.02(5) |
| Cell formula units, Z | 4 |
| Calculated density (g cm⁻³) | 1.310 |
| Absorption coefficient (mm⁻¹) | 0.828 |
| F(000) | 952 |
| Crystal size (mm³) | 0.2 × 0.14 × 0.02 |
| Reflections used for cell measurement | 20050 |
| θ range for cell measurement | 4.0390°-76.6740° |
| Total reflections collected | 44259 |
| Index ranges | −12 ≤ h ≤ 13; −16 ≤ k ≤ 16; −21 ≤ l ≤ 21 |
| θ range for data collection | θₘᵢₙ = 3.472°, θₘₐₓ = 77.778° |
| Completeness to θₘₐₓ | 97.4% |
| Completeness to 0_{full} = 67.684° | 99.9% |
| Absorption correction | multi-scan |
| Transmission coefficient range | 0.956-1.000 |
| Refinement method | full matrix least-squares on F² |
| Independent reflections | 15541 [Rᵢₙₜ = 0.0300, R_{σ} = 0.0313] |
| Reflections [I > 2σ(I)] | 13233 |
| Reflections/restraints/parameters | 15541/3/1230 |
| Goodness-of-fit on F² | S = 1.03 |
| Final residuals [I > 2σ(I)] | R = 0.0574, R_{w} = 0.1550 |
| Final residuals [all reflections] | R = 0.0648, R_{w} = 0.1633 |
| Largest diff. peak and hole (e Å⁻³) | 0.335, −0.266 |
| Max/mean shift/standard uncertainty | 0.000/0.000 |
| Absolute structure determination | Flack parameter: 0.03(8) |

Standard uncertainty is written in crystallographic parenthesis notation: e.g., 0.123 (4) is equivalent to 0.123±0.004. A calculated XRPD pattern of Form A (FIG. 26B) was generated for Cu radiation using MERCURY and the atomic coordinates, space group, and unit cell parameters determined from the single crystal structure analysis. The atomic displacement ellipsoid diagram (FIG. 28) was prepared using MERCURY. Atoms are represented by 50% probability anisotropic thermal ellipsoids.

The quality of the crystal structure obtained for Form A is high, as indicated by the fit residual, R, of 0.0574 (5.74%). R-factors in the range 2%-6% are quoted to be the most reliably determined structures.

### Example 28: Solubility Study of Form A of the Compound of Formula (1) in Solvents

A solubility study of Form A in various solvent systems was conducted according to the following protocol: aliquots of the test solvent systems were added to weighed samples of Form A, prepared according to Example 27, at room temperature. The resulting mixtures were sonicated between solvent additions to facilitate dissolution. Complete dissolution of the test material was determined visually. The solubility was estimated based on the total solvent used to provide complete dissolution. The solubility is expressed as “less than” if dissolution did not occur during the duration of the study. If complete dissolution was achieved after only one aliquot addition, the solubility is expressed as “greater than.” Samples that did not completely dissolve were placed at elevated temperatures to observe if dissolution occurred. The results of the solubility study are summarized in Table 30.

TABLE 30
Approximate Solubility of Form A of the Compound
of Formula (I) in Organic Solvents and Water

| Solvent | Solubility (mg/mL) |
|---|---|
| acetone | >115 |
| acetonitrile | 12 |
| chloroform | <3 |
| diethyl ether | <2 |
| ethanol | 49 |
| ethyl acetate | 26 |
| heptane | <3 |
| isopropyl alcohol | 14 |
| methanol | 103 |
| tetrahydrofuran | >150 |
| toluene | <4 |
| water | <3 |

### Example 29: Water Activity Study of Form A of the Compound of Formula (I)

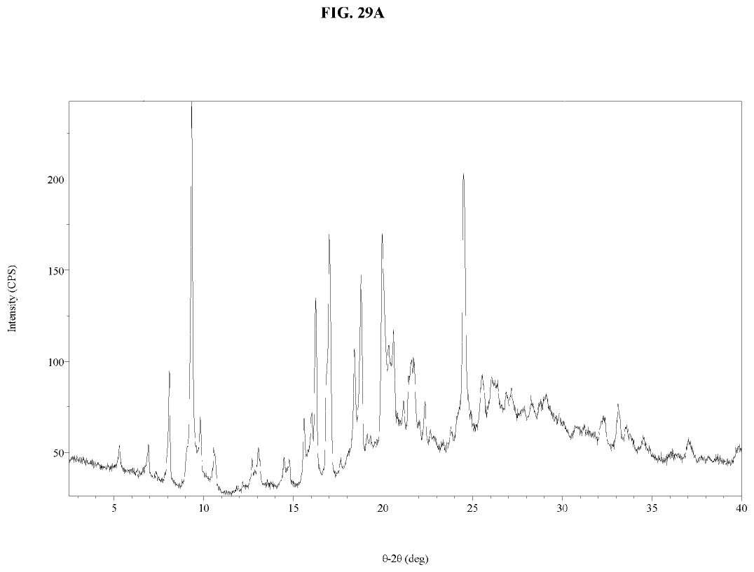

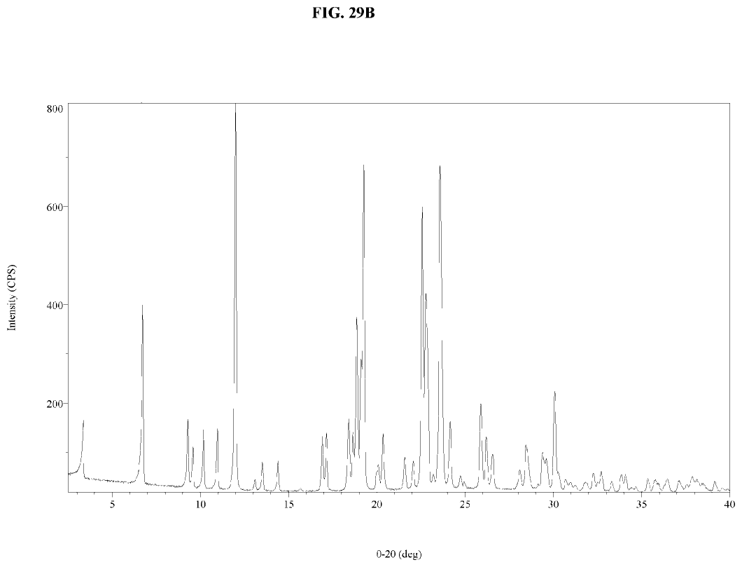

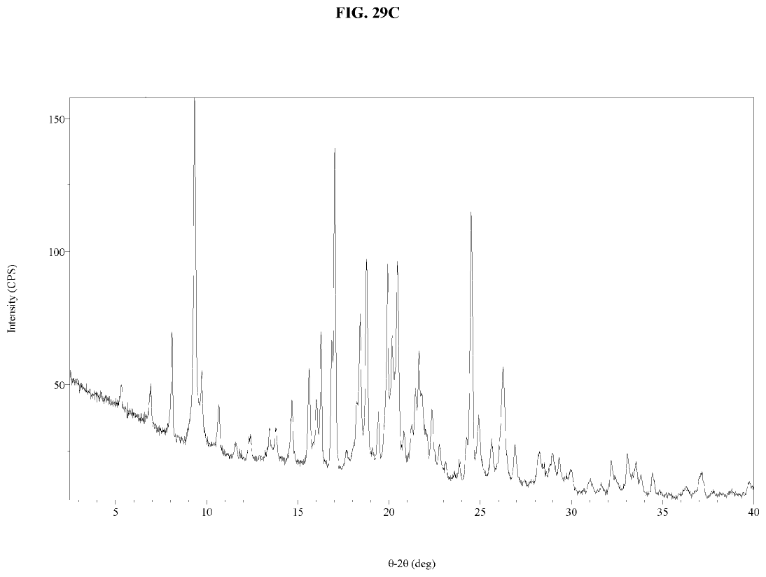

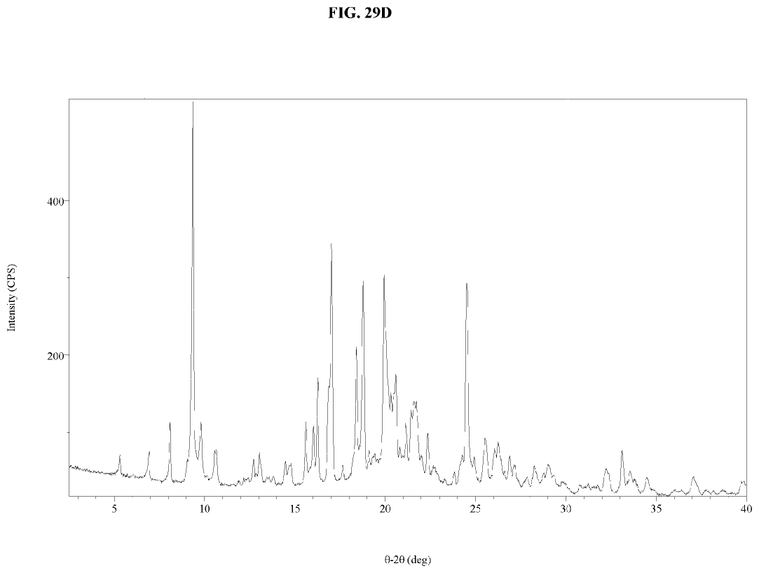

The water activity study was conducted using the following protocol: solutions were prepared by adding a sufficient amount of Form A, prepared according to Example 27, to a given solvent mixture such that excess solids were present. The mixture was then agitated for a predetermined period of time (Table 31) in a sealed vial under ambient conditions. After agitation, the solids were isolated for analysis and characterized using XRPD. The results of the study are tabulated in Table 31. The associated XRPD patterns are shown in FIGS. 29A-29D

TABLE 31
Water Activity Study of Form A of the Compound of Formula (I)

| | Study | | |
|---|---|---|---|
| Solvent | Conditions | Observation | Result |
| water/acetone | ambient, | off white fines, | Form A* |
| 80:20 v/v, 0.96 a_{w} | 4 days | birefringent, | |
| | | analyzed wet | |
| MeCN/water | ambient, | off white fines, | Form C |
| 92:8 v/v, 0.82 a_{w} | 4 days | birefringent | |
| IPA/water | ambient, | fines, | Form H |
| 90:10 v/v, 0.67 a_{w} | 27 days | birefringent | |
| IPA/water | ambient, | off white fines, | Form A* + |
| 94:6 v/v, 0.50 a_{w} | 4 days | birefringent | Form H |

*XRPD pattern of Form A includes low intensity diffraction peaks of an unidentified crystalline material.

### Example 30: Preparation and Characterization of Form B of the Compound of Formula (I)

Form B is an enantiotrope of Material F (see Example 37). The enantiotropes spontaneously interconvert upon passing a reversible transition at approximately 49° C. Form B has lower free energy and is more stable, relative to Material F, below the transition temperature, while the inverse occurs above the transition temperature.

Form B may be prepared using any of the procedures described below.

Method 1: a sample of Form C in a scintillation vial, prepared according to Example 32, was combined with MeCN to form a slurry. The vial was then sealed and the solution stored under ambient conditions for 25 days. The remaining solids were then isolated and analyzed by XRPD and determined to be Form B.

Method 2: to a sample of the compound of formula (I) was added MeCN to form a solution. The solution was stored under ambient conditions for 3 days. The solvent was then evaporated using the method described in General Procedure 2 to induce crystallization. The solids were then isolated and analyzed by XRPD and determined to be a mixture of Form B and Material F.

A representative XRPD pattern of Form B is shown in FIG. 30. Tabulated characteristics of the XRPD pattern of Form B in FIG. 30 are provided in Table 32, which lists diffraction angles (2θ) and relative intensity (expressed as a percentage with respect to the most intense peak).

The XRPD acquisition parameters used to collect the XRPD data presented in Table 32 are as follows: X-ray tube: Cu (1.54059 Å); voltage: 45 kV; amperage: 40 mA; scan range: 1.1-40.0° 2θ; step size: 0.017° 2θ; collection time: 718 s; scan speed: 3.3° per min; slit: DS: fixed slit ½°; SS: null; revolution time: 1.0 s; and mode: transmission.

TABLE 32
XRPD Pattern Data of Form B of the Compound of Formula (I)

| Diffraction | Relative |
|---|---|
| Angles (2θ, °) | Intensity (%) |
| 4.0 | 27 |
| 7.9 | 9 |
| 9.1 | 7 |
| 10.4 | 11 |
| 11.1 | 29 |
| 11.9 | 5 |
| 12.9 | 40 |
| 15.9 | 31 |
| 18.2 | 29 |
| 18.5 | 22 |
| 18.8 | 86 |
| 19.4 | 12 |
| 19.9 | 8 |
| 20.2 | 12 |
| 21.4 | 13 |
| 22.4 | 100 |
| 23.1 | 65 |
| 23.8 | 45 |
| 24.4 | 9 |
| 24.7 | 5 |
| 26.0 | 4 |
| 26.3 | 7 |
| 26.6 | 7 |
| 27.5 | 5 |
| 28.8 | 22 |
| 29.7 | 9 |
| 30.1 | 9 |
| 31.5 | 10 |
| 32.1 | 3 |
| 32.3 | 3 |
| 32.7 | 3 |
| 33.0 | 4 |
| 33.4 | 4 |
| 33.9 | 6 |
| 34.4 | 4 |

DSC and TGA thermograms for Form B are shown in FIG. 31 and FIG. 32, respectively.

The DSC thermogram of Form B shows three endotherms with peak onset temperatures at about 89° C., about 136° C., and about 197° C., an endotherm with a peak maximum at about 65° C., and an exotherm peak onset temperature at about 164° C.

DSC analysis shows that Form B undergoes a phase change to Material F (see Example 37 for characterization data for Material F) at approximately 89° C. Subsequently, Material F was observed to melt at approximately 136° C., and immediately recrystallize as Form A. Upon further heating, Form A exhibits a melt onset at approximately 197° C.

The TGA thermogram of Form B shows an approximate 0.8% weight loss upon heating from about 25° C. to about 190° C.

### Example 31: Variable Temperature Study of Form B of the Compound of Formula (I)

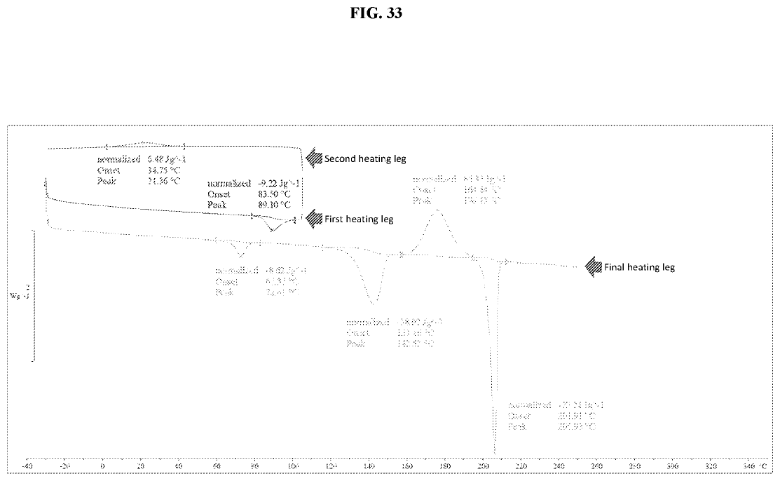

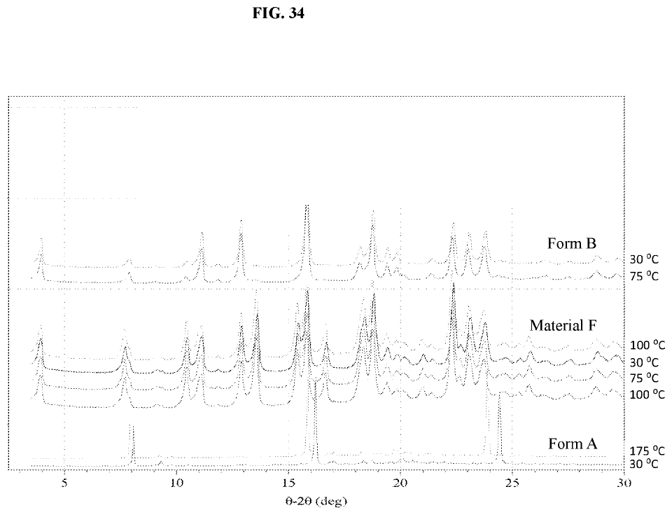

The thermal events associated with heating Form B, prepared according to Example 30, were further investigated by cycling DSC and variable temperature XRPD (FIG. 33 and FIG. 34, respectively). The first DSC heating cycle showed an endotherm at approximately 84° C., which corresponds to the conversion of Form B to Material F (FIG. 33). This phase transition was confirmed by VT-XRPD (see the diffraction pattern obtained at 100° C. in FIG. 34). During the first DSC cooling cycle, an exotherm at approximately 35° C. was observed. This exotherm was attributed to Material F undergoing a phase transition back to Form B. This result supports the conclusion that Form B and Material F are enantiotropically related. During the final DSC heating cycle, Form B first undergoes a phase transition to Material F at approximately 67° C. (endotherm). Material F was then observed to melt at approximately 131° C. (endotherm) and immediately recrystallize as Form A (exotherm onset at approximately 164° C.). Upon further heating, Form A is observed to melt with a melting onset temperature of approximately 202° C. Determining which solid form was obtained after recrystallization was confirmed by VT-XRPD (the pattern obtained at approximately 175° C., FIG. 34). The results of the VT-XRPD study of Form B are summarized in Table 33.

It is believed that this solid state behavior can be explained due to the relative stability of Form B and Material F is reversed in passing the transition temperature. For enantiotropic systems where hysteresis is minimal within the timescale measured, the transition temperature must lie between the onset temperatures of the observed endotherm and exotherm. Therefore, a transition temperature of ~49° C. was estimated from the thermogram with the use of the lever rule.

The XRPD acquisition parameters used to collect the VT-XRPD data presented in Table 33 are as follows: X-ray Tube: Cu (1.54059 Å); voltage: 45 kV; amperage: 40 mA; scan range: 3.50-29.99° 2θ; step size: 0.017° 2θ; scan speed: 1.7° per minute; slit: DS: fixed slit ¼°; SS: fixed slit: ½°; revolution time: 0.0 null; and mode: reflection. The XRPD data collection times for the experiment are specified in Table 33.

TABLE 33
VT-XRPD for Conversion of Starting Material Form B of the Compound
of Formula (I) to other Forms of the Compound of Formula (I)

| | | |
|---|---|---|
| | XRPD data col- | |
| | lection time | |
| Temperature | (seconds) | Result |

| | | | |
|---|---|---|---|
| 30° | C. | 957 | Form B |
| 75° | C. | 953 | Form B |
| 100° | C. | 953 | Material F |
| 30° | C. | 956 | Material F |
| 75° | C. | 955 | Material F |
| 100° | C. | 953 | Material F |
| 175° | C. | 955 | Form A¹ |
| 30° | C. | 955 | Form A |

¹Shifts in position of diffraction peaks attributed to thermal expansion of the unit cell.

### Example 32: Preparation and Characterization of Form C of the Compound of Formula (I)

Form C is an isostructural acetonitrile or acetone solvate.

Form C may be prepared from using any of the procedures described below.

Method 1: to a sample of the compound of formula (I) was added a minimal amount of MeCN to form a saturated solution. The solution was then crystallized by evaporation. The solids were then isolated and analyzed by XRPD to determine their identity as a mixture of Form B and Form C.

Method 2: to a sample of the compound of formula (I) was added MeCN to form a solution. The solution was heated to 65° C. and then slow cooled according to General Procedure 1. Once cooled, to assist crystallization, seeds of a mixture of Form B and Form C, prepared according to Example 32 Method 1, were added to the solution. Once crystallization occurred, the solution was decanted, the resulting wet solids isolated, and then analyzed by XRPD. The solids were determined to be Form C.

Method 3: to a sample of the compound of formula (I) was added acetone to form a solution. The solution was heated to 70° C. and then slow cooled according to General Procedure 1 (refrigerator: 1 day, freezer: 3 days). No change was noted in the solution after the slow cool crystallization attempt. Thereafter, the solution was removed from the freezer, warmed to ambient temperature, and crystallization by fast evaporation of the solvent was performed according to General Procedure 2. The resulting solids were then isolated and analyzed by XRPD to determine their identity as Form C mixed with an unidentified impurity which generated additional peaks in the XRPD pattern.

A representative XRPD pattern of Form C is shown in FIG. 35. Tabulated characteristics of the XRPD pattern of Form C in FIG. 35 are provided in Table 34, which lists diffraction angles (2θ) and relative intensity (expressed as a percentage with respect to the most intense peak).

The XRPD acquisition parameters used to collect the XRPD data presented in Table 34 are as follows: X-ray tube: Cu (1.54059 Å); voltage: 45 kV; amperage: 40 mA; scan range: 1.0-40.0° 2θ; step size: 0.017° 2θ; collection time: 719 s; scan speed: 3.3° per min; slit: DS: fixed slit ½°; SS: null; revolution time: 1.0 s; and mode: transmission.

TABLE 34
XRPD Pattern Data of Form C of the Compound of Formula (I)

| Diffraction | Relative |
|---|---|
| Angles (2θ, °) | Intensity (%) |
| 3.4 | 20 |
| 6.7 | 49 |
| 9.3 | 22 |
| 9.6 | 14 |
| 10.2 | 19 |
| 11.0 | 18 |
| 12.0 | 100 |
| 13.1 | 6 |
| 13.5 | 10 |
| 14.4 | 11 |
| 15.7 | 3 |
| 16.9 | 17 |
| 17.1 | 17 |
| 18.4 | 21 |
| 18.7 | 18 |
| 18.9 | 47 |
| 19.1 | 37 |
| 19.3 | 86 |
| 20.1 | 9 |
| 20.4 | 17 |
| 21.6 | 11 |
| 22.1 | 10 |
| 22.6 | 75 |
| 22.8 | 54 |
| 23.2 | 7 |
| 23.6 | 87 |
| 24.2 | 21 |
| 24.7 | 7 |
| 25.0 | 5 |
| 25.9 | 25 |
| 26.2 | 17 |
| 26.6 | 12 |
| 28.1 | 8 |
| 28.5 | 15 |
| 29.1 | 4 |
| 29.4 | 13 |
| 29.6 | 11 |
| 30.1 | 28 |
| 30.7 | 6 |

DSC and TGA thermograms for Form C are shown in FIG. 36 and FIG. 37, respectively.

The DSC thermogram of Form C shows three endotherms with peak onset temperatures of about 91° C., about 132° C., and about 203° C., and an exotherm with a peak onset at about 157° C.

DSC analysis shows that Form C undergoes a phase transition to Material F at approximately 91° C. (endotherm). Material F then melts at approximately 131° C. (endotherm) and immediately recrystallizes to Form A (exotherm onset at approximately 157° C.). Upon further heating, Form A is observed to melt at approximately 203° C.

The TGA thermogram of Form C shows an approximate 2.6% weight loss upon heating from 25° C. to 199° C.

### Example 33: Physical Stability of Form C of the Compound of Formula (I)

The physical stability of Form C at ambient conditions was investigated using the procedure as follows: to a sample of Form C, prepared according to Example 32, in a scintillation vial was added acetone, MeCN, or MeCN/water to form a mixture. The initial samples were then analyzed by XRPD. Thereafter, the vials were sealed and the samples were allowed to sit at ambient conditions for 9, 13, or 25 days (as designated in Table 35). The samples were then reanalyzed by XRPD.

XRPD analysis of the stored samples showed that, irrespective of the solvent system used to prepare Form C, under ambient conditions Form C desolvates and either partially converts or fully converts to Form B within days. A summary of the results of the physical stability study is provided in Table 35.

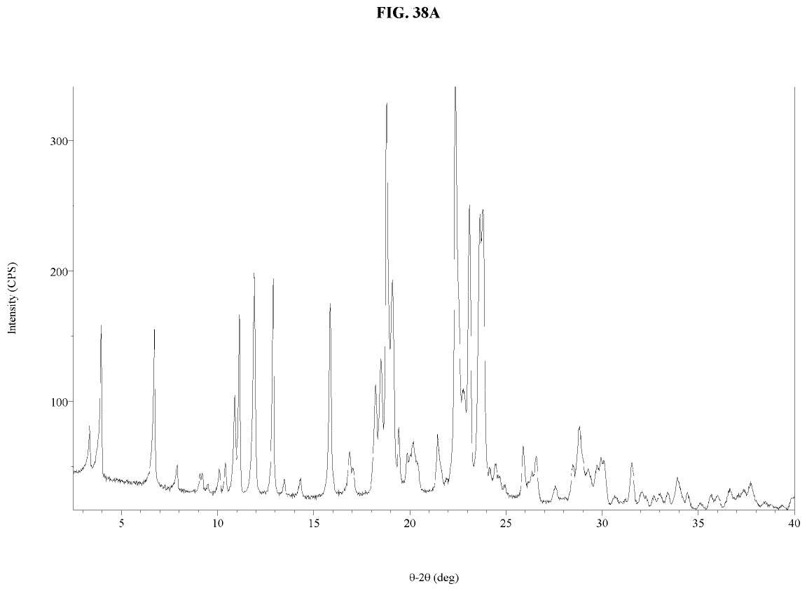

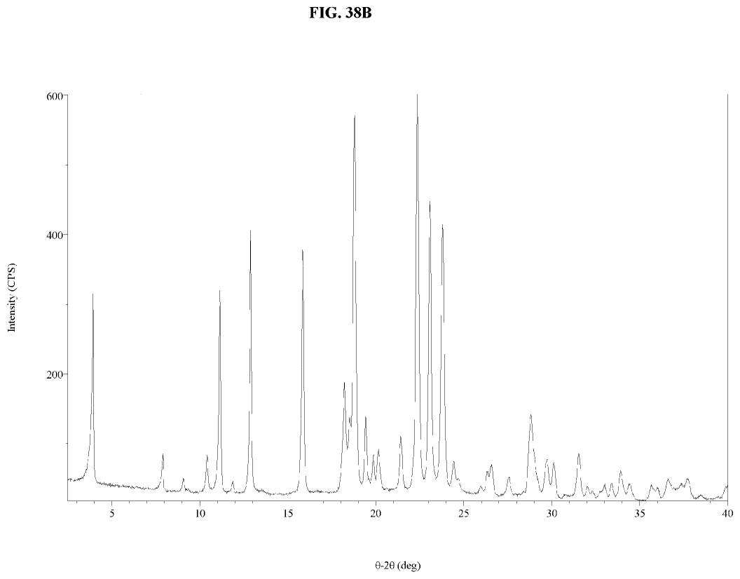

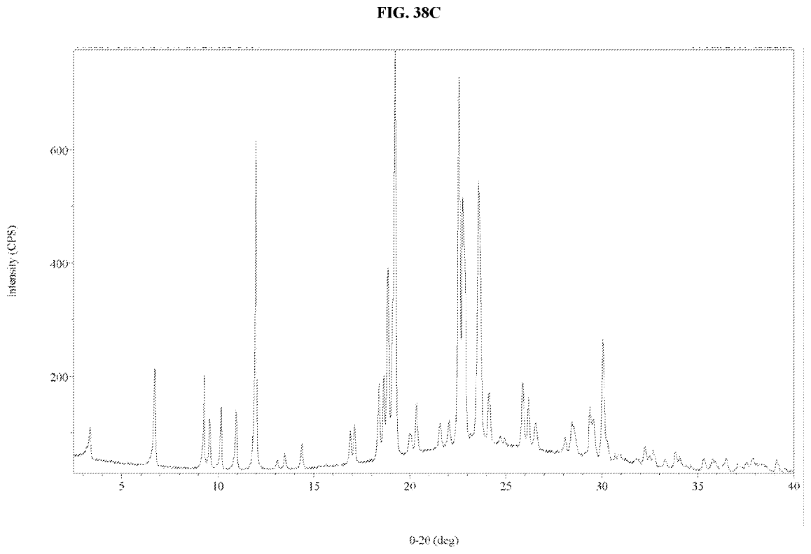

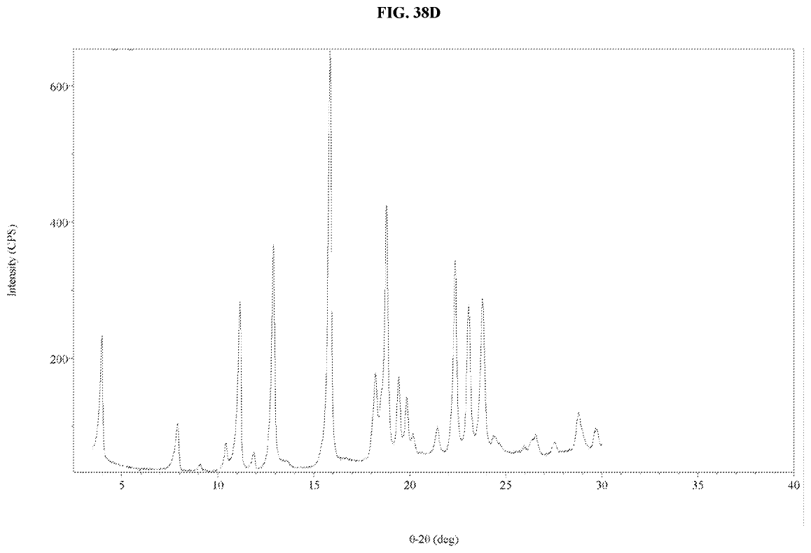

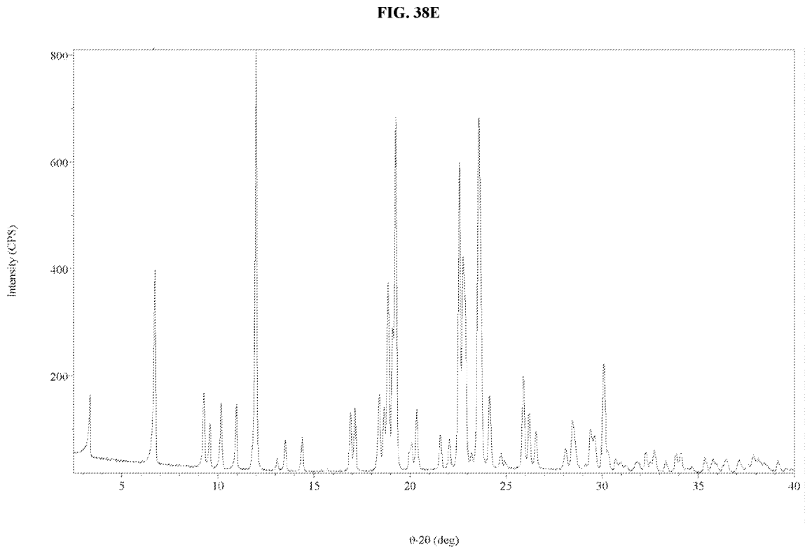

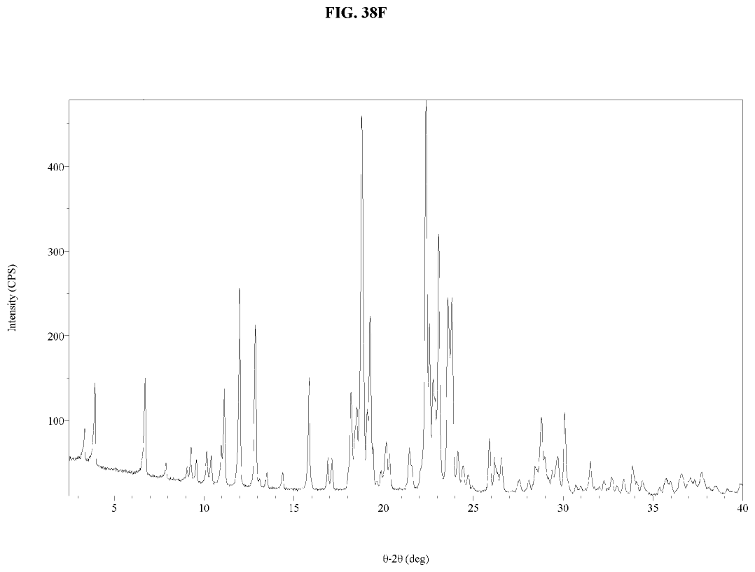

TABLE 35
Physical Stability of Form C of the Compound of Formula (I)

| Solvent System | Conditions Tested | Result |
|---|---|---|
| Acetone | Initial sample | Form B + Form C |
| | | (FIG. 38A) |
| | Ambient conditions, | Form B (FIG. 38B) |
| | stored for 13 days | |
| MeCN | Initial sample | Form C (FIG. 38C) |
| | Ambient conditions, | Form B (FIG. 38D) |
| | stored for 25 days | |
| MeCN/water | Initial Sample | Form C (FIG. 38E) |
| | Ambient conditions, | Form C + Form B |
| | stored for 9 days | (FIG. 38F) |

### Example 34: Preparation and Characterization of Form D of the Compound of Formula (I)

Form D was prepared using the procedure as follows: a sample of Form E, prepared according to Example 35, was desolvated in vacuo at ambient temperature for 1 day. The resulting solids were then isolated and analyzed by XRPD to determine their identity as Form D and an unidentified solid which resulted in an impurity peak in the XRPD pattern.

A representative XRPD pattern of Form D is shown in FIG. 39. Tabulated characteristics of the XRPD pattern of Form D in FIG. 39 are provided in Table 36, which lists diffraction angles (2θ) and relative intensity (expressed as a percentage with respect to the most intense peak).

The XRPD acquisition parameters used to collect the XRPD data presented in Table 36 are as follows: X-ray tube: Cu (1.54059 Å); voltage: 45 kV; amperage: 40 mA; scan range: 1.0-40.0° 2θ; step size: 0.017° 2θ; collection time: 722 s; scan speed: 3.2° per min; slit: DS: fixed slit ½°; SS: null; revolution time: 1.0 s; and mode: transmission.

TABLE 36
XRPD Pattern Data of Form D of the Compound of Formula (I)

| Diffraction | Relative |
|---|---|
| Angles (2θ, °) | Intensity (%) |
| 7.4 | 25 |
| 7.6 | 7 |
| 9.2 | 95 |
| 10.3 | 72 |
| 10.6 | 100 |
| 11.0 | 46 |
| 12.0 | 10 |
| 12.2 | 49 |
| 12.8 | 29 |
| 13.0 | 22 |
| 14.8 | 7 |
| 15.1 | 6 |
| 16.0 | 31 |
| 16.4 | 51 |
| 16.9 | 6 |
| 17.3 | 52 |
| 17.6 | 14 |
| 18.1 | 30 |
| 18.5 | 42 |
| 19.0 | 39 |
| 19.5 | 31 |
| 19.7 | 18 |
| 20.3 | 20 |
| 21.3 | 94 |
| 21.8 | 18 |
| 22.0 | 25 |
| 22.2 | 22 |
| 22.6 | 92 |
| 22.9 | 7 |
| 23.3 | 17 |
| 23.6 | 15 |
| 24.0 | 34 |
| 24.4 | 9 |
| 24.6 | 20 |
| 24.9 | 16 |
| 25.1 | 11 |
| 25.8 | 5 |
| 26.2 | 11 |
| 26.6 | 20 |
| 26.7 | 11 |
| 27.0 | 17 |
| 27.6 | 14 |
| 27.8 | 27 |
| 28.2 | 12 |
| 28.7 | 26 |
| 29.0 | 15 |
| 29.2 | 7 |
| 30.2 | 9 |
| 30.9 | 12 |
| 31.5 | 7 |
| 32.2 | 22 |
| 32.6 | 11 |

DSC and TGA thermograms for Form D are shown in FIG. 40 and FIG. 41, respectively.

The DSC thermogram of Form D shows two endotherms with peak maxima at about 66° C. and about 138° C., and an exotherm with a peak maximum at 157° C.

The TGA thermogram of Form D shows an approximate 1.0% weight loss upon heating from 25° C. to about 108° C.

The solution ¹H NMR spectrum of Form D dissolved in CD₃OD is found to be consistent with the ¹H NMR spectrum of the compound of formula (I).

The DVS isotherm provided in FIG. 42 shows that Form D exhibits limited hygroscopicity from 5 to 95% RH. The weight gain and loss of approximately 1.9% through the sorption/desorption cycle was observed with significant hysteresis above 55% RH. The material recovered from the DVS experiment remained as Form D, as determined by XRPD. Although the significant hysteresis observed is highly suggestive of the formation of a hydrate above 55% RH, XRPD analysis of Form D stored at 97% RH for 20 days at ambient temperature indicated that no phase changed had occurred (Table 37). In conclusion, Form D remained physically stable when exposed to either 75% or 97% RH for an extended period.

TABLE 37
Relative Humidity Stressing of Form
D of the Compound of Formula (I)

| Condition | Observation | Result |
|---|---|---|
| 75% RH, ambient, | Free flowing | Form D |
| 4 days | | |
| 97% RH, ambient, | Free flowing | Form D |
| 20 days | | |

### Example 35: Preparation and Characterization of Form E of the Compound of Formula (I)

Form E may be prepared using any of the procedures described below.

Method 1: to a sample of the compound of formula (I) was added EtOAc to form a solution. The solution was heated to 80° C. and then slow cooled according to General Procedure 1. Once cooled, to assist crystallization, Form D, prepared according to Example 34, was added to the solution as a seed. Upon solid formation, the resulting solids were isolated and analyzed by XRPD to determine their identity as Form E with a minor amount of Material G.

Method 2: to a sample of the compound of formula (I) was added EtOAc to form a solution. The solution was heated to 100° C. and then slow cooled according to General Procedure 1. No crystallization was noted after the slow cool crystallization attempt. Thereafter, the solution was warmed to ambient temperature and then cooled once again. Once cooled, to assist crystallization, heptane was added as an anti-solvent and solid material containing a mixture of Form B and Form C, prepared according to Example 32 Method 1, was added as a seed. These additions resulted in the precipitation of a solid. The resulting mixture was sonicated and then filtered, rinsing with heptane. The resulting solids were then isolated and analyzed by XRPD to determine their identity as Form E mixed with Material G.

A representative XRPD pattern of Form E is shown in FIG. 43. Tabulated characteristics of the XRPD pattern of Form E in FIG. 43 are provided in Table 38, which lists diffraction angles (2θ) and relative intensity (expressed as a percentage with respect to the most intense peak).

The XRPD acquisition parameters used to collect the XRPD data presented in Table 38 are as follows: X-ray tube: Cu (1.54059 Å); voltage: 45 kV; amperage: 40 mA; scan range: 1.0-40.0° 2θ; step size: 0.017° 2θ; collection time: 719 s; scan speed: 3.3° per min; slit: DS: fixed slit ½°; SS: null; revolution time: 1.0 s; and mode: transmission.

TABLE 38
XRPD Pattern Data of Form E of the Compound of Formula (I)

| Diffraction | Relative |
|---|---|
| Angles (2θ, °) | Intensity (%) |
| 6.7 | 98 |
| 7.2 | 8 |
| 8.5 | 99 |
| 8.8 | 90 |
| 10.2 | 56 |
| 11.1 | 6 |
| 11.4 | 48 |
| 12.2 | 34 |
| 13.5 | 5 |
| 14.2 | 7 |
| 14.5 | 25 |
| 15.1 | 8 |
| 16.0 | 27 |
| 16.1 | 40 |
| 16.8 | 31 |
| 17.0 | 22 |
| 17.6 | 29 |
| 17.7 | 14 |
| 18.5 | 55 |
| 19.0 | 76 |
| 19.2 | 23 |
| 19.7 | 10 |
| 20.5 | 22 |
| 21.0 | 29 |
| 21.1 | 51 |
| 21.7 | 21 |
| 22.2 | 19 |
| 22.8 | 100 |
| 23.4 | 49 |
| 24.1 | 18 |
| 24.6 | 25 |
| 25.1 | 17 |
| 25.7 | 39 |
| 26.1 | 37 |
| 26.6 | 14 |
| 27.2 | 25 |
| 27.7 | 19 |
| 28.2 | 6 |
| 29.2 | 10 |
| 29.5 | 6 |
| 30.1 | 7 |
| 30.4 | 10 |
| 30.9 | 8 |
| 31.4 | 12 |

DSC and TGA thermograms of Form E are shown in FIG. 44 and FIG. 45, respectively.

The DSC thermogram of Form E shows three endotherms with peak maxima at about 40° C., about 92° C., and about 137° C., an endotherm with a peak onset temperature at about 202° C., and an exotherm with a peak maximum at about 161° C.

The TGA thermogram of Form E shows approximately 9.0% weight loss upon heating from 25° C. to about 219° C.

### Example 36: Preparation and Characterization of Form H of the Compound of Formula (I)

Form H and Form A (see Example 27) are monotropes; Form H is more thermodynamically stable relative to Form A at ambient conditions.

Form H was prepared using the procedure as follows: to a vial containing IPA/water (90:10 v/v, 0.67 aw) was added enough of Form A, prepared according to Example 27, such that an excess of solids was present and a slurry was formed. The vial was then sealed and the mixture was agitated at ambient temperature for 27 days. The solids were then filtered, isolated, and analyzed by XRPD to determine their identity as Form H.

A representative XRPD pattern of Form H is shown in FIG. 46. Tabulated characteristics of the XRPD pattern of Form H in FIG. 46 are provided in Table 39, which lists diffraction angles (2θ) and relative intensity (expressed as a percentage with respect to the most intense peak).

The XRPD acquisition parameters used to collect the XRPD data presented in Table 39 are as follows: X-ray tube: Cu (1.54059 Å); voltage: 45 kV; amperage: 40 mA; scan range: 1.0-40.0° 2θ; step size: 0.017° 2θ; collection time: 719 s; scan speed: 3.3° per min; slit: DS: fixed slit ½°; SS: null; revolution time: 1.0 s; and mode: transmission.

TABLE 39
XRPD Pattern Data of Form H of the Compound of Formula (I)

| Diffraction | Relative |
|---|---|
| Angles (2θ, °) | Intensity (%) |
| 5.3 | 32 |
| 6.9 | 31 |
| 8.1 | 44 |
| 9.4 | 100 |
| 9.7 | 35 |
| 10.7 | 27 |
| 11.6 | 18 |
| 12.3 | 19 |
| 12.4 | 19 |
| 13.5 | 21 |
| 13.8 | 21 |
| 14.7 | 28 |
| 15.6 | 35 |
| 16.0 | 28 |
| 16.3 | 44 |
| 16.9 | 42 |
| 17.0 | 87 |
| 17.7 | 15 |
| 18.4 | 49 |
| 18.8 | 61 |
| 19.4 | 24 |
| 19.9 | 61 |
| 20.2 | 43 |
| 20.5 | 61 |
| 20.8 | 20 |
| 21.2 | 22 |
| 21.5 | 31 |
| 21.6 | 40 |
| 21.8 | 29 |
| 22.4 | 26 |
| 22.8 | 17 |
| 23.1 | 12 |
| 24.3 | 19 |
| 24.5 | 71 |
| 24.9 | 25 |
| 25.6 | 19 |
| 26.3 | 35 |
| 26.9 | 17 |
| 28.2 | 16 |
| 29.0 | 15 |
| 29.3 | 14 |
| 30.0 | 11 |

A DSC thermogram of Form H is shown in FIG. 47.

The DSC thermogram of Form H shows an endotherm peak onset temperature at about 205° C.

### Example 37: Preparation and Characterization of Material F of the Compound of Formula (I)

Material F and Form B (see Example 30) are enantiotropically related and spontaneously interconvert upon passing a reversible transition at approximately 49° C.; Form B is more thermodynamically stable, relative to Material F, below the transition temperature while the inverse occurs above the transition temperature.

Material F was prepared using the procedure as follows: a sample of Form B, prepared according to Example 30, was heated to 100° C. The resulting solids were analyzed by XRPD at 100° C. and determined to be Material F.

A representative XRPD pattern of Material F is shown in FIG. 48. Tabulated characteristics of the XRPD pattern of Material F in FIG. 48 are provided in Table 40, which lists diffraction angles (2θ) and relative intensity (expressed as a percentage with respect to the most intense peak).

The XRPD acquisition parameters used to collect the XRPD data presented in Table 40 are as follows: X-ray tube: Cu (1.54059 Å); voltage: 45 kV; amperage: 40 mA; scan range: 3.5-30.0° 2θ; step size: 0.017° 2θ; collection time: 957 s; scan speed: 1.7° per min; slit: DS: fixed slit ¼°; SS: fixed slit ½°; revolution time: 1.0 s; and mode: reflection.

TABLE 40
XRPD Pattern Data of Material F of the Compound of Formula (I)

| Diffraction | Relative |
|---|---|
| Angles (2θ, °) | Intensity (%) |
| 4.0 | 45 |
| 7.7 | 37 |
| 9.1 | 15 |
| 9.4 | 13 |
| 10.5 | 46 |
| 11.1 | 47 |
| 11.9 | 15 |
| 12.9 | 58 |
| 13.6 | 69 |
| 15.4 | 61 |
| 15.8 | 92 |
| 16.7 | 42 |
| 18.4 | 67 |
| 18.8 | 90 |
| 19.4 | 37 |
| 19.9 | 30 |
| 20.2 | 27 |
| 21.0 | 30 |
| 21.4 | 25 |
| 22.4 | 100 |
| 22.7 | 39 |
| 23.1 | 65 |
| 23.8 | 61 |
| 24.7 | 26 |
| 25.3 | 26 |
| 25.8 | 32 |
| 26.5 | 23 |
| 27.6 | 25 |
| 28.8 | 32 |
| 29.6 | 29 |

### Example 38: Preparation and Characterization of Material G of the Compound of Formula (I)

Material G is a purported EtOAc solvate. The nature of Material G is inferred based on the experimental conditions from which it was derived.

Material G was prepared using the procedure as follows: to a sample of the compound of formula (I) was added EtOAc to form a solution. The solution was heated to 100° C. and then slow cooled according to General Procedure 1. No crystallization was noted after the slow cool crystallization attempt. Thereafter, the solution was warmed to ambient temperature and then cooled once again. Once cooled, to assist crystallization, heptane was added as an anti-solvent and solid material containing a mixture of Form B and Form C, prepared according to Example 32 Method 1, was added as a seed. These additions resulted in the precipitation of a solid. The resulting mixture was sonicated and then filtered, rinsing with heptane. The solids were set aside and the filtrate reserved at ambient conditions for observation. Precipitate formation was observed in the filtrate and upon sufficient solid formation, the solids were isolated and analyzed by XRPD to determine their identity as Material G.

A representative XRPD pattern of Material G is shown in FIG. 49. Tabulated characteristics of the XRPD pattern of Material G in FIG. 49 are provided in Table 41, which lists diffraction angles (2θ) and relative intensity (expressed as a percentage with respect to the most intense peak).

The XRPD acquisition parameters used to collect the XRPD data presented in Table 41 are as follows: X-ray tube: Cu (1.54059 Å); voltage: 45 kV; amperage: 40 mA, scan range: 1.0-40.0° 2θ, step size: 0.017° 2θ, collection time: 720 s, scan speed: 3.2° per min, slit: DS: fixed slit ½°, SS: null, revolution time: 1.0 s, mode: transmission.

TABLE 41
XRPD Pattern Data of Material G of the Compound of Formula (I)

| Diffraction | Relative |
|---|---|
| Angles (2θ, °) | Intensity (%) |
| 6.9 | 25 |
| 7.3 | 23 |
| 8.7 | 80 |
| 8.9 | 48 |
| 9.5 | 32 |
| 9.9 | 100 |
| 10.4 | 26 |
| 10.7 | 57 |
| 11.0 | 19 |
| 11.3 | 18 |
| 11.5 | 20 |
| 11.7 | 18 |
| 12.3 | 28 |
| 12.8 | 24 |
| 13.8 | 20 |
| 14.8 | 19 |
| 15.2 | 23 |
| 15.3 | 21 |
| 15.7 | 39 |
| 16.5 | 26 |
| 16.8 | 25 |
| 17.5 | 46 |
| 17.8 | 22 |
| 18.6 | 54 |
| 19.0 | 38 |
| 19.4 | 29 |
| 19.8 | 25 |
| 20.5 | 27 |
| 20.8 | 38 |
| 21.3 | 44 |
| 21.5 | 40 |
| 22.1 | 33 |
| 22.3 | 47 |
| 22.6 | 69 |
| 23.7 | 28 |
| 24.1 | 30 |
| 24.4 | 30 |
| 24.7 | 25 |
| 25.0 | 23 |
| 25.3 | 28 |
| 25.6 | 22 |
| 26.1 | 22 |
| 26.7 | 16 |
| 27.0 | 14 |
| 27.5 | 15 |
| 27.9 | 18 |
| 28.3 | 12 |
| 28.9 | 15 |

DSC and TGA thermograms of Material G are shown in FIG. 50 and FIG. 51, respectively.

The DSC thermogram of Material G shows two endotherms with peak onset temperatures at about 124° C. and about 194° C., and an exotherm peak onset temperature at about 124° C.

The TGA thermogram of Material G shows approximately 3.2% weight loss upon heating from 25° C. to about 169° C. Assuming the total loss is due to the volatilization of EtOAc, the weight corresponds to less than 0.2 mol/mol of EtOAc.

### Example 39: Slurry Studies of a Binary Mixture of Forms of the Compound of Formula (I)

Slurries were prepared by adding enough of the solid forms to a given solvent such that an excess of solids was present. The mixture was then agitated in a sealed vial at either ambient or a desired elevated temperature. After a given amount of time, the solids were isolated and analyzed by XRPD. The experimental conditions and results are summarized in Table 42.

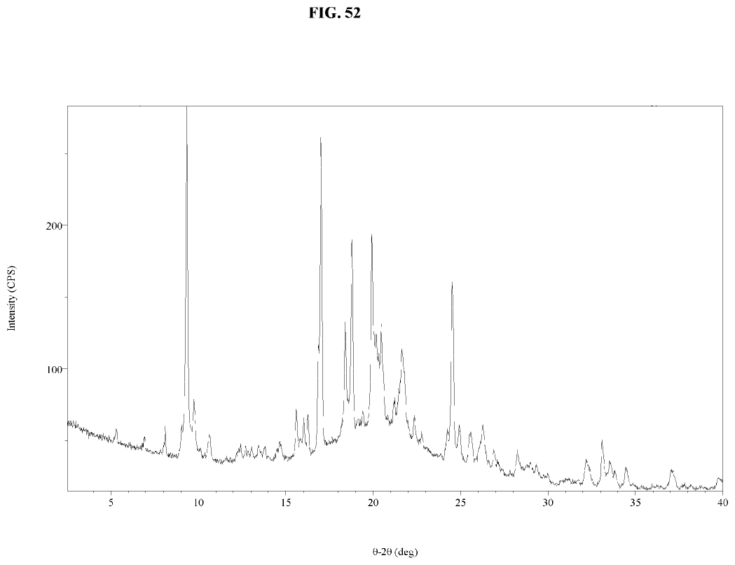

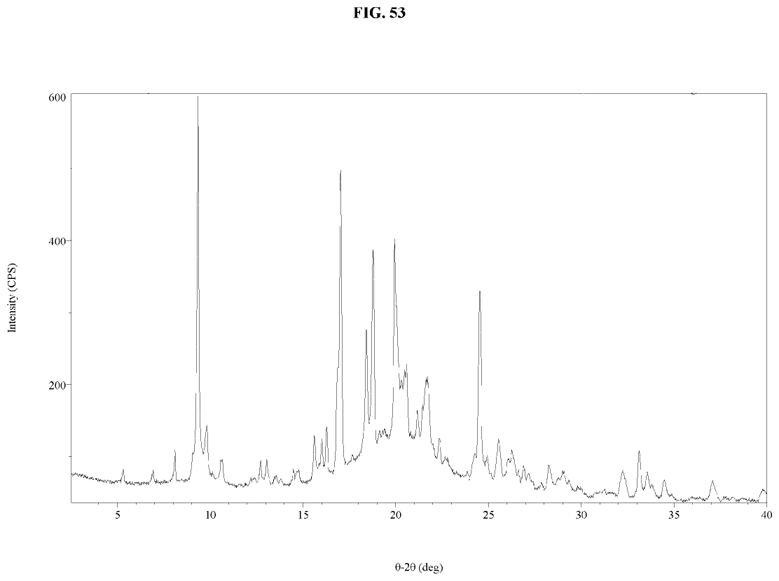

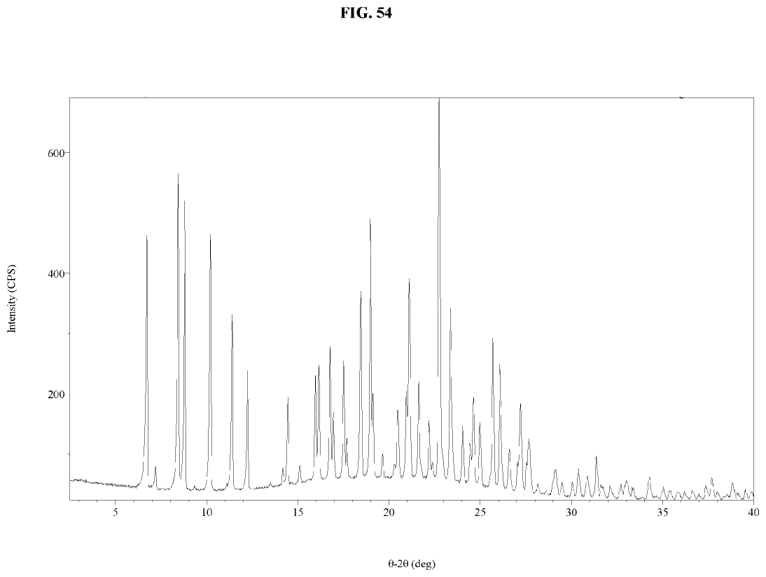

Slurrying a mixture of Form A and Form H, prepared according to Example 27 and Example 36, respectively, at ambient temperature in either IPA, Et₂O/MeOH (92:8 v/v), or H₂O/THF (80:20) provided predominantly Form H with a detectable amount of an unidentified crystalline material. However, under similar conditions with heptane/EtOH (67:33 v/v), slurrying the mixture of Form A and Form H provided a mixture of Form A and Form H, where Form H was the predominant form (FIG. 52). When the mixture of Form A and Form H was slurried in a mixture of IPA/H₂O (98:2 v/v) at ambient temperature, a mixture of Form A and Form H was obtained, where Form A was the predominant form (FIG. 53). When the mixture of Form A and Form H was slurried in EtOAc at ambient temperature, Form E was obtained (FIG. 54).

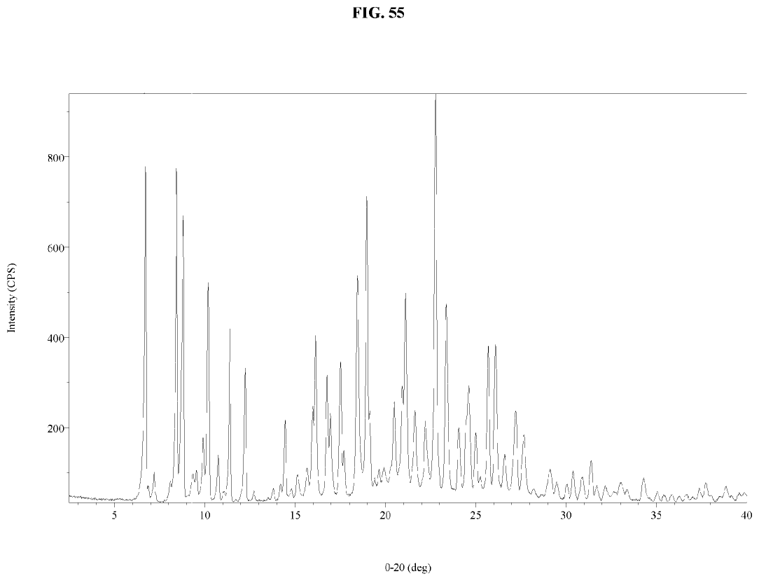

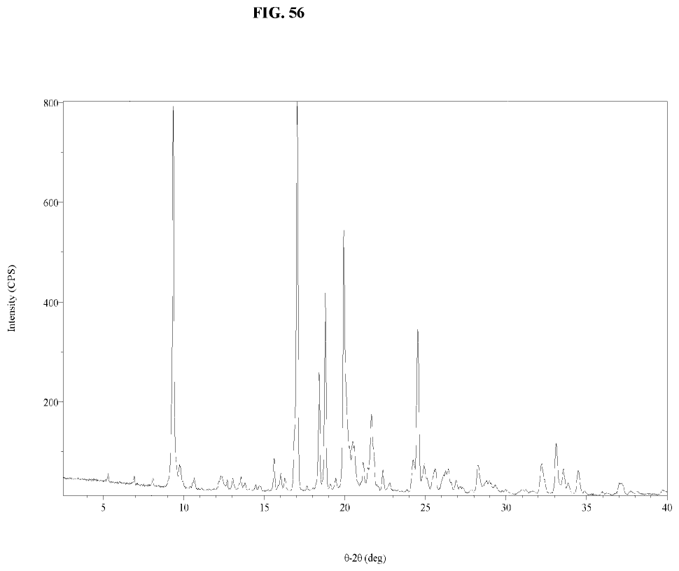

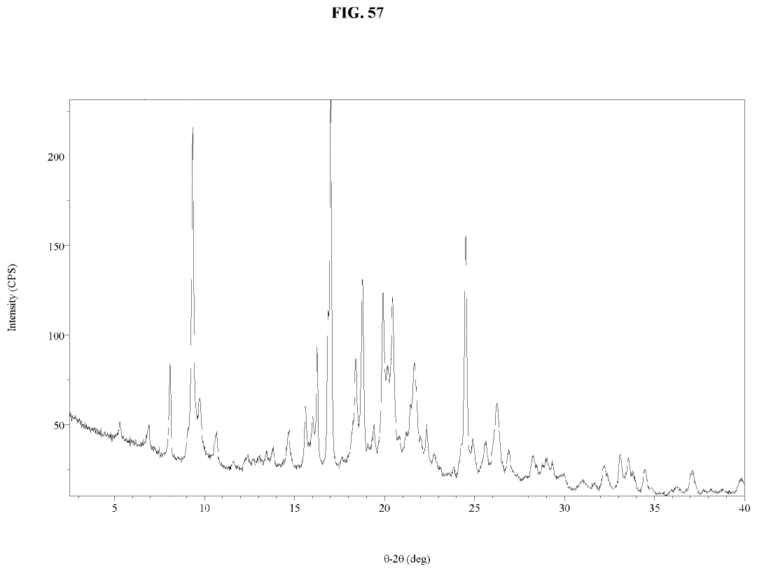

Slurrying a mixture of Form E and Material G at ambient temperature in EtOAc provided a mixture of Form E, Material G, and Form A (FIG. 55). Slurrying a mixture of Form A and Form B at 21° C. in EtOH provided a mixture of Form A and Form H, with Form A being the predominant form (FIG. 56). In EtOAc, slurrying a mixture of Form A and Form B provided a mixture of Form H and Form A, with Form H being the predominant form (FIG. 57). Slurrying a mixture of Form A and Form B in IPA at 21° C. provided Form A with a detectable amount of an unidentified crystalline material. This outcome was also observed when the mixture of Form A and Form B was slurried in EtOH, EtOAc or IPA at 32° C.

TABLE 42
Summary of a Crystal Form Stability
Study of the Compound of Formula (I)

| Forms | | | | | |
|---|---|---|---|---|---|
| (start) | Temperature | Time | | Solvent | Result |
| A + H | ambient | 21 | days | IPA | Form H* |
| A + H | ambient | 21 | days | Et₂O/MeOH | Form H* |
| | | | | 92:8 v/v | |
| A + H | ambient | 21 | days | H₂O/THF | Form H* |
| | | | | 80:20 v/v | |
| A + H | ambient | 21 | days | heptane/EtOH | Form H + Form A |
| | | | | 67:33 v/v | |
| A + H | ambient | 11 | days | IPA/H₂O | Form A + Form H |
| | | | | 98:2 v/v | |
| A + H | ambient | 11 | days | EtOAc | Form E |
| E + G | ambient | 3 | days | EtOAc | Form E + |
| | | | | | Material G + |
| | | | | | Form A |
| A + B | 21° C. | 4 | days | EtOH | Form A + Form H |
| | | | | EtOAc | Form H + Form A |
| | | | | IPA | Form A* |
| A + B | 32° C. | 4 | days | EtOH | Form A* |
| | | | | EtOAc | Form A* |
| | | | | IPA | Form A* |

*XRPD patterns include low intensity diffraction peaks of an unidentified crystalline material. Wherein the results are a mixture of two different Forms of the compound of formula (I), the predominant Form is listed first.

### Example 40: Preparation and Characterization of Crystalline Camsylate Salt Form A of the Compound of Formula (I)

Crystalline camsylate salt Form A was prepared according to the procedure as follows: to a sample of the compound of formula (I) was added EtOAc to form a solution. To a separate vessel containing a molar equivalent of (+)-(1S)-camphor-10-sulfonic acid (CSA) was added EtOAc to form a mixture, resulting in a slurry. The solution and the slurry were combined to form a clear solution. The resulting solution was then sub-divided into two portions and crystallized according to any of the methods detailed below.
Method 1: to the first subdivided solution was added Et₂O as an anti-solvent to induce precipitation. Solids were formed, resulting in a slurry. The slurry was allowed to sit at ambient conditions for 3 days at which time the resulting solids were isolated by filtration and analyzed by XRPD to determine their identity as crystalline camsylate salt Form A.
   a. The filtrate was crystallized by fast evaporation of the solvent according to General Procedure 2. Those resulting solids were also isolated and analyzed by XRPD to determine their identity as crystalline camsylate salt Form A.
Method 2: the second subdivided solution was slow cooled according to General Procedure 1 (refrigerator: 3 days, freezer: overnight). No change was noted in the solution after the slow cool crystallization attempt. A seed crystal obtained from Example 40 Method 1a was added to the solution in the freezer and the sample was slow cooled once more according to General Procedure 1 (freezer: overnight). The resulting solids were filtered, dried under nitrogen, and analyzed by XRPD to determine their identity as crystalline camsylate salt Form A.

A representative XRPD pattern of crystalline camsylate salt Form A is shown in FIG. 58. Tabulated characteristics of the XRPD pattern of crystalline camsylate salt Form A in FIG. 58 are provided in Table 43, which lists diffraction angles (2θ) and relative intensity (expressed as a percentage with respect to the most intense peak).

The XRPD acquisition parameters used to collect the XRPD data presented in Table 43 are as follows: X-ray tube: Cu (1.54059 Å); voltage: 45 kV; amperage: 40 mA; scan range: 1.0-40.0° 2θ; step size: 0.017° 2θ; collection time: 719 s; scan speed: 3.3° per min; slit: DS: fixed slit ½°; SS: null; revolution time: 1.0 s; and mode: transmission.

TABLE 43
XRPD Pattern Data of Crystalline Camsylate
Salt Form A of the Compound of Formula (I)

| Diffraction | Relative |
|---|---|
| Angles (2θ, °) | Intensity (%) |
| 5.2 | 100 |
| 6.9 | 9 |
| 8.5 | 6 |
| 8.9 | 4 |
| 10.5 | 12 |
| 10.8 | 4 |
| 12.3 | 8 |
| 12.6 | 6 |
| 13.2 | 36 |
| 13.4 | 6 |
| 14.0 | 5 |
| 14.1 | 8 |
| 14.7 | 46 |
| 15.1 | 3 |
| 15.7 | 19 |
| 17.0 | 14 |
| 17.2 | 10 |
| 17.6 | 6 |
| 17.8 | 25 |
| 18.1 | 16 |
| 18.2 | 33 |
| 19.3 | 32 |
| 19.8 | 10 |
| 20.2 | 3 |
| 20.6 | 3 |
| 20.9 | 14 |
| 21.1 | 12 |
| 21.6 | 16 |
| 21.8 | 27 |
| 22.5 | 9 |
| 22.9 | 14 |
| 23.9 | 10 |
| 24.9 | 5 |
| 25.4 | 11 |
| 25.6 | 11 |
| 25.9 | 13 |
| 26.3 | 8 |
| 26.6 | 5 |
| 27.1 | 3 |
| 27.5 | 5 |
| 28.5 | 4 |
| 28.9 | 4 |
| 29.4 | 5 |
| 30.1 | 2 |

DSC and TGA thermograms of crystalline camsylate salt Form A are shown in FIG. 59 and FIG. 60, respectively.

The DSC thermogram of crystalline camsylate salt Form A shows three endotherms with peak maxima at about 54° C., about 133° C., and about 174° C.

The TGA thermogram of crystalline camsylate salt Form A shows approximately 2.8% weight loss upon heating from 36° C. to about 189° C.

The solution ¹H NMR of crystalline camsylate salt Form A dissolved in CD₃OD is consistent with the ¹H NMR spectrum of the compound of formula (I). The ¹H NMR spectrum also shows 1:1 ratio of the compound of formula (I) to CSA, which indicates the salt formation.

### Example 41: Preparation and Characterization of Phosphate Salt Material A of the Compound of Formula (I)

Phosphate salt Material A was prepared according to the procedure as follows: to a sample of the compound of formula (I) was added IPA, a molar equivalent of phosphoric acid, and EtOAc to form a clear solution. The solution was then slow cooled according to General Procedure 1 (refrigerator: overnight, freezer: 6 days). No change was noted in the solution after the slow cool crystallization attempt. Thereafter, the solution was removed from the freezer, warmed to ambient temperature, and crystallization by fast evaporation crystallization of the solvent was performed according to General Procedure 2. The resulting solids were then isolated, dried under nitrogen, and analyzed by XRPD to determine their identity as phosphate salt Material A.

A representative XRPD pattern of phosphate salt Material A is shown in FIG. 61. Tabulated characteristics of the XRPD pattern of phosphate salt Material A in FIG. 61 are provided in Table 44, which lists diffraction angles (2θ) and relative intensity (expressed as a percentage with respect to the most intense peak).

The XRPD acquisition parameters used to collect the XRPD data presented in Table 44 are as follows: X-ray tube: Cu (1.54059 Å); voltage: 45 kV; amperage: 40 mA: scan range: 1.0-40.0° 2θ; step size: 0.017° 2θ; collection time: 720 s; scan speed: 3.2° per min; slit: DS: fixed slit ½°; SS: null; revolution time: 1.0 s; and mode: transmission.

TABLE 44
XRPD Pattern Data of Phosphate Salt Material
A of the Compound of Formula (I)

| Diffraction | Relative |
|---|---|
| Angles (2θ, °) | Intensity (%) |
| 3.9 | 100 |
| 5.5 | 3 |
| 7.0 | 3 |
| 7.7 | 5 |
| 13.7 | 7 |
| 14.0 | 4 |
| 16.0 | 7 |
| 16.3 | 11 |
| 17.2 | 11 |
| 24.2 | 7 |

### Example 42: Preparation and Characterization of Phosphate Salt Material B of the Compound of Formula (I)

Phosphate salt Material B was prepared according to the procedure as follows: to a sample of the compound of formula (I) was added EtOAc to form a solution. To the solution was added a seed of phosphate salt Material A, prepared according to Example 41, which did not dissolve in the solution. Then, to the solution was added a molar equivalent of phosphoric acid to afford a thick off-white plug of solid material. The mixture was sonicated to break the plug to afford a slurry comprised of a colorless flocculent and yellow oil. The suspended solid material was isolated and transferred to a separate vessel and the oil was left in the vial. To the oil was added EtOAc and the resulting suspension was sonicated to afford an off-white slurry. The slurry was then allowed to sit in ambient temperature for 3 days at which time the solids were isolated and analyzed via XRPD to determine their identity as phosphate salt Material B.

A representative XRPD pattern of phosphate salt Material B is shown in FIG. 62. Tabulated characteristics of the XRPD pattern of phosphate salt Material B in FIG. 62 are provided in Table 45, which lists diffraction angles (2θ) and relative intensity (expressed as a percentage with respect to the most intense peak).

The XRPD acquisition parameters used to collect the XRPD data presented in Table 45 are as follows: X-ray tube: Cu (1.54059 Å); voltage: 45 kV; amperage: 40 mA; scan range: 1.0-40.0° 2θ; step size: 0.017° 2θ; collection time: 719 s; scan speed: 3.3° per min; slit: DS: fixed slit ½°; SS: null; revolution time: 1.0 s; and mode: transmission.

TABLE 45
XRPD Pattern Data of Phosphate Salt Material
B of the Compound of Formula (I)

| Diffraction | Relative |
|---|---|
| Angles (2θ, °) | Intensity (%) |
| 4.1 | 53 |
| 6.9 | 63 |
| 7.8 | 21 |
| 8.3 | 16 |
| 8.5 | 13 |
| 9.1 | 12 |
| 9.3 | 15 |
| 10.6 | 49 |
| 10.8 | 70 |
| 11.4 | 53 |
| 12.3 | 13 |
| 13.4 | 41 |
| 14.6 | 47 |
| 15.8 | 32 |
| 16.2 | 21 |
| 16.4 | 17 |
| 16.6 | 40 |
| 16.7 | 47 |
| 17.1 | 17 |
| 18.0 | 59 |
| 18.4 | 26 |
| 18.8 | 16 |
| 19.8 | 25 |
| 20.0 | 67 |
| 20.8 | 19 |
| 21.4 | 54 |
| 21.7 | 85 |
| 22.0 | 30 |
| 22.5 | 44 |
| 22.9 | 27 |
| 23.2 | 33 |
| 24.0 | 22 |
| 25.1 | 63 |
| 26.1 | 100 |
| 26.7 | 41 |
| 27.3 | 19 |
| 28.0 | 63 |
| 28.5 | 29 |
| 28.7 | 28 |

DSC and TGA thermograms of phosphate salt Material B are shown in FIG. 63 and FIG. 64, respectively.

The DSC thermogram of phosphate salt Material B shows two endotherms with peaks at about 54° C. and about 90° C., and an endotherm with a peak onset temperature at about 176° C.

The TGA thermogram of phosphate salt Material B shows approximately 3.3% weight loss upon heating from 36° C. to about 149° C.

The solution ¹H NMR of phosphate salt Material B dissolved in CD₃OD is consistent with the ¹H NMR spectrum of the compound of formula (1). The ¹H NMR spectrum also shows the presence of phosphoric acid, which indicates the salt formation, and a trace amount of EtOAc.

### Example 43: A Multicenter, Open-label, Phase 1a Study of Form A of the Hemi-Fumarate Salt of the Compound of Formula (I) in Subjects with Advanced Solid Tumors

### BRIEF SUMMARY

This example describes a first in human, Phase 1a, multi-center, open-label study to establish the maximum tolerated dose (MTD) and evaluate the safety and tolerability of oral dosing of Form A of the hemi-fumarate salt of the compound of formula (I) (Example 11) in a dose-escalating fashion. Form A of the hemi-fumarate salt was orally administered BID every day with food or within 30 minutes of completing a meal, starting at 25 mg, with doses escalating to 50, 100, 200, 400, 600, and 900 mg BID as safety allows for each 3-week treatment cycle.

Up to 36 qualified subjects at 3 to 5 US sites, who have specific tumor types of renal cell carcinoma (RCC), gastric cancer (GC), metastatic breast cancer (MBC), small cell lung cancer (SCLC), and other solid tumors (e.g., non-small cell lung cancer, colorectal cancer, carcinoma of unknown primary) with the exception of rapidly progressing neoplasms (e.g., pancreatic cancer, glioblastoma, hepatocellular carcinoma) received Form A of the hemi-fumarate salt. Every effort was made to ensure approximately 50% of all subjects enrolled were subjects with RCC and GC. The starting dose level was 25 mg twice daily (BID), escalating to 50, 100, and 200 mg BID as safety allows, following the Bayesian Optimal Interval (BOIN) design. The safety monitoring committee (SMC) evaluated the dose-limiting toxicities (DLTs) and cumulative safety and PK data at the end of each cohort. Based on the SMC recommendations after a comprehensive review of PK and safety data for 200 mg BID dose, higher dose levels were evaluated, starting with 400 mg BID. The dose escalated to 600 mg and then 900 mg following the BOIN design starting with 1 subject at each escalated dose, until the MTD was reached or the sponsor or SMC declares the dose most appropriate for clinical development. This Phase 1a will be expanded into a Phase 1b/2a study through a protocol amendment and will then assess the dose and tumor type(s) selected in Phase 1a as the most appropriate for further clinical development. Subjects were dosed until unacceptable toxicity, disease progression per immune-related Response Evaluation Criteria in Solid Tumors (iRECIST), subject withdrawal, any other administrative reasons, or after 2 years of treatment, whichever occurs first. Efficacy was assessed via Response Evaluation Criteria in Solid Tumors 1.1 (RECIST 1.1); computed tomography (CT) scans were conducted every 6 weeks. Safety, including occurrence of dose-limiting toxicities (DLTs), pharmacokinetics (PK), and biomarker parameters were also assessed.

### DETAILED DESCRIPTION

Form A of the hemi-fumarate salt was orally administered BID with food or within 30 minutes of completing a meal, starting at 25 mg, with doses escalating to 50, 100, 200, 400, 600 and 900 mg BID as safety allowed. Up to 24 subjects were enrolled to ensure 12 subjects completed the study at the estimated MTD of Form A of the hemi-fumarate salt. Dosing occurred in 3-week cycles. Subjects spent Cycle 1/Day 1 (C1D1) in the clinic followed by an overnight stay for safety monitoring and PK sampling. Subjects were hospitalized for administration of first 3 doses: CID1 am and pm doses, and Cycle 1/Day 2 (C1D2) am dose; on Days 8, 15, and 21 the am dose was taken in the clinic after the planned PK samples. All other doses were self administered at home. After the initial hospital stay at the start of study, subjects were seen in outpatient clinic on Days 8, 15, and 21 of Cycle 1 for PK assessment and thereafter, the first day of each cycle for physical and laboratory assessments, adverse event (AE), and dosing compliance monitoring; the end of treatment visit was also in person in outpatient clinic.

Following completion of the treatment period of the study, subjects were monitored for survival up for up to 24 months after the last post treatment follow-up visit.

Dose escalation followed the Bayesian Optimal Interval (BOIN) design. The decision to escalate to the next dose level was based on safety assessments after all subjects of a cohort reached the end of Cycle 1/Day 21 (DLT evaluation period). The safety monitoring committee (SMC) was responsible for dose escalation decisions, including whether to modify the dose escalation based on the DLT observations and review of available PK data.

The target toxicity rate of 30%, with limits of 0.236 to 0.359 for escalation/de escalation, was employed to determine the MTD. With these predefined parameters, when the observed toxicity rate in a dose level is less than 0.236, the dose for the next cohort can escalate. If the observed toxicity rate is higher than 0.359, the dose will de escalate. Otherwise, the dose remains the same.

Individual subjects may be considered for treatment at a higher dose than the dose to which they were initially assigned after subject has completed 2 cycles of treatment and 1 postbaseline CT scan and maintained at least a stable disease (SD) response. In order to escalate a dose level, the subject must have tolerated his/her current dose level without experiencing a DLT, and the dose level to which the subject is planned to be escalated must have completed a DLT evaluation period, not exceeded the MTD, and been declared safe. Each subject can go through 2 dose escalations and do not need to go through a DLT evaluation period for either escalation. Intrasubject dose escalation will be considered on a case-by-case basis, each case to be assessed and approved by the sponsor.

### Participation Criteria

### Inclusion Criteria:

1. Have a signed informed consent form prior to any study specific procedures or treatment
2. Be ≥18 years of age (male or female) at the time of consent
3. Have 1 of the following histologically or cytologically confirmed tumor types with qualifying characteristics, and have received a minimum of 2 (and no more than 5) lines of prior therapy for metastatic (Stage IV) disease:
4. RCC (renal cell carcinoma-clear cell or papillary)
5. SCLC (small cell lung cancer) OR have received a minimum of 3 (and no more than 5) lines of prior therapy for metastatic (Stage IV) disease:
6. GC (gastric adenocarcinoma)
7. Human epidermal growth factor receptor positive (HER2+) MBC (metastatic breast cancer)
8. Other solid tumors (e.g., non-small cell lung cancer, colorectal cancer, carcinoma of unknown primary) with the exception of rapidly progressing neoplasms (e.g., pancreatic cancer, glioblastoma, hepatocellular carcinoma)
9. Note: Subjects with RCC and GC are a priority and should constitute approximately 50% (12 subjects) of the enrolled population. Enrollment of all others will be capped when reaching a combined 50%, in order to maintain 12 slots for subjects with RCC and GC.
10. Have at least 1 radiologically measurable lesion as per RECIST v 1.1, defined as a lesion that is at least 10 mm in longest diameter or lymph node and that is at least 15 mm in short axis imaged by CT scan or magnetic resonance imaging (MRI) and obtained by imaging within 28 days prior to screening. Tumor lesions situated in a previously irradiated area are considered measurable if progression has been demonstrated in such lesions
11. Have resolution of all previous treatment related toxicities to Grade 1 severity or lower, except for stable sensory neuropathy (≤Grade 2) and alopecia. If the subject received major surgery or radiation therapy of >30 Gy, they must have recovered from the toxicity and/or complications from the intervention
12. Provided there are suitable and accessible lesions, no biopsy contraindications, minimal risk of complications and a positive informed decision, subject as are willing to provide fresh tissue for biomarker analysis, and, based on the adequacy of the tissue sample quality, for assessment of biomarker status. Two biopsies will be necessary: at baseline (within 30 days prior to first dose) and within 7 days after Cycle 3/Day 1.
13. Newly obtained biopsy specimens are preferred to archived samples and formalin-fixed, paraffin-embedded block specimens are preferred to slides. In the event a fresh pre-treatment biopsy is not able to be provided, the most recent archival biopsy must be provided in its place
14. Have Eastern Cooperative Oncology Group performance status of 0 or 1 and sustained between screening and initiation of dosing on Day 1
15. QT interval corrected for heart rate using Fridericia's (QTcF) method≤450 msec
16. Have an albumin level of ≥3 g/dL at screening
17. Have life expectancy of 3 months or greater as determined by the treating physician
18. Have adequate organ function within 15 days prior to first administration of study drug on Day 1, as defined by meeting all of the following criteria:
19. Total bilirubin≤1.5×upper limit of normal (ULN) OR direct bilirubin≤ULN for subjects with total bilirubin levels>1.5×ULN
20. Aspartate aminotransferase and alanine aminotransferase≤2.5×ULN or ≤5×ULN for subjects with known hepatic metastases
21. Fasting serum glucose within normal range and hemoglobin Alc≤8%
22. Thyroid function tests (thyroid stimulating hormone [TSH] within normal limits for subjects with normal thyroid function and free thyroxine [FT4]) within normal limits for subjects on thyroid treatment
23. Have adequate renal function within 15 days prior to first administration of study drug on Day 1, as defined by creatinine≤1.5×ULN and creatinine clearance≥30 mL/min, as per the below Cockcroft Gault formula
24. Have adequate hematologic function within 15 days prior to first administration of study drug on Day 1, as defined by meeting all of the following criteria:
25. Hemoglobin≥9 g/dL (uncorrected by red blood cell transfusion or erythropoietin support)
26. Absolute neutrophil count≥1.5×109/L
27. Platelet count≥100×109/L
28. Have adequate coagulation function within 15 days prior to first administration of study drug on Day 1, as defined by either of the following criteria:
29. International normalized ratio (INR)<1.5×ULN OR for subjects receiving warfarin or low molecular weight heparin, the subject must, in the investigator's opinion, be clinically stable with no evidence of active bleeding while receiving anticoagulant therapy. The INR for these subjects may exceed 1.5×ULN if that is the goal of anticoagulant therapy
30. Activated partial thromboplastin time (aPTT)<1.5×ULN unless subject is receiving anticoagulant therapy, provided prothrombin time or aPTT is within therapeutic range of intended use of anticoagulants
31. Female subject of childbearing potential must have a negative urine or serum pregnancy within 72 hours prior to receiving the first dose of study medication. If the urine test is positive or cannot be confirmed as negative, a serum pregnancy test will be required
32. Female subject of childbearing potential must be willing to use an adequate form of contraception from the first dose of study medication through 90 days after the last dose of study drug
33. Female subject must agree not to breastfeed and not to donate ova starting at screening and throughout the study treatment, and for 90 days after the last dose of study drug
34. Male subject with a pregnant or breastfeeding partner(s) must agree to remain abstinent or use a condom for the duration of the pregnancy or for the time partner is breastfeeding throughout the study period and for 90 days after the last dose of study drug
35. Male subject with female partner(s) of childbearing potential must not donate sperm during the treatment period and for at least 90 days after the last dose of study drug
36. Male subject with female partner(s) of childbearing potential should agree to use a highly effective method of contraception during the treatment period and for at least 90 days after the last dose of the study drug
37. Be willing and have the ability to comply with scheduled visits (including geographical proximity), treatment plans, laboratory tests, and other study procedures.

### Exclusion Criteria:

1. Had prior chemotherapy, targeted small molecule therapy, or radiation therapy within 2 weeks prior to the first dose of study treatment or who has not recovered from adverse reactions due to a previously administered agent or major surgery
2. Is currently participating and receiving study therapy or has participated in a study of an investigational agent and received study therapy or used an investigational device within 4 weeks of the first dose of treatment
3. Has a diagnosis of immunodeficiency or receiving systemic steroid therapy or any other form of immunosuppressive therapy within 7 days prior to the first dose of study treatment. The use of physiologic doses of corticosteroids (≤30 mg/day of hydrocortisone, ≤10 mg/day of prednisone, ≤2 mg/day of dexamethasone, or equivalent) may be approved after consultation with the sponsor
4. Has taken a medication that is a strong CYP3A4 inhibitor or inducer within 4 weeks of the first dose of treatment (see Appendix 12)
5. Has known history of active tuberculosis
6. Has known history of human immunodeficiency virus (HIV) (HIV 1/2 antibodies)
7. Has known active Hepatitis B (anti hepatitis B surface antibody, anti hepatitis B core antibody, hepatitis B surface antigen [HBsAg]) or Hepatitis C (hepatitis C antibody) infection
8. Has a current diagnosis of severe acute respiratory syndrome coronavirus (SARS COV) 2 infection confirmed by reverse transcription polymerase chain reaction (PCR) test. Subject needs to have a negative PCR test at screening and a negative PCR test within 14 days prior to the first dose of study treatment
9. Has a history of clinically severe autoimmune disease, or a history of organ transplant
10. Has insulin dependent (Type I) diabetes or poorly controlled Type II diabetes (per clinical discretion) with hemoglobin A1c>8%
11. Has known additional malignancy that is progressing or required active treatment within previous 5 years. Exceptions include basal cell carcinoma or squamous cell carcinoma of the skin that has undergone potentially curative therapy, superficial bladder cancer, or in situ cervical cancer. Subjects with other malignancies are eligible if they were cured by surgery alone or surgery plus radiotherapy and have been continuously disease free for at least 5 years
12. Has known active central nervous system metastases and/or carcinomatous meningitis. Subjects with previously treated brain metastases may participate provided they are stable (without evidence of disease progression by imaging for at least 4 weeks prior to the first dose of trial treatment and any neurologic symptoms have returned to baseline), have no evidence of new or enlarging brain metastases, and are not using systemic steroids for at least 7 days prior to trial treatment. This exception does not include carcinomatous meningitis which is excluded regardless of clinical stability
13. Has a history of interstitial lung disease, pneumonitis within 12 months prior to screening or current pneumonitis
14. Has an active infection requiring systemic therapy
15. Has a history or current evidence of any condition, therapy, or laboratory abnormality that might confound the results of the trial, interfere with the subject's participation for the full duration of the trial, or is not in the best interest of the subject to participate, in the opinion of the treating investigator
16. Has a history or ongoing clinically significant cardiovascular disease such as unstable angina, myocardial infarction, or acute coronary syndrome, symptomatic or uncontrolled arrhythmia, congestive heart failure, baseline ECG abnormalities, including, but not limited to, QTc prolongation (prolonged QTcF defined as ≥450 msec) or any Class III or IV cardiac disease as defined by the New York Heart Association Functional Classification
17. Has overt or latent disorders of the exocrine pancreas (such as acute or chronic pancreatitis of any etiology) or chronic (including autoimmune) gastrointestinal disorders such as Crohn's disease, ulcerative colitis, rheumatoid arthritis, lupus, scleroderma, Sjogren's syndrome, and polyarteritis nodosa
18. Has a known psychiatric or substance abuse disorder(s) that would interfere with informed consent or cooperation with the requirements of the trial
19. Is pregnant or breastfeeding or expecting to conceive children within the projected duration of the trial, starting with the prescreening or screening visit through 90 days after the last dose of study drug
20. Is a first degree relative of the investigator, staff, or study sponsor.

### Ages Eligible for Study:

18 Years and older (Adult, Older Adult)

### Sexes Eligible for Study:

All

### Accepts Healthy Volunteers:

No

### Study Plan

Design Details
Primary Purpose: Treatment
Allocation: Non-Randomized
Interventional Model: Sequential Assignment
Masking: None (Open Label)

TABLE 46
Arms and Interventions

| Participant Group/Arm | Intervention/Treatment |
|---|---|
| Experimental: Cohort 1-25 mg | Drug: Form A of the hemi-fumarate |
| 25 mg capsules of Form A of | salt of the compound of formula (I) |
| the hemi-fumarate salt | |
| administered orally twice a | |
| day with food or within 30 | |
| minutes of completing a meal | |
| of each 3-week treatment cycle | |
| Experimental: Cohort 2-50 mg | Drug: Form A of the hemi-fumarate |
| 50 mg capsules of Form A of | salt of the compound of formula (I) |
| the hemi-fumarate salt | |
| administered orally twice a | |
| day with food or within 30 | |
| minutes of completing a meal | |
| of each 3-week treatment cycle | |
| Experimental: Cohort 3-100 mg | Drug: Form A of the hemi-fumarate |
| 100 mg capsules of Form A of | salt of the compound of formula (I) |
| the hemi-fumarate salt | |
| administered orally twice a | |
| day with food or within 30 | |
| minutes of completing a meal | |
| of each 3-week treatment cycle | |
| Experimental: Cohort 4-200 mg | Drug: Form A of the hemi-fumarate |
| 200 mg capsules of Form A of | salt of the compound of formula (I) |
| the hemi-fumarate salt | |
| administered orally twice a | |
| day with food or within 30 | |
| minutes of completing a meal | |
| of each 3-week treatment cycle | |
| Experimental: Cohort 5-400 mg | Drug: Form A of the hemi-fumarate |
| 400 mg capsules of Form A of | salt of the compound of formula (I) |
| the hemi-fumarate salt | |
| administered orally twice a | |
| day with food or within 30 | |
| minutes of completing a meal | |
| of each 3-week treatment cycle | |
| Experimental: Cohort 6-600 mg | Drug: Form A of the hemi-fumarate |
| 600 mg capsules of Form A of | salt of the compound of formula (I) |
| the hemi-fumarate salt | |
| administered orally twice a | |
| day with food or within 30 | |
| minutes of completing a meal | |
| of each 3-week treatment cycle | |
| Experimental: Cohort 7-900 mg | Drug: Form A of the hemi-fumarate |
| 900 mg capsules of Form A of | salt of the compound of formula (I) |
| the hemi-fumarate salt | |
| administered orally twice a | |
| day with food or within 30 | |
| minutes of completing a meal | |
| of each 3-week treatment cycle | |
| Experimental: Cohort 8-300 mg | Drug: Form A of the hemi-fumarate |
| 300 mg capsules of Form A of | salt of the compound of formula (I) |
| the hemi-fumarate salt | |
| administered orally twice a | |
| day with food or within 30 | |
| minutes of completing a meal | |
| of each 3-week treatment cycle | |

### Results

Exposure to Form A of the hemi-fumarate salt of the compound of formula (I) increased roughly proportional to the increase in dose across the 25 mg to 600 mg dose range. Steady-state appears to be achieved by Cycle 1, Day 8 based on similar trough concentration values on Cycle 1, Days 8, 15, and 21. Results of Cohorts 1 to 6 the study are detailed in Tables 47-50 below.

TABLE 47
Geometric mean (% CV) PK parameters following single oral dose
administration of Form A of the hemi-fumarate salt of the compound of the formula (I)
(Cycle 1, Day 1)

| Parameter | 25 mg | | 50 mg | | 100 mg | | 200 mg | | 300 mg | | 400 mg | | 600 mg^{b} |
|---|---|---|---|---|---|---|---|---|---|---|---|---|---|
| (Units) | (N = 3) | | (N = 3) | | (N = 4) | | (N = 5) | | (N = 4) | | (N = 3) | | (N = 1) |
| AUC₀₋ₗₐₛₜ | 850 | (31.8) | 2090 | (137) | 1930 | (94.6) | 5800 | (83.8) | 8610 | (127) | 3830 | (490) | 11600 |
| (h*ng/ml) | | | | | | | | | | | | | |
| AUC₀₋₁₂ | 850 | (31.8) | 2090 | (137) | 2800 | (36.9) | 5800 | (83.8) | 8610 | (127) | 3830 | (490) | 11600 |
| (h*ng/ml) | | | | | | | | | | | | | |
| AUC_{0-∞} | 2200 | | 5650 | (185) | NC | | 18000 | (240) | 4810 | | 16300 | (2.42) | NC |
| (h*ng/mL) | | | | | | | | | | | | | |
| % AUCₑₓₜᵣₐₚ | 46.8 | | 51.8 | (7.85) | NC | | 58.7 | (58.8) | 30.5 | | 33.6 | (18.0) | NC |
| (%) | | | | | | | | | | | | | |
| Cₘₐₓ | 120 | (42.4) | 243 | (150) | 380 | (38.7) | 839 | (103) | 1140 | (109) | 649 | (592) | 1460 |
| (ng/ml) | | | | | | | | | | | | | |
| tₘₐₓª | 8.00 | | 4.00 | | 7.00 | | 4.00 | | 7.00 | | 2.00 | | 2.00 |
| (h) | (4.00-8.00) | | (2.00-6.00) | | (1.00-8.00) | | (2.00-6.00) | | (2.00-8.00) | | (2.00-12.00) | | |
| tₗₐₛₜ^{a} | 12.00 | | 12.00 | | 12.00 | | 12.00 | | 12.00 | | (12.00-12.00) | | 12.00 |
| (h) | (12.00-12.00) | | (12.00-1200) | | (4.00-12.00) | | (12.00-12.00) | | (12.00-12.00) | | | | |
| t_{1/2}^{c} | 8.99 | | 11.1 | (16.6) | NC | | 20.6 | (191) | 6.88 | | 6.79 | (13.4) | NC |
| (h) | | | | | | | | | | | | | |
| CL/F | 11.4 | | 8.85 | (185) | NC | | 11.1 | (240) | 62.4 | | 24.5 | (2.42) | NC |
| (L/h) | | | | | | | | | | | | | |
| Vz/F | 147 | | 141 | (241) | NC | | 331 | (83.1) | 619 | | 240 | (15.8) | NC |
| (L) | | | | | | | | | | | | | |
| DAUC₀₋ₗₐₛₜ | 34.0 | (31.8) | 41.8 | (137) | 19.3 | (94.6) | 29.0 | (83.8) | 28.7 | (127) | 9.58 | (490) | 19.4 |
| (h*ng/mL/mg) | | | | | | | | | | | | | |
| DAUC₀₋₁₂ | 34.0 | (31.8) | 41.8 | (137) | 28.0 | (36.9) | 29.0 | (83.8) | 28.7 | (127) | 9.58 | (490) | 19.4 |
| (h*ng/mL/mg) | | | | | | | | | | | | | |
| DCₘₐₓ | 4.79 | (42.4) | 4.87 | (150) | 3.80 | (38.7) | 4.19 | (103) | 3.80 | (109) | 1.62 | (592) | 2.43 |
| (ng/mL/mg) | | | | | | | | | | | | | |

^{a}Median (min-max) presented
^{b}Point estimate provided (N = 1)
^{c}Interpret with caution. Terminal elimination phase not well captured.
NC = not calculated

TABLE 48
Dose proportionality following single oral dose administration of Form A of
the hemi-fumarate salt of the compound of formula (I) (Cycle 1, Day 1)

| Dose | 25 mg | 50 mg | 100 mg | 200 mg | 300 mg | 400 mg | 600 mg |
|---|---|---|---|---|---|---|---|
| Dose increment | — | 2 | 2 | 2 | 1.5 | 1.33 | 1.5 |
| Increase in mean Cₘₐₓ | — | 2.03 | 1.56 | 2.21 | 1.36 | 0.57 | 2.25 |
| DPF Cₘₐₓ | — | 1.01 | 0.78 | 1.10 | 0.91 | 0.43 | 1.50 |
| Increase in mean | — | 2.46 | 1.34 | 2.07 | 1.48 | 0.44 | 3.03 |
| DPF AUC₀₋₁₂ | — | 1.23 | 0.67 | 1.04 | 0.99 | 0.33 | 2.02 |

Dose proportionality based on preceding dose level = DPF = Dose Proportionality Factor − ratio of fold increase in geometric mean (or point estimate) parameter divided by fold increase in dose

TABLE 49
Geometric mean (% CV) PK parameters following multiple twice daily oral
dose administration of Form A of the hemi-fumarate salt of the compound of formula
(I) (Cycle 1, Day 21)

| Parameter | 25 mg | | 50 mg | | 100 mg | | 200 mg | | 300 mgb | 400 mg | | 600 mgb |
|---|---|---|---|---|---|---|---|---|---|---|---|---|
| (Units) | (N = 3) | | (N = 3) | | (N = 3) | | (N = 4) | | (N = 1) | (N = 2) | | (N = 1) |
| AUC₀₋ₗₐₛₜ | 4110 | (22.2) | 9070 | (159) | 11700 | (17.3) | 27200 | (44.6) | 28400 | 30800 | (82.0) | 60800 |
| (h*ng/ml) | | | | | | | | | | | | |
| AUC_{0-τ} | 4110 | (22.2) | 9070 | (159) | 11700 | (17.3) | 27200 | (44.6) | 28400 | 51200 | | 60800 |
| (h*ng/ml) | | | | | | | | | | | | |
| Cₘₐₓ | 537 | (23.3) | 978 | (136) | 1310 | (24.3) | 2990 | (34.6) | 3030 | 3680 | (39.6) | 5990 |
| (ng/ml) | | | | | | | | | | | | |
| C_{trough} | 359 | (78.8) | 780 | (128) | 777 | (41.6) | 1860 | (60.8) | 1770 | 3060 | (53.3) | 5680 |
| (ng/mL) | | | | | | | | | | | | |
| tₘₐₓ^{a} | 2.00 | | 2.00 | | 4.00 | | 5.00 | | 8.00 | 2.50 | | 12.00 |
| (h) | (0.50-4.00) | | (2.00-2.00) | | (2.00-12.00) | | (2.00-6.00) | | | (1.00-4.00) | | |
| t_{1/2}^{c} | 16.0 | | NC | | 37.6 | | 7.71 | | NC | 10.8 | | NC |
| (h) | | | | | | | | | | | | |
| t_{1/2, eff} | 35.8 | (22.9) | 31.5 | (41.7) | 30.1 | (56.6) | 35.9 | (63.2) | 19.4 | 38.5 | | 39.1 |
| (h) | | | | | | | | | | | | |
| CL/F | 6.08 | (22.2) | 5.51 | (159) | 8.53 | (17.3) | 7.36 | (44.6) | 10.5 | 7.82 | | 9.87 |
| (L/h) | | | | | | | | | | | | |
| Vz/F | 150 | | NC | | 383 | | 79.4 | | NC | 122 | | NC |
| (L) | | | | | | | | | | | | |
| AR_{AUC} | 4.84 | (20.4) | 4.34 | (35.8) | 4.19 | (46.8) | 4.90 | (55.6) | 2.87 | 5.15 | | 5.22 |
| DAUC_{0-τ} | 164 | (22.2) | 181 | (159) | 117 | (17.3) | 136 | (44.6) | 94.8 | 128 | | 101 |
| (h*ng/mL/mg) | | | | | | | | | | | | |
| DAUC₀₋ₗₐₛₜ | 164 | (22.2) | 181 | (159) | 117 | (17.3) | 136 | (44.6) | 94.8 | 77.0 | (82.0) | 101 |
| (h*ng/mL/mg) | | | | | | | | | | | | |
| DCₘₐₓ | 21.5 | (23.3) | 19.6 | (136) | 13.1 | (24.3) | 14.9 | (34.6) | 10.1 | 9.20 | (39.6) | 9.98 |
| (ng/mL/mg) | | | | | | | | | | | | |

^{a}Median (min-max) presented
^{b}Point estimate provided (N = 1)
^{c}Interpret with caution. Terminal elimination phase not well captured.
NC = not calculated

TABLE 50
Dose proportionality following multiple twice daily oral dose administration
of Form A of the hemi-fumarate salt of the compound of formula (I) (Cycle 1, Day 21)

| Dose | 25 mg | 50 mg | 100 mg | 200 mg | 300 mg | 400 mg | 600 mg |
|---|---|---|---|---|---|---|---|
| Dose increment | — | 2 | 2 | 2 | 1.5 | 1.33 | 1.5 |
| Increase in mean Cₘₐₓ | — | 1.82 | 1.34 | 2.28 | 1.01 | 1.21 | 1.63 |
| DPF Cₘₐₓ | — | 0.91 | 0.67 | 1.14 | 0.68 | 0.91 | 1.09 |
| Increase in mean | — | 2.21 | 1.29 | 2.32 | 1.05 | 1.80 | 1.19 |
| DPF AUC_{0-τ} | — | 1.10 | 0.64 | 1.16 | 0.70 | 1.36 | 0.79 |

Dose proportionality based on preceding dose level
DPF = Dose Proportionality Factor − ratio of fold increase in geometric mean (or point estimate) parameter divided by fold increase in dose

### INCORPORATION BY REFERENCE

This application refers to various issued patents, published patent applications, journal articles, and/or other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed:

1. A fumarate salt of a compound of formula (I)

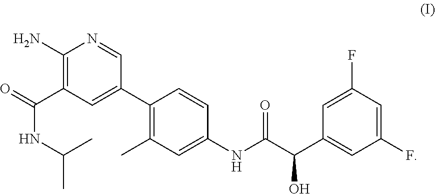 [SMILES: Cc1cc(NC(=O)[C@H](O)c2cc(F)cc(F)c2)ccc1-c1cnc(N)c(C(=O)NC(C)C)c1]

2. The fumarate salt of claim 1, wherein the fumarate salt is a mono-fumarate salt.

3. The fumarate salt of claim 1, wherein the fumarate salt is a hemi-fumarate salt.

4. A crystalline fumarate salt of a compound of formula (I)

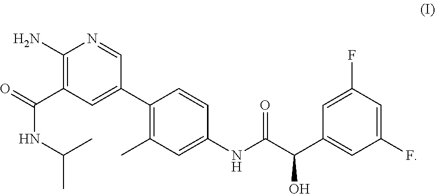 [SMILES: Cc1cc(NC(=O)[C@H](O)c2cc(F)cc(F)c2)ccc1-c1cnc(N)c(C(=O)NC(C)C)c1]

5. The crystalline fumarate salt of claim 4, wherein the crystalline fumarate salt has an X-ray powder diffraction (XRPD) pattern comprising one or more peaks at about 16.9°, about 17.9°, and about 24.1° 2θ.

6. The crystalline fumarate salt of claim 4 or 5, wherein the crystalline fumarate salt is a crystalline mono-fumarate salt.

7. The crystalline fumarate salt of any one of claims 4-6, wherein the crystalline fumarate salt has an XRPD pattern substantially the same as shown in FIG. 1.

8. The crystalline fumarate salt of any one of claims 4-6, wherein the crystalline fumarate salt has an XRPD pattern substantially the same as shown in FIG. 3.

9. The crystalline fumarate salt of any one of claims 4-6, wherein the crystalline fumarate salt has an XRPD pattern substantially the same as shown in FIG. 5.

10. The crystalline fumarate salt of any one of claims 4-6, wherein the crystalline fumarate salt is a crystalline hemi-fumarate salt.

11. The crystalline fumarate salt of any one of claims 4, 5, and 10, wherein the crystalline fumarate salt has an XRPD pattern substantially the same as shown in FIG. 8.

12. The crystalline fumarate salt of claim 4, 5, or 10, wherein the crystalline fumarate salt has an XRPD pattern substantially the same as shown in FIG. 14.

13. The crystalline fumarate salt of any one of claims 4-12, wherein the crystalline fumarate salt is an anhydrous crystalline fumarate salt.

14. The crystalline fumarate salt of any one of claims 4-12, wherein the crystalline fumarate salt is a crystalline hydrate.

15. The crystalline fumarate salt of claim 14, wherein the crystalline hydrate is a crystalline monohydrate.

16. A crystalline mono-fumarate salt of a compound of formula (I)

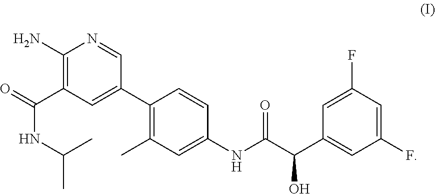 [SMILES: Cc1cc(NC(=O)[C@H](O)c2cc(F)cc(F)c2)ccc1-c1cnc(N)c(C(=O)NC(C)C)c1]

17. The crystalline mono-fumarate salt of claim 16, wherein the crystalline mono-fumarate salt has an XRPD pattern comprising one or more peaks at about 5.2° 2θ.

18. The crystalline mono-fumarate salt of claim 17, wherein the XRPD pattern further comprises one or more peaks at about 7.6° and about 14.4° 2θ.

19. The crystalline mono-fumarate salt of claim 17 or 18, wherein the XRPD pattern further comprises one or more peaks at about 15.8°, about 21.2°, about 24.5°, and about 25.0° 2θ.

20. The crystalline mono-fumarate salt of any one of claims 17-19, wherein the XRPD pattern further comprises one or more peaks at about 15.1°, about 16.7°, about 17.3°, about 17.9°, about 19.4°, about 19.9°, about 20.6°, and about 22.7° 2θ.

21. The crystalline mono-fumarate salt of any one of claims 17-20, wherein the XRPD pattern further comprises one or more peaks at about 30.4° and about 31.4° 2θ.

22. The crystalline mono-fumarate salt of any one of claims 16-21, wherein the crystalline mono-fumarate salt has an XRPD pattern substantially the same as shown in FIG. 1.

23. The crystalline mono-fumarate salt of any one of claims 16-21, wherein the crystalline mono-fumarate salt has an XRPD pattern substantially the same as shown in FIG. 3.

24. The crystalline mono-fumarate salt of any one of claims 16-21, wherein the crystalline mono-fumarate salt has an XRPD pattern substantially the same as shown in FIG. 5.

25. The crystalline mono-fumarate salt of any one of claims 16-24, wherein the crystalline mono-fumarate salt is a crystalline hydrate.

26. The crystalline mono-fumarate salt of claim 25, wherein the crystalline hydrate is a crystalline monohydrate.

27. Form A of a mono-fumarate salt of a compound of formula (I)

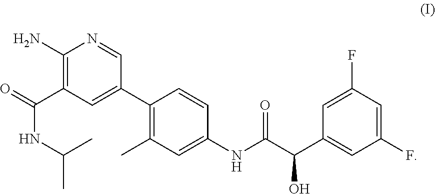 [SMILES: Cc1cc(NC(=O)[C@H](O)c2cc(F)cc(F)c2)ccc1-c1cnc(N)c(C(=O)NC(C)C)c1]

28. The Form A of the mono-fumarate salt of claim 27, wherein Form A of the mono-fumarate salt has an XRPD pattern comprises one or more peaks at about 5.0°, about 7.4°, and about 8.2° 2θ.

29. The Form A of the mono-fumarate salt of claim 28, wherein the XRPD pattern further comprises one or more peaks at about 9.7°, about 10.1°, about 10.7°, about 11.4°, about 11.9°, about 13.2°, about 14.7°, and about 14.8° 2θ.

30. The Form A of the mono-fumarate salt of claim 28 or 29, wherein the XRPD pattern further comprises one or more peaks at about 15.2°, about 16.1°, about 16.6°, about 17.5°, about 19.5°, about 20.2°, about 21.1°, about 23.9°, and about 24.2° 2θ.

31. The Form A of the mono-fumarate salt of any one of claims 28-30, wherein the XRPD pattern further comprises one or more peaks at about 17.9°, about 18.4°, about 19.2°, about 19.9°, about 20.8°, about 21.9°, about 22.9°, about 23.1°, about 23.5°, and about 24.8° 2θ.

32. The Form A of the mono-fumarate salt of any one of claims 28-31, wherein the XRPD pattern further comprises one or more peaks at about 25.1°, about 25.4°, about 27.4°, and about 27.8° 2θ.

33. The Form A of the mono-fumarate salt of any one of claims 28-32, wherein the XRPD pattern further comprises one or more peaks at about 28.9°, about 30.1°, about 30.4°, about 31.3°, about 31.5°, about 32.8°, and about 33.6° 2θ.

34. The Form A of the mono-fumarate salt of any one of claims 28-33, wherein about means ±0.3° 2θ.

35. The Form A of the mono-fumarate salt of any one of claims 27-34, wherein Form A of the mono-fumarate salt has an XRPD pattern substantially the same as shown in FIG. 1.

36. The Form A of the mono-fumarate salt of any one of claims 27-35, wherein Form A of the mono-fumarate salt has a differential scanning calorimetry (DSC) thermogram comprising one or more endotherms with peak onsets at about 32° C. and about 133° C.

37. The Form A of the mono-fumarate salt of any one of claims 27-36, wherein Form A of the mono-fumarate salt has a DSC thermogram substantially the same as shown in FIG. 2.

38. The Form A of the mono-fumarate salt of any one of claims 27-37, wherein Form A of the mono-fumarate salt exhibits a weight loss of less than or equal to about 1.2% wt. upon heating Form A of the mono-fumarate salt from about 25° C. to about 130° C.

39. The Form A of the mono-fumarate salt of any one of claims 27-38, wherein Form A of the mono-fumarate salt is a crystalline hydrate.

40. Form B of a mono-fumarate salt of a compound of formula (I)

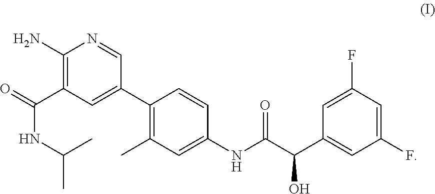 [SMILES: Cc1cc(NC(=O)[C@H](O)c2cc(F)cc(F)c2)ccc1-c1cnc(N)c(C(=O)NC(C)C)c1]

41. The Form B of the mono-fumarate salt of claim 40, wherein Form B of the mono-fumarate salt has an XRPD pattern comprising a peak at about 7.5° 2θ.

42. The Form B of the mono-fumarate salt of claim 41, wherein the XRPD pattern further comprises one or more peaks at about 4.7°, about 9.7°, about 13.3°, about 13.8°, about 14.0°, about 14.2°, and about 15.0° 2θ.

43. The Form B of the mono-fumarate salt of claim 41 or 42, wherein the XRPD pattern further comprises one or more peaks at about 17.4°, about 21.5°, about 22.2°, about 22.6°, about 23.1°, about 23.4°, about 24.4°, and about 24.7° 2θ.

44. The Form B of the mono-fumarate salt of any one of claims 41-43, wherein the XRPD pattern further comprises one or more peaks at about 15.4°, about 15.8°, about 16.6°, about 17.0°, about 17.8°, about 18.5°, about 18.8°, about 19.5°, about 19.8°, about 20.3°, about 20.6°, and about 24.1° 2θ.

45. The Form B of the mono-fumarate salt of any one of claims 41-44, wherein the XRPD pattern further comprises one or more peaks at about 26.8°, about 28.1°, and about 29.7° 2θ.

46. The Form B of the mono-fumarate salt of any one of claims 41-45, wherein the XRPD pattern further comprises one or more peaks at about 25.3°, about 26.0°, about 27.6°, about 30.2°, about 30.8°, about 31.5°, and about 33.5° 2θ.

47. The Form B of the mono-fumarate salt of any one of claims 41-46, wherein about means ±0.3° 2θ.

48. The Form B of the mono-fumarate salt of any one of claims 40-47, wherein Form B of the mono-fumarate salt has an XRPD pattern substantially the same as shown in FIG. 3.

49. The Form B of the mono-fumarate salt of any one of claims 40-48, wherein Form B of the mono-fumarate salt has a DSC thermogram comprising one or more endotherms with peak onsets at about 41° C., about 112° C., and about 122° C.

50. The Form B of the mono-fumarate salt of any one of claims 40-49, wherein Form B of the mono-fumarate salt has a DSC thermogram substantially the same as shown in FIG. 4.

51. The Form B of the mono-fumarate salt of any one of claims 40-50, wherein Form B of the mono-fumarate salt exhibits a weight loss of less than or equal to about 4.6% wt. upon heating Form B of the mono-fumarate salt from about 25° C. to about 155° C.

52. The Form B of the mono-fumarate salt of any one of claims 40-51, wherein Form B of the mono-fumarate salt is a crystalline hydrate.

53. Form C of a mono-fumarate salt of a compound of formula (I)

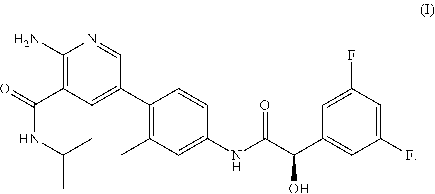 [SMILES: Cc1cc(NC(=O)[C@H](O)c2cc(F)cc(F)c2)ccc1-c1cnc(N)c(C(=O)NC(C)C)c1]

54. The Form C of the mono-fumarate salt of claim 53, wherein Form C of the mono-fumarate salt has an XRPD pattern comprising a peak at about 5.2° 2θ.

55. The Form C of the mono-fumarate salt of claim 54, wherein the XRPD pattern further comprises one or more peaks at about 7.6°, about 10.6°, about 13.8°, and about 14.4° 2θ.

56. The Form C of the mono-fumarate salt of claim 54 or 55, wherein the XRPD pattern further comprises one or more peaks at about 15.8°, about 21.2°, about 24.5°, and about 25.0° 2θ.

57. The Form C of the mono-fumarate salt of any one of claims 54-56, wherein the XRPD pattern further comprises one or more peaks at about 15.1°, about 16.7°, about 17.3°, about 17.9°, about 19.4°, about 19.9°, about 20.6°, and about 22.7° 2θ.

58. The Form C of the mono-fumarate salt of any one of claims 54-57, wherein the XRPD pattern further comprises one or more peaks at about 25.7°, about 26.6°, about 28.9°, about 30.4°, about 31.4°, and about 32.0° 2θ.

59. The Form C of the mono-fumarate salt of any one of claims 54-58, wherein about means ±0.3° 2θ.

60. The Form C of the mono-fumarate salt of any one of claims 53-59, wherein Form C of the mono-fumarate salt has an XRPD pattern substantially the same as shown in FIG. 5.

61. The Form C of the mono-fumarate salt of any one of claims 53-60, wherein Form C of the mono-fumarate salt has a DSC thermogram comprising one or more endotherms with peak onsets at about 30° C. and about 125° C.

62. The Form C of the mono-fumarate salt of any one of claims 53-61, wherein Form C of the mono-fumarate salt has a DSC thermogram substantially the same as shown in FIG. 6.

63. The Form C of the mono-fumarate salt of any one of claims 53-62, wherein Form C of the mono-fumarate salt exhibits a weight loss of less than or equal to about 0.9% wt. upon heating Form C of the mono-fumarate salt from about 25° C. to about 80° C.

64. A crystalline hemi-fumarate salt of a compound of formula (I)

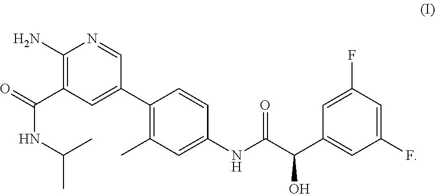 [SMILES: Cc1cc(NC(=O)[C@H](O)c2cc(F)cc(F)c2)ccc1-c1cnc(N)c(C(=O)NC(C)C)c1]

65. The crystalline hemi-fumarate salt of claim 64, wherein the crystalline hemi-fumarate salt has an XRPD pattern comprising one or more peaks at about 8.5°, about 11.0°, and about 12.0° 2θ.

66. The crystalline hemi-fumarate salt of claim 65, wherein the XRPD pattern further comprises one or more peaks at about 16.9°, about 17.9°, and about 24.1° 2θ.

67. The crystalline hemi-fumarate salt of claim 65 or 66, wherein the XRPD pattern further comprises one or more peaks at about 25.7°, about 27.7°, and about 34.1° 2θ.

68. The crystalline hemi-fumarate salt of any one of claims 65-67, wherein about means ±0.3° 2θ.

69. The crystalline hemi-fumarate salt of any one of claims 64-68, wherein the crystalline hemi-fumarate salt has an XRPD pattern substantially the same as shown in FIG. 8.

70. The crystalline hemi-fumarate salt of any one of claims 64-68, wherein the crystalline hemi-fumarate salt has an XRPD pattern substantially the same as shown in FIG. 13.

71. The crystalline hemi-fumarate salt of any one of claims 64-70, wherein the crystalline hemi-fumarate salt is an anhydrous crystalline form.

72. The crystalline hemi-fumarate salt of any one of claims 64-70, wherein the crystalline hemi-fumarate salt is a crystalline hydrate.

73. Form A of a hemi-fumarate salt of a compound of formula (I)

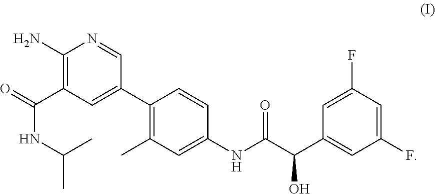 [SMILES: Cc1cc(NC(=O)[C@H](O)c2cc(F)cc(F)c2)ccc1-c1cnc(N)c(C(=O)NC(C)C)c1]

74. The Form A of the hemi-fumarate salt of claim 73, wherein Form A of the hemi-fumarate salt has an XRPD pattern comprising one or more peaks at about 9.9° and about 11.0° 2θ.

75. The Form A of the hemi-fumarate salt of claim 74, wherein the XRPD pattern further comprises one or more peaks at about 6.4°, about 8.3°, about 12.1°, about 13.2°, about 14.3°, and about 14.5° 2θ.

76. The Form A of the hemi-fumarate salt of claim 74 or 75, wherein the XRPD pattern further comprises one or more peaks at about 16.9°, about 17.2°, about 17.8°, about 18.5°, about 20.2°, about 22.1°, and about 22.5° 2θ.

77. The Form A of the hemi-fumarate salt of any one of claims 74-76, wherein the XRPD pattern further comprises one or more peaks at about 15.1°, about 16.1°, about 19.5°, about 19.8°, about 21.2°, about 21.5°, about 23.3°, about 24.3°, about 24.5°, and about 25.0° 2θ.

78. The Form A of the hemi-fumarate salt of any one of claims 74-77, wherein the XRPD pattern further comprises one or more peaks at about 25.5°, about 26.3°, about 27.2°, about 28.9°, and about 31.1° 2θ.

79. The Form A of the hemi-fumarate salt of any one of claims 74-78, wherein the XRPD pattern further comprises one or more peaks at about 27.8°, about 28.0°, about 28.3°, about 29.5°, about 31.9°, about 32.5°, about 32.7°, about 33.6°, about 34.1°, and about 34.4° 2θ.

80. The Form A of the hemi-fumarate salt of any one of claims 74-79, wherein about means #0.3° 2θ.

81. The Form A of the hemi-fumarate salt of any one of claims 73-80, wherein Form A of the hemi-fumarate salt has an XRPD pattern substantially the same as shown in FIG. 8.

82. The Form A of the hemi-fumarate salt of any one of claims 73-81, wherein Form A of the hemi-fumarate salt has a DSC thermogram comprising an endotherm with a peak onset at about 206° C.

83. The Form A of the hemi-fumarate salt of any one of claims 73-82, wherein Form A of the hemi-fumarate salt has a DSC thermogram substantially the same as shown in FIG. 9.

84. The Form A of the hemi-fumarate salt of any one of claims 73-83, wherein Form A of the hemi-fumarate salt exhibits a weight loss of less than or equal to about 0.5% wt. upon heating Form A of the hemi-fumarate salt from about 25° C. to about 150° C.

85. The Form A of the hemi-fumarate salt of any one of claims 73-84, wherein Form A of the hemi-fumarate salt is an anhydrous crystalline form.

86. Form B of a hemi-fumarate salt of a compound of formula (I)

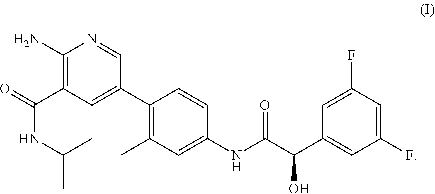 [SMILES: Cc1cc(NC(=O)[C@H](O)c2cc(F)cc(F)c2)ccc1-c1cnc(N)c(C(=O)NC(C)C)c1]

87. The Form B of the hemi-fumarate salt of claim 86, wherein Form B of the hemi-fumarate salt has an XRPD pattern comprising one or more peaks at about 5.4° and 8.7° 2θ.

88. The Form B of the hemi-fumarate salt of claim 87, wherein the XRPD pattern further comprises one or more peaks at about 10.4°, about 11.0°, about 11.9°, and about 12.6° 2θ.

89. The Form B of the hemi-fumarate salt of claim 87 or 88, wherein the XRPD pattern further comprises one or more peaks at about 16.5°, about 16.9°, about 17.4°, about 17.9°, and about 23.9° 2θ.

90. The Form B of the hemi-fumarate salt of any one of claims 87-89, wherein the XRPD pattern further comprises one or more peaks at about 25.7°, about 27.7°, and about 34.1° 2θ.

91. The Form B of the hemi-fumarate salt of any one of claims 87-90, wherein about means ±0.3° 2θ.

92. The Form B of the hemi-fumarate salt of any one of claims 86-91, wherein Form B of the hemi-fumarate salt has an XRPD pattern substantially the same as shown in FIG. 13.

93. The Form B of the hemi-fumarate salt of any one of claims 86-92, wherein Form B of the hemi-fumarate salt has a DSC thermogram comprising one or more endotherms with peak onsets at about 118° C., about 182° C., and about 202° C.

94. The Form B of the hemi-fumarate salt of any one of claims 86-93, wherein Form B of the hemi-fumarate salt has a DSC thermogram substantially the same as shown in FIG. 14.

95. The Form B of the hemi-fumarate salt of any one of claims 86-94, wherein Form B of the hemi-fumarate salt exhibits a weight loss of less than or equal to about 1.1% wt. upon heating Form B of the hemi-fumarate salt from about 25° C. to about 150° C.

96. The Form B of the hemi-fumarate salt of any one of claims 86-95, wherein Form B of the hemi-fumarate salt is a crystalline hydrate.

97. A pharmaceutical composition comprising:
(i) a fumarate salt of a compound of formula (I) 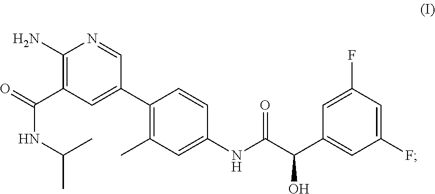 [SMILES: Cc1cc(NC(=O)[C@H](O)c2cc(F)cc(F)c2)ccc1-c1cnc(N)c(C(=O)NC(C)C)c1]
and
(ii) a pharmaceutically acceptable excipient.

98. A pharmaceutical composition comprising:
(i) a crystalline fumarate salt of a compound of formula (I) 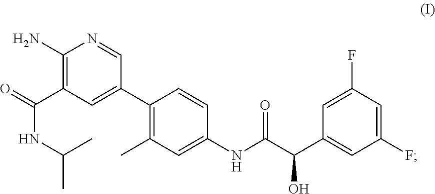 [SMILES: Cc1cc(NC(=O)[C@H](O)c2cc(F)cc(F)c2)ccc1-c1cnc(N)c(C(=O)NC(C)C)c1]
and
(ii) a pharmaceutically acceptable excipient.

99. The pharmaceutical composition of claim 98, wherein the crystalline fumarate salt is the crystalline fumarate salt of any one of claims 4-15.

100. A pharmaceutical composition comprising:
(i) a crystalline mono-fumarate salt of a compound of formula (I) 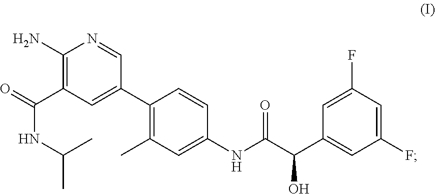 [SMILES: Cc1cc(NC(=O)[C@H](O)c2cc(F)cc(F)c2)ccc1-c1cnc(N)c(C(=O)NC(C)C)c1]
and
(ii) a pharmaceutically acceptable excipient.

101. The pharmaceutical composition of claim 100, wherein the crystalline mono-fumarate salt is the crystalline mono-fumarate salt of any one of claims 16-26.

102. A pharmaceutical composition comprising:
(i) the Form A of the mono-fumarate salt of the compound of formula (I) of any one of claims 27-39; and
(ii) a pharmaceutically acceptable excipient.

103. A pharmaceutical composition comprising:
(i) the Form B of the mono-fumarate salt of the compound of formula (I) of any one of claims 40-52; and
(ii) a pharmaceutically acceptable excipient.

104. A pharmaceutical composition comprising:
(i) the Form C of the mono-fumarate salt of the compound of formula (I) of any one of claims 53-63; and
(ii) a pharmaceutically acceptable excipient.

105. A pharmaceutical composition comprising:
(i) a crystalline hemi-fumarate salt of a compound of formula (I) 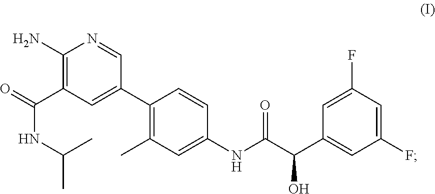 [SMILES: Cc1cc(NC(=O)[C@H](O)c2cc(F)cc(F)c2)ccc1-c1cnc(N)c(C(=O)NC(C)C)c1]
and
(ii) a pharmaceutically acceptable excipient.

106. The pharmaceutical composition of claim 105, wherein the crystalline hemi-fumarate salt is the crystalline hemi-fumarate salt of any one of claims 64-72.

107. A pharmaceutical composition comprising:
(i) the Form A of the hemi-fumarate salt of the compound of formula (I) of any one of claims 73-85; and
(ii) a pharmaceutically acceptable excipient.

108. A pharmaceutical composition comprising:
(i) the Form B of the hemi-fumarate salt of the compound of formula (I) of any one of claims 86-96; and
(ii) a pharmaceutically acceptable excipient.

109. A dosage form comprising a pharmaceutical composition of any one of claims 97-108.

110. The dosage form of claim 109, wherein the dosage form is a solid dosage form.

111. The dosage form of claim 109 or 110, wherein the dosage form is an oral dosage form.

112. The dosage form of any one of claims 109-111, wherein the dosage form is a capsule.

113. A method of treating a cancer in a subject in need thereof, comprising administering to the subject an effective amount of the fumarate salt of the compound of formula (I) of any one of claims 1-3; the crystalline fumarate salt of the compound of formula (I) of any one of claims 4-15; the crystalline mono-fumarate salt of the compound of formula (I) of any one of claims 16-26; the Form A of the mono-fumarate salt of the compound of formula (I) of any one of claims 27-39; the Form B of the mono-fumarate salt of the compound of formula (I) of any one of claims 40-52; the Form C of the mono-fumarate salt of the compound of formula (I) of any one of claims 53-63; the crystalline hemi-fumarate salt of the compound of formula (I) of any one of claims 64-72; the Form A of the hemi-fumarate salt of the compound of formula (I) of any one of claims 73-85; the Form B of the mono-fumarate salt of the compound of formula (I) of any one of claims 86-96; or the pharmaceutical composition of any one of claims 97-108.

114. The method of claim 113, wherein the cancer is selected from the group consisting of a bladder cancer, a breast cancer, a carcinoma, a cervical cancer, a colorectal cancer, a gastric cancer, a hepatocellular cancer, a kidney cancer, a lung cancer, a neuroendocrine cancer, an ovarian cancer, a pancreatic cancer, a prostate cancer, a skin cancer, and a thyroid cancer.

115. The method of claim 114, wherein the breast cancer is triple negative breast cancer or metastatic breast cancer; the carcinoma is carcinoma of unknown primary (CUP), endometrial carcinoma, head and neck squamous cell carcinoma, Merkel cell carcinoma, squamous cell carcinoma, or urothelial carcinoma; the gastric cancer is adenocarcinoma or gastrointestinal stromal tumor; the kidney cancer is a renal cell carcinoma (RCC); the lung cancer is small cell lung cancer (SCLC) or non-small cell lung cancer; the hepatocellular cancer is a hepatocellular carcinoma; or the skin cancer is malignant melanoma.

116. The method of claim 113, wherein the cancer is selected from the group consisting of a renal cell carcinoma, a gastric cancer, a breast cancer, and a small-cell lung cancer.

117. The method of claim 113, wherein the cancer is selected from the group consisting of Classical Hodgkin's lymphoma, primary thymic mediastinal lymphoma, multiple myeloma, and a B cell malignancy.

118. The method of claim 117, wherein the B cell malignancy is non-Hodgkin lymphoma or chronic lymphocytic leukemia.
